(19) United States
(12) Patent Application Publication
(10) Pub. No.: US 2025/0353883 A1
(43) Pub. Date: Nov. 20, 2025

(54) NOVEL RECOMBINANT AAV VP2 FUSION POLYPEPTIDES

(71) Applicant: Novartis AG, Basel (CH)

(72) Inventors: Agostino CIRILLO, Birsfelden (CH); Hilmar EBERSBACH, Zürich (CH); Mir HOSSAIN, Arlington, MA (US)

(21) Appl. No.: 18/862,684

(22) PCT Filed: May 4, 2023
(86) PCT No.: PCT/IB2023/054639

§ 371 Date: Nov. 4, 2024

Related U.S. Application Data
(60) Provisional application No. 63/364,287, filed on May 6, 2022.

(51) Int. Cl. C07K 14/005; C12N 15/86

(52) U.S. Cl. CPC C07K 14/005; C12N 15/86; C07K 2319/00; C12N 2750/14122; C12N 2750/14143

(57) ABSTRACT

This disclosure relates to adeno-associated virus (AAV) VP2 fusion polypeptides comprising an AAV VP2 capsid polypeptide and a polypeptide ligand. The disclosure further relates to rAAV virions comprising such AAV VP2 fusion polypeptides and libraries of nucleic acids encoding such AAV VP2 fusion polypeptides, pharmaceutical compositions comprising such rAAV virions, and related methods and uses.

### TECHNICAL FIELD

The present disclosure provides adeno-associated virus (AAV) VP2 fusion polypeptides comprising an AAV VP2 capsid polypeptide and a polypeptide ligand for improved targeting of AAVs in gene therapy approaches. The disclosure further provides rAAV virions comprising such AAV VP2 fusion polypeptides and libraries of nucleic acids encoding such AAV VP2 fusion polypeptides, and related compositions, methods and uses.

### SEQUENCE LISTING

The instant application contains a Sequence Listing which has been submitted electronically in XML format and is hereby incorporated by reference in its entirety. Said XML copy, created on Jul. 24, 2025, is named PAT059281-US-PCT_SL and is 98,304 bytes in size.

### BACKGROUND

Adeno-associated virus (AAV) vectors are among the most promising gene transfer vectors due to their excellent safety and efficacy profile. Established features of AAV vectors that distinguish them from other vectors include stable long-term expression, broad host range, ability to transduce proliferating and post-mitotic cells, high titers of AAV vectors produced in tissue cultures, derivation from a nonpathogenic virus and low immunogenicity of both wild type virus and recombinant vectors.

However, due to their broad tropism, transduction efficacy of many target organs is low and hence high vector doses need to be applied. It has become increasingly clear that the full potential of this vector system will only be realized with modified AAV vectors exhibiting improved cell transduction rate and specificity leading to an improved safety profile.

Retargeting attempts have focused on the variable regions forming loops of the protrusions due to their exposed positions and their function in receptor binding. However, these sites only accept insertion of small peptides. US20180163229 discloses variant AAV capsid polypeptides comprising a DARPin fused to the N-terminus of AAV VP2.

There remains a high unmet need for further variant AAV capsid polypeptides that can mediate improved AAV characteristics for gene therapy, such as increased transduction of and/or increased tropism in at least one tissue or cell type, improved cell-type selectivity and/or targeting specificity.

### SUMMARY OF THE DISCLOSURE

The present disclosure provides adeno-associated virus (AAV) VP2 fusion polypeptides comprising an AAV VP2 capsid polypeptide and a polypeptide ligand, for example wherein the polypeptide ligand is fused to the N-terminus of the AAV VP2 capsid polypeptide, either directly or via a peptide linker. The polypeptide linker has a molecular weight of up to 10 kDa. When used for rAAV virion assembly, typically together with AAV VP1 and/or VP3 capsid polypeptides, the AAV VP2 fusion polypeptides provided herein may show good decoration levels, meaning that a satisfactory number of AAV VP2 fusion polypeptides is incorporated in the rAAV virion.

The AAV VP2 fusion polypeptides described herein may mediate improved transduction of and/or increased tropism in at least one tissue or cell type, relative to an AAV VP2 capsid polypeptide which is not fused to said polypeptide ligand, but which is otherwise identical to the VP2 capsid polypeptide comprised in the AAV VP2 fusion polypeptide. Improved transduction of and/or increased tropism in at least one tissue or cell type may be mediated by the polypeptide ligand, which may have the ability to bind to a cell surface molecule expressed on the at least one tissue or cell type. rAAV virions comprising the AAV VP2 fusion polypeptide provided herein and displaying the polypeptide ligand on their surface can therefore be used for cell-type specific gene delivery during therapeutic applications and applications in basic research since they provide high cell type selectivity and/or targeting specificity allowing restricted biodistribution and safe gene transfer.

Thus, in one aspect, provided herein are adeno-associated virus (AAV) VP2 fusion polypeptides comprising, e.g., consisting of, an AAV VP2 capsid polypeptide and a polypeptide ligand, wherein the polypeptide ligand is fused to the N-terminus of the AAV VP2 capsid polypeptide and wherein the polypeptide ligand has a molecular weight of up to 10 kDa.

In other aspects, provided herein are nucleic acids encoding such AAV VP2 fusion polypeptides and cells comprising such AAV VP2 fusion polypeptide or nucleic acids encoding same.

In other aspects, provided herein are rAAV virions comprising the AAV VP2 fusion polypeptide disclosed herein and pharmaceutical compositions comprising such rAAV virions.

In other aspects, provided herein are libraries of nucleic acid constructs encoding AAV VP2 fusion polypeptides disclosed herein and methods of generating an AAV VP2 fusion polypeptide with desired characteristics using such library.

In other aspects, provided herein are methods of treatment using rAAV virions comprising the AAV VP2 fusion polypeptide disclosed herein and pharmaceutical compositions comprising such rAAV virions.

In another aspect, provided herein is an AAV VP2 capsid polypeptide, wherein a) the AAV VP2 capsid polypeptide is of the AAV serotype AAV1 and comprises at least one amino acid substitution selected from the group consisting of E147S, P185G, P166R, M211V, G199R, D213A, T162R, and P191N relative to the VP1 amino acid sequence of SEQ ID NO: 7, or any combination thereof, in particular wherein the AAV VP2 capsid polypeptide comprises the amino acid substitution(s) D213A, T162R, and/or P191N, or wherein b) the AAV VP2 capsid polypeptide is of an AAV serotype other than AAV1 and comprises at least one amino acid substitution corresponding to at least one of the amino acid substitution selected from the group consisting of E147S, P185G, P166R, M211V, G199R, D213A, T162R, and P191N relative to the VP1 amino acid sequence of SEQ ID NO: 7, particularly wherein bi) the AAV VP2 capsid polypeptide is of the AAV serotype 8 and comprises at least one amino acid substitution selected from the group consisting of E147S, P186G, P167R, M212V, G200R, D214A, K163R, and P192N relative to the VP1 amino acid sequence of SEQ ID NO: 4, or any combination thereof, in particular wherein the AAV VP2 capsid polypeptide comprises the amino acid substitution(s) D214A, K163R and/or P192N, or wherein bii) the AAV VP2 capsid polypeptide is of the AAV serotype 9 and comprises at least one amino acid substitution selected from the group consisting of E147S, P185G, P166R, G199R, D213A, S162R and P191N relative to the VP1 amino acid sequence of SEQ ID NO: 5, or any combination thereof, in particular wherein the AAV VP2 capsid polypeptide comprises the amino acid substitution(s) D213A, S162R and/or P191N.

### DETAILED DESCRIPTION

### Definitions

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated, that all numerical designations are preceded by the term “about.” The term “about” in relation to a numerical value X means, for example, X±15%, including all the values within this range. It also is to be understood, although not always explicitly stated, that the reagents described herein are merely examples and that equivalents of such are known in the art.

Throughout this specification and the claims which follow, unless the context requires otherwise, the word “comprise”, and variations such as “comprises” and “comprising”, are used herein in their open-ended and non-limiting sense unless otherwise noted.

When used herein “consisting of” excludes any element, step, or ingredient not specified in the aspect, embodiment and/or claim element. When used herein, “consisting essentially of” does not exclude materials or steps that do not materially affect the basic and novel characteristics of the aspect, embodiment and/or claim.

The terms “peptide,” “polypeptide,” and “protein” are used interchangeably, and refer to a compound comprised of amino acid residues covalently linked by peptide bonds. A protein or peptide typically contains at least two amino acids or amino acid variants, and no limitation is placed on the maximum number of amino acids that can be comprised in a protein or polypeptide sequence. Polypeptides include any peptide or protein comprising two or more amino acids or variants joined to each other by peptide bonds. The terms include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The terms “polynucleotide” and “nucleic acid” are used interchangeably herein and refer to a polymeric form of nucleotides of any length. They may include one or more of ribonucleotides or deoxyribonucleotides. Thus, this term includes, but is not limited to, single-, double-, or multi-stranded deoxyribonucleic acids (DNA) or ribonucleic acids (RNA), genomic DNA, cDNA, DNA-RNA hybrids, or a polymer comprising purine and pyrimidine bases or other natural, chemically or biochemically modified, non-natural, or derivatized nucleotide bases, e.g. analogues of natural nucleotides that have similar binding properties as the reference nucleic acid and are metabolized in a manner similar to naturally occurring nucleotides, locked nucleic acids (LNA), peptide nucleic acids (PNA). Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “sequence identity” and “sequence homology” are used interchangeably herein, and as used in connection with a polynucleotide or polypeptide, refer to the percentage of bases or amino acids that are the same, and are in the same relative position, when comparing or aligning two sequences of polynucleotides of polypeptides. Thus, when a subunit position in both of the two molecules is occupied by the same monomeric subunit, e.g., if a position in each of two DNA molecules is occupied by adenine, then they are homologous or identical at that position. The homology between two sequences is a direct function of the number of matching or homologous positions, e.g., if half (e.g., five positions in a polymer ten subunits in length) of the positions in two sequences are homologous, the two sequences are 50% homologous; if 90% of the positions (e.g., 9 of 10) are matched or homologous, the two sequences are 90% homologous. Sequence identity can be determined in a number of different manners. For instance, percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. Sequences may be aligned using various methods and computer programs (e.g., BLAST, T-COFFEE, MUSCLE, MAFFT, etc.). See, e.g., Altschul et al., (1990) J. Mol. Bioi., 215:403-10.

The term “naturally-occurring” or “unmodified” as used herein as applied to, e.g., a nucleic acid, a polypeptide, a cell, or an organism, is one found in nature. For example, a polypeptide or polynucleotide sequence that is present in an organism (such as a virus) is naturally occurring whether present in that organism or isolated from one or more components of the organism.

The term “variant” with regard to polynucleotides or polypeptides refers to polynucleotides or polypeptides differing in at least one residue, i.e., at least one nucleotide for polynucleotides and at least one amino acid for polypeptides, from a parent polynucleotide or polypeptide, also referred to as non-variant polynucleotide or polypeptide sequence.

The term “isolated” in reference to a nucleic acid, polypeptide or virus discussed herein refers to a nucleic acid, polypeptide or virus that has been separated from one or more of the components normally found associated with it in its natural environment. For example, a nucleic acid or a peptide naturally present in a living animal is not “isolated,” but the same nucleic acid or peptide partially or completely separated from the coexisting materials of its natural state is “isolated.” The separation may comprise removal from a larger nucleic acid (e.g., from a gene or chromosome) or from other proteins or molecules normally in contact with the nucleic acid or protein. The term encompasses but does not require complete isolation. Thus, an isolated nucleic acid or protein can exist in substantially purified form, or can exist in a non-native environment such as, for example, a host cell.

As used herein, an isolated nucleic acid comprising a “heterologous nucleic acid sequence” refers to an isolated nucleic acid comprising a portion (i.e., the heterologous nucleic acid portion) that is not normally found operably linked to one or more other components of the isolated nucleic acid in a natural context. For instance, the heterologous nucleic acid may comprise a nucleic acid sequence not originally found in a cell, bacterial cell, virus, or organism from which other components of the isolated nucleic acid (e.g., the promoter) naturally derive or where the other components of the isolated nucleic acid (e.g., the promoter) are not naturally found operably linked with the heterologous nucleic acid in the cell, bacterial cell, virus, or organism. In some embodiments the heterologous nucleic acid includes a transgene. As used herein, a “transgene” is a nucleic acid sequence that encodes a molecule of interest (for example, a therapeutic protein, therapeutic RNA molecule, or a reporter protein) that is not originally associated with one or more components of the nucleic acid molecule. In some embodiments, the heterologous nucleic acid sequence encodes a human protein. In some embodiments, the heterologous nucleic acid sequence encodes an RNA sequence, e.g., an shRNA.

As used herein, the term “reporter sequence” refers to a nucleic acid sequence encoding a reporter protein, such a s a fluorescent protein or an oxidative enzyme, which makes it possible to visualize infection with an rAAV vector comprising such reporter sequence, i.e., to monitor successful transduction of the target cell or target tissue based on the expression of the reporter protein. A preferred oxidative enzyme is firefly luciferase; exemplary fluorescent proteins include GFP and variants thereof, such as eGFP, and sfCherry. A reporter sequence may be packaged into an rAAV virion in addition to or instead of a therapeutic transgene or a nucleic acid encoding the AAV VP2 fusion polypeptide disclosed herein.

The term “barcode sequence”, as used herein, refers to a unique oligonucleotide sequence (e.g., 5, 6, 7, 8, 9, 10, 12, 15, 20, 25, 30, 50, 75, 100 nucleotides) having a particular sequence, that is used as a means of identifying a nucleic acid sequence in which it is incorporated. For instance, the barcode may be used as a means of distinguishing or identifying individual members (e.g., variants) in a library.

A DNA sequence or DNA polynucleotide sequence that “encodes” a particular RNA is a sequence of DNA that is capable of being transcribed into RNA. A DNA polynucleotide may encode an RNA (mRNA) that is translated into a protein, or a DNA polynucleotide may encode an RNA that is not translated into a protein (e.g., tRNA, rRNA, or a guide RNA; also called “non-coding” RNA or “ncRNA”). A DNA sequence or DNA polynucleotide sequence may also “encode” a particular polypeptide or protein sequence, wherein, for example, the DNA directly encodes an mRNA that can be translated into the polypeptide or protein sequence. A “protein coding sequence” or a sequence that encodes a particular protein or polypeptide is a nucleic acid sequence that is capable of being transcribed into mRNA (in the case of DNA) and translated (in the case of mRNA) into a polypeptide in vitro or in vivo when placed under the control of appropriate regulatory sequences. The boundaries of the coding sequence may be determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “promoter” or “promoter sequence” as used herein is a DNA regulatory sequence capable of facilitating transcription (e.g., capable of causing detectable levels of transcription and/or increasing the detectable level of transcription over the level provided in the absence of the promoter) of an operably linked coding or non-coding sequence, e.g., of a downstream (3′ direction) coding or non-coding sequence, e.g., through binding RNA polymerase. In some embodiments, the promoter sequence is bound at its 3′ terminus by the transcription initiation site and extends upstream (5′ direction) to include the minimum number of bases or elements to initiate transcription at levels detectable above background. In some embodiments, a promoter sequence may comprise a transcription initiation site, as well as protein binding domains responsible for the binding of RNA polymerase. In addition to sequences sufficient to initiate transcription, a promoter may also include sequences of other regulatory elements that are involved in modulating transcription (e.g., enhancers, Kozak sequences and introns). Various promoters, including inducible promoters and constitutive promoters, may be used to drive expression from the vectors disclosed herein. Examples of promoters known in the art that may be used in some embodiments, e.g., in nucleic acid molecules and vectors disclosed herein, include the CMV promoter, the 173CMV promoter, the HCMV promoter, the CBh promoter, the CAG promoter, the mCCT promoter, the CBA promoter, the smCBA promoter and those promoters derived from an immunoglobulin gene, SV40, or other tissue specific genes (e.g: RLBP1, RPE, VMD2). In addition, standard techniques are known in the art for creating functional promoters by mixing and matching known regulatory elements. Fragments of promoters, e.g., those that retain at least minimum number of bases or elements to initiate transcription at levels detectable above background, may also be used.

In some embodiments, a promoter can be a constitutively active promoter (i.e., a promoter that constitutively drives expression in any cell type and/or under any conditions). In other embodiments, a promoter can be a constitutively active promoter in a particular tissue context, e.g., in neurons, in cardiac cells, etc. In other embodiments, a promoter can be an inducible promoter (i.e., a promoter whose activity is controlled by an external stimulus, e.g., the presence of a particular temperature, compound, or protein). In some embodiments, a promoter may be a spatially restricted promoter that can drive activity or not depending on the physical context in which the promoter is found. Non-limiting examples of spatially restricted promoters include tissue specific promoter, cell type specific promoter, etc. In some embodiments, a promoter may be a temporally restricted promoter that drives expression depending on the temporal context in which the promoter is found. For example, a temporally restricted promoter may drive expression only at specific stages of embryonic development or during specific stages of a biological process. Non-limiting examples of temporally restricted promoters include hair follicle cycle promoters in mice.

In some embodiments, the promoter is tissue-specific such that, in a multi-cellular organism, the promoter drives expression only in a subset of specific cells. For example, tissue-specific promoters include, but are not limited to, neuron-specific promoters, adipocyte-specific promoters, cardiomyocyte-specific promoters, smooth muscle-specific promoters, photoreceptor-specific promoters, etc. A neuron-specific promoter refers to a promoter that, when administered e.g., peripherally, directly into the central nervous system (CNS), or delivered to neuronal cells, including in vitro, ex vivo, or in vivo, preferentially drives or regulates expression of an operably linked heterologous nucleic acid, e.g., one encoding a protein or peptide or shRNA of interest, in neurons as compared to expression in non-neuronal cells.

The term “operably linked” refers to a functional relationship between two or more polynucleotide (e.g., DNA) segments. Typically, the term refers to the functional relationship of a transcriptional regulatory sequence and a sequence to be transcribed. For example, a promoter or enhancer sequence is operably linked to a coding sequence if it, e.g., stimulates or modulates the transcription of the coding sequence in an appropriate host cell or other expression system. Generally, promoter transcriptional regulatory sequences that are operably linked to a sequence are contiguous to that sequence or are separated by short spacer sequences, i.e., they are cis-acting. However, some transcriptional regulatory sequences, such as enhancers, need not be physically contiguous or located in close proximity to the coding sequences whose transcription they enhance.

The terms “DNA regulatory sequences,” “control elements,” and “regulatory elements,” used interchangeably herein, refer to transcriptional and translational control sequences, such as promoters, enhancers, silencers, polyadenylation signals, terminators, protein degradation signals, and the like, that provide for and/or regulate transcription of a non-coding sequence (e.g., a short hairpin RNA) or a coding sequence (e.g., a transgene) and/or regulate translation of an encoded polypeptide.

The terms “polyadenylation (polyA) signal sequence” and “polyadenylation sequence” refer to a regulatory element that provides a signal for transcription termination and addition of an adenosine homopolymeric chain to the 3′ end of an RNA transcript. The polyadenylation signal may comprise a termination signal (e.g., an AAUAAA sequence or other non-canonical sequences) and optionally flanking auxiliary elements (e.g., a GU-rich element) and/or other elements associated with efficient cleavage and polyadenylation. The polyadenylation sequence may comprise a series of adenosines attached by polyadenylation to the 3′ end of an mRNA. Exemplary polyA signal sequences are BGH and SV40 polyA signal sequences. In some embodiments, DNA regulatory sequences or control elements are tissue-specific regulatory sequences.

The term “post-transcriptional regulatory element” (“PRE”) refers to one or more regulatory elements that, when transcribed into mRNA, regulate gene expression at the level of the mRNA transcript. Examples of such post-transcriptional regulatory elements may include sequences that encode micro-RNA binding sites, RNA binding protein binding sites, etc. Examples of post-transcriptional regulatory element that may be used with the nucleic acid molecules and vectors disclosed herein include the woodchuck hepatitis post-transcriptional regulatory element (WPRE), and the hepatitis post-transcriptional regulatory element (HPRE).

The term “intron” refers to nucleic acid sequence(s), e.g., those within an open reading frame, that are noncoding for one or more amino acids of a polypeptide transcript (e.g., protein of interest) expressed from the nucleic acid. Intronic sequences may be transcribed from DNA into RNA (i.e., may be present in the pre-mRNA), but may be removed before the protein is expressed from the mature mRNA, e.g., through splicing.

The term “exon” refers to nucleic acid sequence(s), e.g., those within an open reading frame (ORF), that are coding for one or more amino acids of a transcript (e.g., a protein of interest) expressed from a nucleic acid. Exonic sequences may be transcribed from DNA into RNA (i.e., may be present in the pre-mRNA), and also may be present in a mature mRNA (i.e., the processed form of RNA (e.g., after splicing)) that is translated to a polypeptide.

In some embodiments, a “vector” is any genetic element (e.g., DNA, RNA, or a mixture thereof) that contains a nucleic acid of interest (e.g., a transgene) that is capable of being expressed in a host cell, e.g., a nucleic acid of interest within a larger nucleic acid sequence or structure suitable for delivery to a cell, tissue, and/or organism, such as a plasmid, phage, transposon, cosmid, chromosome, virus, virion, etc. For instance, a vector may comprise an insert (e.g., a heterologous nucleic acid comprising a transgene encoding a gene to be expressed or an open reading frame of that gene) and one or more additional elements suitable for delivering or controlling expression of the insert. The vector may be capable of replication and/or expression, e.g., when associated with the proper control elements, and it may be capable of transferring genetic information between cells. In some embodiments, a vector may be a vector suitable for expression in a host cell, e.g., an AAV vector. In some embodiments, a vector may be a plasmid suitable for expression and/or replication, e.g., in a cell or bioreactor. In some embodiments, vectors designed specifically for the expression of a heterologous nucleic acid sequence, e.g., a transgene encoding a protein of interest, shRNA, and the like, in the target cell may be referred to as expression vectors, and generally have a promoter sequence that drives expression of the transgene. In other embodiments, vectors, e.g., transcription vectors, may be capable of being transcribed but not translated, meaning that they can be replicated in a target cell but not expressed. Transcription vectors may be used to amplify their insert.

The term “expression vector” refers to a vector comprising a polynucleotide comprising expression control sequences operatively linked to a nucleotide sequence to be expressed. An expression vector may comprise sufficient cis-acting elements for expression, alone or in combination with other elements for expression supplied by the host cell or in an in vitro expression system. Expression vectors include, e.g., cosmids, plasmids (e.g., naked or contained in liposomes) and viruses (e.g., lentiviruses, retroviruses, adenoviruses, and adeno-associated viruses) that incorporate the recombinant polynucleotide.

The term “plasmid” refers to a non-chromosomal (and typically double-stranded) DNA sequence comprising an intact “replicon” such that the plasmid is replicated in a host cell. A plasmid may be a circular nucleic acid. When the plasmid is placed within a unicellular organism, the characteristics of that organism are changed or transformed as a result of the DNA of the plasmid. For example, a plasmid carrying the gene for tetracycline resistance (TcR) transforms a cell previously sensitive to tetracycline into one which is resistant to it.

The term “recombinant virus” as used herein is intended to refer to a non-wild-type and/or artificially produced recombinant virus (e.g., a parvovirus, adenovirus, lentivirus or adeno-associated virus etc.) that comprises a transgene or other heterologous nucleic acid. The recombinant virus may comprise a recombinant viral vector (e.g., comprising a transgene) packaged within a viral (e.g.: AAV) capsid. A specific type of recombinant virus may be a “recombinant adeno-associated virus”, or “rAAV”. The recombinant viral genome packaged in the viral capsid may be a viral vector. In some embodiments, the recombinant viruses disclosed herein comprise viral vectors (e.g., comprising a transgene of interest, e.g., as described herein). Examples of viral vectors include but are not limited to an adeno-associated viral (AAV) vector, a chimeric AAV vector, an adenoviral vector, a retroviral vector, a lentiviral vector, a DNA viral vector, a herpes simplex viral vector, a baculoviral vector, or any mutant or derivative thereof.

“AAV” is an abbreviation for adeno-associated virus and may be used to refer to the virus itself or derivatives thereof. The term covers all subtypes and both naturally occurring and recombinant forms, except where explicitly stated otherwise. The term “rAAV” refers to recombinant adeno-associated virus or recombinant AAV vector.

As used herein, the term “AAV vector” refers to a vector derived from or comprising one or more nucleic acid sequences derived from an adeno-associated virus serotype, including without limitation, an AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5 viral vector. AAV vectors may have one or more of the AAV wild-type genes deleted in whole or part, e.g., the rep and/or cap genes, while retaining, e.g., functional flanking inverted terminal repeat (“ITR”) sequences. In some embodiments, an AAV vector may be packaged in a protein shell or “capsid,” e.g., comprising one or more AAV capsid proteins, which may provide a vehicle for delivery of vector nucleic acid to the nucleus of target cells. In some embodiments, an AAV vector comprises one or more AAV ITR sequences (e.g., AAV2 ITR sequences). In some embodiments, an AAV vector comprises one or more AAV ITR sequences (e.g., AAV2 ITR sequences) but does not contain any additional viral nucleic acid sequence. In some embodiments, the AAV vector components (e.g., ITRs) are derived from a different serotype virus than the rAAV capsid (for example, the AAV vector may comprise ITRs derived from AAV2 and the AAV vector may be packaged into an AAV9 capsid). Embodiments of these vector constructs are provided, e.g., in WO2019/094253 (PCT/US2018/058744), which is incorporated herein by reference in its entirety.

rAAV vectors include single stranded AAV vectors and self-complementary AAV vectors (scAAV). scAAV is termed “self-complementary” because at least a portion of the vector (e.g., at least a portion of the coding region) of the scAAV forms an intra-molecular double-stranded DNA. In some embodiments, the rAAV is an scAAV. In other embodiments, the rAAV is a single stranded AAV. In some embodiments, a viral vector is engineered from a naturally occurring adeno-associated virus (AAV) to provide an scAAV for use in gene therapy. Embodiments of these vector constructs and methods of preparing and purifying them are provided, e.g., in WO2019/094253 (PCT/US2018/058744), which is incorporated herein by reference in its entirety.

As used herein, a “virus” or “virion” indicates a viral particle, comprising a viral vector, e.g., alone or in combination with one or more additional components such as one or more viral capsids. For instance, an AAV virus may comprise, e.g., a linear, single-stranded AAV nucleic acid genome associated with an AAV capsid protein coat.

In some embodiments, terms such as “virus,” “virion,” “AAV virus,” “recombinant AAV virion,” “rAAV virion,” “AAV vector particle,” “full capsids,” “full particles,” and the like refer to infectious, replication-defective virus, e.g., those comprising an AAV protein shell encapsidating a heterologous nucleotide sequence of interest, e.g., in a viral vector which is flanked on one or both sides by AAV ITRs. An rAAV virion may be produced in a suitable host cell which comprises sequences, e.g., one or more plasmids, specifying an AAV vector, alone or in combination with nucleic acids encoding AAV helper functions and accessory functions (such as the rep and the cap gene), e.g., on the same or additional plasmids. In some embodiments, the host cell is rendered capable of encoding AAV polypeptides that provide for packaging the AAV vector (containing a recombinant nucleotide sequence of interest) into infectious recombinant virion particles for subsequent gene delivery.

“Packaging” refers to a series of intracellular events resulting in the assembly of AAV virions or AAV particles which encapsidate a nucleic acid sequence. Packaging can refer to encapsidation of nucleic acid sequence into a capsid comprising the AAV VP2 fusion polypeptide disclosed herein.

An “infectious” virion, virus or viral particle is one comprising a polynucleotide component deliverable into a cell tropic for the viral species. The term does not necessarily allow any conclusion on the replication capacity of the virus. As used herein, an “infectious” virus or viral particle is one that upon accessing a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are well known in the art. Viral infectivity can be expressed as the ratio of infectious viral particles to total viral particles. Total viral particles can be expressed as the number of viral genome copies. The ability of a viral particle to express a heterologous nucleic acid in a cell can be referred to as “transduction”. The ability of a viral particle to express a heterologous nucleic acid in a cell can be assayed using a number of techniques, including assessment of a marker gene, such as a green fluorescent protein (GFP) assay, (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA) or fluorescence-activated cell sorting (FACS).

A “replication-competent” virion or virus (e.g., a replication-competent AAV) refers to an infectious virus which is replicable in an infected cell (i.e., in the presence of a helper virus or helper virus functions). In the case of AAV, replication competence generally requires the presence of functional AAV packaging genes, i.e., cap and rep genes. In some embodiments, AAV vectors, as described herein, lack one or more AAV packaging genes and are replication-incompetent in mammalian cells (such as in human cells). In some embodiments, AAV vectors lack any AAV packaging gene sequences, minimizing the possibility of generating replication competent AAV by recombination between AAV packaging genes and an incoming AAV vector.

The terms “inverted terminal repeat” or “ITR” refer to a stretch of nucleotide sequences that can form a T-shaped palindromic structure, e.g., in adeno-associated viruses (AAV) and/or recombinant adeno-associated viral vectors (rAAV). Muzyczka et al., (2001) Fields Virology, Chapter 29, Lippincott Williams & Wilkins. In recombinant AAV vectors, these sequences may play a functional role in genome packaging and in second-strand synthesis. In some embodiments, the AAV vector includes one or more ITRs which are mutated or truncated.

The term “cap gene” or “capsid gene” refers to a nucleic acid sequence that encodes capsid proteins that form, or contribute to the formation of, the capsid, or protein shell, of the virus. In the case of AAV, the capsid proteins are typically VP1, VP2, and VP3. For other parvoviruses, the names and numbers of the capsid proteins can differ. The terms “AAV VP1 capsid polypeptide”, “AAV VP2 capsid polypeptide” and “AAV VP3 capsid polypeptide” as used herein include wild type AAV capsid polypeptides as well as variants and fragments thereof, in particular functional variants and fragments thereof. Functional AAV capsid polypeptide variants and fragments can be used in AAV capsid assembly.

The term “rep gene” refers to a nucleic acid sequence that encodes the non-structural proteins (rep78, rep68, rep52 and rep40) required for the replication and production of AAV.

The term “AAV helper function” refers to AAV-derived coding sequences which can be expressed to provide AAV gene products, e.g., those that function in trans for productive AAV replication. For instance, AAV helper functions may include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions may be used herein to complement AAV functions in trans that are missing from AAV vectors.

The term “AAV helper construct” refers generally to a nucleic acid molecule that includes nucleotide sequences providing or encoding proteins or nucleic acids that provide AAV functions deleted from an AAV vector, e.g., a vector for delivery of a nucleotide sequence of interest to a target cell or tissue. AAV helper constructs are commonly used to provide transient expression of AAV rep and/or cap genes to complement missing AAV functions for AAV replication. Typically, helper constructs lack AAV ITRs and can neither replicate nor package themselves. AAV helper constructs may be in the form of a plasmid, phage, transposon, cosmid, virus, or virion. A number of AAV helper constructs have been disclosed, such as the commonly used plasmids pAAV/Ad and plM29+45 which encode both Rep and Cap expression products. See, e.g., Samulski et al., (1989) J. Virol., 63:3822-3828; McCarty et al., (1991) J. Virol., 65:2936-2945. A number of other vectors have been disclosed which encode Rep and/or Cap expression products. See, e.g., U.S. Pat. Nos. 5,139,941 and 6,376,237. Embodiments of these vector constructs and methods of preparing and purifying them are provided, e.g., in WO2019/094253 (PCT/US2018/058744), which is incorporated herein by reference in its entirety.

A “helper virus” for AAV refers to a virus allowing AAV replication and packaging in a mammalian cell. A variety of such helper viruses for AAV are known in the art, including adenoviruses, herpesviruses and poxviruses such as vaccinia. Adenoviruses encompass a number of different subgroups, although Adenovirus type 5 of subgroup C is most commonly used as a helper virus.

The term “helper virus function(s)” refers to function(s) encoded in a helper virus genome allowing AAV replication and packaging in a mammalian cell. Helper virus functions for instance include adenovirus helper functions. Such helper virus functions may be provided in a number of ways, including by providing helper virus or by providing, for example, nucleic acid sequences encoding the required function(s) to a producer host cell in AAV manufacturing.

The terms “tropism” and “transduction” are interrelated, but there are differences. The term “tropism” as used herein refers to the ability of an AAV vector or virion to infect one or more specified cell types, but can also encompass how the vector functions to transduce the cell in the one or more specified cell types; i.e. tropism refers to preferential entry of the AAV vector or virion into certain cell or tissue type(s) and/or preferential interaction with the cell surface that facilitates entry into certain cell or tissue types, optionally and preferably followed by expression (e.g., transcription and, optionally, translation) of sequences carried by the AAV vector or virion in the cell, e.g., for a recombinant virus, expression of the heterologous nucleotide sequence(s). As used herein, the term “transduction” refers to the ability of an AAV vector or virion to infect one or more particular cell types; i.e. transduction refers to entry of the AAV vector or virion into the cell and the transfer of genetic material contained within the AAV vector or virion into the cell to obtain expression for the vector genome. In some cases, but not all cases, transduction and tropism may correlate.

The term “tropism profile” as used herein refers to the pattern of transduction of one or more target cells, tissues and/or organs. Different AAV serotypes exhibit deviating tropism profiles and tropism may be changed, e.g., by capsid engineering.

The term “host cell” denotes a cell comprising an exogenous nucleic acid of interest, for example, one or more microorganism, yeast cell, insect cell, or mammalian cell. For instance, the host cell may comprise an AAV helper construct, an AAV vector plasmid, an accessory function vector, and/or other transfer DNA. The term includes the progeny of the original cell which has been transfected. The progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. In certain circumstances, spontaneous or induced changes can occur in karyotype during storage or transfer of such clonal populations. Therefore, cells derived from the cell line referred to may not be precisely identical to the ancestral cells or cultures, and the cell line referred to includes such variants.

The term “transfection” is used to refer to the uptake of foreign DNA by a cell, such that the cell has been “transfected” once the exogenous DNA has been introduced inside the cell membrane. See, e.g., Graham et al., (1973) Virology, 52:456; Sambrook et al., (1989) Molecular Cloning, a laboratory manual, Cold Spring Harbor Laboratories, New York; Davis et al., (1986) Basic Methods in Molecular Biology, Elsevier; Chu et al., (1981) Gene, 13:197. Such techniques can be used to introduce one or more exogenous DNA moieties into suitable host cells. In some embodiments, the term “transduction” is used to refer to the uptake of foreign DNA by a cell, where the foreign DNA is provided by a virus or a viral vector. Consequently, a cell has been “transduced” when exogenous DNA has been introduced inside the cell membrane. In some embodiments, the term “transformation” is used to refer to the uptake of foreign DNA by bacterial cells.

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. A naturally occurring “antibody” is a glycoprotein comprising at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be, but is not limited to, a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass. Throughout this document, the term “antibody” or “antibody molecule” also includes any fragments thereof and any derivatives thereof, unless the context indicates otherwise.

The term “antibody fragment” or “antigen-binding fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies). The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single-chain polypeptide, and wherein the scFv retains the specificity of the intact antibody from which it is derived. Unless specified, as used herein an scFv may have the VL and VH variable regions in either order, e.g., with respect to the N terminal and C-terminal ends of the polypeptide, the scFv may comprise VL-linker-VH or may comprise VH-linker-VL.

The terms “complementarity determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme), or a combination thereof, and ImMunoGenTics (IMGT) numbering (Lefranc, M.-P., The Immunologist, 7, 132-136 (1999); Lefranc, M.-P. et al., Dev. Comp. Immunol., 27, 55-77 (2003); Lefranc et al., (2015) Nucleic Acids Res. 43, D413-422) (“IMGT” numbering scheme). In a combined Kabat and Chothia numbering scheme for a given CDR region (for example, HCDR1, HCDR2, HCDR3, LCDR1, LCDR2 or LCDR3), in some embodiments, the CDRs correspond to the amino acid residues that are defined as part of the Kabat CDR, together with the amino acid residues that are defined as part of the Chothia CDR. As used herein, the CDRs defined according to the “Chothia” number scheme are also sometimes referred to as “hypervariable loops.” Under IMGT, the CDR regions of an antibody can be determined using the program IMGT/DomainGap Align. Generally, unless specifically indicated, the antibody molecules can include any combination of one or more Kabat CDRs and/or Chothia CDRs.

The term “epitope” includes any protein determinant capable of specific binding to an immunoglobulin or otherwise interacting with a molecule. Epitopic determinants generally consist of chemically active surface groupings of molecules such as amino acids or carbohydrate or sugar side chains and can have specific three-dimensional structural characteristics, as well as specific charge characteristics. An epitope may be “linear” or “conformational.” Conformational and linear epitopes are distinguished for example in that the binding to the former but not the latter is lost in the presence of denaturing solvents.

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

The phrase “human antibody,” as used herein, includes antibodies having variable regions in which both the framework and CDR regions are derived from sequences of human origin. The constant region is also derived from human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well-known numbering schemes, e.g., the Kabat numbering scheme, the Chothia numbering scheme, or a combination of Kabat and Chothia, and ImMunoGenTics (IMGT) numbering (see, e.g., Sequences of Proteins of Immunological Interest, U.S. Department of Health and Human Services (1991), eds. Kabat et al.; Al Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mal. Biol. 273:927-948; Lefranc, M.-P., The Immunologist, 7, 132-136 (1999); Lefranc, M.-P. et al., Dev. Comp. Immunol., 27, 55-77 (2003); Lefranc et al., (2015) Nucleic Acids Res. 43, D413-422.

Human antibodies may include amino acid residues not encoded by human sequences (e.g., mutations introduced by random or site-specific mutagenesis in vitro or by somatic mutation in vivo, or a conservative substitution to promote stability or manufacturing). However, the term “human antibody” as used herein, is not intended to include antibodies in which CDR sequences derived from the germline of another mammalian species, such as a mouse, have been grafted onto human framework sequences.

The phrase “recombinant antibody” as used herein, includes all antibodies that are prepared, expressed, created or isolated by recombinant means, and includes recombinant human antibodies such as antibodies isolated from an animal (e.g., a mouse) that is transgenic or transchromosomal for human immunoglobulin genes or a hybridoma prepared therefrom, antibodies isolated from a host cell transformed to express the human antibody, e.g. from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, the term “affinity” refers to the strength of interaction between antibody and antigen at single antigenic sites. Within each antigenic site, the variable regions of the antibody interact through weak non-covalent forces with the antigen at numerous sites; the more interactions, the stronger the affinity. As used herein, the term “high affinity” for an IgG antibody or fragment thereof (e.g., a Fab fragment) refers to an antibody having an affinity of 10−8 M or less, 10−9 M or less, or 10−10 M, or 10−11 M or less, or 10−12 M or less, or 10−13 M or less for a target antigen. However, high affinity binding can vary for other antibody isotypes. For example, high affinity binding for an IgM isotype refers to an antibody having an affinity of 10−7 M or less, or 10−8 M or less.

The term “binding specificity” or “specifically binds” as used herein refers to the ability of an individual antibody combining site to react with one antigenic determinant and not with a different antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

The terms “treat” and “treatment” refer to therapeutic treatment, wherein the object is to slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The terms “prevention”, “prevent” and “preventing” of any particular disease or disorder refers to prophylactic or preventive measures such as the administration of a compound of the present invention to a subject before any symptoms of that disease or disorder are apparent.

The term “subject” refers to an animal, human or non-human, to whom treatment according to the methods of the present invention is provided. Veterinary and nonveterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs. In some preferred embodiments, the subject is a human.

The terms “pharmaceutically acceptable” and “physiologically acceptable” are used interchangeable herein and refer to a biologically acceptable formulation, gaseous, liquid or solid, suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering an rAAV virion as disclosed herein to a subject.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, processes conducted “in vitro” refer to processes which are performed outside of the normal biological environment, for example, studies performed in a test tube, a flask, a petri dish, in artificial culture medium. Processes conducted “in vivo” refer to processes performed within living organisms or cells. for example, studies performed in cell cultures or in mice. Studies performed “ex vivo” refer to studies done in or on tissue from an organism in an external environment, e.g., with minimal alteration of natural conditions, e.g., allowing for manipulation of an organism's cells or tissues under more controlled conditions than may be possible in in vivo experiments.

The term “library” as used herein refers to a multitude, i.e., at least two, different variant linear nucleic acids, plasmids, viral particles or viral vectors, etc.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, prevail. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### AAV VP2 Fusion Polypeptides

Provided herein are AAV VP2 fusion polypeptides comprising, from N to C-terminus, a polypeptide ligand, optionally a peptide linker, and an AAV VP2 capsid polypeptide. rAAV virions comprising said AAV VP2 fusion polypeptide and displaying the polypeptide ligand on their surface can be used for cell-type specific gene delivery during therapeutic applications and applications in basic research since they provide high-cell type selectivity and/or high targeting specificity allowing restricted biodistribution and safe gene transfer.

When used for rAAV virion assembly, typically together with AAV VP1 and/or VP3 capsid polypeptides, the AAV VP2 fusion polypeptides may show good decoration levels, meaning that a satisfactory number of AAV VP2 fusion polypeptides is incorporated in the rAAV virion. It was surprisingly found that good decoration levels may be achieved by N-terminally fusing a polypeptide ligand having a molecular weight of up to 10 kDa to the AAV VP2 capsid polypeptide. The decoration level decreases in AAV VP2 fusion polypeptides comprising an N-terminally located polypeptide ligand with a molecular weight above 10 kDa. AAV VP2 fusion polypeptides comprising an N-terminally located polypeptide ligand with a molecular weight above 15 kDa such as darpins having a molecular weight of about 18 kDa show no or only minimal decoration. Without wishing to be bound by theory it is believed that since the AAV VP2 capsid polypeptide is not essential for capsid assembly, offering an AAV VP2 capsid polypeptide with unfavorable structure, such as an AAV VP2 fusion polypeptide comprising a bulky polypeptide ligand, could result in the AAV VP2 capsid polypeptide not being used in capsid assembly, resulting in rAAV virions with no or very low AAV VP2 fusion polypeptide decoration exclusively or mainly composed of AAV VP1 and VP3 capsid polypeptides.

The AAV VP2 fusion polypeptides described herein may mediate improved transduction of and/or increased tropism in at least one tissue or cell type, relative to an AAV VP2 capsid polypeptide which is not fused to said polypeptide ligand, but which is otherwise identical to the VP2 capsid polypeptide comprised in the AAV VP2 fusion polypeptide. Improved transduction of and/or increased tropism in at least one tissue or cell type may be mediated by the polypeptide ligand, which may have the ability to bind to a cell surface molecule expressed on the at least one tissue or cell type. Suitable polypeptide ligands with a molecular weight below 10 kDa include, but are not limited to, GP2 and Sso7d ligands and affibodies.

The present invention is based, in part, on the incorporation of highly diverse polypeptide ligand libraries, for instance highly diverse Sso7d libraries into rAAV virions by fusing these libraries to the N-terminus of the AAV VP2 capsid polypeptide. AAV libraries comprising a plurality of rAAV virions comprising the AAV VP2 fusion polypeptide in their capsid and comprising a nucleic acid encoding the AAV VP2 fusion polypeptide encapsulated within said capsid were generated. The AAV libraries have high diversity and functional titers and can be used for the in vivo selection of AAV VP2 fusion polypeptides with desired characteristics. Provided herein are AAV VP2 fusion polypeptides obtained by in vivo selection enriching for AAV capsids with high transduction of specific cell types.

The AAV VP2 fusion polypeptides provided herein may be used in the generation of recombinant AAV vectors. Such recombinant AAV vectors are suitable for the delivery of heterologous nucleic acids, such as therapeutic transgenes, into a target cell.

Thus, in one aspect, provided herein are adeno-associated virus (AAV) VP2 fusion polypeptides comprising an AAV VP2 capsid polypeptide and a polypeptide ligand. The polypeptide ligand is fused, either directly or via a linker, to the N-terminus of the AAV VP2 capsid polypeptide. The polypeptide ligand has a molecular weight of up to 10 kDa, including up to 9 kDa, up to 8 kDa, up to 6 kDa or up to 5 kDa, e.g., from 3 to 10 kDa, from 4 to 8 kDa, or from 5 to 7 kDa.

In one aspect, provided herein is and AAV VP2 fusion polypeptide comprising an AAV VP2 capsid polypeptide and a polypeptide ligand, wherein the polypeptide ligand is fused to the N-terminus of the AAV VP2 capsid polypeptide; and wherein the AAV VP2 capsid polypeptide comprises one or more mutations that abolish or reduce binding to Heparan Sulphate Proteoglycan (HSPG) and/or Sialic Acid (SIA). In some embodiments, the polypeptide ligand has a molecular weight of up to 10 kDa.

“AAV” is an abbreviation for adeno-associated virus and may be used to refer to the virus itself or derivatives thereof. The term covers all subtypes and both naturally occurring and recombinant forms, except where required otherwise. The term “AAV” includes, for example, AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10, including AAVrh10), AAV type 12 (AAV12), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, and so on.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession NOs. NC-002077 (AAV1), AF063497 (AAV1), NC-001401 (AAV2), AF043303 (AAV2), NC-001729 (AAV3), NC-001829 (AAV4), U89790 (AAV4), NC-006152 (AAV5), AF513851 (AAV7), AF513852 (AAV8), and NC-006261 (AAV8); or in publications such as WO2005033321 (AAV1-9), the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99: 11854; Moris et al. (2004) Virology 33:375-383; international patent publications WO 00/28061, WO 99/61601, WO 98/11244; and U.S. Pat. No. 6,156,303.

In some embodiments, said polypeptide ligand specifically binds to a cell surface molecule expressed on at least one tissue or cell type. In some embodiments, said AAV VP2 fusion polypeptide mediates increased transduction of and/or increased tropism in at least one tissue or cell type relative to an AAV VP2 capsid polypeptide not comprising said polypeptide ligand but which is otherwise identical to the VP2 capsid polypeptide comprised in the AAV VP2 fusion polypeptide.

In some embodiments, transduction of at least one tissue or cell type is increased by about 10%, 20%, 30%, 40%, 50%, 60%, 70%, 80%, 90%, 100%, 150%, 200%, 300%, 400%, 500%, 600%, 700%, 800%, 900% or 1000%. In some embodiments, the AAV VP2 fusion polypeptide mediates at least 2-fold, at least 5-fold, at least 10-fold, at least 15-fold, at least 20-fold, at least 25-fold, at least 30-fold, at least 40-fold, at least 50-fold, at least 100-fold, at least 1000-fold or more than 1000-fold, increased transduction in at least one tissue or cell type relative to an AAV VP2 capsid polypeptide not comprising said polypeptide ligand but which is otherwise identical to the VP2 capsid polypeptide comprised in the AAV VP2 fusion polypeptide. In some embodiments, tropism in at least one tissue or cell type is increased by about 10%, 20%, 30%, 40%, 50%, 60%, 70%, 80%, 90%, 100%, 150%, 200%, 300%, 400%, 500%, 600%, 700%, 800%, 900% or 1000%.

In some embodiments, said AAV VP2 fusion polypeptide mediates increase transduction of and/or increased tropism cells of whole blood, brain, liver, spleen, kidney, skeletal muscle, heart, lungs, or bone marrow, or multipotent progenitor cells (MPPs), such as multipotent hematopoietic progenitor cells, or hematopoietic stem cells (HSCs), such as long-term hematopoietic stem cells (LT-HSCs)

In one aspect, provided herein are AAV VP2 fusion polypeptides comprising and AAV VP2 capsid polypeptide and a polypeptide ligand, wherein the polypeptide ligand is fused, either directly or via a linker, to the N-terminus of the AAV VP2 capsid polypeptide and is selected from the group consisting of a GP2 polypeptide, an Sso7d polypeptide and an affibody.

The term “GP2 polypetide” as used herein refers to a polypeptide scaffold derived from the 45-residue T7 phage gene 2 protein (Gp2). This polypeptide contains an α-helix opposite a R-sheet with two adjacent loops amenable to mutation. Mutagenesis of this scaffold can yield high-affinity target-specific binders.

The term “Sso7d polypeptide” as used herein refers to a polypeptide derived from the Sso7d protein from the hyperthermophilic archaeon Sulfolobus solfataricus. This protein is an attractive binding scaffold because of its small size (7 kDa), high thermal stability (Tm of 98° C.), and absence of cysteines and glycosylation sites. In some embodiments, the Sso7d polypeptide is derived from a charge-neutralized variant of the S. solfataricus Sso7d protein, in particular from a reduced charge Sso7d (rcSso7d) variant described in Traxlmayr et al. (DOI 10.1074/jbc.M116.741314). As used herein, Sso7d polypeptides also encompasses polypeptides that have over their full length at least about 70%, 75%, 80%, 85%, 90%, 95%, 96%, 97%, 98%, 99% or 100% sequence identity with wild type Sso7d or a rcSso7d variant described in Traxlmayr et al. (DOI 10.1074/jbc.M116.741314).

In some embodiments, said polypeptide ligand is selected from the group consisting of an Sso7d polypeptide of SEQ ID NO: 1 and an Sso7d polypeptide having at least 80%, 85%, 90%, or 95% sequence identity therewith.

In some embodiments, said polypeptide ligand is selected from an Sso7d polypeptide of SEQ ID NO: 1 optionally harboring up to 10, 9, 8, 7, 6, 5, 4, 3, 2 or 1 amino acid substitution(s).

In some embodiments, said polypeptide ligand is selected from the group consisting of an Sso7d polypeptide of SEQ ID NO: 1, in which amino acid residues X at positions 21, 23, 25, 28, 30, 32, 40, 42 and 44 are independently selected from D, R, H, N, A, I, Y and W, and an Sso7d polypeptide having at least 80%, 85%, 90%, or 95% sequence identity therewith.

In some embodiments, said polypeptide ligand is selected from an Sso7d polypeptide of SEQ ID NO: 1, in which amino acid residues X at positions 21, 23, 25, 28, 30, 32, 40, 42 and 44 are independently selected from D, R, H, N, A, I, Y, W and an amino acid substitution and wherein SEQ ID NO:1 optionally harbors up to 10, 9, 8, 7, 6, 5, 4, 3, 2 or 1 amino acid substitution(s).

In some embodiments, the AAV VP2 fusion polypeptide further comprises a peptide linker which is located between the polypeptide ligand and the AAV VP2 capsid polypeptide. In some embodiments, said peptide linker is selected from the group consisting of a glycine-serine (GS) linker and an alanine-proline-serine (APS) linker. The GS linker may for instance be of the formular [GGGGS]n, wherein n is an integer in the range of 1 to 10, for instance wherein n is 1, 2, 3, 4, 5 or 6, particularly wherein n is 1, 2, 3 or 4. The APS linker may for instance be of the formular [APS]n, wherein n is an integer in the range of 1 to 10, for instance wherein n is 1, 2, 3, 4, 5 or 6, particularly wherein n is 2, 3, 4 or 5.

In some embodiments, the AAV VP2 capsid polypeptide comprised in the AAV VP2 fusion polypeptide is of an AAV serotype selected from the group consisting of AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5. In specific embodiments, the AAV VP2 capsid polypeptide comprised in the AAV VP2 fusion polypeptide is of an AAV serotype selected from the group consisting of AAV1, AAV6, AAV8 and AAV9.

In some embodiments, the AAV VP2 capsid polypeptide comprised in the AAV VP2 fusion polypeptide comprises at least one mutation in at least one binding site for its natural receptor present on a target cell of an AAV virion comprising the AAV VP2 capsid polypeptide. As used herein, the term “natural receptor” refers, for example, to heparan sulfate proteoglycan (HSPG), which has been shown to be the primary cellular receptor for AAV of serotype 2; to N-linked sialic acid containing glycans, which have been shown to be the primary cellular receptor for AAV of serotypes 1, 5 and 6; to O-linked sialic acid containing glycans, which has been shown to be the primary cellular receptor for AAV of serotype 4 and 9; to αVβ5 integrin, α5βI integrin, CD9, and hepatocyte growth factor receptor, which act as secondary receptors or rather co-receptors for AAV of serotype 2; to basic fibroblast growth factor receptor and 37/67 kDa laminin receptor (LamR), which act as secondary receptors or rather co-receptors for AAV of serotypes 2, 3, 8, and 9; and/or to the platelet derived growth factor receptor (PDGFR), which act as secondary receptors or rather co-receptors for serotype AAV-5. In some embodiments, at least one essential binding site of the AAV VP2 capsid polypeptide for its natural receptor is mutated. In some embodiments, said at least one binding site for the natural receptor of the AAV VP2 capsid polypeptide is located within the VP3 region of the AAV VP2 capsid polypeptide, i.e. in the region that is shared between the VP1, VP2 and VP3 capsid polypeptides. The regions encoding the VP1 and VP2 proteins represent N-terminal extensions of the region encoding the VP3 protein. Thus, the reading frames of the regions encoding VP1, VP2 and VP3 are overlapping, such that mutations in the VP3 region are present in all of the three capsid proteins. In some embodiments, the AAV VP2 fusion polypeptide is of the AAV serotype AAV6 and comprises at least one amino acid substitution within its VP3 region selected from the group consisting of K531E, V473D, K459S, N500E, G266A, N269Q, and D590A relative to the VP1 amino acid sequence of SEQ ID NO: 3, or any combination thereof. In some embodiments, the AAV VP2 fusion polypeptide is of the AAV serotype AAV6 and comprises the amino acid substitutions i) K531E and V473D; ii) K531E, K459S, V473D and N500E; iii) G266A and N269Q; iv) G266A, N269Q and D590A; v) K531E, V473D, G266A and N269Q; or vi) K531E, K459S, V473D, N500E, G266A, N269Q and D590A relative to the VP1 amino acid sequence of SEQ ID NO: 3.

In some embodiments, the AAV VP2 fusion polypeptide is of the AAV serotype AAV8 and comprises at least one amino acid substitution within its VP3 region selected from the group consisting of G268E, N271Q, S387A, A592Q and A592D relative to the VP1 amino acid sequence of SEQ ID NO: 4, or any combination thereof. In some embodiments, the AAV VP2 fusion polypeptide is of the AAV serotype AAV8 and comprises the amino acid substitution(s) i) G268E and N271Q; ii) S387A; iii) G268E, N271Q and S387A; iv) A592Q; or v) A592D relative to the VP1 amino acid sequence of SEQ ID NO: 4.

In some embodiments the AAV VP2 fusion polypeptide is of the AAV serotype AAV9 and comprises at least one amino acid substitution within its VP3 region selected from the group consisting of Q590A, W503A, N562A and E563A relative to the VP1 amino acid sequence of SEQ ID NO: 5, or any combination thereof. In some embodiments, AAV VP2 fusion polypeptide is of the AAV serotype AAV9 and comprises the amino acid substitution(s) i) W503A; ii) N562A and E563A; iii) Q590A and W503A; or iv) Q590A, W503A, N562A and E563A relative to the VP1 amino acid sequence of SEQ ID NO: 5.

In some embodiments, the AAV VP2 fusion polypeptide is of the AAV serotype AAV2 and comprises the amino acid substitution R585A relative to the VP1 amino acid sequence of SEQ ID NO: 6 within its VP3 region.

In some embodiments, the AAV VP2 fusion polypeptide is of the AAV serotype AAV1 and comprises at least one amino acid substitution within its VP3 region selected from the group consisting of V473D, N500E and R514A relative to the VP1 amino acid sequence of SEQ ID NO: 7, or any combination thereof. In some embodiments, the AAV VP2 fusion polypeptide is of the AAV serotype AAV1 and comprises the amino acid substitution(s) i) V473D and N500E; ii) R514A; or iii) V473D, N500E and R514A relative to the VP1 amino acid sequence of SEQ ID NO: 7.

In some embodiments, the AAV VP2 capsid polypeptide comprised in the AAV VP2 fusion polypeptide comprises at least once amino acid substitution relative to its respective wild type AAV VP2 capsid polypeptide. The present inventors have shown that certain amino acid substitutions in the AAV VP2 capsid polypeptide may result in improved receptor binding, e.g., improved binding of an AAV VP2 fusion polypeptide comprising the mutated AAV VP2 capsid polypeptide to the receptor for the ligand comprised in the AAV VP2 fusion polypeptide. The amino acid substitutions in the AAV VP2 capsid polypeptide may also result in improved transduction. Without wishing to be bound by theory it is hypothesized that the amino acid substitutions identified by the present inventors as improving receptor binding and/or transduction result in a higher portion of the VP2 N-termini present in an AAV virion facing the outside of the virus particle. Under natural conditions, the majority of VP2 N-termini present in an AAV virion face the inside of the AAV capsid, thereby limiting the decoration level of the AAV virion with a ligand that is fused to the VP2 N-terminus. By increasing the portion of VP2 N-termini present in the AAV virous facing the outside of the virus particle, i.e. by increasing the VP2 exposure of the AAV virion, the decoration level with a ligand fused to the VP2 N-terminus is increased, which my lead to improved receptor binding and transduction levels.

Thus, in some embodiments, the AAV VP2 fusion polypeptide is of the AAV serotype AAV1 and comprises at least one amino acid substitution selected from the group consisting of E147S, P185G, P166R, M211V, G199R, D213A, T162R, and P191N relative to the VP1 amino acid sequence of SEQ ID NO: 7, or any combination thereof. In particular such embodiments, the AAV VP2 fusion polypeptide comprises the amino acid substitution(s) D213A, T162R, and/or P191N, particularly, the AAV VP2 fusion polypeptide comprises all three amino acid substitutions D213A, T162R, and P191N.

In some embodiments, the AAV VP2 fusion polypeptide of the AAV serotype AAV1 further comprises the amino acid substitutions V473D and N500E relative to the VP1 amino acid sequence of SEQ ID NO: 7.

In other embodiments, the AAV VP2 fusion polypeptide is of an AAV serotype other than AAV1 and comprises at least one amino acid substitution corresponding to at least one of the amino acid substitution selected from the group consisting of E147S, P185G, P166R, M211V, G199R, D213A, T162R, and P191N relative to the VP1 amino acid sequence of SEQ ID NO: 7. In particular such embodiments, the AAV VP2 fusion polypeptide is of the AAV serotype 8 and comprises at least one amino acid substitution selected from the group consisting of E147S, P186G, P167R, M212V, G200R, D214A, K163R, and P192N relative to the VP1 amino acid sequence of SEQ ID NO: 4, or any combination thereof. Particularly, the AAV VP2 fusion polypeptide comprises the amino acid substitution(s) D214A, K163R and/or P192N, particularly, all three amino acid substitutions D214A, K163R and P192N.

In other such embodiments, the AAV VP2 fusion polypeptide is of the AAV serotype 9 and comprises at least one amino acid substitution selected from the group consisting of E147S, P185G, P166R, G199R, D213A, S162R and P191N relative to the VP1 amino acid sequence of SEQ ID NO: 5, or any combination thereof. In particular such embodiments, the AAV VP2 fusion polypeptide comprises the amino acid substitution(s) D213A, S162R and/or P191N, particularly all three amino acid substitutions D213A, S162R and P191N.

In some embodiments, the nucleic acid encoding the AAV VP2 fusion polypeptide does not comprise the natural start codon of the AAV VP2 capsid polypeptide. The natural start codon of the AAV VP2 capsid polypeptide may be mutated, e.g., deleted, in order to avoid expression of the native VP2 polypeptide by inhibiting transcription of the VP2 open reading frame. Preferably, the mutation does not change the reading frame. In some embodiments, the AAV VP2 capsid polypeptide comprised in the AAV VP2 fusion polypeptide misses its first natural amino acid, e.g., the first threonine of the corresponding wild type AAV VP2 capsid polypeptide.

In some embodiments, the polypeptide ligand comprised in the AAV VP2 fusion polypeptide comprises up to 3 cysteine residues, up to 2 cysteine residues, up to 1 cysteine residue, or no cysteine residue.

### Nucleic Acids Encoding the AAV VP2 Fusion Polypeptides and AAV Cargo Nucleic Acid Sequences

In one aspect, provided herein are nucleic acids encoding the AAV VP2 fusion polypeptides described herein. The nucleic acid encoding the AAV VP2 fusion polypeptide may be RNA, such as mRNA or DNA, such as cDNA, linear DNA or circular DNA, e.g., plasmid DNA.

Also provided herein are nucleic acids that may be encapsulated by an AAV capsid comprising the AAV VP2 fusion polypeptide described herein and rAAV virions comprising such nucleic acids. These nucleic acids are referred to herein as “AAV genome”, “AAV vector genome”, “AAV vector”, “AAV vector nucleic acid”, “AAV cargo”, or “cargo plasmid”, which terms are used interchangeably herein. Such AAV cargos or AAV vector genomes typically comprise two AAV inverted terminal repeat sequences. The AAV cargos may encode the AAV VP2 fusion polypeptide described herein and/or may comprise a reporter sequence and/or a barcode sequence, or the AAV cargos may encode a therapeutic RNA or protein, including a therapeutic antibody or fragment thereof.

The AAV vector genome may be single stranded or self-complementary. “Self-complementary AAV” or “scAAV” refers a construct in which a coding region carried by a recombinant AAV nucleic acid sequence has been designed to form an intra-molecular double-stranded DNA template. Without being bound by theory, upon infection, rather than waiting for cell mediated synthesis of the second strand, the two complementary halves of scAAV may associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254 (incorporated by reference in its entirety). Self-complementary AAVs are described in, e.g., U.S. Pat. Nos. 6,596,535; 7,125,717; and 7,456,683, each of which is incorporated herein by reference in its entirety. For example, the 5′ ITR can be mutated, for example, by deleting the terminal resolution site to allow hairpin formation of the genome.

In some embodiments, the rAAV vectors disclosed herein lack one or more (e.g., all) AAV rep and/or cap genes. An AAV vector may comprise (e.g., in its ITRs) nucleic acid sequences (e.g., DNA) from any suitable AAV serotype. Suitable AAV serotypes include, but are not limited to, AAV serotypes AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5. For instance, an AAV vector, e.g., an scAAV vector, may comprise nucleic acid sequences from an AAV-2, e.g., ITR sequences from an AAV-2. An AAV vector, e.g., an scAAV vector, may also comprise nucleic acids from more than one serotype. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., Virol., 45: 555-564 {1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Williams, (2006) Mol. Ther., 13(1): 67-76; and the AAV-11 genome is provided in Mori et al., (2004) Virology, 330(2): 375-383.

In some embodiments, functional inverted terminal repeat (ITR) sequences may be used to support, e.g., the rescue, replication and packaging of the AAV virion. Thus, an AAV vector disclosed herein may include sequences that in cis provide for replication and packaging (e.g., functional ITRs) of the virus. The ITRs can be but need not be the wild-type nucleotide sequences, and may be altered, e.g., by the insertion, deletion or substitution of nucleotides, so long as the sequences provide for functional rescue, replication and packaging. The ITRs may be from any AAV serotype for which a recombinant virus can be derived including, but not limited to, AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5.

In some embodiments, the rAAV vector disclosed herein comprises one or more ITRs, e.g., two ITRs, with one upstream and the other downstream of a heterologous nucleic acid (e.g., encoding the AAV VP2 fusion polypeptide described herein and/or a reporter protein or a therapeutic RNA or protein) and/or the other nucleic acid elements discussed above. In some embodiments, a nucleic acid disclosed herein, e.g., in an scAAV vector, comprises a first ITR that is disposed 5′ and a second ITR that is disposed 3′ of the other vector elements, wherein the ITRs are independently 1, 2, 3, 4, 5, 6, 7, 8, 9, 10, 11, 12, 13, 14, 15, 16, 17, 18, 19, 20, 50, 100, 150, 200, 250 nucleotides 5′ and/or 3′ of the other elements. An ITR sequence may be wild-type, or it may comprise one or more mutations, e.g., as long as it retains one or more function of a wild-type ITR. In some embodiments, wild-type ITR may be modified to comprise a deletion of a terminal resolution site. In some embodiments, an scAAV as disclosed herein may comprise two ITR sequences, where both independently are wild-type, variant, or modified AAV ITR sequences. In some embodiments, at least one ITR sequence is a wild-type, variant or modified AAV ITR sequence. In some embodiments, the two ITR sequences are both wild-type, variant or modified AAV ITR sequences. In some embodiments, the “left” or 5′-ITR is a modified AAV ITR sequence that allows for production of self-complementary genomes, and the “right” or 3′-ITR is a wild-type AAV ITR sequence. In some embodiments, the “right” or 3′-ITR is a modified AAV ITR sequence that allows for the production of self-complementary genomes, and the “left” or 5′-ITR is a wild-type AAV ITR sequence. In some embodiments, both ITRs are AAV2 ITRs (optionally one of which is wild-type and the other modified to remove a terminal resolution site or both are wild-type). Embodiments of AAV ITRs provided in WO/2019/094253 (PCT/US2018/058744), which is incorporated herein by reference in its entirety, may also be used for any AAV ITR disclosed herein.

The polynucleotide sequences can be produced by de novo synthesis (e.g. solid-phase DNA synthesis) or by PCR using existing sequence as template. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., Nucleic Acids Res. 19:967, 1991; and Eckert et al., PCR Methods and Applications 1:17, 1991.

Also provided herein are vectors (e.g., expression vectors) comprising a nucleic acid encoding an AAV VP2 fusion polypeptide described herein and/or comprising a reporter sequence and/or comprising a barcode sequence or comprising a nucleic acid encoding a therapeutic RNA or protein, including a therapeutic antibody or fragment thereof. Such vectors may be used to express and/or produce the RNA or protein of interest, such as the AAV VP2 fusion polypeptide, for example, in cells in vitro, ex vivo or in vivo, for example in a tissue or tissues of interest in an organism. Various expression vectors can be employed to express an RNA or protein of interest, such as the AAV VP2 fusion polypeptide described herein. Both viral-based and nonviral expression vectors can be used to produce an RNA or protein of interest in a cell, for example a mammalian cell. Nonviral vectors and systems include plasmids, episomal vectors, typically with an expression cassette for expressing a protein or RNA, and human artificial chromosomes (see, e.g., Harrington et al., Nat Genet. 15:345, 1997). Such non-viral vectors may be delivered to a cell of interest using transfection or transduction methods known in the art, for example, using lipids (e.g., lipofectamine), electroporation, mechanical cell membrane distortion, and the like. The term “expression vector” refers to a carrier nucleic acid molecule into which a desired coding sequence can be inserted for introduction into a cell where it can be expressed. The vector can be a DNA vector, an RNA vector, a plasmid, a cosmid, or a viral vector, or artificial chromosomes (see, e.g., Harrington et al., Nat Genet 15:345, 1997). For example, non-viral vectors useful for expression of RNA and proteins of interest, such as the AAV VP2 fusion polypeptide described herein, in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing proteins. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses. Useful viral vectors include vectors based on any one of the following viruses: retroviruses (e.g., lentivirus), lentiviruses adenoviruses, adeno-associated viruses, herpes viruses (e.g., Herpes Simplex Virus (HSV)), vectors based on SV40, papilloma virus, HBP Epstein Barr virus, vaccinia virus, Sinbis virus, influenza virus, reovirus, Newcastle disease virus (NDV), measles virus, vesicular stomatitis virus (VSV), parvovirus, poliovirus, poxvirus, Seneca Valley virus, coxsackievirus, enterovirus, myxoma virus, maraba virus, or Semliki Forest virus (SFV). See, Brent et al., supra; Smith, Annu. Rev. Microbiol. 49:807, 1995; and Rosenfeld et al., Cell 68:143, 1992. In some embodiments, the vector is an adeno-associated virus (AAV) vector, e.g., a recombinant AAV (rAAV) vector.

In some embodiments, the vector can be a recombinant DNA molecule comprising a nucleic acid encoding the AAV VP2 fusion polypeptide as described herein and/or comprising a reporter sequence and/or comprising a barcode sequence or comprising a nucleic acid encoding a therapeutic RNA or protein, including a therapeutic antibody or fragment thereof “Recombinant” as used herein means that a vector, polynucleotide, polypeptide or cell is the product of various combinations of cloning, restriction or ligation steps (e.g., relating to a polynucleotide or polypeptide comprised therein), and/or other procedures that result in a construct that is distinct from a product found in nature. A recombinant virus or vector is a viral particle comprising a recombinant polynucleotide. The terms respectively include replicates of the original polynucleotide construct and progeny of the original virus construct.

The recombinant vector typically includes one or more regulatory sequences operatively linked to the nucleic acid sequence to be expressed. The term “regulatory sequence” includes promoters, enhancers, and other expression control elements (e.g., polyadenylation signals). A person skilled in the art readily recognizes that expression of one or more components of the vector in a target cell may require a regulatory sequence. Regulatory sequences include those which direct constitutive expression of a nucleotide sequence, as well as tissue-specific regulatory and/or inducible sequences. Expression vectors can also include elements designed to optimize messenger RNA stability and translatability in host cells, and/or drug selection markers for establishing permanent, stable cell clones expressing the AAV VP2 fusion polypeptide as described herein. The design of the expression vector can depend on such factors as the choice of the host cell to be transformed, the level of expression of protein desired, and the like. General methods for generating such recombinant expression vectors can be found in Sambrook and Russell eds. (2001) Molecular Cloning: A Laboratory Manual, 3rd edition; the series Ausubel et al. eds. (2007 with updated through 2010) Current Protocols in Molecular Biology, among others known in the art.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

Expression can employ any appropriate host cells known in the art, for example, mammalian host cells, bacterial host cells, yeast host cells, insect host cells, etc. Both prokaryotic and eukaryotic expression systems are widely available. In some embodiments, the expression system is a mammalian cell expression, such as a HEK293 expression system. In some embodiments, a nucleic acid may be codon-optimized to facilitate expression in a desired host cell. It will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (2001).

Eukaryotic RNA molecules may undergo RNA splicing to remove introns from the primary transcripts. Vectors containing genomic eukaryotic sequences may require donor and/or acceptor splicing sites to ensure proper processing of the transcript for protein expression (see Chandler et al., 1997, Proc. Natl. Acad. Sci. USA, 94(8):3596-601).

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly A) to the 3′ end of the transcript. RNA molecules modified with this polyA tail appear to more stable and are translated more efficiently. Thus, in other embodiments involving eukaryotes, it is preferred that the terminator comprises a signal for the cleavage of the RNA, and it is more preferred that the terminator signal promotes polyadenylation of the mRNA. The terminator and/or polyadenylation site elements can serve to enhance mRNA levels and/or to minimize read through from the cassette into other sequences. Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

To propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually, the inclusion of a drug selection marker aids in the cloning and identification of transformants, for example, genes that confer resistance to kanamycin, neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (HSV-tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

The choice of expression vector depends on the intended cells in which one or more components of the vector is to be expressed. Typically, the vectors contain one or more regulatory sequences, such as a promoter and other regulatory sequence (e.g., enhancers) that are operably linked to the AAV VP2 fusion polypeptide open reading frame.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned”, “operatively linked”, “under control”, and “under transcriptional control” mean that a promoter is in a correct functional location and/or orientation in relation to a nucleic acid sequence to control transcriptional initiation and/or expression of that sequence. A promoter may or may not be used in conjunction with an “enhancer”, which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous”. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring”, i.e., containing different elements of different transcriptional regulatory regions and/or mutations that alter expression. In addition to producing nucleic acid sequences of promoters and enhancers synthetically, sequences may be produced using recombinant cloning and/or nucleic acid amplification technology, for example PCR, in connection with the compositions disclosed herein (see U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

The promoters employed can be constitutive, inducible, synthetic, tissue- or cell-specific, and/or useful under the appropriate conditions to direct high-level expression of the introduced DNA segment, such as is advantageous in the large-scale production of recombinant proteins and/or peptides. In addition, other regulatory elements may also be incorporated to improve expression of a nucleic acid encoding the AAV VP2 fusion polypeptide described herein, e.g., enhancers, ribosomal binding sites, transcription termination sequences, and the like.

In some embodiments, a constitutive promoter is employed to provide constant expression of the AAV VP2 fusion polypeptide described herein or of a reporter sequence or a therapeutic RNA or protein encoded by a nucleic acid comprised in the rAAV virion described herein. Examples of a constitutive promoter include, but not limited to, the immediate early cytomegalovirus (CMV) promoter, the simian virus 40 (SV40) early promoter, mouse mammary tumor virus (MMTV) promoter, human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1 promoter, the hemoglobin promoter, and the creatine kinase promoter.

In some embodiments, a ubiquitous promoter is employed. Non-limiting examples of ubiquitous promoters include CMV, CBA (including derivatives CAG, CBh, etc.), EF-1a, PGK, UBC, GUSB (hGBp), and UCOE (promoter of HNRPA2B1-CBX3).

In some embodiments, an inducible promoter is employed to prevent expression of inserted sequences except under inducing conditions. The use of an inducible promoter provides a molecular switch capable of turning on expression of the polynucleotide sequence with which it is operatively linked when such expression is desired or turning off the expression when expression is not desired. Examples of inducible promoters include, but are not limited to, e.g., an arabinose promoter, a lacZ promoter, a tetracycline promoter, a metallothionein promoter, a glucocorticoid promoter, a progesterone promoter, or a heat shock promoter.

In some embodiments, a tissue- or cell-specific promoter is employed to provide expression of the AAV VP2 fusion polypeptide described herein or the reporter protein, the therapeutic RNA or protein encoded by a nucleic acid comprised by the rAAV virion described herein only in specific tissues or cells. The identity of tissue- or cell-specific promoters or elements, as well as assays to characterize their activities, is well known to those of skill in the art. Examples include the human LIMK2 gene (Nomoto et al. 1999, Gene, 236(2):259-271), the somatostatin receptor 2 gene (Kraus et al., 1998, FEES Lett., 428(3): 165-170), murine epididymal retinoic acid-binding gene (Lareyre et al., 1999, J. Biol. Chem., 274(12):8282-8290), human CD4 (Zhao-Emonet et al., 1998, Biochirn. Biophys. Acta, 1442(2-3): 109-119), mouse alpha2 (XI) collagen (Tsumaki, et al., 1998, J. Biol. Chem., 273(36):22861-22864), DIA dopamine receptor gene (Lee, et al., 1997, J. Auton. Nerv. Syst., 74(2-3):86-90), insulin-like growth factor II (Wu et al., 1997, Biochem. Biophys. Res. Commun., 233(1):221-226), human platelet endothelial cell adhesion molecule-1 (Almendro et al., 1996, J. Immunol., 157(12):5411-5421), muscle creatine kinase (MCK) promoter (Wang et al., Gene Ther. 2008 November; 15(22):1489-99).

In some embodiments, promoters which are not cell specific are employed. In some embodiments, a strong promoter or a weak promoter (classified according to its affinity and other promoters' affinity for RNA polymerase and/or sigma factor) is employed.

In some embodiments, a synthetic promoter is employed to provide expression of the AAV VP2 fusion polypeptide described herein or of a therapeutic RNA or protein encoded by a nucleic acid comprised by the rAAV virion described herein. Synthetic promoters can greatly exceed the transcriptional potencies of natural promoters. For example, the synthetic promoters that do not get shut off or reduced in activity by the endogenous cellular machinery or factors can be selected. Other elements, including trans-acting factor binding sites and enhancers may be inserted into the synthetic promoter to improve transcriptional efficiency. Synthetic promoters can be rationally designed and chemically synthesized to combine the best features of both synthetic and biological promoters. Synthetic oligos are annealed and ligated through several processes to generate the full-length chemically synthesized promoter. Synthetic promoters can be inducible or cell-type specific promoters.

In some embodiments, the promoter operably linked to the AAV VP2 fusion polypeptide ORF, or to the nucleic acid sequence encoding the therapeutic nucleic acid or protein, or to the reporter sequence is selected from a173CMV promoter, a HCMV promoter, a CBh promoter, a CAG promoter, and an mCCT promoter.

A person skilled in the art readily recognizes that specific promoters are especially suitable for expressing the AAV VP2 fusion polypeptide as described herein, a reporter sequence or a therapeutic RNA or protein in a target cell, including, but not limited to, a promoter that is species-specific, inducible, tissue-specific, or cell cycle-specific (Parr et al., Nat. Med. 3: 1145-9 (1997); the contents of which are herein incorporated by reference in its entirety).

In some embodiments, the promoter operably linked to the AAV VP2 fusion polypeptide described herein, to a reporter sequence or to a therapeutic RNA or protein encoded by a nucleic acid comprised in the rAAV virion described herein is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 bp. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 bp.

In one embodiment, the promoter operably linked to the AAV VP2 fusion polypeptide described herein, to a reporter sequence or to a therapeutic RNA or protein encoded by a nucleic acid comprised in the rAAV virion described herein may be a combination of two or more components, regions or sequences of the same or different promoters such as, but not limited to, CMV and CBA. Each component may have a length of 200, 210, 220, 230, 240, 250, 260, 270, 280, 290, 300, 310, 320, 330, 340, 350, 360, 370, 380, 381, 382, 383, 384, 385, 386, 387, 388, 389, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 bp. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800 bp.

In some embodiments, the promoter operably linked to the AAV VP2 fusion polypeptide described herein, to a reporter sequence or to a therapeutic RNA or protein encoded by a nucleic acid comprised in the rAAV virion described herein is a combination of a CMV-enhancer sequence, for example an immediate/early CMV enhancer sequence (for example a 382-nucleotide CMV-enhancer sequence) and a chicken beta-actin (CBA)-promoter sequence (for example 260 nucleotide CBA promoter sequence).

In addition to promoters, other regulatory elements may also be required or desired for efficient expression of the AAV VP2 fusion polypeptide described herein or of a reporter protein, a therapeutic RNA or a therapeutic protein encoded by a nucleic acid comprised in the rAAV virion described herein. These elements include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

In some embodiments, the AAV vector provided herein comprises an intron, optionally disposed between a promoter element and the polynucleotide to be expressed. Without being bound by theory, inclusion of a 5′ intron may enhance the level and steady state of the expressed mRNA. Non-limiting examples of introns include SV40 derived introns, CBA-MVM derived introns, MVM (67-97 bps), FIX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

In one embodiment, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500 nucleotides.

The expression vectors may also provide a secretion signal sequence position to form a fusion protein with the protein to be expressed. More often, the inserted sequences encoding the protein to be expressed are linked to a signal sequence before inclusion in the vector.

Generation of an expression vector can utilize a vector that includes a multiple cloning site (MCS), which is a nucleic acid region that contains multiple restriction enzyme sites, any one of which can be used in conjunction with standard recombinant technology to digest the vector. See Carbonelli et al., 1999, Levenson et al., 1998, and Cocea, 1997. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

In some embodiments, the nucleic acid encoding an AAV VP2 fusion polypeptide described herein further comprises a regulatory sequence. In some embodiments, the nucleic acid encoding an AAV VP2 fusion polypeptide described herein further comprises a promoter. In some embodiments, the nucleic acid encoding an AAV VP2 fusion polypeptide described herein comprises a regulatory sequence, particularly a promoter, efficient for driving expression in a target cell.

In some embodiments the open reading frame of the AAV VP2 fusion polypeptide is operably linked to said promoter. In some embodiments, the promoter is selected from a HCMV promoter, a 173CMV promoter, a CAG promoter, a CBh promoter and an mCCT promoter. In some embodiments, the nucleic acid further comprises a polyadenylation signal, which may for instance be selected from a BGH or an SV40 polyadenylation signal. In one embodiment, the nucleic acid comprises a sequence of SEQ ID NO: 8.

In some embodiments, the nucleic acid encoding the AAV VP2 fusion polypeptide further comprises a reporter sequence. In some embodiments, the reporter sequence is operably linked to a promoter, e.g., to a second promoter. In some embodiments, the reporter sequence encodes a fluorescent or a luminescent reporter protein. A number of reporter proteins are known in the art, and include green fluorescent protein (GFP), variant of green fluorescent protein (GFP10), enhanced GFP (eGFP), TurboGFP, GFPS66T, TagGFP2, mUKGEmerald GFP, Superfolder GFP, GFPuv, destabilised EGFP (dEGFP), Azami Green, mWasabi, Clover, mClover3, mNeonGreen, NowGFP, Sapphire, T-Sapphire, mAmetrine, photoactivatable GFP (PA-GFP), Kaede, Kikume, mKikGR, tdEos, Dendra2, mEosFP2, Dronpa, blue fluorescent protein (BFP), eBFP2, azunte BFP, mTagBFP, mKalamal, mTagBFP2, shBFP, cyan fluorescent protein (CFP), eCFP, Cerulian CFP, SCFP3A, destabilised ECFP (dECFP), CyPet, mTurquoise, mTurquoise2, mTFPI, photoswitchable CFP2 (PS-CFP2), TagCFP, mTFPI, mMidonishi-Cyan, aquamanne, mKeima, mBeRFP, LSS-mKate2, LSS-mKate1, LSS-mOrange, CyOFP I, Sandercyanin, red fluorescent protein (RFP), eRFP, mRaspberry, mRuby, mApple, mCardinal, mStable, mMaroonl, mGarnet2, tdTomato, mTangerine, mStrawberry, TagRFP, TagRFP667, TagRFP675, mKate2, HcRed, t-HcRed, HcRed-Tandem, mPlum, mNeptune, NirFP, Kindling, far red fluorescent protein, yellow fluorescent protein (YFP), eYFP, destabilised EYFP (dEYFP), TagYFP, Topaz, Venus, SYFP2, mCherry, PA-mCherry, sfCherry, sfCherry2, Citrine, mCitrine, Ypet, IANRFP-AS83, mPapayal, mCyRFPI, mHoneydew, mBanana, mOrange, Kusabira Orange, Kusabira Orange 2, mKusabira Orange, mOrange 2, mKOv, mKO2, mGrape1, mGrape2, zsYellow, eqFP611, Sirius, Sandercyanm, shBFP-N168S/L1731, near infrared proteins, iFP1.4, iRFP713, iRFP670, iRFP682, iRFP702, iRFP720, iFP2.0, mIFP, TDsmURFP, miRFP670, Brilliant Violet (BV) 421, BV 605, BV 510, BV 711, BV786, PerCP, PerCP/Cy5.5, DsRed, DsRed2, mRFPI, pocilloporin, Renilla GFP, Monster GFP, paGFP, or a Phycobihprotein, or a biologically active variant or fragment of any one thereof. The reporter protein may for instance be selected from the group consisting of EGFP, mCherry, sfCherry, sfCherry2, mClover3, mRuby3, mApple, iRFP, tdTomato, mVenus, YFP, RFP, firefly luciferase, and nanoluciferase, in particular from EGFP and sfCherry2.

In some embodiments, the vector, e.g., the expression vector, is an adeno-associated vector (AAV). In some embodiments, the AAV vector comprises an open reading frame encoding the AAV VP2 fusion polypeptide described herein that is flanked on one or both sides by an inverted terminal repeat (ITR) sequence. The nucleic acid may additionally comprise one or more additional elements such as, for example, a promoter, an enhancer, one or more intron sequences, a poly(A) sequence, a stuffer sequence, for instance a HPRT intron derived stuffer sequence, and combinations thereof. In some embodiments, the vector comprises a polynucleotide AAV vector encoding the AAV VP2 fusion polypeptide as described herein, encapsulated in an AAV capsid.

In some embodiments, the vector comprises an open reading frame encoding the AAV VP2 fusion polypeptide described herein, operably linked to at least one target cell compatible regulatory sequence, e.g., a promoter. In some embodiments, the promoter is selected from a 173CMV promoter, a HCMV promoter, a CBh promoter, a CAG promoter and an mCCT promoter.

In some embodiments, the ITRs in the AAV vector are derived from the same AAV serotype. In some embodiments, the ITRs in the AAV vector are derived from different AAV serotypes. In some embodiments, the ITRs are the same. In some embodiments, the ITRs are different.

In some embodiments the ITRs in the AAV vector are derived from the same AAV serotype as the AAV capsid. In some embodiments, the ITRs in the AAV vector are derived from a serotype different from that of the AAV capsid. In some embodiments, the ITRs are derived from AAV2 and the AAV capsid is derived from a serotype other than AAV2, for example, AAV9.

In some embodiments, the nucleic acid encoding the AAV VP2 fusion polypeptide comprises two AAV inverted terminal repeat (ITR) sequences located upstream and downstream of the AAV VP2 fusion polypeptide open reading frame. In some embodiments, the two ITR sequences are located at the 5′ and the 3′ termini of the nucleic acid molecule. In some of these embodiments, the nucleic acid further comprises a reporter sequence. The reporter sequence may for instance encode a fluorescent or a luminescent reporter protein. For instance, the reporter protein may be selected from the group consisting of EGFP, mCherry, sfCherry, sfCherry2, mClover3, mRuby3, mApple, iRFP, tdTomato, mVenus, YFP, RFP, firefly luciferase, and nanoluciferase, in particular from EGFP and sfCherry2. In some embodiments, the reporter sequence is operably linked to a second promoter. In some embodiments, the nucleic acid encoding the AAV VP2 fusion polypeptide further comprises a posttranscriptional regulatory element, for instance WPRE or a derivative thereof. In some embodiments, the nucleic acid further comprises a polyadenylation signal, for instance selected from a BGH and a SV40 polyadenylation signal. In one embodiment, the nucleic acid encoding the AAV VP2 fusion polypeptide comprises the sequence of SEQ ID NO: 2.

In one aspect, provided herein is a kit comprising the isolated nucleic acid encoding said AAV VP2 fusion polypeptide.

### Host Cells and AAV Manufacture

Methods for introducing expression vectors containing a polynucleotide sequence of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods/gene gun, virosomes, immunoliposomes, polycation:nucleic acid conjugates, naked DNA, artificial virions, fusion to the herpes virus structural protein VP22, agent-enhanced uptake of DNA, ex vivo transduction, protoplast fusion, retroviral transduction, viral transfection, lipid based transfection or other conventional techniques. In the case of protoplast fusion, the cells are grown in media and screened for the appropriate activity. For long-term, high-yield production of recombinant proteins, stable expression will often be desired. For example, cell lines which stably express polypeptides can be prepared using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. Methods and conditions for culturing the resulting transfected cells and for recovering the produced antibody are known to those skilled in the art and may be varied or optimized depending upon the specific expression vector and mammalian host cell employed, based upon the present description.

In one aspect, provided herein are cells comprising the AAV VP2 fusion polypeptide described herein or the nucleic acid encoding same. Such cells may for instance be a host cells or therapeutic cells. The terms “host cell” and “recombinant host cell” are used interchangeably herein, which refer to not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Host cells can be used to produce or express the AAV VP2 fusion polypeptide and optionally to assemble AAV virions comprising same. Accordingly, the disclosure also features methods for producing an AAV VP2 fusion polypeptide and methods for producing AAV virions comprising an AAV VP2 fusion polypeptide using a host cell. In some embodiments, the methods include culturing the host cell (into which a recombinant expression vector encoding the AAV VP2 fusion polypeptide has been introduced) in a suitable medium, such that the AAV VP2 fusion polypeptide is produced. In some embodiments, the method further includes isolating the AAV VP2 fusion polypeptide or the AAV virion comprising same from the medium or the host cell.

In one embodiment, the host cells are genetically engineered to comprise nucleic acids encoding the AAV VP2 fusion polypeptide. In one embodiment, the host cells are genetically engineered by using an expression cassette. The phrase “expression cassette” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The host cells can be, but are not limited to, a eukaryotic cell or a prokaryotic cell, such as a bacterial cell, an insect cell, or a mammalian cell, such as a human cell. E. coli is one prokaryotic host useful for cloning and expressing the polynucleotides. Other microbial hosts suitable for use include bacilli, such as Bacillus subtilis, and other enterobacteriaceae, such as Salmonella, Serratia, and various Pseudomonas species. In these prokaryotic hosts, one can also replicate expression vectors, which typically contain control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express the AAV VP2 fusion polypeptide described herein. Eukaryotic host cell lines capable of secreting intact heterologous proteins have been developed including the CHO cell lines, various Cos cell lines, HeLa cells, myeloma cell lines, transformed B-cells and hybridomas. Suitable eukaryotic host cells include, but are not limited to, Vero cells, HeLa cells, COS cells, CHO cells, HEK293 cells, BHK cells and MDCKII cells. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP pol III promoter, the constitutive MPSV promoter, the tetracycline-inducible CMV promoter (such as the human immediate-early CMV promoter), the constitutive CMV promoter, and promoter-enhancer combinations known in the art.

In some embodiments, the host cell is suitable for AAV virion assembly. Thus, also disclosed herein are methods of producing rAAV virions.

Naturally occurring AAV comprises different sequences directing viral DNA replication (rep), encapsidation/packaging and host cell chromosome integration. Typically, three AAV promoters (named p5, p19, and p40 for their relative map locations) drive the expression of the two AAV internal open reading frames encoding rep and cap genes in wild-type virus. The two rep promoters (p5 and p19), coupled with the differential splicing of the single AAV intron (at nucleotides 2107 and 2227), may result in the production of four rep proteins (rep 78, rep 68, rep 52, and rep 40) from the rep gene. Rep proteins possess multiple enzymatic properties that are ultimately responsible for replicating the viral genome. The cap gene is typically expressed from the p40 promoter. Alternative splicing and non-consensus translational start sites are responsible for the production of the three related capsid proteins VP1, VP2, and VP3. A single consensus polyadenylation site is located at map position 95 of the AAV genome. The life cycle and genetics of AAV are reviewed in Muzyczka, (1992) Curr. Topics Microbiol. Imm., 158: 97-129.

In some embodiments, a nucleic acid, e.g., a plasmid, encoding the AAV VP2 fusion polypeptide is used for producing rAAV virions. In some embodiments, at least one further nucleic acid, e.g., plasmid, comprising an AAV rep gene and/or an AAV cap gene is used in preparing the rAAV virion. In some embodiments, the VP2 start codon in the cap gene is mutated, whereby the nucleic acid encoding the AAV VP2 fusion polypeptide is the only source for VP2 subunits, and hence, all VP2 capsid polypeptides in the generated rAAV virion are fused to the polypeptide ligand. Also disclosed herein are nucleic acid sequences, e.g., plasmids, comprising at least one adenovirus helper function gene. In some embodiments, the nucleic acids encoding the AAV rep, AAV cap, and/or adenovirus helper genes may be present in the same structure, e.g., a single plasmid, or they may be present in separate structures. In some embodiments, the one or more plasmids are co-transfected with the cargo nucleic acid, i.e., the nucleic acid to be encapsulated within the rAAV virion into competent cells, and the cells are then cultured to produce the rAAV virions. In some cases, the cargo plasmid comprising the nucleic acid sequence to be encapsulated (the “AAV genome”) and the plasmid(s) comprising AAV rep and/or cap genes are transferred to cells permissible for infection with a helper virus of AAV (e.g., adenovirus, El-deleted adenovirus or herpesvirus). Techniques to produce rAAV particles, in which an AAV genome to be packaged, rep and cap genes, and helper virus functions are provided to a cell are known in the art and may include, e.g., electroporation. In some embodiments, production of rAAV involves the following components present within a single cell (denoted herein as a packaging cell): a rAAV vector, AAV rep and cap genes separate from (i.e., not in) the rAAV vector, and helper virus functions. Production of pseudotyped rAAV is disclosed in, for example, WO 01/83692 which is incorporated by reference herein in its entirety. In various embodiments, AAV capsid proteins may be modified to enhance delivery of the recombinant vector. Modifications to capsid proteins are generally known in the art. See, for example, US 2005/0053922 and US 2009/0202490, the disclosures of which are incorporated by reference herein in their entirety.

In various embodiments, general principles of viral vector production may be utilized to produce the vectors and virus, e.g., rAAV, disclosed herein. Carter, (1992) Curr. Opinions Biotech., 1533-539; Muzyczka, (1992) Curr. Topics Microbial. Immunol., 158:97-129. Various approaches are disclosed in Ratschin et al., (1984) Mol. Cell. Biol., 4: 2072; Hennonat et al., (1984) Proc. Natl. Acad. Sci. USA, 81: 6466; Tratschin et al., (1985) Mol. Cell. Biol., 5: 3251; McLaughlin et al., (1988) J. Virol., 62: 1963; Lebkowski et al., (1988) Mol. Cell. Biol., 7:349; Samulski et al. (1989) J. Virol., 63:3822-3828; U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., (1995) Vaccine, 13: 1244-1250; Paul et al., (1993) Hum. Gene Ther., 4: 609-615; Clark et al. (1996) Gene Therapy, 3: 1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

An exemplary method of generating a packaging cell is to create a cell line that stably expresses some or all necessary components for AAV particle production. For example, a plasmid (or multiple plasmids) encoding a rAAV vector lacking AAV rep and cap genes, AAV rep and cap genes separate from the rAAV vector, and a selectable marker, such as a neomycin resistance gene, are integrated into the genome of a cell. AAV genomes have been introduced into bacterial plasmids by procedures such as GC tailing (Samulski et al., (1982) Proc. Natl. Acad. Sci. USA, 79: 2077-2081), addition of synthetic linkers containing restriction endonuclease cleavage sites (Laughlin et al., (1983) Gene, 23:65-73) or by direct, blunt-end ligation (Senapathy et al., (1984) J. Biol. Chem., 259: 4661-4666). The packaging cell line is then infected with a helper virus such as adenovirus and/or a plasmid encoding a helper virus. The advantages of this method are that the cells are selectable and are suitable for large-scale production of rAAV. Other examples of suitable methods employ adenovirus or baculovirus rather than plasmids to introduce rAAV vectors and/or rep and cap genes into packaging cells.

In some embodiments, a method of producing recombinant virus comprises providing a nucleic acid to be packaged. In some embodiments, the nucleic acid is a plasmid. In other embodiments, the nucleic acid comprises a heterologous nucleic acid sequence interposed between a first AAV terminal repeat and a second AAV terminal repeat. In some embodiments, the heterologous nucleic acid encodes the AAV VP2 fusion polypeptide described herein and/or a reporter protein. In some embodiments, the heterologous nucleic acid encodes a therapeutic RNA or a therapeutic protein, including a therapeutic antibody. In some embodiments, where the heterologous nucleic acid does not encode the AAV VP2 fusion polypeptide, the method of producing recombinant virus comprises providing an additional nucleic acid encoding the AAV VP2 fusion polypeptide. In some embodiments, the method of producing recombinant virus comprises providing one or more additional nucleic acids. In some embodiments, the one or more additional nucleic acids comprises an AAV rep gene and/or an AAV cap gene. In some embodiments, the one or more additional nucleic acids comprises an AAV rep gene derived from an AAV serotype 1, AAV serotype 2, AAV serotype 3, AAV serotype 4, AAV serotype 5, AAV serotype 6, AAV serotype 7, AAV serotype 8, or AAV serotype 9. In some embodiments, the one or more additional nucleic acids comprises an AAV cap gene derived from an AAV serotype 1, AAV serotype 2, AAV serotype 3, AAV serotype 4, AAV serotype 5, AAV serotype 6, AAV serotype 7, AAV serotype 8, or AAV serotype 9. In some embodiments, the one or more additional nucleic acids comprises one or more of an adenovirus helper function gene.

In some embodiments, the nucleic acids are co-transfected into competent cells or packaging cells. Methods of co-transfection are known in the art, and include, but are not limited to, transfection by lipofectamine, electroporation, and polyethylenimine. Competent cells or packaging cells may be non-adherent cells cultured in suspension or adherent cells. In one embodiment any suitable packaging cell line may be used, such as HeLa cells, HEK 293 cells and PerC.6 cells (a cognate 293 line). In one embodiment, the packaging cells are human cells. In one embodiment, the packaging cells are HEK 293 cells. In one embodiment, the packaging cells are insect cells. In one embodiment, the packaging cells are Sf9 cells. In some embodiments, the method comprises culturing the transfected cells to produce recombinant virus. In some embodiments, the method comprises recovering the recombinant virus. Methods of recovering recombinant virus include, e.g., those disclosed in U.S. Pat. Nos. 6,143,548 and 9,408,904. In some embodiments, recombinant virus is secreted into cell culture media and purified from the media. In some embodiments, packaging cells are lysed, and the contents purified to recover the recombinant virus. In some embodiments, the virus is recovered from the packaging cell by filtration or centrifugation. In some embodiments, the virus is recovered from the packaging cell by chromatography.

In some embodiments, mammalian host cells are used to express the AAV VP2 fusion polypeptide and to incorporate it into AAV virions, typically together with AAV VP1 and VP3 polypeptides. For this purpose, HEK293 or derivatives thereof such as HEK293T/17 or AAV293 cells may be transfected with the nucleic acid encoding the AAV VP2 fusion polypeptide and nucleic acids encoding the AAV VP1, VP2 and rep polypeptides. In some embodiments, the HEK293 cells are further transfected with nucleic acids encoding adenovirus helper functions for AAV replication, such as adenovirus E2, E4 and/or VA gene products. In some embodiments, the HEK293 cells are co transfected with one nucleic acid molecule encoding the AAV VP2 fusion polypeptide, one nucleic acid molecule encoding the AAV VP1, VP2 and rep polypeptides, particularly wherein the AAV VP1 and VP2 polypeptides are encoded by the AAV cap gene, more particularly wherein the AAV VP2 start codon in the AAV cap gene is mutated, one nucleic acid molecule encoding at least one, at least two, at least three or at least for adenoviral helper functions for AAV replication, particularly wherein said nucleic acid molecule encodes adenovirus E2, E4 and VA gene products, and optionally a further nucleic acid molecule encoding a therapeutic RNA or protein. In some embodiments, the nucleic acid molecule encoding the therapeutic RNA or protein is encapsulated in an AAV capsid composed of the AAV VP2 fusion polypeptide and AAV VP1 and VP3 polypeptides. In these embodiments, the nucleic acid molecule encoding the therapeutic RNA or protein typically further comprises flanking AAV ITR sequences. In other embodiments, a nucleic acid molecule encoding the AAV VP2 fusion polypeptide is encapsulated in an AAV capsid composed of the AAV VP2 fusion polypeptide and AAV VP1 and VP3 polypeptides. In these embodiments, the nucleic acid molecule encoding the AAV VP2 fusion polypeptide further comprises flanking AAV ITR sequences. In other embodiments, a nucleic acid molecule comprising a barcode sequence and flanking AAV ITR sequences is encapsulated in an AAV capsid composed of the AAV VP2 fusion polypeptide and AAV VP1 and VP3 polypeptides.

In some embodiments, the method of producing rAAV virions comprises transfecting an insect cell. Suitable insect cells include, but are not limited to, Sf9 cells. In some embodiments, the method comprises transfecting an insect cell with a baculovirus comprising the nucleic acids as disclosed herein. In some embodiments, the method comprises transfecting an insect cell with baculovirus comprising a nucleic acid comprising a heterologous nucleic acid sequence interposed between a first AAV terminal repeat and a second AAV terminal repeat. In some embodiments, the method comprises transfecting an insect cell with a baculovirus comprising one or more additional nucleic acids. In some embodiments, the one or more additional nucleic acids comprises an AAV rep gene and/or an AAV cap gene. In some embodiments, the one or more additional nucleic acids comprises one or more of an adenovirus helper function gene. In some embodiments, where the heterologous nucleic acid does not encode the AAV VP2 fusion polypeptide described herein, the method further comprises transfecting the insect cell with an additional nucleic acid encoding the AAV VP2 fusion polypeptide described herein. In some embodiments, the insect cells are cultivated under conditions suitable to produce recombinant virus. In some embodiments, the virus is recovered from the insect cell. In some embodiments, the virus is recovered from the insect cell by filtration or centrifugation. In some embodiments, the virus is recovered from the insect cell by chromatography.

In some embodiments, the cell is selected from a mammalian cell including but not limited to a murine cell, a non-human primate cell or a human cell. The mammalian cell may be selected from the group consisting of a liver cell, a brain cell, a spleen cell, a kidney cell, a blood cell, a lung cell, a muscle cell, a heart cell, a bone marrow cell, a multipotent progenitor cell (MPP), such as a multipotent hematopoietic progenitor cell, and a hematopoietic stem cell (HSC), such as a long-term hematopoietic stem cell (LT-HSC).

rAAV Virions Comprising the AAV VP2 Fusion Polypeptide

In one aspect, provided herein are recombinant AAV (rAAV) virions comprising the AAV VP2 fusion polypeptide described herein.

The abbreviation “rAAV” refers to recombinant adeno-associated virus. An “rAAV vector” as used herein refers to an AAV vector comprising a polynucleotide sequence not of AAV origin (i.e., a polynucleotide heterologous to AAV), typically a sequence of interest for the genetic transformation of a cell. In some embodiments, the heterologous nucleic acid encodes the AAV VP2 fusion polypeptide described herein. In some embodiments, the heterologous nucleic acid encodes a therapeutic nucleic acid, a therapeutic protein or a therapeutic antibody or antibody fragment. In some embodiments, the heterologous polynucleotide may be flanked by at least one, and sometimes by two, AAV inverted terminal repeat (ITR) sequences. The term rAAV vector encompasses both rAAV vector particles and rAAV vector nucleic acids. An rAAV vector may either be single-stranded (ssAAV) or self-complementary (scAAV). An “AAV virus” or “AAV viral particle” or “AAV virion” refers to a viral particle composed of at least one AAV capsid protein (typically by all of the capsid proteins of or derived from one or more wild-type AAV) and an encapsulated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle”, “rAAV virion” or simply an “rAAV vector.”

In some embodiments, the AAV virion disclosed herein comprises one or more AAV capsid proteins. Typically, in AAV three capsid proteins, VP1, VP2 and VP3, multimerize to form the capsid. The rAAV virion disclosed herein comprises the AAV VP2 fusion polypeptide and optionally further comprises AAV VP1 and/or VP3 polypeptides. Typically, the capsid of the rAAV virion disclosed herein is composed of VP1, VP3 and VP2 fusion polypeptide subunits. Typically, the capsid of the rAAV virion disclosed herein does not comprise AAV VP2 capsid polypeptides which are not fused to a polypeptide ligand, meaning that typically, all VP2 subunits are AAV VP2 fusion polypeptides as described herein. The AAV capsid polypeptides comprised in the rAAV virion disclosed herein may be from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5, or any variants thereof or variants thereof, for instance having at least 80%, 85%, 90%, 95%, 96%, 97%, 98%, or 99% sequence identity over the full length of the VP1, VP2 and/or VP3 capsid polypeptide.

The polypeptide sequences of capsid proteins are known in the art and can also be derived from the genome of the AAV. These can be used as exemplary capsids in the AAV virus compositions disclosed herein.

In some embodiments, the AAV VP2 capsid polypeptide comprised in the AAV VP2 fusion polypeptide described herein and/or the AAV VP1 polypeptides and/or the AAV VP3 polypeptides comprised in the rAAV virion described herein may be naturally occurring or mutant/variant capsid polypeptides independently selected from the group including, but not limited to, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r 11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAV A3.3, AAV A3.4, AAV A3.5, AAV A3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC 12, AAV-2-pre-miRNA-1O1, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu. 1, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPEN AAV10 and/or Japanese AAV10 capsid polypeptides, or variants thereof, for instance having at least 80%, 85%, 90%, 95%, 96%, 97%, 98%, or 99% sequence identity over the full length of the VP1, VP2 and/or VP3 capsid polypeptide.

In some embodiments, the AAV VP2 capsid polypeptide comprised in the AAV VP2 fusion polypeptide described herein and/or the AAV VP1 polypeptides and/or the AAV VP3 polypeptides comprised in the rAAV virion described herein may be of or derived from an AAV serotype as described in United States Publication No. US20030138772, the contents of which are herein incorporated by reference in their entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772), AAV16.3 (US20030138772 SEQ ID NO: 10), AAV29.3/bb. 1 (US20030138772 SEQ ID NO: 11), AAV29.4 (US20030138772 SEQ ID NO: 12), AAV29.5/bb.2 (US20030138772 SEQ ID NO: 13), AAV1.3 (US20030138772 SEQ ID NO: 14), AAV13.3 (US20030138772 SEQ ID NO: 15), AAV24.1 (US20030138772 SEQ ID NO: 16), AAV27.3 (US20030138772 SEQ ID NO: 17), AAV7.2 (US20030138772 SEQ ID NO: 18), AAVC1 (US20030138772 SEQ ID NO: 19), AAVC3 (US20030138772 SEQ ID NO: 20), AAVC5 (US20030138772 SEQ ID NO: 21), AAVF1 (US20030138772 SEQ ID NO: 22), AAVF3 (US20030138772 SEQ ID NO: 23), AAVF5 (US20030138772 SEQ ID NO: 24), AAVH6 (US20030138772 SEQ ID NO: 25), AAVH2 (US20030138772 SEQ ID NO: 26), AAV42-8 (US20030138772 SEQ ID NO: 27), AAV42-15 (US20030138772 SEQ ID NO: 28), AAV42-5b (US20030138772 SEQ ID NO: 29), AAV42-lb (US20030138772 SEQ ID NO: 30), AAV42-13 (US20030138772 SEQ ID NO: 31), AAV42-3a (US20030138772 SEQ ID NO: 32), AAV42-4 (US20030138772 SEQ ID NO: 33), AAV42-5a (US20030138772 SEQ ID NO: 34), AAV42-10 (US20030138772 SEQ ID NO: 35), AAV42-3b (US20030138772 SEQ ID NO: 36), AAV42-11 (US20030138772 SEQ ID NO: 37), AAV42-6b (US20030138772 SEQ ID NO: 38), AAV43-1 (US20030138772 SEQ ID NO: 39), AAV43-5 (US20030138772 SEQ ID NO: 40), AAV43-12 (US20030138772 SEQ ID NO: 41), AAV43-20 (US20030138772 SEQ ID NO: 42), AAV43-21 (US20030138772 SEQ ID NO: 43), AAV43-23 (US20030138772 SEQ ID NO: 44), AAV43-25 (US20030138772 SEQ ID NO: 45), AAV44.1 (US20030138772 SEQ ID NO: 46), AAV44.5 (US20030138772 SEQ ID NO: 47), AAV223.1 (US20030138772 SEQ ID NO: 48), AAV223.2 (US20030138772 SEQ ID NO: 49), AAV223.4 (US20030138772 SEQ ID NO: 50), AAV223.5 (US20030138772 SEQ ID NO: 51), AAV223.6 (US20030138772 SEQ ID NO: 52), AAV223.7 (US20030138772 SEQ ID NO: 53), AAV A3.4 (US20030138772 SEQ ID NO: 54), AAV A3.5 (US20030138772 SEQ ID NO: 55), AAV A3.7 (US20030138772 SEQ ID NO: 56), AAV A3.3 (US20030138772 SEQ ID NO: 57), AAV42.12 (US20030138772 SEQ ID NO: 58), AAV44.2 (US20030138772 SEQ ID NO: 59), AAV42-2 (US20030138772 SEQ ID NO: 9), or variants thereof.

In some embodiments, the AAV VP2 capsid polypeptide comprised in the AAV VP2 fusion polypeptide described herein and/or the AAV VP1 polypeptides and/or the AAV VP3 polypeptides comprised in the rAAV virion described herein may be of or derived from an AAV serotype described in United States Publication No. US20150159173, the contents of which are herein incorporated by reference in their entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV VP2 capsid polypeptide comprised in the AAV VP2 fusion polypeptide described herein and/or the AAV VP1 polypeptides and/or the AAV VP3 polypeptides comprised in the rAAV virion described herein may be of or derived from an AAV serotype as described in U.S. Pat. No. 7,198,951, the contents of which are herein incorporated by reference in their entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV 8 (SEQ ID NO: 7 of U.S. Pat. No. 7,198,951).

In some embodiments, the AAV VP2 capsid polypeptide comprised in the AAV VP2 fusion polypeptide described herein and/or the AAV VP1 polypeptides and/or the AAV VP3 polypeptides comprised in the rAAV virion described herein may be derived from AAV serotype which may be, or have, a mutation in the AAV9 sequence as described by N Pulicherla et al. (Molecular Therapy 19(6): 1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84. In some embodiments, the AAV capsid comprises one or more sequences engineered to deliver the vector across the blood-brain barrier (See, e.g., B. E. Deverman et al, Nature Biotech, Vol. 34, No. 2, p 204-211 (published online 1 Feb. 2016) and Caltech press release, A. Wetherston, www.neurology-cenfrd.com/2016/02/10/successfd/brain-barrier; See, also, WO 2016/0492301 and U.S. Pat. No. 8,734,809 (the contents of each of these are incorporated by reference in their entirety).

In some embodiments, the AAV VP2 capsid polypeptide comprised in the AAV VP2 fusion polypeptide described herein and/or the AAV VP1 polypeptides and/or the AAV VP3 polypeptides comprised in the rAAV virion described herein may be of or derived from an AAV serotype as described in U.S. Pat. No. 6,156,303, the contents of which are herein incorporated by reference in their entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In some embodiments, the AAV VP2 capsid polypeptide comprised in the AAV VP2 fusion polypeptide described herein and/or the AAV VP1 polypeptides and/or the AAV VP3 polypeptides comprised in the rAAV virion described herein may be of or derived from an AAV serotype as described in United States Publication No. US20140359799, the contents of which are herein incorporated by reference in their entirety, such as, but not limited to, AAV 8 (SEQ ID NO: 1 of US20140359799), AAVDJ (SEQ ID NO: 2 and 3 of US20140359799), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype selected from AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in International Publication No. WO 1998011244, the contents of which are herein incorporated by reference in their entirety, such as, but not limited to AAV4 (SEQ ID NO: 1-20 of WO 1998011244).

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype which may be, or have, a mutation in the AAV2 sequence to generate AAV2G9 as described in International Publication No. WO2014144229 and herein incorporated by reference in its entirety.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu. 19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV 8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi. 1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in International Publication No. WO2015168666, the contents of which are herein incorporated by reference in their entirety, such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in International Publication No. WO2018/160582, the contents of which are herein incorporated by reference in their entirety, such as, but not limited to, AAVhu68 (e.g., SEQ ID NO: 2 of WO2018160582), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in U.S. Pat. No. 9,233,131, the contents of which are herein incorporated by reference in their entirety, such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ ID NO:47 of U.S. Pat. No. 9,233,131), AAVhEr1.16 (SEQ ID NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 9,233,131), AAVhEr1.7 (SEQ ID NO:51 of U.S. Pat. No. 9,233,131), AAVhEr1.36 (SEQ ID NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID NO:53 of U.S. Pat. No. 9,233,131), AAVhEr3.1 (SEQ ID NO:59 of U.S. Pat. No. 9,233,131), AAV2.5T (SEQ ID NO:42 of U.S. Pat. No. 9,233,131), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in United States Patent Publication No. US20150376607, the contents of which are herein incorporated by reference in their entirety, such as, but not limited to, AAV-PAEC (SEQ ID NO: 1 of US20150376607), AAV-LKOl (SEQ ID NO:2 of US20150376607), AAV-LK02 (SEQ ID NO:3 of US20150376607), AAV-LK03 (SEQ ID NO:4 of US20150376607), AAV-LK04 (SEQ ID NO:5 of US20150376607), AAV-LK05 (SEQ ID NO:6 of US20150376607), AAV-LK06 (SEQ ID NO:7 of US20150376607), AAV-LK07 (SEQ ID NO:8 of US20150376607), AAV-LK08 (SEQ ID NO:9 of US20150376607), AAV-LK09 (SEQ ID NO: 10 of US20150376607), AAV-LK10 (SEQ ID NO: 1 of US20150376607), AAV-LK11 (SEQ ID NO: 12 of US20150376607), AAV-LK12 (SEQ ID NO: 13 of US20150376607), AAV-LK13 (SEQ ID NO: 14 of US20150376607), AAV-LK14 (SEQ ID NO: 15 of US20150376607), AAV-LK15 (SEQ ID NO: 16 of US20150376607), AAV-LK16 (SEQ ID NO: 17 of US20150376607), AAV-LK17 (SEQ ID NO: 18 of US20150376607), AAV-LK18 (SEQ ID NO: 19 of US20150376607), AAV-LK19 (SEQ ID NO:20 of US20150376607), AAV-PAEC2 (SEQ ID NO:21 of US20150376607), AAV-PAEC 4 (SEQ ID NO:22 of US20150376607), AAV-PAEC6 (SEQ ID NO:23 of US20150376607), AAV-PAEC7 (SEQ ID NO:24 of US20150376607), AAV-PAEC 8 (SEQ ID NO:25 of US20150376607), AAV-PAEC11 (SEQ ID NO:26 of US20150376607), AAV-PAEC 12 (SEQ ID NO:27, of US20150376607), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in United States Patent Publication No. US20150376240, the contents of which are herein incorporated by reference in their entirety, such as, but not limited to, AAV-8h (SEQ ID NO: 6 of US20150376240), AAV-8b (SEQ ID NO: 5 of US20150376240), AAV-h (SEQ ID NO: 2 of US20150376240), AAV-b (SEQ ID NO: 1 of US20150376240), or variants thereof.

In some embodiments, the AAV particles of the present invention may comprise or be derived from AAV serotype which may be, or have, a sequence as described in United States Patent Publication No. US20160017295, the contents of which are herein incorporated by reference in their entirety, such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ ID NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in United States Patent Publication No. US20150238550, the contents of which are herein incorporated by reference in their entirety, such as, but not limited to, BNP61 AAV (SEQ ID NO: 1 of US20150238550), BNP62 AAV (SEQ ID NO: 3 of US20150238550), BNP63 AAV (SEQ ID NO: 4 of US20150238550), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in United States Patent Publication No. US20150315612, the contents of which are herein incorporated by reference in their entirety, such as, but not limited to, AAVrh.50 (SEQ ID NO: 108 of US20150315612), AAVrh.43 (SEQ ID NO: 163 of US20150315612), AAVrh.62 (SEQ ID NO: 114 of US20150315612), AAVrh.48 (SEQ ID NO: 115 of US20150315612), AAVhu.19 (SEQ ID NO: 133 of US20150315612), AAVhu. 11 (SEQ ID NO: 153 of US20150315612), AAVhu.53 (SEQ ID NO: 186 of US20150315612), AAV4-8/rh.64 (SEQ ID No: 15 of US20150315612), AAVLG-9/hu.39 (SEQ ID No: 24 of US20150315612), AAV54.5/hu.23 (SEQ ID No: 60 of US20150315612), AAV54.2/hu.22 (SEQ ID No: 67 of US20150315612), AAV54.7/hu.24 (SEQ ID No: 66 of US20150315612), AAV54.1/hu.21 (SEQ ID No: 65 of US20150315612), AAV54.4R/hu.27 (SEQ ID No: 64 of US20150315612), AAV46.2/hu.28 (SEQ ID No: 68 of US20150315612), AAV46.6/hu.29 (SEQ ID No: 69 of US20150315612), AAV128.1/hu.43 (SEQ ID No: 80 of US20150315612), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be of or derived from an AAV serotype as described in International Publication No. WO2015121501, the contents of which are herein incorporated by reference in their entirety, such as, but not limited to, true type AAV (ttAAV) (SEQ ID NO: 2 of WO2015121501), “UPenn AAV10” (SEQ ID NO: 8 of WO2015121501), “Japanese AAV10” (SEQ ID NO: 9 of WO2015121501), or variants thereof.

In some embodiments, the AAV VP2 capsid polypeptide comprised in the AAV VP2 fusion polypeptide and/or the AAV VP1 capsid polypeptide and/or the AAV VP3 capsid polypeptide comprised in the rAAV virion described herein may be selected from the capsid polypeptides disclosed in International Publication No. WO2015191508, the contents of which are herein incorporated by reference in their entirety.

In some embodiments, the AAV VP2 capsid polypeptide comprised in the AAV VP2 fusion polypeptide and/or the AAV VP1 and/or VP3 polypeptides comprised in the rAAV virion described herein may be of a hybrid serotypes with enhanced transduction of or tropism in specific cell types of interest, prolonged transgene expression and/or an improved safety profile. The hybrid serotypes may be generated by transcapsidation, adsorption of bi-specific antibody to capsid surface, mosaic capsid, and chimeric capsid, and/or other capsid protein modifications.

In some embodiments, the AAV VP2 capsid polypeptide comprised in the AAV VP2 fusion polypeptide and/or the AAV VP1 and/or VP3 polypeptides comprised in the rAAV virion described herein may be further modified toward a specific therapeutic application by rational mutagenesis of capsid proteins (see, e.g., Pulicherla et al, Mol Ther, 201 1, 19: 1070-1078), incorporation of peptide ligands to the capsid, and directed evolution to produce new AAV variants.

The AAV VP2 capsid polypeptide comprised in the AAV VP2 fusion polypeptide and/or the AAV VP1 and/or VP3 polypeptides comprised in the rAAV virion described herein may be of an AAV serotype which may be selected from or derived from a variety of species, including but not limited to human, non-human primates, avian and bovine. In one embodiment, the AAV may be an avian AAV (AAAV). The AAAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV VP2 capsid polypeptide comprised in the AAV VP2 fusion polypeptide and/or the AAV VP1 and/or VP3 polypeptides comprised in the rAAV virion described herein may be of an AAV serotype which may be or derived from a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In some embodiments, the AAV VP2 capsid polypeptide comprised in the AAV VP2 fusion polypeptide and/or the AAV VP1 and/or VP3 polypeptides comprised in the rAAV virion described herein may be of an AAV serotype which may be or derived from a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In some embodiments, the AAV VP2 capsid polypeptide comprised in the AAV VP2 fusion polypeptide and/or the AAV VP1 and/or VP3 polypeptides comprised in the rAAV virion described herein may be of an AAV serotype which may be engineered as a hybrid AAV from two or more parental serotypes. In some embodiments, the serotype may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In some embodiments, the AAV VP2 capsid polypeptide comprised in the AAV VP2 fusion polypeptide and/or the AAV VP1 and/or VP3 polypeptides comprised in the rAAV virion described herein may be of an AAV serotype which may be generated by the AAV9 capsid library with mutations in amino acids 390-627 (VP1 numbering) as described by Pulicherla et al. (Molecular Therapy 19(6): 1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and 1479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T5821), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L5111, L537M), AAV9.55 (T1605A; F535L), AAV9.58 (C1475T, C1579A; T4921, H527N), AAV.59 (T1336C; Y446H), AAV9.61 (A1493T; N4981), AAV9.64 (C1531A, A1617T; L5111), AAV9.65 (C1335T, T1530C, C1568A; A523D), AAV9.68 (C1510A; P504T), AAV9.80 (G1441A, G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K5281), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV VP2 capsid polypeptide comprised in the AAV VP2 fusion polypeptide and/or the AAV VP1 and/or VP3 polypeptides comprised in the rAAV virion described herein may comprise the capsid sequences of SEQ ID NOs: 1 and 3 of International Publication No. WO2014160092, the content of which is incorporated herein by reference in its entirety.

In some embodiments, the AAV VP2 capsid polypeptide comprised in the AAV VP2 fusion polypeptide and/or the AAV VP1 and/or VP3 polypeptides comprised in the rAAV virion described herein may comprise AAV capsid sequence of SEQ ID NO: 1 or SEQ ID NOs: 2 to 4 of International Publication No. WO2014052789, the content of which is herein incorporated by reference in its entirety.

In some embodiments, the AAV VP2 capsid polypeptide comprised in the AAV VP2 fusion polypeptide and/or the AAV VP1 and/or VP3 polypeptides comprised in the rAAV virion described herein may have increased capacity to cross the blood-brain barrier in CNS as disclosed in U.S. Pat. No. 8,927,514, the content of which is incorporated herein by reference in its entirety. The amino acid sequences and nucleic acid sequences of such capsid proteins may include, but are not limited to, SEQ ID NOs: 2-17 and SEQ ID NOs: 25-33, respectively, of U.S. Pat. No. 8,927,514.

In some embodiments, the AAV VP2 capsid polypeptide comprised in the AAV VP2 fusion polypeptide and/or the AAV VP1 and/or VP3 polypeptides comprised in the rAAV virion described herein be AAV5 capsid proteins or variants thereof, for instance those of U.S. Pat. No. 7,056,502, the content of which is incorporated herein by reference in its entirety.

In some embodiments, the AAV VP2 capsid polypeptide comprised in the AAV VP2 fusion polypeptide and/or the AAV VP1 and/or VP3 polypeptides comprised in the rAAV virion described herein may be AAV6 capsid proteins or variants thereof.

In some embodiments, the AAV VP2 capsid polypeptide comprised in the AAV VP2 fusion polypeptide and/or the AAV VP1 and/or VP3 polypeptides comprised in the rAAV virion described herein may be AAV8 capsid proteins or variants thereof, for instance comprising the amino acid sequence of SEQ ID NO: 2 of U.S. Pat. No. 8,318,480, the content of which is herein incorporated by reference in its entirety.

In some embodiments, the AAV VP2 capsid polypeptide comprised in the AAV VP2 fusion polypeptide and/or the AAV VP1 and/or VP3 polypeptides comprised in the rAAV virion described herein may be AAV9 capsid proteins or variants thereof, for instance comprising the amino acid sequence of SEQ ID NO: 2 of U.S. Pat. No. 7,198,951, the content of which is incorporated herein by reference in its entirety or the sequences of SEQ ID NOs: 2, 4 or 6 as disclosed in US patent publication No. US20130224836, the content of which is incorporated herein by reference in its entirety, in which at least one of surface-exposed tyrosine residues in the amino acid sequence is substituted with another amino acid residue.

In some embodiments, the AAV VP2 capsid polypeptide comprised in the AAV VP2 fusion polypeptide and/or the AAV VP1 and/or VP3 polypeptides comprised in the rAAV virion described herein may be variant capsid proteins engineered to attain a specific property. Such methods for obtaining engineered capsids are described in, for example, patent publication WO2011038187, the contents of which are incorporated herein by reference in their entirety. Such methods and vectors are also described in, for example, patent publication WO2012112832 and patent application publication WO2015054653, the contents of which are incorporated herein by reference in their entirety. Such variant capsids include, for example, SEQ ID NO: 23 of WO2015054653, or a variant thereof. Additional variant capsids include, for example, those capsid sequences described in patent publication WO2017/019994, the contents of which are incorporated by reference in their entirety. In embodiments, the AAV capsid polypeptides may comprise an Anc80 AAV capsid sequence (e.g., SEQ ID NO: 1 of WO2017019994), for example, Anc80L65 (e.g., SEQ ID NO: 23 of WO2017019994), or, for example, Anc110 (e.g., SEQ ID NO: 42 of WO2017019994).

In some embodiments, the AAV VP2 capsid polypeptide comprised in the AAV VP2 fusion polypeptide and/or the AAV VP1 and/or VP3 polypeptides comprised in the rAAV virion described herein may be AAVrh10 capsid proteins or variants thereof, for instance comprising the amino acid sequence of SEQ ID NO: 81 of EP patent NO: 2341068.

In some embodiments, the AAV VP2 capsid polypeptide comprised in the AAV VP2 fusion polypeptide and/or the AAV VP1 and/or VP3 polypeptides comprised in the rAAV virion described herein may be AAVDJ capsid proteins, AAVDJ/8 capsid proteins, or variants thereof. In some embodiments, AAVDJ capsid proteins and/or AAVDJ/8 capsid proteins may comprise an amino acid sequence comprising a first region that is derived from a first AAV serotype (e.g., AAV2), a second region that is derived from a second AAV serotype (e.g., AAV8), and a third region that is derived from a third AAV serotype (e.g., AAV 9), wherein the first, second and third region may include any amino acid sequences that are disclosed in this description.

In some embodiments, the serotype of the AAV capsid polypeptides may depend on the desired distribution, transduction efficiency and cellular targeting required. As described by Sorrentino et al. (comprehensive map of CNS transduction by eight adeno-associated virus serotypes upon cerebrospinal fluid administration in pigs, Molecular Therapy accepted article preview online 7 Dec. 2015; doi: 10.1038/mt.2015.212; the contents of which are herein incorporated by reference in its entirety), AAV serotypes provided different distributions, transduction efficiencies and cellular targeting. In order to provide the desired efficacy, the AAV serotype needs to be selected that best matches not only the cells to be targeted but also the desired transduction efficiency and distribution.

In some embodiments, the rAAV virion exhibits increased transduction of and/or increased tropism in at least one tissue or cell type relative to an rAAV virion comprising an identical AAV VP2 capsid polypeptide which is however not fused to said polypeptide ligand.

In some embodiments, transduction of at least one tissue or cell type is increased by about 10%, 20%, 30%, 40%, 50%, 60%, 70%, 80%, 90%, 100%, 150%, 200%, 300%, 400%, 500%, 600%, 700%, 800%, 900% or 1000%. In some embodiments, the rAAV virion exhibits at least 2-fold, at least 5-fold, at least 10-fold, at least 15-fold, at least 20-fold, at least 25-fold, at least 30-fold, at least 40-fold, at least 50-fold, at least 100-fold, at least 1000-fold or more than 1000-fold, increased transduction in at least one tissue or cell type relative to an rAAV virion comprising an identical AAV VP2 capsid polypeptide which is however not fused to said polypeptide ligand. In some embodiments, tropism in at least one tissue or cell type is increased by about 10%, 20%, 30%, 40%, 50%, 60%, 70%, 80%, 90%, 100%, 150%, 200%, 300%, 400%, 500%, 600%, 700%, 800%, 900% or 1000%.

In some embodiments, all AAV VP2 capsid polypeptides comprised in said rAAV virion are fused to the polypeptide ligand, either directly or via a linker.

In some embodiments, the rAAV virion further comprises a nucleic acid sequence selected from the group consisting of a non-coding nucleic acid, a protein or an RNA coding sequence, an expression cassette, a multi-expression cassette, a sequence for homologous recombination, and a genomic gene targeting cassette.

In some embodiments, the rAAV virion further comprises a nucleic acid sequence encoding the AAV VP2 fusion polypeptide described herein. In some of these embodiments, the AAV VP2 fusion polypeptide comprised in the rAAV virion and the AAV VP2 fusion polypeptide encoded by said nucleic acid sequence comprised in the rAAV virion are the same.

In some embodiments, the rAAV virion comprises a nucleic acid sequence encoding a therapeutic nucleic acid, a therapeutic protein or a therapeutic antibody or antibody fragment.

The therapeutic nucleic acid may be selected from the group consisting of an mRNA, siRNA, miRNA, shRNA, and an antisense oligonucleotide.

The antibody fragment may for instance be selected from the group consisting of a Fab, Fab′, F(ab′)2, Fv, single domain antibody (dAb), and a single chain variable fragment (scFv).

In some embodiments, the rAAV virion comprising the AAV VP2 fusion polypeptide further comprises AAV VP1 and VP3 polypeptides. In some embodiments, the AAV VP2 fusion polypeptide and said AAV VP1 and VP3 polypeptides are of an AAV serotype independently selected from the group consisting of AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV. In some embodiments, the AAV VP2 fusion polypeptide and said AAV VP1 and VP3 polypeptides are of an AAV serotype selected from the group consisting of AAV1, AAV6, AAV8 and AAV9. In some of these embodiments, the AAV VP2 fusion polypeptide and said AAV VP1 and VP3 polypeptides are of the same AAV serotype.

In some embodiments, the AAV VP1, VP2 and VP3 polypeptides comprised in the rAAV virion comprise at least one mutation in at least one binding site for their natural receptor present on a target cell of an AAV virion composed of said AAV VP1, VP2 and VP3 polypeptides. In particular embodiments, at least one essential binding site for the natural receptor is mutated in the AAV VP1, VP2 and VP3 polypeptides. In some embodiments, the AAV VP1, VP2 and VP3 polypeptides comprise the same at least one mutation in the shared VP3 region of each AAV capsid polypeptide. In some of these embodiments, the AAV VP2 fusion polypeptide and said AAV VP1 and VP3 polypeptides are of the AAV serotype AAV6 and each comprise at least one amino acid substitution within the shared VP3 region selected from the group consisting of K531E, V473D, K459S, N500E, G266A, N269Q, and D590A relative to the VP1 amino acid sequence of SEQ ID NO: 3, or any combination thereof. In some of these embodiments, the AAV VP2 fusion polypeptide and said AAV VP1 and VP3 polypeptides each comprise the amino acid substitutions i) K531E and V473D; ii) K531E, K459S, V473D and N500E; iii) G266A and N269Q; iv) G266A, N269Q and D590A; v) K531E, V473D, G266A and N269Q; or vi) K531E, K459S, V473D, N500E, G266A, N269Q and D590A relative to the VP1 amino acid sequence of SEQ ID NO: 3.

In some embodiments, the AAV VP2 fusion polypeptide and said AAV VP1 and VP3 polypeptides are of the AAV serotype AAV8 and each comprise at least one amino acid substitution within the shared VP3 region selected from the group consisting of G268E, N271Q, S387A, A592Q and A592D relative to the VP1 amino acid sequence of SEQ ID NO: 4, or any combination thereof. In some of these embodiments, the AAV VP2 fusion polypeptide and said AAV VP1 and VP3 polypeptides each comprise the amino acid substitution(s) i) G268E and N271Q; ii) S387A; iii) G268E, N271Q and S387A; iv) A592Q; or v) A592D relative to the VP1 amino acid sequence of SEQ ID NO: 4.

In some embodiments, the AAV VP2 fusion polypeptide and said AAV VP1 and VP3 polypeptides are of the AAV serotype AAV9 and each comprise at least one amino acid substitution within the shared VP3 region selected from the group consisting of Q590A, W503A, N562A and E563A relative to the VP1 amino acid sequence of SEQ ID NO: 5, or any combination thereof. In some of these embodiments, the AAV VP2 fusion polypeptide and said AAV VP1 and VP3 polypeptides each comprise the amino acid substitution(s) i) W503A; ii) N562A and E563A; iii) Q590A and W503A; or iv) Q590A, W503A, N562A and E563A relative to the VP1 amino acid sequence of SEQ ID NO: 5.

In some embodiments, the AAV VP2 fusion polypeptide and said AAV VP1 and VP3 polypeptides are of the AAV serotype AAV2 and each comprise the amino acid substitution R585A relative to the VP1 amino acid sequence of SEQ ID NO: 6 within the shared VP3 region.

In some embodiments, the AAV VP2 fusion polypeptide and said AAV VP1 and VP3 polypeptides are of the AAV serotype AAV1 and each comprise at least one amino acid substitution within the shared VP3 region selected from the group consisting of V473D, N500E and R514A relative to the VP1 amino acid sequence of SEQ ID NO: 7, or any combination thereof. In some of these embodiments, the AAV VP2 fusion polypeptide and said AAV VP1 and VP3 polypeptides each comprise the amino acid substitution(s) i) V473D and N500E; ii) R514A; or iii) V473D, N500E and R514A relative to the VP1 amino acid sequence of SEQ ID NO: 7.

In one aspect, provided herein are cells comprising the rAAV virion described herein.

In some embodiments, the cell is a host cell. In some embodiments, the rAAV virion is assembled in said host cell. The host cell may be selected from the group consisting of an insect cell (e.g., an Sf9 cell) and a HEK293 cell or a derivative thereof, such as a HEK293T/17 cell or an AAV293 cell.

In some embodiments, the cell is selected from a mammalian cell including but not limited to a murine cell, a non-human primate cell or a human cell. In some embodiments, the mammalian cell is selected from the group consisting of a liver cell, a brain cell, a spleen cell, a kidney cell, a blood cell, a lung cell, a muscle cell, a heart cell, a bone marrow cell, a multipotent progenitor cell (MPP), such as a multipotent hematopoietic progenitor cell, and a hematopoietic stem cell (HSC), such as a long term hematopoietic stem cell (LT-HSC).

### Pharmaceutical Compositions and Methods of Treatment

In one aspect, provided herein are pharmaceutical compositions comprising the rAAV virion described herein and a pharmaceutically acceptable excipient.

The rAAV virion described herein can be formulated to prepare pharmaceutically useful compositions. Exemplary formulations include, for example, those disclosed in U.S. Pat. Nos. 9,051,542 and 6,703,237, which are incorporated by reference in their entirety. The compositions of the disclosure can be formulated for administration to a mammalian subject, e.g., a human. In some embodiments, delivery systems may be formulated for intramuscular, intradermal, mucosal, subcutaneous, intravenous, intrathecal, injectable depot type devices, or topical administration.

In some embodiments, when the delivery system is formulated as a solution or suspension, the delivery system is in an acceptable carrier, e.g., an aqueous carrier. A variety of aqueous carriers may be used, e.g., water, buffered water, 0.8% saline, 0.3% glycine, hyaluronic acid and the like. These compositions may be sterilized and/or sterile filtered. The resulting aqueous solutions may be packaged for use as is, or lyophilized. In some embodiments, the lyophilized preparation is combined with a sterile solution prior to administration.

In some embodiments, the compositions, e.g., pharmaceutical compositions, may contain pharmaceutically acceptable auxiliary substances to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. In some embodiments, the pharmaceutical composition comprises a preservative. In some other embodiments, the pharmaceutical composition does not comprise a preservative.

Pharmaceutical compositions are typically formulated to be compatible with its intended route of administration. Non-limiting examples of routes of administration include parenteral, e.g., intravenous, intraarterial, subcutaneous, intraperitoneal, intrathecal, intramuscular, or injection or infusion administration. Methods of formulating suitable pharmaceutical compositions are known in the art, see, e.g., Remington: The Science and Practice of Pharmacy, 21st ed, 2005; and the books in the series Drugs and the Pharmaceutical Sciences: a Series of Textbooks and Monographs (Dekker, NY).

Also provided herein rAAV virions or pharmaceutical compositions comprising same for use as a medicament. For example, the rAAV virion disclosed herein may be administered to deliver a heterologous nucleic acid encoding a therapeutic RNA or protein to a target cell. Thus, the terms “therapeutic application” or “treatment” as used herein include gene therapy for the treatment or prevention of a disease, wherein the term “gene therapy” can be broadly defined as the concept of directed introduction of foreign genetic material into a cell, tissue or organ for correction of defective genes with the goal to improve the clinical status of a patient. For example, the heterologous nucleic acid may replace a gene in the target cell which is mutated or lost resulting in a genetic disease. Administering the rAAV virion disclosed herein may serve to treat, prevent, delay, slow, or ameliorate such genetic disease. In some embodiments, “gene therapy” only refers to “somatic therapy” and not to “germ line therapy”, which would induce heritable changes passed from generation to generation, wherein the somatic therapy restricts the therapeutic effect to the treated individual. The rAAV virion described herein may be administered in vivo or ex vivo. The terms “treat” and “treatment” as used herein comprise the step of administering an effective dose, or effective multiple doses, of a composition comprising the rAAV virion disclosed herein, to an animal (including a human being) in need thereof. If the dose is administered prior to development of a disorder/disease, the administration is prophylactic. If the dose is administered after the development of a disorder/disease, the administration is therapeutic. In some embodiments, an effective dose is a dose that detectably alleviates (either eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. The term encompasses but does not require complete treatment (i.e., curing) and/or prevention.

In one aspect, provided herein is a method of delivering a transgene to a cell, the method comprising contacting the cell with the rAAV virion described herein or the pharmaceutical composition comprising same.

In one aspect, provided herein is a method of delivering a transgene to a cell, wherein the cell expresses a cell surface molecule to which said polypeptide ligand specifically binds.

In some embodiments, the cell is in a living subject. In some embodiments, the subject is a mammalian subject, for instance a human.

In one aspect, provided herein is a method of using the rAAV virion described herein or the pharmaceutical composition comprising same in a therapeutic treatment regimen or as vaccine.

In one aspect, provided herein is a method of using the rAAV virion described herein or the pharmaceutical composition comprising same in a method to reduce the amount of total rAAV virions administered to a subject in a method of treatment, the method comprising administering a reduced amount of total rAAV virions to a subject as compared to the administered amount of an rAAV comprising the identical AAV VP2 capsid polypeptide, which is however not fused to said polypeptide ligand, which is capable of achieving a similar therapeutic effect.

In one aspect, provided herein is a method of using the rAAV virion described herein or the pharmaceutical composition comprising same in a method to increase transduction efficiency of rAAV virions administered to a subject in a method of treatment, the method comprising administering an amount of rAAV virions to a subject, wherein the rAAV virions comprising an AAV VP2 fusion polypeptide is capable of transducing a cell type or tissue at a higher rate as compared to rAAV virions comprising the identical AAV VP2 capsid polypeptide which is however not fused to said polypeptide ligand.

### AAV VP2 Fusion Polypeptide Libraries

In one aspect, provided herein is a library construct comprising a nucleic acid sequence encoding an AAV VP2 fusion polypeptide comprising, e.g., consisting of, an AAV VP2 capsid polypeptide and a polypeptide ligand, wherein the polypeptide ligand is fused to the N-terminus of the AAV VP2 capsid polypeptide and wherein the polypeptide ligand has a molecular weight of up to 10 kDa.

In some embodiments, the polypeptide ligand encoded by the library construct comprises randomized amino acids at at least 1, 2, 3, 4, 5, 6, 7, 8, 9, or 10, amino acid positions.

In one aspect, provided herein is a library construct comprising a nucleic acid sequence encoding an AAV VP2 fusion polypeptide comprising, e.g., consisting of, an AAV VP2 capsid polypeptide and a polypeptide ligand, wherein the polypeptide ligand is fused to the N-terminus of the AAV VP2 capsid polypeptide and wherein said polypeptide ligand is selected from the group consisting of a GP2 polypeptide, an Sso7d polypeptide and an affibody.

In some embodiments, said polypeptide ligand is selected from the group consisting of an Sso7d polypeptide of SEQ ID NO: 1 and an Sso7d polypeptide having at least 80%, 85%, 90%, or 95% sequence identity therewith.

In some embodiments, the polypeptide ligand is selected from an Sso7d polypeptide of SEQ ID NO: 1 optionally harboring up to 10, 9, 8, 7, 6, 5, 4, 3, 2 or 1 amino acid substitution(s).

In some embodiments, said polypeptide ligand is selected from the group consisting of an Sso7d polypeptide of SEQ ID NO: 1, in which amino acid residues X at positions 21, 23, 25, 28, 30, 32, 40, 42 and 44 are independently selected from D, R, H, N, A, I, Y and W, and an Sso7d polypeptide having at least 80%, 85%, 90%, or 95% sequence identity therewith.

In some embodiments, the polypeptide ligand is selected from an Sso7d polypeptide of SEQ ID NO: 1, in which amino acid residues X at positions 21, 23, 25, 28, 30, 32, 40, 42 and 44 are independently selected from D, R, H, N, A, I, Y, W and an amino acid substitution and wherein SEQ ID NO: 1 optionally harbors up to 10, 9, 8, 7, 6, 5, 4, 3, 2 or 1 amino acid substitution(s).

In some embodiments, the encoded AAV VP2 fusion further comprises a peptide linker between the polypeptide ligand and the AAV VP2 capsid polypeptide. In some of these embodiments, said peptide linker is selected from the group consisting of a glycine-serine (GS) linker and an alanine-proline-serine (APS) linker. In some of these embodiments, the GS linker is of the formular [GGGGS]n, wherein n is an integer in the range of 1 to 10, for instance wherein n is 1, 2, 3, 4, 5 or 6, particularly wherein n is 1, 2, 3 or 4. In some embodiments, the APS linker is of the formular [APS]n, wherein n is an integer in the range of 1 to 10, for instance wherein n is 1, 2, 3, 4, 5 or 6, particularly wherein n is 2, 3, 4 or 5.

In some embodiments, the nucleic acid encoding the AAV VP2 fusion polypeptide does not comprise the natural start codon of the AAV VP2 capsid polypeptide. The natural start codon of the AAV VP2 capsid polypeptide may be mutated, e.g., deleted. In some embodiments, the AAV VP2 capsid polypeptide comprised in the AAV VP2 fusion polypeptide misses its first natural amino acid, e.g., the first threonine of the corresponding wild type AAV VP2 capsid polypeptide.

In one aspect, provided herein are libraries comprising a plurality of library constructs described herein. In some embodiments, the library comprises at least two different library constructs described herein. In some embodiments, the at least two different library constructs comprised in the library differ in the nucleic acid sequence encoding the polypeptide ligand.

In some embodiments, the library comprises at least 10², 10¹, 10⁴, 10¹, 10⁶, 10⁷, 10⁸, or 10⁹ unique library constructs. In some embodiments, the library comprises 10²-10³, 10₃-10⁴, 10⁴-10¹, 10⁵-10⁶, 10⁶-10⁷, 10⁷-10⁸, or 10⁸-10⁹ unique library constructs.

In some embodiments, the library comprises at least 10², 10¹, 10⁴, 10¹, 10⁶, 10⁷, 10⁸, 10⁹, or 10¹⁰ members. In some embodiments, the library comprises 10²-10³, 10³-10⁴, 10⁴-10¹, 10⁵-10⁶, 10⁶-10⁷, 10⁷-10⁸, 10⁸-10⁹ or 10⁹-10¹⁰ members.

In some embodiments, the library comprises library constructs encoding all possible variants of the polypeptide ligand.

In some embodiments, the library construct is an RNA molecule, for instance an mRNA molecule, or a DNA molecule, for instance a cDNA molecule, a linear DNA molecule or a circular DNA molecule, e.g., a plasmid DNA molecule. In some embodiments, the library construct further comprises a promoter. In some embodiments the open reading frame of the AAV VP2 fusion polypeptide is operably linked to said promoter.

In some embodiments, the library construct is a plasmid DNA molecule comprising said nucleic acid sequence encoding said AAV VP2 fusion polypeptide operably linked to a promoter. In some embodiments, the library construct further comprises a reporter sequence. In some embodiments, the reporter sequence encodes a fluorescent or a luminescent reporter protein. In some embodiments, the reporter protein is selected from the group consisting of EGFP, mCherry, sfCherry, sfCherry2, mClover3, mRuby3, mApple, iRFP, tdTomato, mVenus, YFP, RFP, firefly luciferase, and nanoluciferase.

In some embodiments, the library construct further comprises a barcode sequence. In some of these embodiments, each specific AAV VP2 fusion polypeptide sequence is assigned a specific barcode sequence, meaning that each specific barcode sequence corresponds to one specific AAV VP2 fusion polypeptide sequence.

In some embodiments, the library is an AAV library, meaning that each library construct is present within an rAAV virion.

In some embodiments, the rAAV virion comprises, e.g., in its capsid, an AAV VP2 fusion polypeptide described herein. In some embodiments, the AAV VP2 fusion polypeptide comprised in a given rAAV virion, e.g., in its capsid, and the AAV VP2 fusion polypeptide encoded by the library construct present in said given rAAV virion are identical. In some embodiments, the majority of the rAAV virions comprise a library construct encoding an AAV VP2 fusion polypeptide which is identical to the AAV VP2 fusion polypeptide comprised in the given rAAV virion. This is referred to as genotype-phenotype linkage or capsid-genome correlation of the AAV library.

In some embodiments, the AAV library comprises at least 10², 10¹, 10⁴, 10¹, 10⁶, 10⁷, 10⁸, or 10⁹ unique AAV variants. In some embodiments, the AAV library comprises 10²-10³, 10³-10⁴, 10⁴-10¹, 10⁵-10⁶, 10⁶-10⁷, 10⁷-10⁸, or 10⁸-10⁹ unique AAV variants.

In some embodiments, the AAV library comprises at least 10², 10¹, 10⁴, 10¹, 10⁶, 10⁷, 10⁸, 10⁹, or 10¹⁰ AAV members. In some embodiments, the AAV library comprises 10²-10³, 10³-10⁴, 10⁴-10⁵, 10⁵-10⁶, 10⁶-10⁷, 10⁷-10⁸, 10⁸-10⁹ or 10⁹-10¹⁰ AAV members.

The AAV libraries described herein may be used for phenotypic selection of capsid polypeptides, e.g., an AAV VP2 fusion polypeptide, with desired characteristics. The AAV VP2 fusion polypeptides are encoded in cis from replicating AAV genomes. This allows for the recovery of the capsid DNA after phenotypic selection. Thus, it is desired to minimize the random mixing of capsomers and the encapsidation of nonmatching viral genomes during the production of the viral libraries. This may for instance be achieved by choosing appropriate conditions for rAAV virion production, e.g., by using appropriate ratios of the nucleic acid molecules encoding the AAV VP2 fusion polypeptide and the nucleic acid molecules encoding the AAV VP1 and VP3 capsid polypeptides for the transfection of the host cells for rAAV virion production. Typically, VP1 and VP3 encoding nucleic acids are used in excess relative to the AAV VP2 fusion polypeptide encoding nucleic acid in the transfection reaction. The AAV VP2 fusion polypeptide encoding nucleic acid is used in such amounts that on average each host cell is only transfected with a single AAV VP2 fusion polypeptide encoding nucleic acid.

In some embodiments, all AAV VP2 capsid polypeptides comprised in the rAAV virions of the AAV library are fused to the polypeptide ligand, either directly or via a linker.

In some embodiments, each library construct of an AAV library comprises two AAV inverted terminal repeat (ITR) sequences located upstream and downstream of the AAV VP2 fusion polypeptide open reading frame, for instance at the 5′ and 3′ termini of the library construct. In some embodiments, the AAV VP2 fusion polypeptide open reading frame is operably linked to a promoter. In some embodiments, the library construct further comprises a reporter sequence. In some embodiments, the reporter sequence encodes a fluorescent or a luminescent reporter protein. In some embodiments, the reporter protein is selected from the group consisting of EGFP, mCherry, sfCherry, sfCherry2, mClover3, mRuby3, mApple, iRFP, tdTomato, mVenus, YFP, RFP, firefly luciferase, and nanoluciferase. In some embodiments, the reporter sequence is operably linked to a promoter, for instance to a second promoter comprised by the library construct.

In some embodiments, the rAAV virions of the AAV library comprise the AAV VP2 fusion polypeptide described herein and further comprise AAV VP1 and VP3 polypeptides. In some embodiments, the AAV VP2 fusion polypeptide and said AAV VP1 and VP3 polypeptides comprised in the rAAV virions are of an AAV serotype independently selected from the group consisting of AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5. In some embodiments, the AAV VP2 fusion polypeptide and said AAV VP1 and VP3 polypeptides comprised in the rAAV virions are of an AAV serotype independently selected from the group consisting of AAV1, AAV6, AAV8 and AAV9. In some embodiments, the AAV VP2 fusion polypeptide and said AAV VP1 and VP3 polypeptides comprised in the rAAV virions are of the same AAV serotype.

In some embodiments, the AAV VP1, VP2 and VP3 polypeptides comprised in the rAAV virions of the AAV library comprise at least one mutation in at least one binding site for their natural receptor present on a target cell of an AAV virion composed of said AAV VP1, VP2 and VP3 polypeptides. In particular embodiments, at least one essential binding site for the natural receptor is mutated in the AAV VP1, VP2 and VP3 polypeptides. In some embodiments, the AAV VP1, VP2 and VP3 polypeptides comprise the same at least one mutation in the shared VP3 region of each AAV capsid polypeptide.

In some embodiments, the AAV VP2 fusion polypeptide and said AAV VP1 and VP3 polypeptides are of the AAV serotype AAV6 and each comprise at least one amino acid substitution within the shared VP3 region selected from the group consisting of K531E, V473D, K459S, N500E, G266A, N269Q, and D590A relative to the VP1 amino acid sequence of SEQ ID NO: 3, or any combination thereof. In some of these embodiments, the AAV VP2 fusion polypeptide and said AAV VP1 and VP3 polypeptides each comprise the amino acid substitutions i) K531E and V473D; ii) K531E, K459S, V473D and N500E; iii) G266A and N269Q; iv) G266A, N269Q and D590A; v) K531E, V473D, G266A and N269Q; or vi) K531E, K459S, V473D, N500E, G266A, N269Q and D590A relative to the VP1 amino acid sequence of SEQ ID NO: 3.

In some embodiments, the AAV VP2 fusion polypeptide and said AAV VP1 and VP3 polypeptides are of the AAV serotype AAV8 and each comprise at least one amino acid substitution within the shared VP3 region selected from the group consisting of G268E, N271Q, S387A, A592Q and A592D relative to the VP1 amino acid sequence of SEQ ID NO: 4, or any combination thereof. In some of these embodiments, the AAV VP2 fusion polypeptide and said AAV VP1 and VP3 polypeptides each comprise the amino acid substitution(s) i) G268E and N271Q; ii) S387A; iii) G268E, N271Q and S387A; iv) A592Q; or v) A592D relative to the VP1 amino acid sequence of SEQ ID NO: 4.

In some embodiments, the AAV VP2 fusion polypeptide and said AAV VP1 and VP3 polypeptides are of the AAV serotype AAV9 and each comprise at least one amino acid substitution within the shared VP3 region selected from the group consisting of Q590A, W503A, N562A and E563A relative to the VP1 amino acid sequence of SEQ ID NO: 5, or any combination thereof. In some of these embodiments, the AAV VP2 fusion polypeptide and said AAV VP1 and VP3 polypeptides each comprise the amino acid substitution(s) i) W503A; ii) N562A and E563A; iii) Q590A and W503A; or iv) Q590A, W503A, N562A and E563A relative to the VP1 amino acid sequence of SEQ ID NO: 5.

In some embodiments, the AAV VP2 fusion polypeptide and said AAV VP1 and VP3 polypeptides are of the AAV serotype AAV2 and each comprise the amino acid substitution R585A relative to the VP1 amino acid sequence of SEQ ID NO: 6 within the shared VP3 region.

In some embodiments, the AAV VP2 fusion polypeptide and said AAV VP1 and VP3 polypeptides are of the AAV serotype AAV1 and each comprise at least one amino acid substitution within the shared VP3 region selected from the group consisting of V473D, N500E and R514A relative to the VP1 amino acid sequence of SEQ ID NO: 7, or any combination thereof. In some of these embodiments, the AAV VP2 fusion polypeptide and said AAV VP1 and VP3 polypeptides each comprise the amino acid substitution(s) i) V473D and N500E; ii) R514A; or iii) V473D, N500E and R514A relative to the VP1 amino acid sequence of SEQ ID NO: 7.

In some embodiments, the AAV VP2 fusion polypeptides comprised in the rAAV virions of the AAV library are of the AAV serotype AAV1 and comprise at least one amino acid substitution selected from the group consisting of E147S, P185G, P166R, M211V, G199R, D213A, T162R, and P191N relative to the VP1 amino acid sequence of SEQ ID NO: 7, or any combination thereof. Particularly, the AAV VP2 fusion polypeptides comprise the amino acid substitution(s) D213A, T162R, and/or P191N, particularly all three amino acid substitutions D213A, T162R, and P191N.

In other embodiments, the AAV VP2 fusion polypeptide comprised in the rAAV virions of the AAV library are of an AAV serotype other than AAV1 and comprise at least one amino acid substitution corresponding to at least one of the amino acid substitution selected from the group consisting of E147S, P185G, P166R, M211V, G199R, D213A, T162R, and P191N relative to the VP1 amino acid sequence of SEQ ID NO: 7.

In particular such embodiments, said AAV VP2 fusion polypeptides comprised in said rAAV virion are of the AAV serotype 8 and comprise at least one amino acid substitution selected from the group consisting of E147S, P186G, P167R, M212V, G200R, D214A, K163R, and P192N relative to the VP1 amino acid sequence of SEQ ID NO: 4, or any combination thereof. Particularly, the AAV VP2 fusion polypeptide comprises the amino acid substitution(s) D214A, K163R and/or P192N, particularly all three substitutions D214A, K163R and P192N.

In other such embodiments, said AAV VP2 fusion polypeptide comprised in said rAAV virion is of the AAV serotype 9 and comprises at least one amino acid substitution selected from the group consisting of E147S, P185G, P166R, G199R, D213A, S162R and P191N relative to the VP1 amino acid sequence of SEQ ID NO: 5, or any combination thereof. Particularly, the AAV VP2 fusion polypeptide comprises the amino acid substitution(s) D213A, S162R and/or P191N, particularly all three amino acid substitutions D213A, S162R and P191N.

In one aspect, provided herein are libraries comprising a plurality of rAAV virions each comprising a variant AAV VP2 fusion polypeptide as described herein.

In some embodiments, the library comprises at least two different rAAV virions each comprising a variant AAV VP2 fusion polypeptide as described herein. In some embodiments, the at least two different rAAV virions differ in the polypeptide ligand comprised in the AAV VP2 fusion polypeptide.

In some embodiments, the AAV library comprises at least 10², 10¹, 10⁴, 10¹, 10⁶, 10⁷, 10⁸, or 10⁹ unique rAAV virions. In some embodiments, the AAV library comprises 10²-10³, 10³-10⁴, 10⁴-10⁵, 10⁵-10⁶, 10⁶-10⁷, 10⁷-10⁸, or 10⁸-10⁹ unique rAAV virions.

In some embodiments, the AAV library comprises at least 10², 10¹, 10⁴, 10¹, 10⁶, 10⁷, 10⁸, 10⁹, or 10¹⁰ rAAV virion members. In some embodiments, the AAV library comprises 10²-10³, 10³-10⁴, 10⁴-10⁵, 10⁵-10⁶, 10⁶-10⁷, 10⁷-10⁸, 10⁸-10⁹ or 10⁹-10¹⁰ rAAV virion members.

In some embodiments, the rAAV virions comprised in said library comprise a nucleic acid comprising a barcode sequence. In some embodiments, each specific AAV VP2 fusion polypeptide is assigned a specific barcode sequence, meaning that an rAAV virion comprising an AAV VP2 fusion polypeptide of a given amino acid sequence comprises a nucleic acid comprising the corresponding barcode sequence. The barcode sequence may for instance comprise 5, 6, 7, 8, 9, 10, 11, 12, 13, 14, 15, 16, 17, 18 or more nucleotides.

### Methods of Identifying AAV VP2 Fusion Polypeptide Variants

In one aspect, provided herein are methods of generating an AAV VP2 fusion polypeptide with at least one desired characteristic, the method comprising screening the library described herein for an encoded AAV VP2 fusion polypeptide with said at least one desired characteristic.

In some embodiments, said at least one desired characteristic is increased transduction of and/or increased tropism in at least one tissue or cell type mediated by said AAV VP2 fusion polypeptide relative to an AAV VP2 capsid polypeptide not comprising said polypeptide ligand but otherwise identical to the AAV VP2 capsid polypeptide comprised in the AAV VP2 fusion polypeptide.

In one aspect, provided herein are methods of generating an AAV VP2 fusion polypeptide with at least one desired characteristic, comprising the steps: b) contacting the library described herein with a plurality of cells, c) isolating nucleic acid molecules from at least a part of said cells, and d) determining at least a part of the sequence encoding the polypeptide ligand or a fragment thereof of at least one nucleic acid molecule isolated in step b). In some embodiments, the plurality of cells in step b) is present within a non-human model animal, for instance a murine model or a non-human primate model.

In some embodiments, the method further comprises a step a) of providing an AAV library as described herein before step b). In some embodiments, the library is packaged in HEK293 cells or HEK293 derivative cells, where the helper functions (e.g., E2A, E4, VA, E1A and E1B) are supplied in trans. In some embodiments, the AAV rep function comprises rep78, rep 68, rep 52, and rep40 gene products. In some embodiments, the rep gene is supplied in trans. In some embodiments, the start codon of the rep78 and/or the rep68 open reading frame is altered from ACG to ATG to increase replication of the capsid library construct containing inverted terminal repeats (ITRs), thereby improving AAV library manufacturing yield. In some embodiments, the cap gene is supplied in trans. In some embodiments, the cap gene is controlled by the p40 promoter such that it is only expressed during manufacturing in HEK293 cells in the presence of helper virus functions. In some embodiments, the start codon of the VP2 open reading frame in the cap gene provided in trans is mutated. In some embodiments, the nucleic acid encoding the AAV VP2 fusion polypeptide is supplied as payload to the production cell line in AAV manufacture.

In some embodiments, step a) of providing an AAV library comprises ai) transfecting cells with a nucleic acid sequence encoding AAV rep, a nucleic acid sequence encoding an AAV VP1 capsid polypeptide, a nucleic acid sequence encoding an AAV VP3 capsid polypeptide and a library of nucleic acids encoding the AAV VP2 fusion polypeptide described herein, wherein each library construct further comprises two AAV inverted terminal repeat (ITR) sequences at its 5′ and 3′ termini, and aii) incubating the cell under conditions suitable for AAV virion formation, optionally wherein the cells are mammalian cells, particularly HEK293 cells, or insect cells, particularly Sf9 cells.

In some embodiments, said AAV VP1 capsid polypeptide and said AAV VP3 capsid polypeptide are encoded by the same nucleic acid sequence comprising an AAV cap gene, particularly wherein the start codon of VP2 in the cap gene is mutated.

In some embodiments, the plurality of cells in step b) is present within a non-human model animal. In some embodiments, the animal is treated with the AAV library via intravenous, intracranial, or intrathecal injection, or by injection by some other route (e.g., nasal, hepatic, intracerebroventricular, intracisternal, intravitreal, intracochlear, etc.). Following a sufficient time for the AAV to traffic to the desired tissue or organ (for example, 7, 10, 14, 18, 21, 24, 28, 30 days or more), the animal is sacrificed, and the tissue/organ of interest is harvested. In some embodiments, the tissue/organ of interest which is harvested is selected from CNS, brain, heart, lung, trachea, esophagus, muscle, bone, cartilage, bone marrow, stomach, pancreas, intestine, liver, spleen, bladder, kidney, ureter, urethra, uterus, fallopian tube, ovary, testes, prostate, eye, blood, lymph, and oral mucosa. In some embodiments, the tissue/organ of interest which is harvested is selected from whole blood, brain, liver, spleen, kidney, skeletal muscle, heart, lungs, and bone marrow.

In some embodiments, the cells in the tissues are monitored for the expression of the reporter sequence in the cytoplasm of the cell or in the organelle of interest. In some embodiments, the organelle of interest is the nucleus. In some embodiments, the organelles comprising the AAV payload as evidenced by the expression of the reporter sequence are isolated from the tissue. In some embodiments, the organelle is the nucleus. In some embodiments, the method further comprises a step of selecting cells in which said reporter sequence is expressed.

In some embodiments, in step c) nucleic acids from cells of a predefined tissue or cell type are isolated. In some embodiments, the cells of predefined tissue are selected from cells from the CNS, brain, heart, lung, trachea, esophagus, muscle, bone, cartilage, bone marrow, stomach, pancreas, intestine, liver, spleen, bladder, kidney, ureter, urethra, uterus, fallopian tube, ovary, testes, prostate, eye, blood, lymph, and oral mucosa. In some embodiments, the cells of predefined tissue are selected from whole blood, brain, liver, spleen, kidney, skeletal muscle, heart, lungs, and bone marrow. In some embodiments, the cells of a predefined cell type are selected from multipotent progenitor cells (MPPs), such as multipotent hematopoietic progenitor cells, and hematopoietic stem cells (HSCs), such as long-term hematopoietic stem cells (LT-HSCs). In some embodiments, said cells of a predefined tissue or cell type are isolated 1-21 days, 1-14 days, 7-21 days, 2-10 days, 2-5 days after contacting the plurality of cells with said library.

In some embodiments, nucleic acid is extracted from the organelle (e.g., nucleus). In some embodiments, the nucleic acid extracted is RNA, while in some embodiments, the nucleic acid extracted is DNA. In some embodiments, the extracted RNA is subject to reverse transcription to generate cDNA which is then amplified. In some embodiments, primers specific to the barcoded region are used in amplification. In some embodiments, at least part of the sequence encoding the variable polypeptide ligand is amplified. In some embodiments, the amplified cDNA is sequenced. In some embodiments, enrichment of specific AAV variants in specific tissues/organs is observed following selection in vivo.

In some embodiments, step c) comprises isolating RNA from at least a part of said cells and step d) comprises the sub-steps di) reverse transcription of said isolated RNA, dii) PCR amplification of at least a part of the sequence encoding the polypeptide ligand or a fragment thereof from the cDNA generated in step di) and diii) sequencing at least a part of the polypeptide ligand sequence, for instance by next generation sequencing.

In some embodiments, the method further comprises step e) of determining the relative occurrence of a specific polypeptide ligand sequence in one tissue or cell type relative to the total number of polypeptide ligand sequences in said tissue or cell type.

In some embodiments, the in vivo selection of AAV VP2 fusion polypeptide variants may be performed in 1, 2, 3, 4, 5 or more rounds, in each case pooling the AAV variants obtained from the previous round or synthesizing a subset of variants that were enriched and re-selecting in in vitro or in vivo.

In some embodiments, the method further comprises step f) of generating a second library of nucleic acid sequences encoding an AAV VP2 fusion polypeptide from the nucleic acid molecules isolated in step c) and repeating steps b), c), d) and optionally a) and e) with said second library.

In some embodiments, following the desired number of selection rounds, the obtained variants are analyzed. In some embodiments, the individual variants are used to deliver a transgene, such as a reporter gene, to a desired cell or organ in vivo. After analysis of the delivery capability of the variants, the best candidates are selected for future use.

In some embodiments, the selected candidates are subjected to functional maturation by random mutagenesis and in vivo or in vitro selection of variants with improved characteristics such as improved transduction of cells of a specific cell type or tissue.

In one aspect, provided herein are AAV VP2 fusion polypeptides generated by the method described herein.

Also provided herein are rAAV virions comprising the AAV VP2 fusion polypeptide generated by the method described herein and pharmaceutical compositions comprising same. Said rAAV virions and pharmaceutical compositions may be used as medicament.

AAV VP2 Capsid Polypeptides with Improved Receptor Binding and/or Transduction Levels

In one aspect, provided herein are AAV VP2 capsid polypeptides of the AAV serotype AAV1 comprising at least one amino acid substitution selected from the group consisting of E147S, P185G, P166R, M211V, G199R, D213A, T162R, and P191N relative to the VP1 amino acid sequence of SEQ ID NO: 7, or any combination thereof. In some embodiments, the AAV VP2 capsid polypeptide comprises the amino acid substitution(s) D213A, T162R, and/or P191N, particularly all three amino acid substitutions D213A, T162R, and P191N.

In another aspect, provided herein are AAV VP2 capsid polypeptides of an AAV serotype other than AAV1 which comprise at least one amino acid substitution corresponding to at least one of the amino acid substitutions selected from the group consisting of E147S, P185G, P166R, M211V, G199R, D213A, T162R, and P191N relative to the VP1 amino acid sequence of SEQ ID NO: 7.

In some embodiments, the AAV VP2 capsid polypeptide is of the AAV serotype 8 and comprises at least one amino acid substitution selected from the group consisting of E147S, P186G, P167R, M212V, G200R, D214A, K163R, and P192N relative to the VP1 amino acid sequence of SEQ ID NO: 4, or any combination thereof. Particularly, the AAV VP2 capsid polypeptide comprises the amino acid substitution(s) D214A, K163R and/or P192N, particularly all three amino acid substitutions D214A, K163R and P192N.

In other embodiments, the AAV VP2 capsid polypeptide is of the AAV serotype 9 and comprises at least one amino acid substitution selected from the group consisting of E147S, P185G, P166R, G199R, D213A, S162R and P191N relative to the VP1 amino acid sequence of SEQ ID NO: 5, or any combination thereof. Particularly, the AAV VP2 capsid polypeptide comprises the amino acid substitution(s) D213A, S162R and/or P191N, particularly all three amino acid substitutions D213A, S162R and/or P191N.

Also provided herein are AAV VP2 fusion polypeptides comprising one of the above listed AAV VP2 capsid polypeptides and a polypeptide ligand, wherein the polypeptide ligand is fused to the N-terminus of the AAV VP2 capsid polypeptide.

### Embodiments

1. An adeno-associated virus (AAV) VP2 fusion polypeptide comprising, e.g., consisting of, an AAV VP2 capsid polypeptide and a polypeptide ligand, wherein the polypeptide ligand is fused to the N-terminus of the AAV VP2 capsid polypeptide and wherein the polypeptide ligand has a molecular weight of up to 10 kDa.

2. The AAV VP2 fusion polypeptide of embodiment 1, wherein said polypeptide ligand specifically binds to a cell surface molecule expressed on at least one tissue or cell type and/or wherein said AAV VP2 fusion polypeptide mediates increased transduction of and/or increased tropism in at least one tissue or cell type relative to an AAV VP2 capsid polypeptide not comprising said polypeptide ligand.

3. The AAV VP2 fusion polypeptide of embodiment 1 or 2, wherein said polypeptide ligand is selected from the group consisting of a GP2 polypeptide, an Sso7d polypeptide and an affibody.

4. The AAV VP2 fusion polypeptide of embodiment 3, wherein said polypeptide ligand is selected from the group consisting of an Sso7d polypeptide of SEQ ID NO: 1 and an Sso7d polypeptide having at least 80%, 85%, 90%, or 95% sequence identity therewith.

5. The AAV VP2 fusion polypeptide of embodiment 4, wherein said polypeptide ligand is selected from the group consisting of an Sso7d polypeptide of SEQ ID NO: 1, in which the amino acid residues X at positions 21, 23, 25, 28, 30, 32, 40, 42 and 44 are independently selected from D, R, H, N, A, I, Y and W, and an Sso7d polypeptide having at least 80%, 85%, 90%, or 95% sequence identity therewith.

6. The AAV VP2 fusion polypeptide of any one of the preceding embodiments, further comprising a peptide linker between the polypeptide ligand and the AAV VP2 capsid polypeptide, in particular wherein said peptide linker is selected from the group consisting of a glycine-serine (GS) linker and an alanine-proline-serine (APS) linker, more particularly wherein the GS linker is of the formular [GGGGS]n and the APS linker is of the formular [APS]n, wherein n is an integer in the range of 1 to 10, in particular wherein n is 1, 2, 3, 4, 5 or 6, more particularly wherein n is 1, 2, 3 or 4 for the GS linker and 2, 3, 4 or 5 for the APS linker.

7. The AAV VP2 fusion polypeptide of any one of the preceding embodiments, wherein said AAV VP2 capsid polypeptide is of an AAV serotype selected from the group consisting of AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5, particularly from the group consisting of AAV1, AAV6, AAV8 and AAV9.

8. The AAV VP2 fusion polypeptide of embodiment 7, wherein the AAV VP2 capsid polypeptide comprises at least one mutation in at least one binding site for its natural receptor, particularly wherein
a) the AAV VP2 fusion polypeptide is of the AAV serotype AAV6 and comprises at least one amino acid substitution within its VP3 region selected from the group consisting of K531E, V473D, K459S, N500E, G266A, N269Q, and D590A relative to the VP1 amino acid sequence of SEQ ID NO: 3, or any combination thereof, in particular wherein the AAV VP2 fusion polypeptide comprises the amino acid substitutions ai) K531E and V473D; aii) K531E, K459S, V473D and N500E; aiii) G266A and N269Q; aiv) G266A, N269Q and D590A; av) K531E, V473D, G266A and N269Q; or avi) K531E, K459S, V473D, N500E, G266A, N269Q and D590A relative to the VP1 amino acid sequence of SEQ ID NO: 3; wherein
b) the AAV VP2 fusion polypeptide is of the AAV serotype AAV8 and comprises at least one amino acid substitution within its VP3 region selected from the group consisting of G268E, N271Q, S387A, A592Q and A592D relative to the VP1 amino acid sequence of SEQ ID NO: 4, or any combination thereof, in particular wherein the AAV VP2 fusion polypeptide comprises the amino acid substitution(s) bi) G268E and N271Q; bii) S387A; biii) G268E, N271Q and S387A; biv) A592Q; or bv) A592D relative to the VP1 amino acid sequence of SEQ ID NO: 4; wherein
c) the AAV VP2 fusion polypeptide is of the AAV serotype AAV9 and comprises at least one amino acid substitution within its VP3 region selected from the group consisting of Q590A, W503A, N562A and E563A relative to the VP1 amino acid sequence of SEQ ID NO: 5, or any combination thereof, in particular wherein the AAV VP2 fusion polypeptide comprises the amino acid substitution(s) ci) W503A; cii) N562A and E563A; ciii) Q590A and W503A; or civ) Q590A, W503A, N562A and E563A relative to the VP1 amino acid sequence of SEQ ID NO: 5; wherein
d) the AAV VP2 fusion polypeptide is of the AAV serotype AAV2 and comprises the amino acid substitution R585A relative to the VP1 amino acid sequence of SEQ ID NO: 6 within its VP3 region; or wherein
e) the AAV VP2 fusion polypeptide is of the AAV serotype AAV1 and comprises at least one amino acid substitution within its VP3 region selected from the group consisting of V473D, N500E and R514A relative to the VP1 amino acid sequence of SEQ ID NO: 7, or any combination thereof, in particular wherein the AAV VP2 fusion polypeptide comprises the amino acid substitution(s) ei) V473D and N500E; eii) R514A; or eiii) V473D, N500E and R514A relative to the VP1 amino acid sequence of SEQ ID NO: 7.

9. The AAV VP2 fusion polypeptide of any one of the preceding embodiments, wherein a) the AAV VP2 fusion polypeptide is of the AAV serotype AAV1 and comprises at least one amino acid substitution selected from the group consisting of E147S, P185G, P166R, M211V, G199R, D213A, T162R, and P191N relative to the VP1 amino acid sequence of SEQ ID NO: 7, or any combination thereof, in particular wherein the AAV VP2 fusion polypeptide comprises the amino acid substitution(s) D213A, T162R, and/or P191N, or wherein b) the AAV VP2 fusion polypeptide is of an AAV serotype other than AAV1 and comprises at least one amino acid substitution corresponding to at least one of the amino acid substitution selected from the group consisting of E147S, P185G, P166R, M211V, G199R, D213A, T162R, and P191N relative to the VP1 amino acid sequence of SEQ ID NO: 7, particularly wherein bi) the AAV VP2 fusion polypeptide is of the AAV serotype 8 and comprises at least one amino acid substitution selected from the group consisting of E147S, P186G, P167R, M212V, G200R, D214A, K163R, and P192N relative to the VP1 amino acid sequence of SEQ ID NO: 4, or any combination thereof, in particular wherein the AAV VP2 fusion polypeptide comprises the amino acid substitution(s) D214A, K163R and/or P192N, or wherein bii) the AAV VP2 fusion polypeptide is of the AAV serotype 9 and comprises at least one amino acid substitution selected from the group consisting of E147S, P185G, P166R, G199R, D213A, S162R and P191N relative to the VP1 amino acid sequence of SEQ ID NO: 5, or any combination thereof, in particular wherein the AAV VP2 fusion polypeptide comprises the amino acid substitution(s) D213A, S162R and/or P191N.

10. The AAV VP2 fusion polypeptide of embodiment 9a), wherein the AAV VP2 fusion polypeptide further comprises the amino acid substitutions V473D and N500E relative to the VP1 amino acid sequence of SEQ ID NO: 7.

11. The AAV VP2 fusion polypeptide of any one of the preceding embodiments, wherein said AAV VP2 capsid polypeptide lacks the first threonine of the corresponding wild type AAV VP2 capsid polypeptide.

12. The AAV VP2 fusion polypeptide of any one of the preceding embodiments, wherein said polypeptide ligand comprises up to 3 cysteine residues, particularly up to 2 cysteine residues, more particularly up to 1 cysteine residue, most particularly no cysteine residue.

13. An isolated nucleic acid encoding the AAV VP2 fusion polypeptide of any one of the preceding embodiments.

14. The isolated nucleic acid of embodiment 13, further comprising a promoter.

15. The isolated nucleic acid of embodiment 13 or 14, further comprising two AAV inverted terminal repeat (ITR) sequences located upstream and downstream of the AAV VP2 fusion polypeptide open reading frame (ORF) and optionally further comprising a reporter sequence, particularly wherein the reporter sequence encodes a fluorescent or a luminescent reporter protein, more particularly wherein the reporter protein is selected from the group consisting of EGFP, mCherry, sfCherry, sfCherry2, mClover3, mRuby3, mApple, iRFP, tdTomato, mVenus, YFP, RFP, firefly luciferase, and nanoluciferase.

16. A cell comprising the AAV VP2 fusion polypeptide of any one of embodiments 1 to 12 or the nucleic acid of any one of embodiments 13 to 15.

17. The cell of embodiment 16, wherein the cell is selected from the group consisting of an insect cell (e.g., an Sf9 cell) and a HEK293 cell.

18. The cell of embodiment 16, wherein the cell is selected from a mammalian cell including but not limited to a murine cell, a non-human primate cell or a human cell selected from the group consisting of a liver cell, a brain cell, a spleen cell, a kidney cell, a blood cell, a lung cell, a muscle cell, a heart cell, a bone marrow cell, a multipotent progenitor cell (MPP), such as a multipotent hematopoietic progenitor cell, and a hematopoietic stem cell (HSC), such as a long term hematopoietic stem cell (LT-HSC).

19. A recombinant AAV (rAAV) virion comprising the AAV VP2 fusion polypeptide of any one of embodiments 1 to 12.

20. The rAAV virion of embodiment 19, in which all AAV VP2 capsid polypeptides are fused to said polypeptide ligand.

21. The rAAV virion of embodiment 19 or 20, further comprising a nucleic acid sequence selected from the group consisting of a non-coding nucleic acid, a protein or an RNA coding sequence, an expression cassette, a multi-expression cassette, a sequence for homologous recombination, and a genomic gene targeting cassette.

22. The rAAV virion of embodiment 21, wherein said expression cassette encodes the AAV VP2 fusion polypeptide of any one of embodiments 1 to 12, in particular wherein the AAV VP2 fusion polypeptide comprised in the rAAV virion and the AAV VP2 fusion polypeptide encoded by said expression cassette are identical.

23. The rAAV virion of embodiment 21, wherein said expression cassette encodes a therapeutic nucleic acid including but not limited to a therapeutic RNA, mRNA, siRNA, miRNA, shRNA, and an antisense oligonucleotide, a therapeutic protein or a therapeutic antibody or antibody fragment.

24. The rAAV virion according any one of embodiment 19 to 23, wherein the virion further comprises AAV VP1 and VP3 polypeptides, in particular wherein the AAV VP2 fusion polypeptide and said AAV VP1 and VP3 polypeptides are of the same AAV serotype selected from the group consisting of AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5, particularly from the group consisting of AAV1, AAV6, AAV8 and AAV9.

25. The rAAV virion of embodiment 24, wherein the AAV VP1, VP2 and VP3 polypeptides comprise at least one mutation in at least one binding site for the natural receptor of the AAV VP1, VP2 and VP3 capsid polypeptides, particularly wherein the AAV VP1, VP2 and VP3 polypeptides comprise the same at least one mutation in the shared VP3 region of each AAV capsid polypeptide, more particularly wherein
a) the AAV VP2 fusion polypeptide and said AAV VP1 and VP3 polypeptides are of the AAV serotype AAV6 and each comprise at least one amino acid substitution within the shared VP3 region selected from the group consisting of K531E, V473D, K459S, N500E, G266A, N269Q, and D590A relative to the VP1 amino acid sequence of SEQ ID NO: 3, or any combination thereof, in particular wherein the AAV VP2 fusion polypeptide and said AAV VP1 and VP3 polypeptides each comprise the amino acid substitutions ai) K531E and V473D; aii) K531E, K459S, V473D and N500E; aiii) G266A and N269Q; aiv) G266A, N269Q and D590A; av) K531E, V473D, G266A and N269Q; or avi) K531E, K459S, V473D, N500E, G266A, N269Q and D590A relative to the VP1 amino acid sequence of SEQ ID NO: 3; wherein
b) the AAV VP2 fusion polypeptide and said AAV VP1 and VP3 polypeptides are of the AAV serotype AAV8 and each comprise at least one amino acid substitution within the shared VP3 region selected from the group consisting of G268E, N271Q, S387A, A592Q and A592D relative to the VP1 amino acid sequence of SEQ ID NO: 4, or any combination thereof, in particular wherein the AAV VP2 fusion polypeptide and said AAV VP1 and VP3 polypeptides each comprise the amino acid substitution(s) bi) G268E and N271Q; bii) S387A; biii) G268E, N271Q and S387A; biv) A592Q; or bv) A592D relative to the VP1 amino acid sequence of SEQ ID NO: 4; wherein
c) the AAV VP2 fusion polypeptide and said AAV VP1 and VP3 polypeptides are of the AAV serotype AAV9 and each comprise at least one amino acid substitution within the shared VP3 region selected from the group consisting of Q590A, W503A, N562A and E563A relative to the VP1 amino acid sequence of SEQ ID NO: 5, or any combination thereof, in particular wherein the AAV VP2 fusion polypeptide and said AAV VP1 and VP3 polypeptides each comprise the amino acid substitution(s) ci) W503A; cii) N562A and E563A; ciii) Q590A and W503A; or civ) Q590A, W503A, N562A and E563A relative to the VP1 amino acid sequence of SEQ ID NO: 5; wherein
d) the AAV VP2 fusion polypeptide and said AAV VP1 and VP3 polypeptides are of the AAV serotype AAV2 and each comprise the amino acid substitution R585A relative to the VP1 amino acid sequence of SEQ ID NO: 6 within the shared VP3 region; or wherein
e) the AAV VP2 fusion polypeptide and said AAV VP1 and VP3 polypeptides are of the AAV serotype AAV1 and each comprise at least one amino acid substitution within the shared VP3 region selected from the group consisting of V473D, N500E and R514A relative to the VP1 amino acid sequence of SEQ ID NO: 7, or any combination thereof, in particular wherein the AAV VP2 fusion polypeptide and said AAV VP1 and VP3 polypeptides each comprise the amino acid substitution(s) ei) V473D and N500E; eii) R514A; or eiii) V473D, N500E and R514A relative to the VP1 amino acid sequence of SEQ ID NO: 7.

26. A pharmaceutical composition comprising the rAAV virion of any one of embodiments 19 to 21 and 23 to 25 and a pharmaceutically acceptable excipient.

27. The rAAV virion of any one of embodiments 19 to 21 and 23 to 25 or the pharmaceutical composition of embodiment 26 for use as a medicament.

28. A method of delivering a transgene to a cell, the method comprising contacting the cell with the rAAV virion of any one of embodiments 19 to 21 and 23 to 25 or the pharmaceutical composition of embodiment 26.

29. The method of embodiment 28, wherein the cell is in a living subject, particularly wherein the subject is a mammalian subject, more particularly a human.

30. A method of using the rAAV virion of any one of embodiments 19 to 21 and 23 to 25 or the pharmaceutical composition of embodiment 26 in a therapeutic treatment regimen or as vaccine.

31. A library construct comprising a nucleic acid sequence encoding an AAV VP2 fusion polypeptide comprising, e.g., consisting of, an AAV VP2 capsid polypeptide and a polypeptide ligand, wherein the polypeptide ligand is fused to the N-terminus of the AAV VP2 capsid polypeptide and wherein the polypeptide ligand has a molecular weight of up to 10 kDa.

32. The library construct of embodiment 31, wherein the polypeptide ligand comprises randomized amino acids at at least 1, 2, 3, 4, 5, 6, 7, 8, 9, or 10 amino acid positions.

33. The library construct of embodiment 31 or 32, wherein said polypeptide ligand is selected from the group consisting of a GP2 polypeptide, an Sso7d polypeptide and an affibody.

34. The library construct of embodiment 33, wherein said polypeptide ligand is selected from the group consisting of an Sso7d polypeptide of SEQ ID NO: 1 and an Sso7d polypeptide having at least 80%, 85%, 90%, or 95% sequence identity therewith.

35. The library construct of embodiment 34, wherein said polypeptide ligand is selected from the group consisting of an Sso7d polypeptide of SEQ ID NO: 1, in which the amino acid residues X at positions 21, 23, 25, 28, 30, 32, 40, 42 and 44 are independently selected from D, R, H, N, A, I, Y and W, and an Sso7d polypeptide having at least 80%, 85%, 90%, or 95% sequence identity therewith.

36. A library comprising a plurality of library constructs of any one of embodiments 31 to 35.

37. The library of embodiment 36, wherein the library comprises at least 10², 10¹, 10⁴, 10¹, 10⁶, 10⁷, 10⁸, or 10⁹ unique library constructs.

38. The library of embodiment 36 or 37, wherein the library constructs are plasmid DNA molecules comprising said nucleic acid sequence encoding said AAV VP2 fusion polypeptide operably linked to a promoter and optionally further comprising a reporter sequence, particularly wherein the reporter sequence encodes a fluorescent or a luminescent reporter protein, more particularly wherein the reporter protein is selected from the group consisting of EGFP, mCherry, sfCherry, sfCherry2, mClover3, mRuby3, mApple, iRFP, tdTomato, mVenus, YFP, RFP, firefly luciferase, and nanoluciferase.

39. The library of embodiment 36 or 37, wherein each library construct is present within an rAAV virion, particularly wherein said rAAV virion comprises an AAV VP2 fusion polypeptide of embodiment 4 or 5 and wherein the AAV VP2 fusion polypeptide comprised in a given rAAV virion and the AAV VP2 fusion polypeptide encoded by the library construct present in said given rAAV virion are identical.

40. The library of embodiment 39, wherein each library construct further comprises a promoter, two AAV inverted terminal repeat (ITR) sequences located upstream of the promoter and downstream of the nucleic acid sequence encoding the AAV VP2 fusion polypeptide and optionally a reporter sequence, particularly wherein the reporter sequence encodes a fluorescent or a luminescent reporter protein, more particularly wherein the reporter protein is selected from the group consisting of EGFP, mCherry, sfCherry, mClover3, mRuby3, mApple, iRFP, tdTomato, mVenus, YFP, RFP, firefly luciferase, and nanoluciferase.

41. The library of embodiment 39 or 40, wherein said rAAV virion comprises an AAV VP2 fusion polypeptide of embodiment 4 or 5 and further comprises AAV VP1 and VP3 polypeptides, in particular wherein the AAV VP2 fusion polypeptide and said AAV VP1 and VP3 polypeptides comprised in said rAAV virion are of the same AAV serotype selected from the group consisting of AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV or avian AAV.5, particularly from the group consisting of AAV1, AAV6, AAV8 and AAV9.

42. The library of embodiment 41, wherein the AAV VP1, VP2 and VP3 polypeptides comprised in said rAAV virion comprise at least one mutation in at least one binding site for the natural receptor of the AAV VP1, VP2 and VP3 capsid polypeptides, particularly wherein the AAV VP1, VP2 and VP3 polypeptides comprise the same at least one mutation in the shared VP3 region of each AAV capsid polypeptide, more particularly wherein
a) the AAV VP2 fusion polypeptide and said AAV VP1 and VP3 polypeptides are of the AAV serotype AAV6 and each comprise at least one amino acid substitution within the shared VP3 region selected from the group consisting of K531E, V473D, K459S, N500E, G266A, N269Q, and D590A relative to the VP1 amino acid sequence of SEQ ID NO: 3, or any combination thereof, in particular wherein the AAV VP2 fusion polypeptide and said AAV VP1 and VP3 polypeptides each comprise the amino acid substitutions ai) K531E and V473D; aii) K531E, K459S, V473D and N500E; aiii) G266A and N269Q; aiv) G266A, N269Q and D590A; av) K531E, V473D, G266A and N269Q; or avi) K531E, K459S, V473D, N500E, G266A, N269Q and D590A relative to the VP1 amino acid sequence of SEQ ID NO: 3; wherein
b) the AAV VP2 fusion polypeptide and said AAV VP1 and VP3 polypeptides are of the AAV serotype AAV8 and each comprise at least one amino acid substitution within the shared VP3 region selected from the group consisting of G268E, N271Q, S387A, A592Q and A592D relative to the VP1 amino acid sequence of SEQ ID NO: 4, or any combination thereof, in particular wherein the AAV VP2 fusion polypeptide and said AAV VP1 and VP3 polypeptides each comprise the amino acid substitution(s) bi) G268E and N271Q; bii) S387A; biii) G268E, N271Q and S387A; biv) A592Q; or bv) A592D relative to the VP1 amino acid sequence of SEQ ID NO: 4; wherein
c) the AAV VP2 fusion polypeptide and said AAV VP1 and VP3 polypeptides are of the AAV serotype AAV9 and each comprise at least one amino acid substitution within the shared VP3 region selected from the group consisting of Q590A, W503A, N562A and E563A relative to the VP1 amino acid sequence of SEQ ID NO: 5, or any combination thereof, in particular wherein the AAV VP2 fusion polypeptide and said AAV VP1 and VP3 polypeptides each comprise the amino acid substitution(s) ci) W503A; cii) N562A and E563A; ciii) Q590A and W503A; or civ) Q590A, W503A, N562A and E563A relative to the VP1 amino acid sequence of SEQ ID NO: 5; wherein
d) the AAV VP2 fusion polypeptide and said AAV VP1 and VP3 polypeptides are of the AAV serotype AAV2 and each comprise the amino acid substitution R585A relative to the VP1 amino acid sequence of SEQ ID NO: 6 within the shared VP3 region; or wherein
e) the AAV VP2 fusion polypeptide and said AAV VP1 and VP3 polypeptides are of the AAV serotype AAV1 and each comprise at least one amino acid substitution within the shared VP3 region selected from the group consisting of V473D, N500E and R514A relative to the VP1 amino acid sequence of SEQ ID NO: 7, or any combination thereof, in particular wherein the AAV VP2 fusion polypeptide and said AAV VP1 and VP3 polypeptides each comprise the amino acid substitution(s) ei) V473D and N500E; eii) R514A; or eiii) V473D, N500E and R514A relative to the VP1 amino acid sequence of SEQ ID NO: 7.

43. The library of embodiment 41, wherein a) said AAV VP2 fusion polypeptides comprised in said rAAV virion is of the AAV serotype AAV1 and comprises at least one amino acid substitution selected from the group consisting of E147S, P185G, P166R, M211V, G199R, D213A, T162R, and P191N relative to the VP1 amino acid sequence of SEQ ID NO: 7, or any combination thereof, in particular wherein the AAV VP2 fusion polypeptide comprises the amino acid substitution(s) D213A, T162R, and/or P191N, or wherein b) said AAV VP2 fusion polypeptide comprised in said rAAV virion is of an AAV serotype other than AAV1 and comprises at least one amino acid substitution corresponding to at least one of the amino acid substitution selected from the group consisting of E147S, P185G, P166R, M211V, G199R, D213A, T162R, and P191N relative to the VP1 amino acid sequence of SEQ ID NO: 7, particularly wherein bi) said AAV VP2 fusion polypeptide comprised in said rAAV virion is of the AAV serotype 8 and comprises at least one amino acid substitution selected from the group consisting of E147S, P186G, P167R, M212V, G200R, D214A, K163R, and P192N relative to the VP1 amino acid sequence of SEQ ID NO: 4, or any combination thereof, in particular wherein the AAV VP2 fusion polypeptide comprises the amino acid substitution(s) D214A, K163R and/or P192N, or wherein bii) said AAV VP2 fusion polypeptide comprised in said rAAV virion is of the AAV serotype 9 and comprises at least one amino acid substitution selected from the group consisting of E147S, P185G, P166R, G199R, D213A, S162R and P191N relative to the VP1 amino acid sequence of SEQ ID NO: 5, or any combination thereof, in particular wherein the AAV VP2 fusion polypeptide comprises the amino acid substitution(s) D213A, S162R and/or P191N.

44. A method of generating an AAV VP2 fusion polypeptide with at least one desired characteristic, the method comprising screening the library of any one of embodiments 36 to 43 for an encoded AAV VP2 fusion polypeptide with said at least one desired characteristic.

45. The method of embodiment 44, wherein said at least one desired characteristic is increased transduction of and/or increased tropism in at least one tissue or cell type mediated by said AAV VP2 fusion polypeptide relative to an AAV VP2 capsid polypeptide not comprising said polypeptide ligand.

46. The method of embodiment 44 or 45, comprising the steps: b) contacting the library of any one of embodiments 36 to 43 with a plurality of cells, c) isolating nucleic acid molecules from at least a part of said cells, and d) determining at least a part of the sequence encoding the polypeptide ligand or a fragment thereof of at least one nucleic acid molecule isolated in step b).

47. The method of embodiment 46, further comprising a step a) of providing a library of embodiment 39 before step b) by ai) transfecting cells with a nucleic acid sequence comprising an AAV rep gene, a nucleic acid sequence encoding an AAV VP1 capsid polypeptide, a nucleic acid sequence encoding an AAV VP3 capsid polypeptide and the library of embodiment 36, wherein each library construct further comprises two AAV inverted terminal repeat (ITR) sequences located upstream and downstream of the nucleic acid sequence encoding the AAV VP2 fusion polypeptide, and aii) incubating the cell under conditions suitable for AAV virion formation, optionally wherein the cells are mammalian cells, particularly HEK293 cells, or insect cells, particularly Sf9 cells.

48. The method of embodiment 47, wherein said AAV VP1 capsid polypeptide and said AAV VP3 capsid polypeptide are encoded by the same nucleic acid sequence comprising an AAV cap gene, particularly wherein the start codon of VP2 in the cap gene is mutated.

49. The method of any one of embodiments 46 to 48, wherein the plurality of cells in step b) is present within a non-human model animal.

50. The method of embodiment 49, wherein in step c) nucleic acids from cells of a predefined tissue or cell type are isolated, particularly wherein said cells of a predefined tissue or cell type are isolated 1-21 days, 1-14 days, 7-21 days, 2-10 days, 2-5 days after contacting the plurality of cells with said library.

51. The method of any one of embodiments 44 to 50, further comprising a step of selecting cells in which said reporter sequence is expressed.

52. The method of any one of embodiments 46 to 51, wherein step c) comprises isolating RNA from at least a part of said cells and step d) comprises the sub-steps di) reverse transcription of said isolated RNA, dii) PCR amplification of at least a part of the sequence encoding the polypeptide ligand or a fragment thereof from the cDNA generated in step di) and diii) sequencing at least a part of the polypeptide ligand sequence, particularly by next generation sequencing.

53. The method of any one of embodiments 46 to 52, further comprising step e) of determining the relative occurrence of a specific polypeptide ligand sequence in one tissue or cell type relative to the total number of polypeptide ligand sequences in said tissue or cell type.

54. The method of any one of embodiments 46 to 53, further comprising step f) of generating a second library of nucleic acid sequences encoding an AAV VP2 fusion polypeptide from the nucleic acid molecules isolated in step c) and repeating steps b), c), d) and optionally a) and e) with said second library.

55. An AAV VP2 fusion polypeptide generated by the method of any one of embodiments 44 to 54.

56. A method of generating an AAV virion comprising a AAV VP2 fusion polypeptide, the method comprising transducing a packaging cell with the nucleic acid of embodiment 14 or 15, wherein said method results in the packaging cell producing the AAV virion.

57. A method of generating an AAV virion comprising a AAV VP2 fusion polypeptide, the method comprising culturing the cell of embodiment 16 or 17, wherein said method results in the packaging cell producing the AAV virion.

58. An AAV virion produced by the method of embodiment 56 or 57.

59. A method of making a pharmaceutical composition comprising the AAV virion of embodiment 53 or 58, the method comprising formulating the AAV virion with a pharmaceutically acceptable carrier.

60. A kit comprising the isolated nucleic acid of any one of embodiments 13 to 15.

61. An AAV VP2 fusion polypeptide comprising an AAV VP2 capsid polypeptide and a polypeptide ligand, wherein the polypeptide ligand is fused to the N-terminus of the AAV VP2 capsid polypeptide and wherein the polypeptide ligand has a molecular weight of up to 10 kDa.

62. An AAV VP2 fusion polypeptide comprising an AAV VP2 capsid polypeptide and a polypeptide ligand, wherein the polypeptide ligand is fused to the N-terminus of the AAV VP2 capsid polypeptide, and wherein the AAV VP2 capsid polypeptide comprises one or more mutations that abolish or reduce binding to Heparan Sulphate Proteoglycan (HSPG) and/or Sialic Acid (SIA).

63. The AAV VP2 fusion polypeptide of embodiment 62, wherein the polypeptide ligand has a molecular weight of up to 10 kDa.

64. An AAV VP2 fusion polypeptide comprising an AAV VP2 capsid polypeptide and a polypeptide ligand, wherein the polypeptide ligand is fused to the N-terminus of the AAV VP2 capsid polypeptide, and wherein a) the AAV VP2 capsid polypeptide is of the AAV serotype AAV1 and comprises at least one amino acid substitution selected from the group consisting of E147S, P185G, P166R, M211V, G199R, D213A, T162R, and P191N relative to the VP1 amino acid sequence of SEQ ID NO: 7, or any combination thereof, in particular wherein the AAV VP2 capsid polypeptide comprises the amino acid substitution(s) D213A, T162R, and/or P191N, or wherein b) the AAV VP2 capsid polypeptide is of an AAV serotype other than AAV1 and comprises at least one amino acid substitution corresponding to at least one of the amino acid substitution selected from the group consisting of E147S, P185G, P166R, M211V, G199R, D213A, T162R, and P191N relative to the VP1 amino acid sequence of SEQ ID NO: 7, particularly wherein bi) the AAV VP2 capsid polypeptide is of the AAV serotype 8 and comprises at least one amino acid substitution selected from the group consisting of E147S, P186G, P167R, M212V, G200R, D214A, K163R, and P192N relative to the VP1 amino acid sequence of SEQ ID NO: 4, or any combination thereof, in particular wherein the AAV VP2 capsid polypeptide comprises the amino acid substitution(s) D214A, K163R and/or P192N, or wherein bii) the AAV VP2 capsid polypeptide is of the AAV serotype 9 and comprises at least one amino acid substitution selected from the group consisting of E147S, P185G, P166R, G199R, D213A, S162R and P191N relative to the VP1 amino acid sequence of SEQ ID NO: 5, or any combination thereof, in particular wherein the AAV VP2 capsid polypeptide comprises the amino acid substitution(s) D213A, S162R and/or P191N.

65. The AAV VP2 fusion polypeptide of embodiment 64a), wherein the AAV VP2 capsid polypeptide further comprises the amino acid substitutions V473D and N500E relative to the VP1 amino acid sequence of SEQ ID NO: 7.

66. An AAV VP2 capsid polypeptide, wherein a) the AAV VP2 capsid polypeptide is of the AAV serotype AAV1 and comprises at least one amino acid substitution selected from the group consisting of E147S, P185G, P166R, M211V, G199R, D213A, T162R, and P191N relative to the VP1 amino acid sequence of SEQ ID NO: 7, or any combination thereof, in particular wherein the AAV VP2 capsid polypeptide comprises the amino acid substitution(s) D213A, T162R, and/or P191N, or wherein b) the AAV VP2 capsid polypeptide is of an AAV serotype other than AAV1 and comprises at least one amino acid substitution corresponding to at least one of the amino acid substitution selected from the group consisting of E147S, P185G, P166R, M211V, G199R, D213A, T162R, and P191N relative to the VP1 amino acid sequence of SEQ ID NO: 7, particularly wherein bi) the AAV VP2 capsid polypeptide is of the AAV serotype 8 and comprises at least one amino acid substitution selected from the group consisting of E147S, P186G, P167R, M212V, G200R, D214A, K163R, and P192N relative to the VP1 amino acid sequence of SEQ ID NO: 4, or any combination thereof, in particular wherein the AAV VP2 capsid polypeptide comprises the amino acid substitution(s) D214A, K163R and/or P192N, or wherein bii) the AAV VP2 capsid polypeptide is of the AAV serotype 9 and comprises at least one amino acid substitution selected from the group consisting of E147S, P185G, P166R, G199R, D213A, S162R and P191N relative to the VP1 amino acid sequence of SEQ ID NO: 5, or any combination thereof, in particular wherein the AAV VP2 capsid polypeptide comprises the amino acid substitution(s) D213A, S162R and/or P191N.

67. An rAAV virion comprising the AAV VP2 capsid polypeptide of embodiment 66.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic representation of the AAV VP2 fusion polypeptide expression plasmids generated in Example 1.

FIG. 2 is a schematic representation of the cargo expression plasmid pNN001.

FIG. 3 shows SDS-PAGE (denaturing) on Darpins, VHHs and miniprotein decorated AAV8 preparations in comparison to CTR w.t. AAV8. Minor VP2 w.t. contamination was observed in sample 4 (likely due to affinity purification spill-over from AAV8 trans-VP2 production) as confirmed by MS.

FIG. 4 shows an AAV sandwich ELISA on EGFR and TIGIT recombinant protein using decorated AAV8 preparations.

FIG. 5 shows AAV8 decoration levels of VP2 fused to different scaffolds.

FIG. 6 shows the in vitro infectivity of decorated AAV8 on an EGFR over-expressing HKB11 cell line. Shown is the number of full particles versus the percentage of infected (EGFP+) cells.

FIG. 7 shows the in vitro infectivity of the anti-EGFR Sso7d-AAV8 VP2 fusion on GBM neurospheres (EGFR enhanced clone HF-2927).

FIG. 8 shows the in vitro infectivity of decorated AAV8 on a receptor over-expressing cell line.

FIG. 9 shows the in vitro infectivity of decorated AAV6Sil2 on EGFR over-expressing HKB11 and HKB11 w.t. cell line.

FIG. 10 shows the in vitro infectivity of anti-EGFR Sso7 VP2 fusion decorated AAV9 on GBM neurospheres.

FIG. 11 shows the in vitro infectivity of decorated AAV1Sil1 on EGFR over-expressing HKB11 and HKB11 w.t. cell line.

FIG. 12 shows the amino acid frequency at targeted positions on Lib8 Sso7d phage plasmid library. The results represent MiSeq NGS outcome on 500-700K filtered reads (Q>30).

FIG. 13 shows the results of occurrences analysis on the Lib8 Sso7d library after the third selection round.

FIG. 14 shows the results of FACS analysis of six Sso7d constructs on EGFR-overexpressing and EGFR knock out cells.

FIG. 15 is a schematic representation of the cargo plasmid SK0033.

FIG. 16 shows the amino acid frequency at randomized positions in the Lib8 Sso7d-AAV1Sil1 plasmid library (SK0037).

FIG. 17A shows the results of occurrences analysis on Lib8 Sso7d-VP2 AAV1Sil1.

FIG. 17B shows the amino acid frequencies at targeted positions on the Lib8 Sso7d-VP2 AAV1Sil1 plasmid library.

FIG. 17C shows the amino acid frequencies at targeted positions on the Lib8 Sso7d-VP2 AAV1Sil1 AAV library.

FIG. 17D shows the Lib8 Sso7d-VP2 AAV1Sil1 AAV library production bias at the targeted positions.

FIG. 18 shows the percentages of human cells in different tissues of humanized mice.

FIG. 19 shows the biodistribution of Lib8 Sso7d-VP2 AAV1Sil1 and the undecorated control in humanized mice.

FIG. 20 shows flash-gel analysis of the RT-PCR reactions (expected size: 403 bp).

FIG. 21 shows the distribution of single rcSso7d variant sequences in different organs/cells from Lib8 Sso7d-VP2 AAV1Sil1 injected mice.

FIG. 22 shows the distribution of enriched hits in the different organs.

FIG. 23 is a schematic representation of the barcoded cargo plasmids generated in example 8.

FIG. 24A shows the yield and the percentage of full particles obtained from single barcoded Sso7d-AAV1Sil1.

FIG. 24B shows the proportion of VP2 fusion and VP1 on single barcoded Sso7d-AAV1Sil1 and undecorated control.

FIG. 25 shows the in vitro infectivity of Sso7d decorated AAV1Sil1 on HEK293 suspension cells.

FIG. 26 shows the library distribution in the input AAV-471 preparation used for mouse injection.

FIG. 27 shows the distribution of the barcoded Sso7d-VP2 AAV1Sil1 library in different tissues.

FIG. 28 shows the flash-gel analysis of RT-PCR reactions (expected sized: 403 pb).

FIG. 29 shows the flash-gel analysis of amplicon fragments for NGS (expected size: 243 bp).

FIG. 30 shows the tissue distribution of the top 12 enriched hits in the LT-HSC population identified after three rounds of selection humanized mice using the Lib8 Sso7d-VP2 AAV1Sil1 library.

FIG. 31 is a schematic representation of the acceptor cargo plasmids generated in example 10.

FIG. 32 shows the flash-gel analysis of RT-PCR reactions (expected size: 608-668 bp).

FIG. 33 shows the flash-gel analysis of amplicon PCR reactions (expected size: 484-529 bp).

FIG. 34 shows the V-Exp library selection counts distribution.

FIG. 35 shows the variation analysis on V-Exp targeted positions after positive and negative selection (the data represent the percentage of w.t. amino acid at each given position).

FIG. 36 shows the amino acid distribution at prioritized VP2 positions after positive selection (the data represent the percentage of amino acid at the top nine prioritized positions—selected AA mutations are framed).

FIG. 37 shows the linker and amino acid variation at prioritized VP2 positions after positive selection.

FIG. 38 shows a bar graph depicting the fold change between the EC50 values of EGFP positive cells in EGFR overexpressing and knockout cells.

FIG. 39 shows a bar graph depicting binding to EGFR of the different anti-EGFR Sso7d decorated AAV1 Sil1 VP2 variants (background removed ELISA signal at 5E+11 vp).

FIG. 40 shows the in vitro infectivity of anti-EGFR Sso7d decorated ssAAV1Sil1 on HKB11 wild type cells.

FIG. 41 shows the in vitro infectivity of anti-EGFR Sso7d decorated ssAAVSil1 on receptor over-expressing HKB11 cells.

### EXAMPLES

### Example 1: Initial Evaluation of AAV VP2 Fusion Polypeptide Decoration Levels in AAV8 Capsids Using VHH, DARPIN and Miniprotein Scaffolds Genetically Fused to VP2 N-Terminus

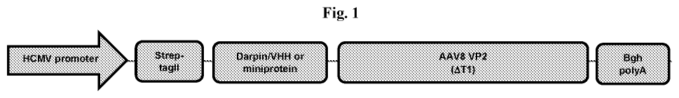

In order to evaluate the feasibility of VP2 N-terminal genetic fusion to generate decorated AAVs as described in Munch et al., Nat Commun 6, 6246 (2015), different scaffolds were cloned into the AAV8 VP2 expression plasmid AgC159 at the N-terminus of the VP2 open reading frame lacking the start codon encoding threonine (VP2 DT). The resulting AAV VP2 fusion polypeptide expression plasmid, in which the AAV VP2 fusion polypeptide open reading frame is operably linked to a HCMV promoter, is graphically depicted in FIG. 1.

Cloning of Scaffolds into VP2 Expression Plasmids:

The scaffold sequences listed in Table-1 were ordered as string DNA at GeneArt adding 5′ and 3′ extensions suitable for Gibson based cloning. In the case of VHHs, sequences were amplified by PCR using internal expression plasmids as template. The fragments were cloned into HindIII-AgeI digested VP2 expression plasmid AgCl 159 and correct assembly was confirmed by Sanger sequencing.

TABLE 1
List of scaffolds and linkers used in Example 1. A strep-TagII-GS sequence at the N-
terminus and G4S linkers at the C-terminus were added in all constructs.

| | | | Molecular weight |
|---|---|---|---|
| | | | of final scaffold |
| Scaffold ID | Reference | *Final scaffold sequence | (kDa) |
| Anti-Her2 | https://doi. | MRGSAWSHPQFEKGSDLGKKLLEAARAG | 18.7 |
| Darpin (9_29) | org/10.1016/j. | QDDEVRILMANGADVNAHDFYGITPLHLA | |
| | jmb.2008.07.085 | ANFGHLEIVEVLLKHGADVNAFDYDNTPL | |
| | | HLAADAGHLEIVEVLLKYGADVNASDRD | |
| | | GHTPLHLAAREGHLEIVEVLLKNGADVN | |
| | | AQDKFGKTAFDISIDNGNEDLAEILQGGG | |
| | | GS | |
| Anti-EGFR | https://doi. | MRGSAWSHPQFEKGSDLGKKLLEAARAG | 18.7 |
| (ErbB1) | org/10.1016/j. | QDDEVRILMANGADVNADDTWGWTPLH | |
| Darpin (E_01) | jmb.2008.07.085 | LAAYQGHLEIVEVLLKNGADVNAYDYIG | |
| | | WTPLHLAADGHLEIVEVLLKNGADVNAS | |
| | | DYIGDTPLHLAAHNGHLEIVEVLLKHGAD | |
| | | VNAQDKFGKTAFDISIDNGNEDLAEILQG | |
| | | GGGS | |
| Anti-MBP | https://doi. | MRGSAWSHPQFEKGSDLGRKLLEAARAG | 18.5 |
| Darpin (Off7) | org/10.1038/ | QDDEVRILMANGADVNAADNTGTTPLHL | |
| | nbt962 | AAYSGHLEIVEVLLKHGADVDASDVFGYT | |
| | | PLHLAAYWGHLEIVEVLLKNGADVNAMD | |
| | | SDGMTPLHLAAKWGYLEIVEVLLKHGAD | |
| | | VNAQDKFGKTAFDISIDNGNEDLAEILQG | |
| | | GGGS | |
| Anti-TIGIT | Internal | | 15.6 |
| VHH | | | |
| (NEG974) | | | |
| Anti-GPR126 | Internal | | 15.3 |
| VHH | | | |
| (NEG1713) | | | |
| Anti-HA | https://doi. | MRGSAWSHPQFEKGSTSGVRATSKFAALIA | 6.4 |
| miniprotein | org/10.1038/ | AEIAREFGYTVDVQEKNGEWRVVFDGGG | |
| | nature23912 | GS | |

*in bold original seq w/o N- or C-term additions

### Decorated AAVs Production:

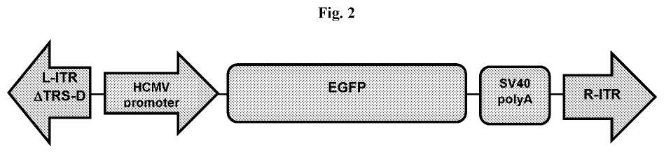

Generated VP2 fusion plasmids were transfected into suspension HEK293T/17F cells together with a rep/cap VP2 KO plasmid (comprising the AAV2 rep gene and the AAV8 cap gene with a mutation in the VP2 open reading frame start codon thus encoding AAV8 VP1 and VP3 but not VP2), pHelper (encoding adenoviral proteins E2A, E4 and VA RNAs) and HCMVprom-EGFP-SV40 pA scAAV (pNN001; encoding eGFP under the control of the HCMV promoter and flanked by AAV2 ITRs and schematically shown in FIG. 2) as cargo.

Briefly, 100 ml (350 ml for AAV8 w.t. production) of HEK293T/17F suspension cells were diluted to 1.5E+06 cells/ml and transfected using PEI MAX (PEI:DNA ratio=3:1) with 0.8 μg DNA mix per 1E+06 cells. The plasmid mix was composed of equimolar quantities of the either three or four plasmids as described in Table 2. Six days post transfection, samples were centrifuged for 20 min at 4000 rpm and the supernatant was loaded at 4 ml/min on POROS GoPure AAVX 1 ml 0.5 cm×5 cml (ThermoFisher #A36652) mounted on an AKTA Pure instrument. Bound AAV particles were washed with 10 column volumes (CV) of Wash Buffer, pH 7.3 (50 mM Tris-Cl, 0.5 M NaCl, 0.01% Pluronic F-68) and eluted with 5 CV of Glycine elution buffer, pH 2.7 (0.1 M Glycine, 0.2 M NaCl, 0.25 M L-Arginine). Elution fractions (0.25 ml) were immediately neutralized with 25 μl of 1 M Tris-Cl, pH 10. Fractions containing AAV (as assessed by UV280 signal) were pooled and dialyzed using Float-A-Lyzer Dialysis Device 100 kDa (Spectrum™ #G235071) overnight against PBS, pH 7.4+0.001% Pluronic F-68.

qPCR methodology was used to assess full particle concentration (vg/ml). qPCR was conducted on a Qiagen RotorGene instrument on DNAse pre-treated samples using the KAPA Probe Fast qPCR kit and the SV40 pA oligo set listed in Table 3. Plasmid pNN001 was used for standard curve generation. qPCR results are summarized in Table 4.

TABLE 2
List of rep/cap VP2 KO and VP2 expression plasmids used
in this example in addition to pHelper and pNN001 cargo

| Sample ID | Rep/Cap plasmid | VP2 expression plasmid |
|---|---|---|
| AAV8 w.t. | pRep2Cap8 | none |
| AAV8 VP2 KO | *(AgC1157) pAAV | none |
| | rep2_cap8 VP2 KO | |
| AAV8 trans- | (AgC1157) pAAV | (AgC1159) pR5a_AAV8 VP2 |
| VP2 | rep2_cap8 VP2 KO | (no fusion) |
| AAV8− Anti- | (AgC1157) pAAV | (AgC1168) pR5a_Anti-ErbB1 (E_01) |
| EGFR Darpin | rep2_cap8 VP2 KO | DARPin_AAV8 VP2 |
| AAV8− Anti- | (AgC1157) pAAV | (AgC1169) pR5a_Anti-MBP Off7 |
| MBP Darpin | rep2_cap8 VP2 KO | DARPin_AAV8 VP2 |
| AAV8− Anti- | (AgC1157) pAAV | (AgC1170) pR5a_Anti HER2 9_29 |
| Her2 Darpin | rep2_cap8 VP2 KO | Darpin_AAV8 VP2 |
| AAV8− Anti-HA | (AgC1157) pAAV | (AgC1171) pR5a_anti HA |
| miniprotein | rep2_cap8 VP2 KO | miniprotein_AAV8 VP2 |
| AAV8− Anti- | (AgC1157) pAAV | (AgC1172) pR5a_anti-TIGIT VHH |
| TIGIT VHH | rep2_cap8 VP2 KO | (NEG974)_AAV8 VP2 |
| AAV8− Anti- | (AgC1157) pAAV | (AgC1173) pR5a_anti GPR126 |
| GPR126 VHH | rep2_cap8 VP2 KO | (NEG1731) VHH_AAV8 VP2 |

*VP2 KO rep2/Cap8 plasmid (AgC1157) was generated by mutating first Threonine of VP2 to Alanine (T138A)

TABLE 3
Oligo set used for AAV genome concentration (vg/ml)
measurement by qPCR

| Oligo ID | 5′-3′ sequence |
|---|---|
| SV40pA region-For | AGCAATAGCATCACAAATTTCACAA |
| SV40pA region-Rev | CCAGACATGATAAGATACATTGATGAGTT |
| SV40pA region-Probe | FAM-AGCATTTTTTTCACTGCATTCTAG |
| | TTGTGGTTTGTC-BHQ1 |

TABLE 4
qPCR results for decorated AAV8

| Sample ID | vg/ml | Yield (vg/cell) |
|---|---|---|
| AAV8 w.t. | 7.10E+13 | 1.62E+05 |
| AAV8 VP2 KO | 1.61E+13 | 1.29E+05 |
| AAV8 trans-VP2 | 2.39E+13 | 1.91E+05 |
| AAV8− Anti-EGFR Darpin | 1.07E+13 | 8.56E+04 |
| AAV8− Anti-MBP Darpin | 3.03E+12 | 2.42E+04 |
| AAV8− Anti-Her2 Darpin | 4.88E+12 | 3.90E+04 |
| AAV8− Anti-HA miniprotein | 1.12E+13 | 8.96E+04 |
| AAV8− Anti-TIGIT VHH | 3.19E+12 | 2.55E+04 |
| AAV8− Anti-GPR126 VHH | 6.06E+12 | 4.85E+04 |

The absence of VP2 (sample AAV8 VP2 KO) and the provision of VP2 in trans (sample AAV8 trans-VP2) had only marginal effects on the AAV titer. Genetic fusion of the various scaffolds to the VP2 N-terminus lead to a 2-7 fold decrease in AAV titer.

### Evaluation of Decoration Level:

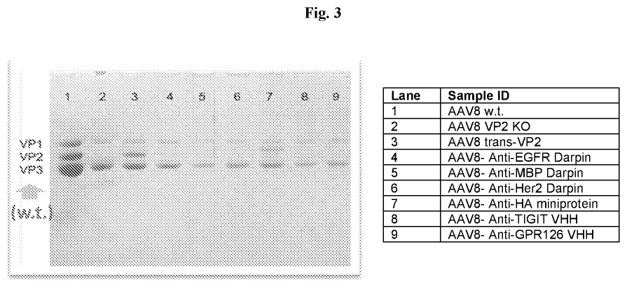

20 μl of the affinity purified AAV preparations were analyzed by SDS-PAGE under denaturing conditions to assess the presence of VP2 fusions. The SDS page is shown in FIG. 3. A molecular weight shift of VP2 could only be observed for the miniprotein decorated sample whereas none of the other samples (VHH and Darpin fusions) showed detectable levels of VP2-fusions.

Additionally, a CE-SDS analysis (Agilent 2200 Tapestation system, P200 protein kit using denaturing conditions) was performed to evaluate the relative amounts of the different VP proteins in the purified samples in a semi-quantitative way. The results confirm the molecular weight shift of VP2 in the AAV8-Anti-HA miniprotein sample and no detectable presence of VP2 fusions in the other samples. The AAV8-Anti-HA miniprotein sample showed similar relative amounts of the different VP proteins as the AAV8 w.t. sample. A slightly increased amount of VP2 was observed in the AAV8 trans-VP2 sample. The results are summarized in Table 5.

TABLE 5
Molecular Weight and estimated relative VP amounts of Darpin,
VHH and miniprotein decorated AAV8 preparations (Tapestation)

| | | | |
|---|---|---|---|
| Sample ID | VP3 | VP2 | VP1 |

Table 5a: Observed MW of VPs (kDa)

| | | | |
|---|---|---|---|
| AAV8 w.t. | 61 | 70.4 | 93.5 |
| AAV8 VP2 KO | 61.9 | | 94.2 |
| AAV8 trans-VP2 | 61.5 | 70.8 | 94.2 |
| AAV8− Anti-EGFR Darpin | 61.9 | | 95.1 |
| AAV8− Anti-MBP Darpin | 61.8 | | 94.7 |
| AAV8− Anti-Her2 Darpin | 62.3 | | 95.2 |
| AAV8− Anti-HA miniprotein | 62.2 | 78.7 | 94.2 |
| AAV8− Anti-TIGIT VHH | 61.6 | | 94.3 |
| AAV8− Anti-GPR126 VHH | 61.6 | | 93.8 |

Table 5b: VP distribution (assuming
a total of 60 VPs per AAV particle)

| | | | |
|---|---|---|---|
| AAV8 w.t. | 50.6 | 6.1 | 3.3 |
| AAV8 VP2 KO | 56.8 | | 3.2 |
| AAV8 trans-VP2 | 48.3 | 8.6 | 3.1 |
| AAV8− Anti-EGFR Darpin | 56.0 | | 4.0 |
| AAV8− Anti-MBP Darpin | 55.7 | | 4.3 |
| AAV8− Anti-Her2 Darpin | 56.6 | | 3.4 |
| AAV8− Anti-HA miniprotein | 51.7 | 6.0 | 2.2 |
| AAV8− Anti-TIGIT VHH | 56.6 | | 3.4 |
| AAV8− Anti-GPR126 VHH | 55.4 | | 4.6 |

### Mass Spectrometry Analysis:

Due to the potential co-migration of the VHH and Darpin VP2 fusions with the VP1 band, all preparations were analyzed using LC-MS. 5 μl of non-treated AAV samples were injected on a UPLC-I class coupled to a Synapt G2S (WATERS) quadrupole time-of-flight mass spectrometer. The samples were denatured on column and chromatographically separated on a Waters Bioresolve RP 1×50 mm column. The column was held at 80° C. with a gradient consisting of 30 to 38% of organic mobile phase over 40 min with a flow rate of 0.1 ml/min. The aqueous mobile phase consisted of 0.1% formic acid in water and the organic consisted of 0.1% formic acid in acetonitrile.

VP1 and VP3 expected mass could be confirmed in all tested samples, the presence of VP2 w.t. or VP2 fusion was only confirmed in AAV8 w.t., AAV8 trans-VP2 and AAV8-Anti-HA miniprotein samples confirming SDS and CE-SDS results.

### AAV8 Sandwich ELISA:

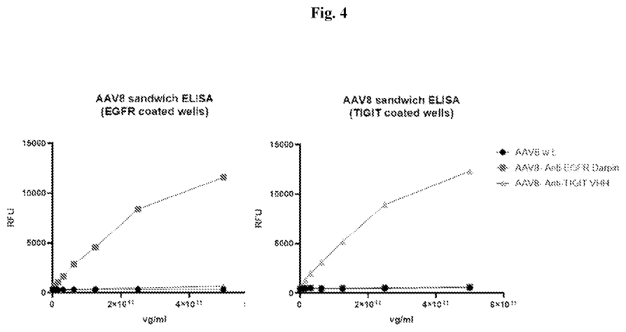

In order to further assess decoration efficiency and correct exposure of the VP2 fused scaffold a more sensitive sandwich ELISA assay was performed. hEGFR and hTIGIT extracellular domain (ECD) recombinant proteins were used to coat 96 well plates at 5 μg/ml. After over night incubation and blocking with 3% BSA/TBST buffer, different amounts of decorated AAVs vg were added to the wells and incubated for 2 h at RT. After washing steps (3×), wells were incubated for 1 h at room temperature (RT) with anti-AAV8 monoclonal mIgG2 (Origene AM32478SU-N). Wells were washed again (3×), incubated with anti-mouse IgG-AP conjugated (Sigma-Aldrich A5153), additionally washed (3×) and signal developed using Attophos substrate (Roche 00000011681982001). Results are showed in FIG. 4.

Contrary to MS and CE-SDS, AAV8 sandwich Elisa detected Darpin and VHH on the surface of assembled AAV. It is suspected that only a marginal portion of generated AAVs incorporate VP2 fused to either of these two scaffolds, which were only detectable by sandwich Elisa but not by MS and CE-SDS.

### Example 2: Evaluation of AAV8 Decoration Levels Using Small Scaffolds Genetically Fused to VP2 N-Terminus

As observed in Example 1, AAV8-Anti-HA miniprotein seems to be the only AAV decorated preparation with a wildtype like VP2 decoration level. In order to assess whether this phenomenon is due to the small size of the scaffold we cloned different anti-EGFR binding domains as listed in Table 6 into the AAV8 VP2 expression plasmid AgC1159.

TABLE 6
List of scaffold IDs and linkers used in this example

| | | | | Molecular |
|---|---|---|---|---|
| | | | | weight of |
| | | | | final |
| | | | AAV8-VP2 fusion | scaffold |
| Scaffold ID | Reference | *Final scaffold sequence | expression plasmid ID | (kDa) |
| Anti-EGFR | https://doi. | MRGSAWSHPQFEKGSDLGK | (AgC1168) pR5a_Anti- | 18.5 |
| Darpin (E_01) | org/10.1016/ | KLLEAARAGQDDEVRILM | ErbB1 (E_01) | |
| | j.jmb.2008. | ANGADVNADDTWGWTPL | DARPin_AAV8 VP2 | |
| | 07.085 | HLAAYQGHLEIVEVLLKN | | |
| | | GADVNAYDYIGWTPLHLA | | |
| | | ADGHLEIVEVLLKNGADV | | |
| | | NASDYIGDTPLHLAAHNG | | |
| | | HLEIVEVLLKHGADVNAQ | | |
| | | DKFGKTAFDISIDNGNEDL | | |
| | | AEILQGGGGS | | |
| Anti-EGFR | doi: 10.1016/ | MVSDVPRDLEVVAATPTSL | (AgC1275)_pRS5a_Ant | 11.9 |
| Fibronectin | j.jmb.2010. | LISWFDYA VTYYRITYGET | i- | |
| (E.6.2.6) | 06.004 | GGNSPVQEFTVPGWISTAT | EGFR_Fibronectin_G4S | |
| | | ISGLKPGVDYTITVYAVTD | x3_AAV8_VP2 | |
| | | NSRWPFRSTPISTNYRTEID | | |
| | | KPPQGGGGSGGGGSGGGGS | | |
| Anti-EGFR | DOI | MATVKLTYQGEEKQVDIS | (AgC1274)_pRS5a_Ant | 7.8 |
| Sso7d (E.18.4.5) | 10.1074/ | KITYVDRAGQFIWFEYDEG | i- | |
| | jbc.M116.7 | GGALGTGWVSEKDAPKEL | EGFR_Sso7d_G4Sx3_ | |
| | 41314 | LQMLEKQGGGGSGGGGSG | AAV8_VP2 | |
| | | GGGS | | |
| **Anti-EGFR | doi: 10.1016/ | MVDNKFNKEMWAAWEEI | (AgC1276)_pRS5a_Ant | 7.4 |
| Affibody | j.jmb.2007 | LHLPNLNGWQMTAFIASL | i- | |
| (ZEGFR-1907) | 12.060 | VDDPSQSANLLAEAKKLN | EGFR_Affibody_G4Sx | |
| | | DAQAPKGGGGSGGGGSGG | 3_AAV8_VP2 | |
| | | GGS | | |
| Anti-EGFR GP2 | http://dx.do | MKFWATVSRGDSYWFEVP | (AgC1273)_pRS5a_Ant | 6.7 |
| (GaEGFR2.2.3) | i.org/10.10 | VYAETLDEALELAERQYP | i- | |
| | 16/j.chembi | MYHIYYVTRVRPGGGGSG | EGFR_GP2_G4Sx3_A | |
| | ol.2015.06. | GGGSGGGGS | AV8_VP2 | |
| | 012 | | | |
| Anti-HA | https://doi. | MRGSAWSHPQFEKGSTSGV | (AgC1171) pR5a_anti | 6.4 |
| miniprotein | org/10.1038/ | RATSKFAALIAAEIAREFG | HA miniprotein _AAV8 | |
| | nature23912 | YTVDVQEKNGEWRVVFDG | VP2 | |
| | | GGGS | | |

*in bold original seq w/o N- or C-terminal addition
**Anti-EGFR Affibody (Z_{EGFR}-1907) sample partially lost during final production step; an accurate yield evaluation was not possible. Based on peak height during affinity purification, yield is expected to have been similar to the other purified preps

### Decorated AAV Production:

Cloning, expression, purification and assessment of viral concentration of the different decorated AAV8 particle preparations was performed as described in Example 1 with the following exceptions:
The transfection volume was 300 ml for each sample
Cells plus supernatant were collected 4 days post transfection
Final concentrations of 0.5% Triton, 2 mM MgCl₂ and 12.5 U/ml of Benzonase were added to the bulk harvest; lysis occurred for 2 h at 37° C., afterwards 0.5 M NaCl was added, samples were centrifuged for 30 min at 3500 g. The lysates were filtered, clarified and then loaded on a POROS AAVx 1 ml resin.

As controls new preparations of AAV8-Anti-EGFR Darpin and AAV8-Anti HA Miniprotein were generated in parallel in a similar manner. qPCR as described in Example 1 was used to assess full particle concentration (vg/ml). The results are summarized in Table 7c.

### Evaluation of Decoration Levels and Total AAV Yield:

CE-SDS analysis (using the Agilent 2100 Bioanalyzer system and the Protein 230 kit under denaturing conditions) was performed in order to evaluate VP distribution in the purified samples and total VP yield.

Calculation of total AAV particles/ml (vp/ml) and VP ratio
ng/μl (=μg/ml) values are divided by the observed MW and then multiplied by 1E+06 (μg to g conversion) to calculate moles/ml values
moles/ml values are multiplied by 6.022E+23 (avogadro nr) to calculate the number of molecules/ml
VP distribution: calculated based on molecules/ml values assuming one AAV is made of 60 VPs in total
AAV particles/ml (vp/ml): Sum of VP1+VP2+VP3 molecules ml are divided by 60

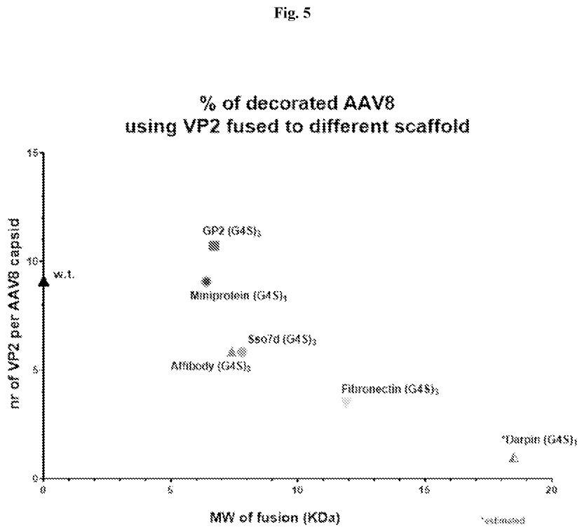

The results are summarized in Table 7 and FIG. 5.

TABLE 7
Bioanalyzer results on AAV8 decorated samples
a: Observed MW of VPs (kDa)

| Sample ID | VP3 | VP2 | VP1 |
|---|---|---|---|
| Anti-EGFR Darpin (E_01) | 76.5 | | 108.8 |
| Anti-EGFR Fibronectin (E.6.2.6) | 76.8 | 105.8 | 109.2 |
| Anti-EGFR Sso7d (E.18.4.5) | 76.7 | 99.6 | 109.4 |
| Anti-EGFR Affibody (Z_{EGFR}-1907) | 77.3 | 99.4 | 109.5 |
| Anti-EGFR GP2 (GaEGFR_{2.2.3}) | 76.5 | 98.5 | 109.2 |
| Anti-HA miniprotein | 77.3 | 97.9 | 109.4 |
| AAV8 w.t. | 77.0 | 91.6 | 109.8 |

b: VP distribution (assuming a total of 60 VPs per AAV particle)

| Sample ID | VP3 | VP2 | VP1 |
|---|---|---|---|
| Anti-EGFR Darpin (E_01) | 53.1 | *(1) | *6.9 |
| Anti-EGFR Fibronectin (E.6.2.6) | 50.6 | 3.5 | 6.0 |
| Anti-EGFR Sso7d (E.18.4.5) | 49.6 | 5.8 | 4.6 |
| Anti-EGFR Affibody (Z_{EGFR}-1907) | 49.0 | 5.9 | 5.1 |
| Anti-EGFR GP2 (GaEGFR_{2.2.3}) | 44.5 | 10.7 | 4.7 |
| Anti-HA miniprotein | 45.8 | 9.1 | 5.1 |
| AAV8 w.t. | 46.0 | 9.1 | 4.9 |

c: Viral genome (vg) and viral particle (vp) yield per transfected cell

| Sample ID | vg/cell | vp/cell |
|---|---|---|
| Anti-EGFR Darpin (E_01) | 2.32E+04 | 4.04E+04 |
| Anti-EGFR Fibronectin (E.6.2.6) | 2.96E+04 | 1.20E+05 |
| Anti-EGFR Sso7d (E.18.4.5) | 2.29E+04 | 8.54E+04 |
| Anti-EGFR GP2 (GaEGFR_{2.2.3}) | 2.77E+04 | 1.49E+05 |
| Anti-HA miniprotein | 1.34E+04 | 8.20E+04 |
| AAV8 w.t. | 3.69E+04 | 1.20E+05 |

*For Darpin sample VP1 and VP2 fusion may potentially co-migrate and no exact value for the number of VP2 units could be assigned. An arbitrary value of 1 VP2-fusion per AAV was assigned based on the slight increase of the VP1 value.

Successful incorporation of VP2 fusion polypeptides in AAV capsids seems to correlate with the molecular weight of the scaffolds fused to the VP2 polypeptide. Satisfactory decoration levels (>60% of w.t. VP2 level) were obtained using scaffolds below 10 kDa. Above this threshold a drop in VP2 fusion incorporation was observed, particularly severe in the case of 18 kDa darpin. A marginal drop in yield was observed with all tested anti-EGFR scaffolds used in this experiment (<2 fold compared to w.t.).

### In Vitro Infectivity of Decorated AAV8 on EGFR Over-Expressing Cell Line:

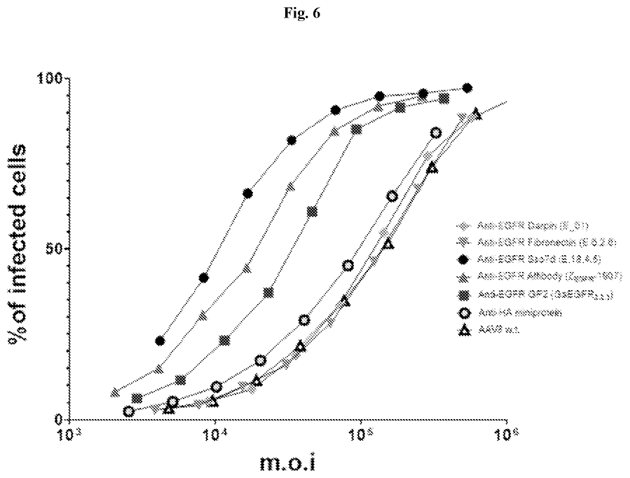

In order to evaluate the effect of the different decoration levels of the anti-EGFR decorated AAV8 preps, an infectivity assay was performed on HKB11 cells stably over-expressing hEGFR (HKB11_EGFR). At the day of infection, cells were resuspended in DMEM to 5E+05 cells/ml and 50 μl/well were seeded in a 96 well plate (2.5E+04 cells/well). AAV8 decorated preparations were diluted in DMEM media and different concentrations ranging from 2E+06 to 1.56E+04 moi based on total vp were applied to the seeded cells (final volume 100 μl). Four days post transduction cells were detached and FACS analysis was performed to calculate the percentage of EGFP positive cells. Full particles (vg) were back-calculated. FIG. 6 depicts the number of full particles versus the percentage of infected (EGFP+) cells.

The anti-EGFR VP2 fusion polypeptides with high decoration levels showed an increased uptake in HKB11_EGFR cells (Sso7d>Affibody>GP2). In contrast, Anti-EGFR DARPin and Fibronectin scaffold fusions showed no increase in infectivity as compared to the undecorated AAV8 w.t. control, which is likely due to the low decoration levels obtained with these two scaffolds.

### In Vitro Infectivity of Decorated AAV8 on Glioblastoma Multiforme (GBM) Neurospheres:

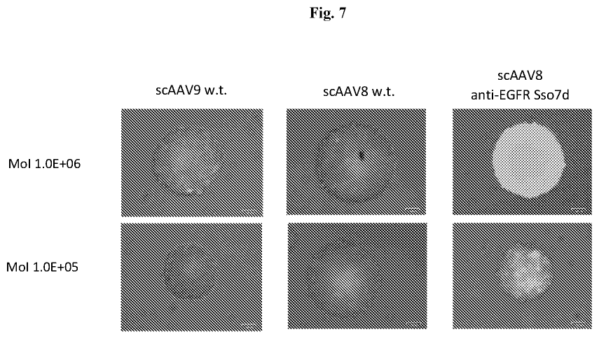

GBM neurosphere cultures were obtained from Henri Ford Health System and grown in suspension at 37° C. and 5% CO2 in DMEM/HamF12 (Gibco #11039-021) supplemented with Gibco N2 supplement (100×; #17202-048), BSA (Sigma #A4919; final conc 0.5 mg/ml), Gentamicin (Gibco #15710-064; final conc 0.025 mg/ml), Antibiotic-Antimycotic (100×) (Invitrogen #15240-062), EGF (Peprotech #100-15, final conc 20 ng/ml) and FGF (Peprotech #100-18B, final conc 20 ng/ml). In order to verify the infectivity results obtained in the EGFR over-expressing HKB11 cell line, Anti-EGFR Sso7d (E.18.4.5)-AAV8 was used to infect an GBM neurosphere cell line with enhanced EGFR expression (HF-2927). GBM cells were seeded at approx. 40′000 cells/well in 96-well plate without disrupting neurospheres (the cell number was determined by taking 200 μl of cell train and disrupting the spheres in order to estimate cell concentration). Seeded neurospheres were infected with 1E+06 and 1E+05 moi of anti-EGFR Sso7d decorated AAV8 and compared to undecorated AAV8 and AAV9 w.t. preparations containing the same EGFP cargo. Three days post transduction the EGFP signal was monitored using ZOE Fluorescent Cell Imager (Bio-Rad). Pictures of representative Neurosphere are shown in FIG. 7.

AAV particles displaying the anti-EGFR Sso7d VP2 fusion polypeptide showed a significantly increase cellular uptake in GBM neurosphere cultures of EGFR overexpressing cell line HF-2927 as compared to undecorated AAV8 and AAV9 preparations.

### Example 3: Evaluation of AAV8 Decoration Levels Using Additional GP2 and Affibody Binders Genetically Fused to VP2 N-Terminus

### Introduction

Additional exemplary GP2 and Affibody sequences were extracted from literature and cloned into AAV8 VP2 expression plasmids as described in Example 1.

TABLE 8
List of scaffold IDs and linkers used in this example

| | | | AAV8-VP2 fusion | Molecular |
|---|---|---|---|---|
| | | | expression plasmid | weight of |
| Scaffold ID | Reference | *Final scaffold sequence | ID | scaffold (kDa) |
| Anti-Her2 | DOI: | MVDNKFNKEMRNAYWEIAL | (SK0046) pR5a_anti- | 7.6 |
| Affibody | 10.1158/0008- | LPNLNNQQKRAFIRSLYDDP | PDGFR_affibody_3x | |
| (ZHER2:342) | 5472.CAN- | SQSANLLAEAKKLNDAQAPK | G4S-AAV8_VP2 | |
| | 05-3521 | GGGSGGGSGGGS | | |
| Anti- | DOI: | MVDNKENKELVKAAAEIDAL | (SK0047) pR5a_anti- | 7.3 |
| PDGFRb | 10.1016/j.jmb. | PNLNRRQWNAFIKSLVDDPS | HER2_affibody_3xG | |
| Affibody | 2011.01.033 | QSANLLAEAKKLNDAQAPK | 4S-AAV8_VP2 | |
| (Z02483) | | GGGSGGGSGGGS | | |
| Anti- InsR | doi: | MKFWATVCSGHDGYCFEVP | (SK0048) | 6.7 |
| GP2 (#1) | 10.1158/1535- | VYAETLDEALELAEWQYDST | pR5a_GP2_C11_3xG | |
| | 7163.MCT- | YYDYAVTRVRPGGGSGGGSG | 4S_AAV8_VP2 | |
| | 16-0685 | GGS | | |
| Anti- InsR | doi: | MKFWATVDCLYNDTAFEVP | (SK0049) | 6.5 |
| GP2 (#5) | 10.1158/1535- | VYAETLDEALELAEWQYDP | pR5a_GP2_C15_3xG | |
| | 7163.MCT- | NYCIVTRVRPGGGSGGGSGG | 4S_AAV8_VP2 | |
| | 16-0685 | GS | | |

*in bold original seq w/o N- or C-terminal addition

### Decorated AAV8 Production:

A DNA mix containing an equimolar ratio of pHelper, (DL144) CAG-EGFP-NOX single stranded cargo, (AgC1157) pAAV rep2_cap8 VP2 KO and AAV8 VP2 fusion expression plasmids described above was prepared and transfected into 200 ml of HEK293 suspension cells at a concentration of 2E+06 vc/ml (1.1 μg total DNA/1E+06 cells) using the FectoVIR-AAV Transfection reagent (Polyplus #101000022); DNA:FectoVIR=1 μg: 1 μl. Glucose at a final concentration of 2 g/l was added to the transfection mix after transfection and the mixture was incubated at 37° C., 110 rpm, 6% CO2. Two additional transfections with undecorated AAV1Sil1 in combination with its corresponding barcoded cargo plasmid (A02) were also performed. Three days post transfection, Benzonase to a final concentration of 0.1 U/μl and 20 ml of lysis buffer (500 mM HEPES, 10% Tween 20, 20 mM MgCl₂) were added to the transfection reactions and the mixtures were incubated at 37° C. in agitation for 3 h. After this incubation step, 24 ml of sucrose salt solution (5 M NaCl, 7% sucrose) were added to the lysate and incubated for additional 20 min. Samples were centrifuged for 15 min at 3500 g, supernatants were filtered using a 0.22 μM filter, supplemented with EDTA at a final concentration of 5 mM and loaded at 1 ml/min on a AAVX Pre-packed Column, 0.5×5 cm, 1 ml (ThermoFisher #A36652) mounted on an AKTA Pure instrument. Bound AAV particles were washed with 10 CV of wash buffer, pH 7.3 (50 mM Tris-Cl, 0.5 M NaCl, 0.01% Pluronic F-68) and eluted with 5 CV of glycine elution buffer, pH 2.7 (0.1 M Glycine, 0.2 M NaCl, 0.25 M L-arginine). Elution fractions (0.25 ml) were immediately neutralized with 25 μl of 1 M Tris-Cl, pH 10. Fractions containing AAV (as assessed by UV280 signal) were pooled and dialyzed, using the Float-A-Lyzer Dialysis Device 100 KD (Spectrum™ #G235071), O/N against PBS, pH 7.4+0.0010% Pluronic F-68 and then passed through a 0.22 μm filter unit.

### Titer and Decoration Level Determination:

The QX200 droplet digital PCR (ddPCR) system (Bio-Rad) was used to determine the titer of AAV vectors using the SV40 pA specific oligo set described in Table 3. Purified preparations were pretreated as follows: 5 μl of AAV were incubated with 1 unit of DNase and 1× DNase buffer (Merck #04716728001) in 20 μl total volume for 30 min at 37° C. 1 μl of Proteinase K at 20 mg/ml (Life technologies #E00491) was added to the mixture and incubated for 1 h at 55° C. followed by an inactivation step at 95° C. for 15 min. Different 1/10 serial dilutions of treated preparations were prepared in 1×PCR Buffer II (Life technologies #N8080010), 2.5 mM MgCl₂, 0.05% Pluronic F-68 (Sigma Aldrich #K4894), 2 ng/μl Sheared Salmon Sperm DNA (Invitrogen #15632011) and used as template for PCR amplification.

Droplets were generated as follow: 5.5 μl of pre-treated AAV dilutions, 900 nM of forward and reverse SV40 pA primers each, 125 nM SV40 pA probe and 11 μl of 2× ddPCR supermix for probes (BioRad #1863024) were mixed in 22 μl final volume. Technical duplicates were performed for each sample. 20 μl of each ddPCR assay mixture was loaded into a disposable droplet generator cartridge (Bio-Rad). Then, 70 μL of droplet generation oil for probes (BioRad #1863005) was loaded into each of the eight oil wells. The cartridge was then placed inside the QX200 droplet generator (Bio-Rad). When droplet generation was completed, 40 μl volume was transferred to a 96-well PCR plate using a multichannel pipet.

The plate was heat-sealed with foil and placed into the C1000 Touch Thermal Cycler (Bio-Rad). Thermal cycling conditions were as follows: 95° C. for 10 min, then 40 cycles of 95° C. for 30 s and 60° C. for 1 min followed by a 10 min incubation at 98° C. and a 4° C. indefinite hold. FAM fluorescent signal, labeling the AAV genome DNA sequence in each droplet were counted using a QX200 digital droplet reader, and analyzed by QuantaSoft analysis software (Bio-Rad). For calculation of AAV titers, the number of droplets was transformed by multiplying with the respective total dilution factor and additionally by a factor of 2 to take into account possible reannealing of single stranded genomes.

CE-SDS analysis (Agilent 2100 Bioanalyzer system, Protein 230 kit using denaturing conditions) was performed in order to evaluate the VP distribution in the purified samples and the total VP yield.

Calculation of total AAV particles/ml (vp/ml) and VP ratio
ng/μl (=μg/ml) values are divided by the observed MW and then multiplied by 1E+06 (μg to g conversion) to calculate moles/ml values
moles/ml values are multiplied by 6.022E+23 (avogadro nr) to calculate the number of molecules/ml
VP distribution: calculated based on molecules/ml values assuming one AAV is made of 60 VPs in total
AAV particles/ml (vp/ml): Sum of VP1+VP2+VP3 molecules ml are divided by 60

ddPCR and CE-SDS results are reported in Table 9.

TABLE 9
QC on Affibody and GP2 decorated AAV8 preps

| | VP2 fusion | | |
|---|---|---|---|
| Sample ID | per AAV | vg/cell | vp/cell |
| (AAV-701) ssAAV8-CAG-eGFP- | 15.7 | 1.04E+04 | 2.72E+05 |
| NOX-VP2-anti-PDGFRb-affibody | | | |
| (AAV-702) ssAAV8-CAG-eGFP- | 12.2 | 2.85E+03 | 5.65E+04 |
| NOX-VP2-anti-HER2-affibody | | | |
| (AAV-703) ssAAV8-CAG-eGFP- | 18.5 | 7.88E+03 | 7.77E+04 |
| NOX-VP2-GP2_Cl1 | | | |
| (AAV-704) ssAAV8-CAG-eGFP- | 14.1 | 9.65E+03 | 1.19E+05 |
| NOX-VP2-GP2_Cl5 | | | |

### In Vitro Infectivity of Decorated AAV8 on Receptor Over-Expressing Cell Lines:

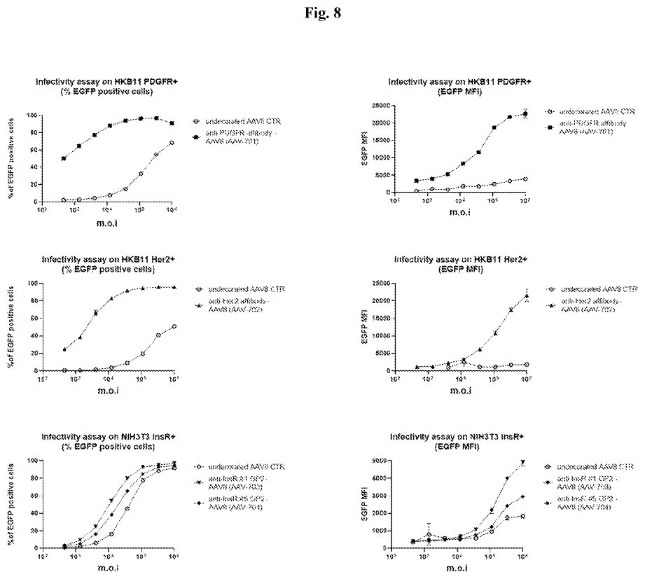

In order to evaluate the receptor specificity of decorated AAV8 preparations, an infectivity assay was performed on HKB11 cells over-expressing human PDGFRb (HKB11 PDGFR+) or human HER2 (HKB11 Her2+) and on NIH3T3 cells over-expressing human Insulin Receptor (NiH3T3 InsR+). At the day of infection, cells were resuspended in DMEM to 5E+05 cells/ml and 50 μl/well were seeded in a 96 well plate (2.5E+04 cells/well). AAV8 decorated preparations and an undecorated AAV8 control, produced in a separate experiment using the same cargo and methodology, were diluted in DMEM medium and different concentrations ranging from 1E+06 to 4.57E+02 moi based on viral genome concentration were applied to the seeded cells (final volume 100 μl). Four days post transduction cells were detached and FACS analysis was performed to calculate the percentage of EGFP positive cells and EGFP MFI. FIG. 8 depicts the number of full particles versus the percentage of infected (EGFP+) cells or EGFP MFI.

All tested Affibodies and GP2 sequences fused to AAV8 VP2 showed enhancement of transduction on receptor over-expressing cell lines compared to the undecorated control.

### Example 4: Evaluation of Sso7d Scaffold as VP2 Fusion on Different AAV Backbones

The aim of this experiment was to assess the possibility to graft a ligand scaffold into different AAV serotypes using N-terminal VP2 fusion. In this example, a functional anti-EGFR Sso7d sequence was used as ligand scaffold.

### Sso7d-AAV6Sil2 Assessment:

First, AAV6Sil2 was used. This backbone contains a set of mutations summarized in Table 10 to abolish or reduce binding to Heparan Sulphate Proteoglycan (HSPG) and Sialic Acid (SIA), which serve as primary attachment sites for AAV6. The listed mutations were incorporated in both the rep2/cap6 VP2 KO plasmid (T138A) and the anti-EGFR Sso7d (E.18.4.5) VP2 fusion expression plasmid (anti-EGFR Sso7d fused to VP2 N-terminus via G4S or (G4S)3) as described in Table 10 and 11. Cloning, expression, purification and assessment of viral concentration of the different preparations was performed as described in Example 2 with the following exceptions:
CAG-EGFP-NOX single stranded cargo (DL144) was used
Samples were harvested 3 days post transfection

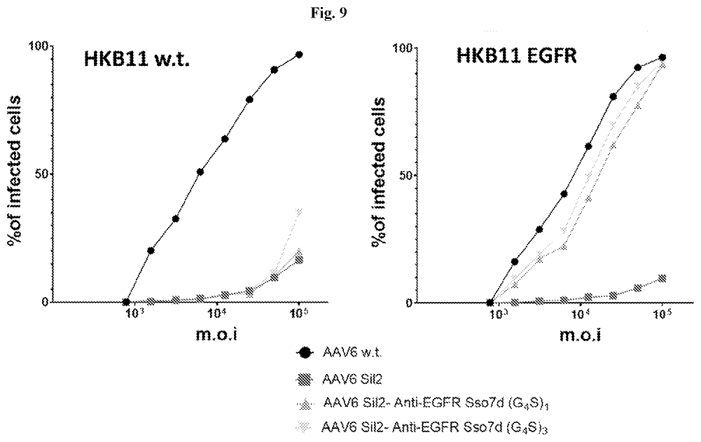

In order to evaluate functionality of the decorated AAV6Sil2 preparation, an infectivity assay was conducted on the HKB11 wildtype (w.t.) and on HKB11_EGFR over-expressing cell line using the same protocol described in Example 2 (1E+05 as starting moi in vg). Results are reported in FIG. 9.

TABLE 10
Overview of AAV6Sil2 mutation set

| | | | Expected | |
|---|---|---|---|---|
| | Silenced | Silencing | blocked | |
| Serotype | ID | mutations | pathway | Reference |
| AAV6 | AAV6_Sil2 | K531E, K459S, | HSPG and | DOI: 10.1128/JVI.00161-16 and |
| | | V473D, N500E | SIA | https://doi.org/10.1016/j.virol.2011.08.011 |

TABLE 11
List of rep/cap VP2 KO and VP2 expression plasmids used in this
example (AAV6_Sil2) in addition to pHelper and DL144 cargo

| Sample ID | Rep/Cap plasmid | VP2 expression plasmid |
|---|---|---|
| AAV6 w.t. | pRep2Cap6 | none |
| AAV6 Sil2 | (AgC1227) pR2_C6_Sil2 | none |
| AAV6 Sil2− | (AgC1248) | *(AgC1281)_pRS5a_anti- |
| Anti-EGFR | pR2_C6_VP2KO_Sil2 | EGFR_Ssod7_G4Sx1_AAV6_VP2_Sil2_D590A |
| Sso7d G₄S | | |
| AAV6 Sil2− | (AgC1248) | *(AgC1278)_pRS5a_anti- |
| Anti-EGFR | pR2_C6_VP2KO_Sil2 | EGFR_Ssod7_G4Sx3_AAV6_VP2_Sil2_D590A |
| Sso7d (G₄S)₃ | | |

*D590A mutation was unintentionally present in the VP2 expression plasmids, this mutation in VP2 only protein doesn't affect transduction as confirmed in HKB11 w.t. infectivity profiling showed in FIG. 9

Ablation of the HSPG and Sialic acid interaction on the AAV6 backbone (AAV6_Sil2 mutation set) reduces the in-vitro viral uptake by roughly 100-fold as compared to the parental sequence. Decoration of the AAV6_Sil2 backbone with the anti-EGFR Sso7d scaffold restores AAV6 w.t. infectivity on the EGFR over-expressing cell line. The two linkers between Sso7d and VP2 tested in this experiment appear not to have an effect on infectivity.

### Sso7d-AAV9 Assessment:

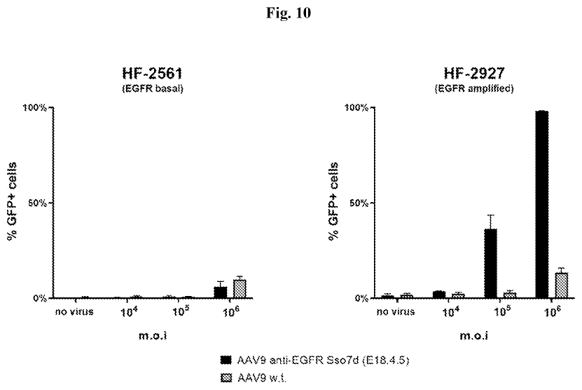

The second backbone evaluated was AAV9. Again, two different capsid expression plasmids were generated, a rep2/Cap9 VP2 KO (T138A) and an anti-EGFR Sso7d (E.18.4.5) AAV9 VP2 fusion expression plasmid (fusion of anti-EGFR Sso7d at the N-terminus of AAV9 VP2 linked by (G4S)3) as outlined in Table 12. Cloning, expression, purification and assessment of viral concentration of the different prep was performed as described in this example for AAV6Sil2 (harvest time: 2 days post-transfection). In order to evaluate functionality of decorated AAV9 preparations, an infectivity assay on GBM cells (as described in Example 2) was performed. In this experiment we used two different clones having EGFR enhanced expression (HF-2927) and basal EGFR level (HF-2561), respectively. For the infectivity assay, 350 HF-2561 cells or 500 HF-2927 cells were seeded per well in a 384-well plate (Corning, #3830). To adjust for growth differences between the different neurosphere cell lines, 16 extra wells were seeded per cell line to determine an average cell count per culture two days after seeding upon sphere formation. These wells were stained with Hoechst 33342 after two days of culture to quantify the cells per well (1 h with 10 μg/ml; Sigma B2261). The cell number was determined with the Opera Phenix High-Content Screening System (PerkinElmer) and virus preparations were added to the medium of the unstained spheres to reach the desired MOI per cell line (no virus, MOI 10E4, 10E5, 10E6). 5 days after infection, the spheres were stained with Hoechst for 1 h and imaged with the Opera Phenix for Hoechst (cell count) and GFP (viral infection). Imaging was performed with the 10× air objective in confocal mode to cover 10 mm in Z (18 planes). Image analysis was run with the Harmony software on the maximal projections and the percentage of GFP-positive cells was determined after nuclear segmentation (GFP+ cells with >1500 nuclear GFP signal). Results are reported in FIG. 10.

TABLE 12
List of rep/cap VP2 KO and VP2 expression plasmids used in
this example (AAV9) in addition to pHelper and DL144 cargo

| Sample ID | Rep/Cap plasmid | VP2 expression plasmid |
|---|---|---|
| AAV9 w.t. | pRep2Cap9 | none |
| AAV9 - | (NC036) | (NC043) pRS5a_G4Sx3_anti- |
| Anti-EGFR | pRep2_p5_Cap9_VP2 | EGFR_Sso7d_AAV9-VP2 |
| Sso7d (G₄S)₃ | KO | |

Fusion of anti-EGFR Sso7d at the VP2 N-terminus of AAV9 leads to increased infectivity in GBM neurospheres with high EGFR expression (HF-2927) but shows no improvement using a clone with basal EGFR expression (HF-2561).

### Sso7d-AAV1Sil1 Assessment:

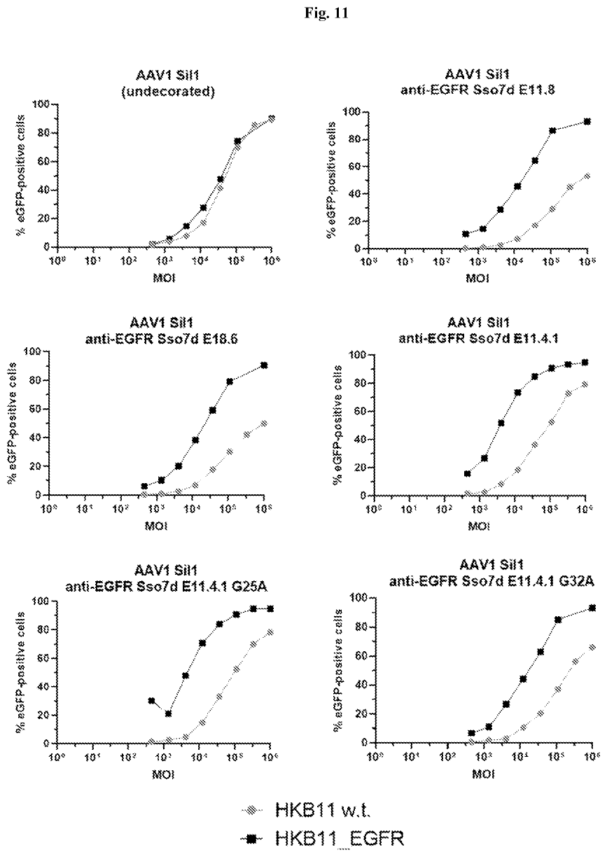

Finally, we tested AAV1Sil1 as backbone for ligand decoration by VP2 N-terminal fusion. This backbone contains a set of mutations as described in Table 13 to abolish or reduce binding to Sialic Acid (SIA) which serves as primary attachment site for AAV1. The listed mutations were incorporated in both the rep2/cap1 VP2 KO expression plasmid (T138A) as well as the different anti-EGFR Sso7d-VP2 fusion expression plasmids used in this experiment and listed in Table 14 and 15 (different anti-EGFR Sso7d sequences fused to AAV1 VP2 N-terminus linked by (G4S)3). Cloning, expression, purification and assessment of viral concentration of the different prep was performed as described in Example 2. In order to evaluate the functionality of the decorated AAV1Sil1 preparations, an infectivity assay was conducted on HKB11 w.t. and HKB11_EGFR over-expressing cell line using the same protocol as described in Example 2. The results are graphically depicted in FIG. 11.

TABLE 13
Overview of AAV1Sil1 mutation set

| | | | Expected | |
|---|---|---|---|---|
| | | Silencing | blocked | |
| Serotype | Silenced ID | mutations | pathway | Reference |
| AAV1 | AAV1_Sil1 | V473D, | SIA | DOI: 10.1128/ |
| | | N500E | | JVI.00161-16 |

TABLE 14
Epitope/affinity characteristics of anti-EGFR Sso7d sequences used in this Example;
Sequence, epitope and affinity data from Traxlmayr et al. (DOI 10.1074/jbc.M116.741314)
and Salzer et al. (doi.org/10.1038/s41467-020-17970-3):

| | | Final sequence fused to | Kd on | Kd on |
|---|---|---|---|---|
| clone ID | Epitope | AAV1Sill VP2 N-term | A431 | Jurkat |
| *E18.4.5 | Compete with | MATVKLTYQGEEKQVDISKITYVDRAGQFI | 19 nM | n.a |
| | Cetuximab (D3 binder) | WFEYDEGGGALGTGWVSEKDAPKELLQM | | |
| | | LEKQGGGGSGGGGSGGGGS | | |
| E11.8 | Do not compete with | MATVKFTYQGEEKQVDISKIKRVDRYGQSI | 67 nM | n.a |
| | Cetuximab and with | HFNYDEGGGAYGWGWVSEKDAPKELLQM | | |
| | E18.6 | LEKQGGGGSGGGGSGGGGS | | |
| E18.6 | Do not compete with | MATVKFTYQGEEKQVDISKIKNVLRYGQQI | 73 nM | n.a |
| | Cetuximab and with | VFSYDEGGGAGGNGFVSEKDAPKELLQML | | |
| | E11.8 | EKQGGGGSGGGGSGGGGS | | |
| E11.4.1 | Compete with | MATVKFTYQGEEKQVDISKIMYVIRGGQRI | 17 nM | 13 nM |
| | Cetuximab (D3 binder) | AFGYDEGDGAWGDGIVSEKDAPKELLQML | | |
| | | EKQGGGGSGGGGSGGGGS | | |
| E11.4.1 | Compete with | MATVKFTYQGEEKQVDISKIMYVIRAGQRI | n.a | 125 nM |
| G25A | Cetuximab (D3 binder) | AFGYDEGDGAWGDGIVSEKDAPKELLQML | | |
| | | EKQGGGGSGGGGSGGGGS | | |
| E11.4.1 | Compete with | MATVKFTYQGEEKQVDISKIMYVIRGGQRI | n.a | 877 nM |
| G32A | Cetuximab (D3 binder) | AFAYDEGDGAWGDGIVSEKDAPKELLQML | | |
| | | EKQGGGGSGGGGSGGGGS | | |

*Not used in this experiment

TABLE 15
List of rep/cap VP2 KO and VP2 expression plasmids used in this
example (AAV1Sil1) in addition to pHelper and DL144 cargo

| Sample ID | Rep/Cap plasmid | VP2 expression plasmid |
|---|---|---|
| AAV1 Sil1 | (NC056)\pRep2_Cap1_AAV1\ | none |
| | Sil1_V473D_N500E | |
| AAV1 Sil1 | (NC057)\pRep2_Cap1_AAV1\ | (HS0004) pRS5a_anti-EGFR |
| anti-EGFR | Sil1_VP2\KO | Sso7d E11.8_(G4S3)_AAV1 |
| Sso7d E11.8 | | Sil1 VP2 |
| AAV1 Sil1 | (NC057)\pRep2_Cap1_AAV1\ | (HS0005) pRS5a_anti-EGFR |
| anti-EGFR | Sil1_VP2\KO | Sso7d E18.6_(G4S3)_AAV1 |
| Sso7d E18.6 | | Sil1 VP2 |
| AAV1 Sil1 | (NC057)\pRep2_Cap1_AAV1\ | (HS0001) pRS5a_anti-EGFR |
| anti-EGFR | Sil1_VP2\KO | Sso7d E11.4.1_(G4S3)_AAV1 |
| Sso7d E11.4.1 | | Sil1 VP2 |
| AAV1 Sil1 | (NC057)\pRep2_Cap1_AAV1\ | (HS0002) pRS5a_anti-EGFR |
| anti-EGFR | Sil1_VP2\KO | Sso7d E11.4.1 |
| Sso7d E11.4.1 | | G25A_(G4S3)_AAV1 Sil1 VP |
| G25A | | |
| AAV1 Sil1 | (NC057)\pRep2_Cap1_AAV1\ | (HS0003) pRS5a_anti-EGFR |
| anti-EGFR | Sil1_VP2\KO | Sso7d E11.4.1 |
| Sso7d E11.4.1 | | G32A_(G4S3)_AAV1 Sil1 VP |
| G32A | | |

All tested anti-EGFR Sso7d scaffold variants fused to AAV1Sil1 VP2 showed increased transduction of EGFR over-expressing cell line as compared to undecorated parental cells.

### Example 5: Creation of Sso7d Phage Library and Phage Display on Human EGFR

### Example 5.1. Library Preparation

As phage display vector, pPD7-1_ompA_Sso7d_APP with an Amber stop and Ampicillin resistance was chosen.

The Sso7d library (Lib8) was designed as follow: rcSso7d positions 22, 24, 26, 29, 31, 33, 41, 43 and 45 relative to the rcSso7d polypeptide sequence including the first methionine were randomized by eight amino acids (D, R, H, N, A, I, Y and W) equally represented at each position. Additionally, putative glycosylation (NxS NxT—x not P), integrin binding (RGD, RYD, KGD, NGR, LDV, DGE) and CD11c/CD18 binding (GPR) motifs were avoided to reduce post-translational modification events and un-specific binding and AgeI, BamHI and BsaI restriction sites were removed from the design for cloning reason. The rcSso7d backbone used for randomization is shown below:

(M)ATVKFTYQGEEKQVDISKIKXVXRXGQXIXFXYDEGGGAXGXGXVSE
KDAPKELLQMLEKQ
Underlined positions: rcSso7d substitutions K7T,
K9Q, K28Q, Q40A plus KK C-terminal truncation
compared to w.t. Sso7d sequence described in
Traxlmayr et al. ((DOI 10.1074/jbc.M116.741314).
X represent randomized positions.

Library inserts were amplified by PCR introducing NruI and EcoRI restriction sites, using Q5 Hot Start High-Fidelity DNA Polymerase (NEB, M0493L).

PCR-reactions contained 5 μl of 5× Q5 reaction buffer, 0.5 μl of 50 mM dNTPs, 250 μg of template DNA of Sso7d library, 10 μmol of each primer, and 0.25 μl of Q5 Hot Start High-Fidelity DNA Polymerase per 25 μl reaction volume. The 265 bp fragment was amplified using following cycling protocol: Initial denaturation step for 30 sec at 98° C.; 98° C. for 7 sec of denaturation, annealing at 62° C. for 15 sec, elongation for 15 seconds at 72° C. for 25 cycles; final elongation for 2 minutes at 72° C. For removal of potentially remaining template DNA and primers, the PCR reactions were loaded on a 2% TAE agarose gel. The correct band was excised and purified with Wizard® SV Gel and PCR Clean-Up System, Promega, A9281.

The amplified Sso7d library was digested with restriction enzymes NruI-HF ((NEB R3192L) and EcoRI-HF (NEB R3101L) for 3 h at 37° C. For removal of digested overhangs, another cleanup step was done with the Wizard kit mentioned above.

For the preparation of the phage display vector, pPD7-1_ompA_Sso7d_APP with an Amber stop, vector DNA was digested with EcoRI-HF/NruI-HF, for 3 h at 37° C. For the separation of vector and insert fragment, the digested vector was loaded on a 1% TAE agarose gel and after electrophoresis the vector band with the correct size of 4482 bp was been excised. Melting and cleanup was performed as described above.

2 μg of digested vector DNA and 434 ng of digested insert DNA were ligated using 1.6 U of T4 DNA Ligase (ThermoFisher Scientific, No. 15224041) per ug of Vector-DNA (which corresponds to a 4-fold molar excess of insert DNA). Ligation was done for 16 h at 16° C., followed by a 10 minutes heat inactivation step at 65° C.

Desalting of the ligated library was conducted with Glycogen (Ultrapure Glycogen, ThermoFisher, No 10814-010) and 2-Butanol (Sigma, No. 19440) precipitation, followed by a wash step with 70% ethanol. The ligated DNA was then dissolved in ddH2O.

30 transformation reaction using electrocompetent TG1 [F′ traD36 proAB lacIqZ ΔM15] supE thi-1 Δ(lac-proAB) Δ(mcrB-hsdSM)5(rK−mK−), Lucigen, No. 60502-2, were performed in Gene Pulser/MicroPulser electroporation cuvettes, 0.1 cm gap, No. 1652089, BioRad, with 66.7 ng of ligated vector and 25 μl of cells per transformation, with the following settings on BioRad Genepulser Xcell instrument: 1.8 kV, 600 Ohm, 10 ρF. After transformation, cells were resuspended in 2.5 ml prewarmed Terrific Broth Recovery media per transformation (TB modified, contains 1.2% BactoTryptone, 2.4% yeast extract, 54 mM K2HPO4, 16 mM KH2PO4, 0.4% glycerol). The recovery time was 1 h at 37° C. in a shaking incubator. The library was amplified in 1.5 1 LB medium containing 100 μg/ml carbenicillin and 1% glucose, for 15 h at 21° C. with shaking at 220 rpm. The starting OD600 nm for over night culture was 0.23; the final harvest was done after 15 h at an OD600 nm of 1.86.

As quality control, the vector background was determined by ligation of the vector fragment with ligase only, without the insert fragment, and transformation.

Library size and vector background were identified by doing dilution series after a recovery time of 1 h, by plating on LB/100 μg/ml Carb/Gluc Agar plates. The final library size was determined to be 2.67×10E9 members, which corresponds to a 20-fold oversampling of the theoretical diversity of the library, with 1.34×10E8 members. A very low vector background of 0.08% was determined.

Sanger Sequencing of 94 clones of the library showed 94 different Sso7d sequences and no sequence of vector backbone.

The library was harvested by a centrifugation step and the pellet was resuspended in LB/100 μg/ml carb/1% glucose/20% Glycerol. Then aliquots (containing ˜8-fold the diversity of the library) were snap-frozen in liquid nitrogen, and then stored at −80° C.

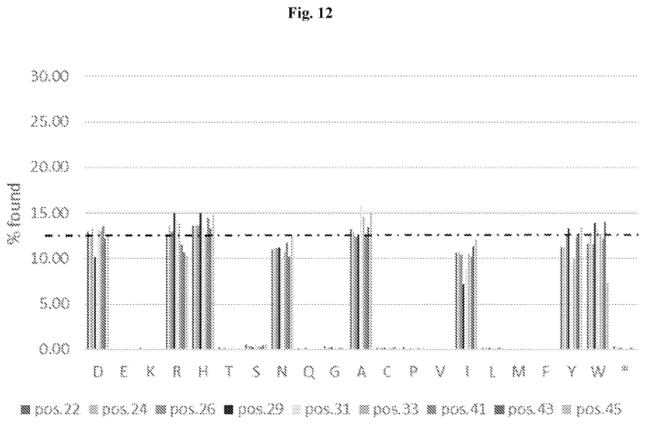

The library was sequenced on a MiSeq System (Illumina) using the Illumina MiSeq Reagent Kit v3 (150 cycles, #MS-102-3001). Generated fastq files were initially filtered by quality (Q>30, where Q indicates Phred quality score) and length (120-130 bp). After quality trim, sequences were processed aiming to map Sso7d specific boundaries (DISK to DAPK). Amino acid distribution at targeted positions was assessed on final filtered sequences: ˜500-700K reads were assessed. The results are shown in FIG. 12.

The amino acid distribution at the randomized positions was as expected with a frequency of about 12.5% for each of the eight amino acids used for randomization.

### Example 5.2 Preparation of Phages Using the Sso7d Plasmid Library

Glycerol stock containing ˜8 fold the diversity of the library was inoculated in 200 ml of 2YT/100 μg/ml carbenicillin/1% glucose, at a starting OD600 nm of 0.24. Cells were grown at 37° C. and 220 rpm until an OD600 of 0.59. Helper phage infection was done by adding Hyperphage, M13 K07ΔpIII, Progen, PRHYPE, with a MOI (Multiplicity of infection) of 20, first in a waterbath at 37° C. without shaking, and then at 37° C. with shaking at 220 rpm. After preparation of dilution series as infection controls (Hyperphage confers Kanamycin resistance to infected E. coli cells) and plating of different dilutions on both agar plates LB/100 μg/ml carbenicillin/1% glucose and on LB/50 μg/ml kanamycin/1% glucose, culture was centrifuged to remove any remaining Hyperphage and the pellet containing helperphage infected E. coli was resuspended in 11 2×YT/100 μg/ml carbenicillin/50 μg/ml kanamycin/0.25 mM IPTG, and incubated O/N at 22° C. in a shaker.

On the next day, control agar plates showed nearly the same number of colonies on both antibiotic resistances, which demonstrates that Helperphage infection was efficient.

The culture was centrifuged and phages were precipitated from the supernatant by a 3h incubation step with % volume of ice cold 20% PEG6000/2.5 M NaCl (polyethylenglycol/NaCl), centrifugation, and after resuspension and dissolving with PBS over night at 4° C. with a second precipitation step. A second resuspension step in PBS was conducted at room temperature for 2 h, in order to get all phages dissolved completely. For removal of still remaining cell debris, another centrifugation step at 4° C. was performed.

Phage titer was determined by infection of TG1F′. For titer determination in general TG1F′ cells need to be grown before infection on M9 agar plates (containing 200 ml 5× M9-minimum salts, 780 ml bacto agar solution, 2 ml of 1 M MgSO4, 0.1 ml of 1 M CaCl₂), 20 ml of 20% glucose, 400 μl of 1% thiaminehydrochloride) to develop their F-pili which are necessary for infection with phages. Then, a liquid culture of these cells in 2×YT medium was inoculated from an M9 plate, as they need grow exponentially (up to an OD600 nm of 0.6-0.8) for titer determination. After preparation of dilution series of phages, grown TG1F′ are added, and after an infection time of 30 minutes at 37° C., dilutions are plated on LB/30 μg/ml carbenicillin/1% glucose plates.

On the next day, colonies of different dilutions were counted, and the titer was calculated. After addition of 20% glycerol for freezing, a phage titer of 3.5×10E12 phages/ml was determined. Aliquots were stored at −80° C.

### Example 5.3 Phage Display on Human EGFR

To assess library quality a phage display campaign was conducted. Human EGFR was chosen as target. Three rounds of differential cell panning were done. HKB11 suspension cells stably transfected with human EGFR with a viability >98% were used in the first and third panning round. In the second round a biotinylated human EGFR-protein fused to an Fc was used. Cell cultivation was performed in appropriate medium, with addition of 200 μg/ml of zeocin and 1% FBS, with splitting 3× per week to 3×10E5 cells/ml.

For cell panning rounds one and three, 1×10E7 cells were prepared by three washing and centrifugation steps with precooled PBS/5% FBS. After a blocking time of two hours for phages and target cells with same buffer, cells were centrifuged and resuspended with phages and incubated for another 2 h. The amount of input phages amounted to at least ˜500 fold the diversity of the library, or of the output of the previous panning round. After phage incubation, in the first cell panning round cells were washed and centrifuged twice without any incubation time, in the third round three wash steps were performed with 5 minutes incubation time for each. In both cases, elution was done with low and high pH, using 100 mM glycine-HCl/500 mM NaCl, pH 2.2, and 100 mM triethylamine, pH 11.5. Direct neutralization was performed after each step. All steps for cell panning were performed at 4° C., except for the final elution of phages which was performed at RT.

The second panning round was performed with biotinylated human EGFR-protein, fused to an Fc-tag. Pre-blocked phages were incubated directly with 500 nM biotinylated protein for 1 h. Then the biotinylated hEGFR-Fc-protein with the bound phages was captured in a neutravidin coated plate for another hour. Wells were washed 3 times with PBS+0.05% Tween 20, and then with 3×PBS, without any incubation period. Elution was conducted in the same way as for cell panning.

The phage titer of the output was determined as described above for phage preparations. The results are summarized in Table 16.

TABLE 16
Phage titers in panning rounds

| | 1st round | | 2nd round | | 3rd round | |
|---|---|---|---|---|---|---|
| | Input | Output | Input | Output | Input | Output |
| EGFR | WCP | | LP | | WCP | |
| | (HKB11-EGFR transfected) | | (Neutr. + hsEGFR_hFc1P) | | (HKB11-EGFR transfected) | |
| Phage titers | 6.9E+10 | 5.6E+06 | 1.6E+10 | 5.4E+06 | 4.5E+10 | 3.2E+09 |
| Preblocking | | | Neutravidin | | | |
| of phages | | | | | | |
| Washing | Low stringent | | Low Stringent | | Standard | |
| conditions | | | | | | |

| | | |
|---|---|---|
| 2x FACS buffer, quick | 3x PBST, quick | 3x FACS Buffer, 5 min |

3x PBS, quick

### Phage Infection of Output and Preparation for Following Panning Rounds:

For further amplification of phages, exponentially grown TG1F′ at an OD600 nm of 0.6-0.8 were infected with the phage output with 10-20 fold excess of TG1F′-volume to elution volume, for 45 minutes at 37° C. in a water bath. After a centrifugation step for pelleting of TG1F′, the pellet with the infected bacteria was transferred into 100 ml of 2×YT media containing 100 μg/ml carbenicillin and 1% glucose and grown O/N at 25° C. under shaking at 220 rpm in shake flasks. On the next day, after a centrifugation step, glycerol stocks were prepared by resuspension of the pellets in fresh culture media containing additionally 20% glycerol, and a new phage preparation was started from the glycerol stock.

For this, a new 10 ml culture containing 2YT/100 μg/ml carb/1% glucose was inoculated with a glycerol stock to an OD600 nm of 0.2-0.3. The culture was grown for 30-60 minutes at 37° C. in a shaking incubator at 220 rpm. Hyperphage infection was performed in 5 ml of culture at a moi of 20, for 30 minutes in a water bath at 37° C. without shaking, and then another 30 minutes in a shaker at 37° C. and 220 rpm. This process was controlled by plating 50 μl of the infected solution on an LB/50 μg/ml kanamycin/1% glucose agar plate. After a centrifugation step for the removal of Hyperphage, the resulting pellet was transferred into 10 ml 2×YT/100 μg/ml carbenicillin/50 μg/ml kanamycin/0.25 mM IPTG medium and incubated under shaking at 22° C. and 220 rpm O/N for phage production.

On the following day, successful infection was demonstrated by a lawn on the agar plate. Phage containing culture supernatant was separated from E. coli cells by a centrifugation step, and phages carrying Sso7d were precipitated with ˜¼ volume of ice cold 20% PEG6000/NaCl for 30 minutes on ice. The suspension was centrifuged for 30 minutes at 16000×g at 4° C. in a pre-cooled centrifuge, the supernatant was discarded and the precipitated phages were resuspended in PBS. Remaining cell debris was removed by another centrifugation step in an Eppendorf centrifuge for 5 minutes at 16000 rpm. Titer determination was done as described above.

After the third panning round and the O/N amplification of the output a DNA-Miniprep was done using the Qiagen Minikit, No. 27106, according to their recommended protocol.

### Example 5.4: Assessment of Sso7d Library Composition by NGS

### Library Preparation and NGS on Illumina's MiSeq:

Amplicon libraries were generated from DNA preparations of the output after three panning rounds in one single PCR reaction using primers containing Illumina's adapter. The forward primer was used in combination with a reverse primer containing the Illumina TruSeq index to allow for identification of individual samples. The list of primers used in this example is showed in Table 17.

TABLE 17
List of primers used in this example

| Sample | |
|---|---|
| ID | Primer seq 5′-3′ |
| LC0034 | AATGATACGGCGACCACCGAGATCTACAC |
| (for | TCTTTCCCTACACGACGCTCTTCCGAT |
| primer) | CTcaggtggacatcagcaagatcaag |
| LC0086 | CAAGCAGAAGACGGCATACGAGATCGTAC |
| (rev | GGTGACTGGAGTTCAGACGTGTGCTCTTC |
| primer) | CGATCTCTTTAGGGGCGTCCTTTTCGGACAC |

PCR reactions were prepared using Q5 Hot Start High-Fidelity Master Mix (NEB #M0494), 10 pmol of each primer and 100 ng of DNA preparation after the 3rd panning round as template in 25 μl total reaction volume. PCR was carried out with the following cycling protocol. 2 min at 98° C.; 15 cycles of 98° C. for 10 s, 55° C. for 15 s, 72° C. for 25 s; followed by final amplification at 72° C. for 2 min and then holding at 4° C. PCR products were separated by agarose gel electrophoresis on a Sybr-Safe prestained 1.5% agarose gel (30 min at 120 V run). Bands at the expected size were then cut and purified using Qiaquick Gel Extraction Kit (Qiagen #28704) and eluted in 50 μl of water. DNA concentration was measured on a Qubit instrument, while purity and size of the product was assessed using Agilent Bioanalyzer 2100 (Agilent DNA 1000 kit). After quantitation, libraries were equimolarly pooled at a final concentration of 8 μM and sequenced on a MiSeq System (Illumina) using the Illumina MiSeq Reagent Kit v3 (150 cycles, #MS-102-3001).

### Data Analysis Using Custom Script:

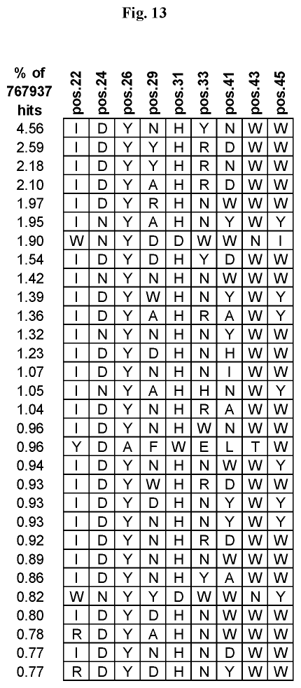

Generated fastq files were initially filtered by quality (Q>30, where Q indicates Phred quality score) and length (120-130 bp). After quality trim, sequences were processed aiming to map Sso7d specific boundaries (DISK to DAPK). Single sequence occurrences and AA distribution at targeted positions were assessed on final filtered sequences. Results are shown in FIG. 13.

NGS showed high enrichment of some library constructs, the most enriched one having an occurrence of 4.56%. NGS also demonstrated that the amino acid residues in the 9 randomized positions were relatively conserved, especially in some of the positions (positions 1, 3, 6, 8 and 9 of mutated positions, corresponding to positions 22, 26, 33, 43 and 45 of the Sso7d peptide sequence including the first methionine.

### Example 5.5: FACS Screening

Small scale protein expression was performed in BL21(DE3) cells from Agilent, No. 200131 (Genotype E. coli B F—dcm ompT hsdS(rB—mB—) gal λ(DE3)), with IPTG induction.

DNA was transformed into chemical competent BL21(DE3) cells by heat pulse according to users protocol. The cells were grown O/N on corresponding LB/100 μg/ml carbenicillin/1% glucose agar plates. On the next day, 4×96 single colonies were picked and transferred into 2×YT/100 μg/ml carb/1% glucose in masterplates and grown O/N at 37° C. in a plate shaker. One the next day, the same media additionally containing 30% glycerol was added for storage and the masterplates were stored at −80° C.

Protein expression for FACS screening was performed at 100 μl scale. Culture plates containing 2×YT/100 μg/ml carb/0.11% glucose were inoculated with replicators from thawn masterplates and grown at 37° C. in a plate shaker until an OD600 nm of ˜0.6. After induction with a final concentration of 0.25 mM IPTG, plates were further incubated in the plate shaker at 22° C. (RT) O/N. On the next day, lysis was conducted with BEL buffer, which consists of 2×BBS/EDTA, Teknova, No. B0205, and Lysozyme, Merck, No. 10837059001. The final concentration in wells was 200 mM boric acid, 150 mM NaCl, 2.5 mM EDTA and 2.5 mg/ml lysozyme. After a 1h incubation time period on a shaker, the plates were centrifuged, and BEL extracts containing secreted and soluble Sso7d protein were tested for cell binding.

For FACS analysis, the picked clones were tested on EGFR positive and a negative cell lines. The positive cell line was the same as described for Cell Panning, i.e. HKB11 cells stably transfected with human EGFR. As negative control, a house made HKB11 EGFR knockout cell line was used to be able to exclude cross reactivity on HKB11-cells (since EGFR is endogenously expressed on HKB11 wildtype cells). FACS analysis was performed on a FACS Calibur instrument with a high throughput sampler (BD Biosciences) in 96-well format. As FACS buffer, PBS containing 5% FBS and 0.04% NaN3 was used for all wash and incubation steps. The cell viability was >98%. 5×10E5 cells/well were prepared as described for panning with three wash and centrifugation steps. All steps were performed at 4° C.

In a first staining step, 50 μl of the BEL extracts were preincubated with an internally produced mouse-anti-APP-antibody at a final concentration of 24 μg/ml for 1h on a plate shaker. In a second step, 5×10E5 cells/well were added and further incubated for another hour. After 2 wash steps with cold FACS buffer, the cells were resuspended in a 1:100 dilution of the goat-anti-mouse-IgG-RPE-antibody R-Phycoerythrin AffiniPure F(ab′)₂ Fragment Goat Anti-Mouse IgG, F(ab′)₂ fragment specific (min X Hu, Bov, Hrs Sr Prot), Jackson ImmunoResearch, No. 115-116-072, and incubated for another 30 minutes on a plate shaker. After two final wash steps, stained cells were resuspended in cold PBS, and 5000 cells per well were measured with a FACS Calibur instrument.

81 primary hits out of 384 tested clones showed median values >10 on the EGFR overexpressing cells compared to the control EGFR knock out cell line (55 having a median of 10-30, 17 having a median of 30-60 and 9 having a median of 60-887). Sequence analysis showed enrichment of some sequences found up to ten times. In the end 41 different novel sequences could be identified. For around half of these, binding could be confirmed, either in another FACS, or in an ELISA on biotinylated protein.

To confirm specific binding of selected Sso7d variants to EGFR-transfected HKB11-cells, small scale E. coli protein expression and purification of six EGFR-binding Sso7d constructs was performed. Expression was done in freshly transformed BL21(DE3) in 20 ml culture. Cells were grown at 37° C. in a shaking incubator, induced with 0.25 mM IPTG and further incubated under shaking at 22° C. O/N. On the next day, the cultures were centrifuged, the pellet was frozen at −80° C. for two days and only the soluble fraction was purified from the pellet.

For protein purification, anti-APP-columns (anti-APP-antibody coupled to Sepharose resin, Novartis internally produced) were prepared in Poly-Prep chromatography Columns (BioRad, No. 731-1550), with 0.5 ml column volume per column.

Lysis was performed with 3.5 ml Bugbuster Mastermix (Millipore, No. 71456-3), containing complete EDTA-free protease inhibitor cocktail (Roche, 11873580001), benzonase (Novagen, No. 70746) and DNAse (Roche, No. 04 536 282 001) for 30 minutes at RT on a rotator. After separation of cellular debris and expressed Sso7d by a centrifugation and sterile filtration step, samples were diluted 1:10 with PBS before loading on the column, to avoid any potential damaging effect of Bugbuster on the APP-columns.

The equilibration and wash steps were performed with PBS pH 7.4 and the elution with 1-1.5 ml of 100 mM glycine, pH 3. Buffer exchange and concentration of purified protein was done with Amicon® Ultra-4 centrifugal filter units, 3 kDa MWCO, No. UFC800324. Quality of purified protein was analyzed with protein gels.

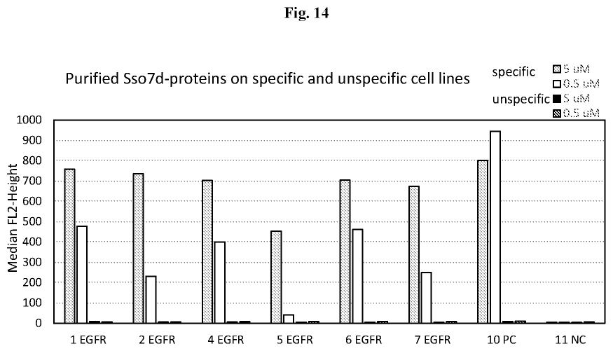

Specific cell binding of all six purified Ss07d constructs could be confirmed by FACS analysis. The results are shown in FIG. 14. The selected clones are characterized in Table 18.

In FACS analysis of all six Sso7d constructs, specific cell binding could be confirmed

TABLE 18
Sso7d constructs selected for FACS analysis. The amino acid positions in
column 6 refer to the AS position of Ss07d without the first methionine.

| | | Hit name | Clone | | | | | | | | | | | | | |
|---|---|---|---|---|---|---|---|---|---|---|---|---|---|---|---|---|
| Purif. | Sample | (pos. on | found on | Found in NGS | | | | | | | | | | | | |
| No. | Name | MP) | plate | (Top30) | | 5 | 20 | 21 | 23 | 25 | 28 | 30 | 32 | 40 | 42 | 44 |
| 1 | EGFR | 4.1A02 | 1x | no | | F | K | I | D | Y | W | H | N | Y | W | W |
| 2 | EGFR | 4.1A03 | 4x | Hit NO. 3 | 2.18% | F | K | I | D | Y | Y | H | R | N | W | W |
| 4 | EGFR | 4.1H02 | 2x | no | | F | K | I | D | Y | W | H | R | D | W | W |
| 5 | EGFR | 4.2A08 | 4x | Hit 1 NGS | 4.56% | F | K | I | D | Y | N | H | Y | N | W | W |
| 6 | EGFR | 4.2B11 | 10x | Hit No. 24 | 0.89% | F | K | I | D | Y | N | H | N | W | W | W |
| 7 | EGFR | 4.4E10 | 3x | no | | F | K | I | D | Y | Y | H | Y | D | W | W |
| 10 PC | C2013 | pPD7-1_Sso7d- | | | | L | T | Y | D | A | F | W | E | L | T | W |
| | | EGFR_APP | | | | | | | | | | | | | | |
| 11 NC | C2014 | pPD7-1_Sso7d- | | | | F | K | K | W | V | M | S | T | T | R | A |
| | | wt-libr.8_APP | | | | | | | | | | | | | | |

### Example 6: Lib8 Sso7d-VP2 AAV1Sil1 Library Generation

### Example 6.1: Lib8 Sso7d-AAV1Sil1 Plasmid Library Generation

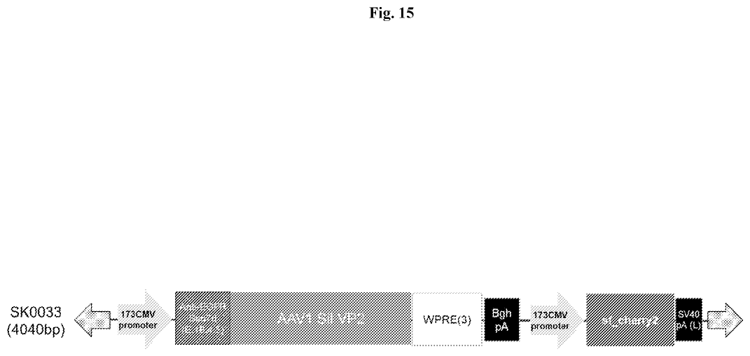

Genetic fusion of the Sso7d scaffold to the VP2 N-terminus gives the possibility to generate AAV libraries that can be used for in-vivo and in-vitro selection. Before generating the final Sso7d library, cargo construct SK033 was generated. SK033 comprises Anti-EGFR Sso7d (E.18.4.5.) fused to the N-terminus of AAV1Sil1 VP2 via a G4S linker. A small version of WPRE element (WPRE3) was added after the CDS in order to enhance VP2-fusion in cell expression. The fusion polypeptide is operably linked to a small 173CMV promoter. In addition, an sfCherry2 cassette under the control of a small 173CMV promoter and a short SV40 polyA (SV40L) was added to the construct allowing for direct selection/validation using FACS. The SK033 cargo design was selected based on good yield, percentage of full AAV particles and improved infectivity profile as compared to other cargo designs. AAV1Sil1 was primarily chosen for high viral yield in HEK293T/17 cells and for potential reduced in-vivo off-target activity due to absence/reduction of Sialic acid binding. SK033 cargo design is schematically represented in FIG. 15.

Acceptor plasmid SK0036 was generated by replacing the entire anti-EGFR Sso7d (E.18.4.5) sequence plus the initial sequence of AAV1 Sil1 VP2 present in SK0033 by a double BsaI restriction site placed in opposite direction. 35 pb homology arms were added to both ends of the library fragment insert described below.

The Sso7 (Lib8) fragment was ordered at Twist Bioscience. It comprises a 35 bp 5′UTR region, a randomized rcSSo7d scaffold (Human codon usage optimized), a G4S linker and the first 182 bp of AAV1 VP2 (DT1, i.e. having a deletion of the VP2 start codon coding for threonine).

The Sso7d library (Lib8) was designed as follow: rcSso7d positions 22, 24, 26, 29, 31, 33, 41, 43 and 45 (relative to the rcSso7d polypeptide sequence including the first methionine that is cleaved off) were randomized by eight amino acids (D, R, H, N, A, I, Y and W) equally represented at each position, additionally, putative glycosylation (NxS NxT—x not P), integrin binding (RGD, RYD, KGD, NGR, LDV, DGE) and CD11c/CD18 binding (GPR) motifs were avoided to reduce post-translational modification events and un-specific binding, and AgeI, BamHI and BsaI restriction sites were removed from the design for cloning reason. The rcSso7d backbone used for randomization is shown below.

rcSso7d backbone sequence used for library
generation
(M)ATVKFTYQGEEKQVDISKIKXVXRXGQXIXFXYDEGGGAXGXGXVSE
KDAPKELLQMLEKQ
Underlined positions: rcSso7d substitutions K7T,
K9Q, K28Q, Q40A plus KK C-terminal truncation
compared to w.t. Sso7d sequence described in
Traxlmayr et al. ((DOI 10.1074/ jbc.M116.741314).
X represent randomized positions. First methionine
is cleaved off

A fragment containing TGA stop codons at each randomized position was used as template for library synthesis leading to a fragment of 451 bp with a final complexity of 1.34E+08 variants. In order to remove Twist flanking sequence at each end of the fragment, the library was PCR amplified using LC0052 (5′ CCCGGGGAACTCCTCCCGAGTCGAAATTCGCCACC 3′) and LC0053 (5′ GTTGCTGGAGGTT CTCCGAGAGGTTGTGGGTCGGG 3′) primers. 4×PCR reactions were prepared using Q5 Hot Start High-Fidelity Master Mix (NEB #M0494), 10 pmol of each primer and 10 ng of the TWIST fragment as template in 25 μl total reaction volume at the following cycling protocol: 2 min at 95° C.; 10 cycles of 98° C. for 10 s; 65° C. for 15 s; 72° C. for 30 s followed by 72° C. for 2 min. To generate the Lib8 Sso7d-AAV1Sil1 plasmid library, 520 ng of PCR product (403 bp) and 4 μg of BsaI digested SK0036 acceptor plasmid were assembled using NEBuilder HiFi DNA Assembly Master Mix (NEB; E2621) in a final volume of 400 μl. The reaction product was then treated with Plasmid Safe (PS) DNase (Epicentre; E3105K) to digest any unassembled fragments and purified using a Wizard SV Gel and PCR Clean-up System (Promega #A9280). This reaction yielded 570 ng of assembled plasmid (as defined by the amount of DNA remaining after the PS DNase digestion step). One Shot TOP10 electrocompetent E. coli (ThermoFisher; C4040-52) were transformed with the assembled plasmid library in 42×50 μl vials (˜10 ng DNA/vial). 100 μl of pooled transformation were removed from the flask and different dilutions were plated on LB+kanamycin plates. The plates were incubated O/N at 37° C. The Library practical size as determined by the number of CFU on agar plates was found to be 4.12E+08. Colonies were then used to extract plasmid DNA (n=192) using the NucleoSpin 96 Plasmid kit (Macherey-Nagel #740625.4) and analyzed by Sanger sequencing using a reverse oligo mapping at the beginning of the VP2 region. The results are summarized in Table 19 and FIG. 16. The rest of the TopTen transformation sample was inoculated directly in 500 ml of TB+kanamycin media and plasmid DNA was extracted after O/N growth at 37° C. using Maxiprep Kit (QIAGEN #12163) yielding the (SK0037) pCargo_173CMV_SSo7d_lib8_AAV1sil1-VP2_173CMV_sfCherry plasmid library.

TABLE 19
Sanger QC and AA frequency on Lib8 Sso7d-
AAV1Sil1 plasmid library (SK0037)

| Parameters | Nr of clones (%) | |
|---|---|---|
| Readable sequences | 181 | |
| In-frame/expected sequences | 173 | (95.6%) |
| SK0036 backbone | 0 | (0%) |
| Sequences with stops/frameshift | 8 | (4.4%) |
| Unique sequences | 181 | (100%) |

Sanger sequencing showed a high degree of in-frame sequences (95.6%), no redundancy in the library and good distribution of the eight selected amino acids at randomized positions. A more in-depth analysis was conducted in Example 5.4 using NGS sequencing.

### Example 6.2: Lib8 Sso7d-VP2 AAV1Sil1 Library Generation and QC

16 liters of HEK293T/17 suspension cells (1.6E+10 total viable cells) were transfected with pHelper (˜30K plasmid/cell), (NC057) pRep2_Cap1_AAV1 Sil1 VP2 KO (˜30K plasmid/cell) and the SK0037 plasmid library (420 plasmids/cell) at 0.8 μg of total DNA/1E+06 cells using PEI Max reagent (3:1 PEI:DNA). The cargo plasmid was employed in low amounts to avoid chimerism and to improve phenotype-genotype linkage. Based on internal experience and external references about 400 plasmids per cell were identified as optimal quantity to achieve a good quality of the AAV library. Two days post transfection (yielding ˜35% cherry positive cells as measured by FACS), cells were pelleted and lysed in 51 of 50 mM Tris, pH 7.5, 0.5% Triton, 0.01% Pluronic F-68, 2 mM MgCl₂ and 12.5 U/ml of Nuclease (ThermoFisher #88702). After 2 h at 37° C., NaCl was added to a final concentration of 0.5 M, the lysates were incubated for additional 30 min and centrifuged for 30 min at 3500 g. Clarified lysates were filtered using 0.22 μM filter units and loaded at 10 ml/min on a POROS™ GoPure™ AAVX Pre-packed Column, 0.8×10 cm, 5 ml (ThermoFisher #A36651) mounted on an AKTA Pure instrument. Bound AAV particles were washed with 10 CV of Wash Buffer, pH 7.3 (50 mM Tris-Cl, 0.5 M NaCl, 0.01% Pluronic F-68) and eluted with 5 CV of glycine elution buffer, pH 2.7 (0.1 M glycine, 0.2 M NaCl, 0.25 M L-arginine). Elution fractions (0.25 ml) were immediately neutralized with 25 μl of 1 M Tris-Cl, pH 10. Due to high yield after the first affinity purification, flow-through was re-loaded twice and additional fractions were collected. Fractions containing AAV particles (as assessed by UV280 signal) were pooled (Lib8 Sso7d-VP2 AAV1Sil1_pool) and full and empty particles were separated on an iodixanol ultracentrifugation gradient and concentrated/buffer exchanged in PBS, pH 7.4+0.001% Pluronic F-68 using an Amicon Ultra-15 MWCO 100,000 Filter unit (Merck #UFC910008) and then passed through an 0.22 μm filter unit (final volume=1 ml).

CE-SDS and ddPCR assays were used to assess decoration levels, as well as total and full particle concentrations. Briefly, the QX200 droplet digital PCR (ddPCR) system (Bio-Rad) was used to determine the titer of AAV vectors using the SV40 pA specific oligo set described in Table 20. Purified preparations were pretreated as follow: 5 μl of AAV were incubated with 1 unit of DNase and 1× DNase buffer (Merck #04716728001) in 20 μl total volume for 30 min at 37° C. 1 μl of Proteinase K at 20 mg/ml (Life technologies #E00491) was added to the mixture and incubated for 1 h at 55° C. followed by an inactivation step at 95° C. for 15 min. Different 1/10 serial dilutions of treated preparations were prepared in 1×PCR Buffer II (Life technologies #N8080010), 2.5 mM MgCl₂, 0.05% Pluronic F-68 (Sigma Aldrich #K4894), 2 ng/μl Sheared Salmon Sperm DNA (Invitrogen #15632011) and used as template for PCR amplification.

Droplets were generated as follow: 5.5 μl of pre-treated AAV dilutions, 900 nM of forward and reverse SV40 pA primers each, 125 nM SV40 pA probe and 11 μl of 2× ddPCR supermix for probes (BioRad #1863024) were mixed in 22 μl final volume. Technical duplicates were performed for each sample. 20 μl of each ddPCR assay mixture was loaded into a disposable droplet generator cartridge (Bio-Rad). Then, 70 μl of droplet generation oil for probes (BioRad #1863005) was loaded into each of the eight oil wells. The cartridge was then placed inside the QX200 droplet generator (Bio-Rad). When droplet generation was completed, 40 μl were transferred to a 96-well PCR plate using a multichannel pipet.

The plate was heat-sealed with foil and placed into the C1000 Touch Thermal Cycler (Bio-Rad). Thermal cycling conditions were as follows: 95° C. for 10 min, then 40 cycles of 95° C. for 30 s and 60° C. for 1 min followed by a 10 min incubation at 98° C. and 4° C. indefinite hold. FAM fluorescent signal, labeling the AAV genome DNA sequence in each droplet were counted using a QX200 digital droplet reader, and analyzed by QuantaSoft analysis software (Bio-Rad). For calculation of AAV titers, the number of droplets were transformed by multiplying with the respective total dilution factor and additionally by a factor of 2 to take into account possible reannealing of single stranded genomes.

CE-SDS (Bioanalyzer) as previously described was used to assess decoration levels and total particle concentrations. The results are summarized in Table 21.

TABLE 20
oligo set used for AAV genome concentration (vg/ml)
measurement by ddPCR

| Oligo ID | 5′-3′ sequence |
|---|---|
| SV40pA133- | TGCTTTATTTGTGAAATTTGTGATGCT |
| For | |
| SV40pA133- | CCCTGAACCTGAAACATAAAATGA |
| Rev | |
| SV40pA133-P | FAM-TGTAACCATTATAAGCTGCAATAA |
| | ACAAGTTAACAACAACA-BHQ1 |

TABLE 21
Bioanalyzer and ddPCR results of
Lib8 Sso7d-VP2 AAV1Sil1 library
a: Observed MW of VPs (kDa)

| Sample ID | VP3 | VP2 | VP1 |
|---|---|---|---|
| Lib8 Sso7d-VP2 AAV1Sil1 (pool) | 66.2 | 89.0 | 99.1 |
| Lib8 Sso7d-VP2 AAV1Sil1 (full) | 66.5 | 89.7 | 99.7 |
| Lib8 Sso7d-VP2 AAV1Sil1 (empty) | 67.2 | 90.8 | 100.8 |

b: VPs distribution (assuming total of 60VPs per AAV particle)

| Sample ID | VP3 | VP2 | VP1 |
|---|---|---|---|
| Lib8 Sso7d-VP2 AAV1Sil1 (pool) | 54.5 | 0.5 | 5.0 |
| Lib8 Sso7d-VP2 AAV1Sil1 (full) | 53.3 | 1.5 | 5.2 |
| Lib8 Sso7d-VP2 AAV1Sil1 (empty) | 55.6 | 0.4 | 4 |

c: Viral genome (vg) and viral particle (vp) yield per transfected cells

| Sample ID | vg/cell | vp/cell | % full |
|---|---|---|---|
| Lib8 Sso7d-VP2 AAV1Sil1 (pool) | 4.29E+02 | 6.49E+04 | 0.7% |
| Lib8 Sso7d-VP2 AAV1Sil1 (full) | 3.04E+02 | 1.41E+03 | 21.5% |
| Lib8 Sso7d-VP2 AAV1Sil1 (empty) | 1.33E+01 | 9.19E+03 | 0.1% |

AAV-260: Lib8 Sso7d-VP2 AAV1Sil1 (full)
Concentration = 4.87E+12 vg/ml
Decoration level = *1.5 Sso7d/AAV
% full = 21.5%
Viral genome recovery after UC = 71%
Full particle enrichment after UC = 32.6-fold
*Likely underestimated due to the presence of ~80% empty undecorated AAVs

The low percentage of full particles is explained by the large excess of rep/Cap VP2 KO and pHelper plasmids transfected. The majority of the cells only contain these two plasmids but no cargo plasmid and therefore generate a progeny of undecorated empty particles. This also explains the relatively low decoration levels observed, since CE-SDS estimates the average number of decoration without taking into account the presence of undecorated/empty AAVs.

### Example 6.3: Assessment of Lib8 Sso7d-AAV1Sil1 Naive Library Composition by NGS

### Library Preparation and NGS on Illumina's MiSeq:

Amplicon libraries were generated from Plasmid (SK037) and AAV (AAV-260) libraries in one single PCR reaction using two different set of primers containing Illumina's adapter. The forward primer was used in combination with a reverse primer containing the Illumina TruSeq index to allow for identification of individual samples. The list of primers used in this example is showed in Table 22.

TABLE 22
List of primers used in this example
(bold: index, underlined: annealing region)

| Sample ID | Primer seq 5′-3′ |
|---|---|
| LC0034 | AATGATACGGCGACCACCGAGATCTACACTCTTTCCCTACA |
| (for primer) | CGACGCTCTTCCGATCTCAGGTGGACATCAGCAAGATCAAG |
| LC0038 | CAAGCAGAAGACGGCATACGAGATTGGTCAGTGACTGGAGTTCA |
| (rev | GACGTGTGCTCTTCCGATCTCTTTAGGGGCGTCCTTTTCAGACAC |
| primer/AAV | |
| library) | |
| LC0042 | CAAGCAGAAGACGGCATACGAGATTCAAGTGTGACTGGAGTTCA |
| (rev | GACGTGTGCTCTTCCGATCTCTTTAGGGGCGTCCTTTTCAGACAC |
| primer/Plasmid | |
| library) | |

PCR reactions were prepared using Q5 Hot Start High-Fidelity Master Mix (NEB #M0494), 10 pmol of each primer and 100 ng of plasmid library or 5E+10 viral genomes for the AAV library (corresponding to approx. 123 ng of dsDNA) as template in 25 μl total reaction volume. PCR was carried out with the following cycling protocol: 2 min at 95° C.; 20 cycles of 98° C. for 10 s, 55° C. for 15 s, 72° C. for 25 s; followed by final amplification at 72° C. for 2 min and then holding at 4° C. PCR products were separated by agarose gel electrophoresis on a Sybr-Safe pre-stained 1.5% agarose gel (30 min at 120 V run). Bands at the expected size (243 bp) were then cut and purified using Qiaquick Gel Extraction Kit (Qiagen #28704) and eluted in 50 μl of water. DNA concentration was measured on a Qubit instrument, while purity and size of the product was assessed using Agilent Bioanalyzer 2100 (Agilent DNA1000 kit). After quantitation, libraries were equimolarly pooled at a final concentration of 8 μM and sequenced on a MiSeq System (Illumina) using the Illumina MiSeq Reagent Kit v3 (150 cycles, #MS-102-3001).

### Data Analysis Using Custom Script:

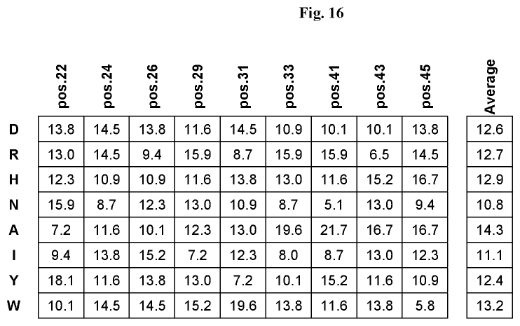

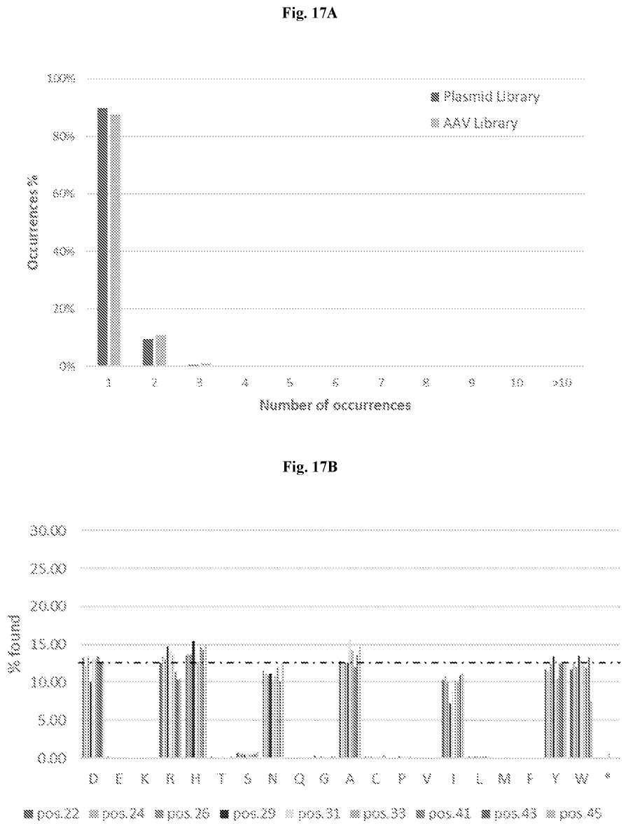

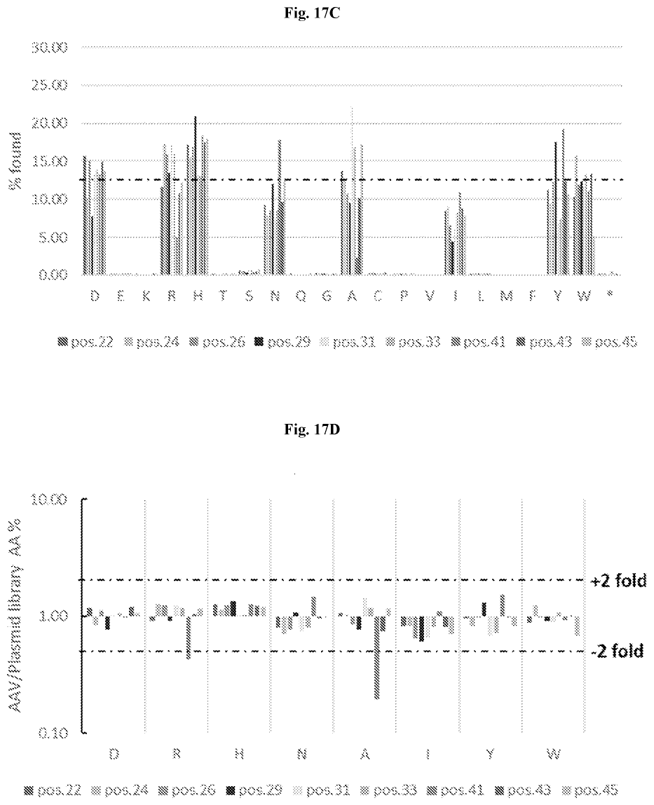

Generated fastq files were initially filtered by quality (Q>30, where Q indicates Phred quality score) and length (120-130 bp). After quality trim, sequences were processed aiming to map Sso7d specific boundaries (DISK to DAPK). Single sequence occurrences and amino acid distribution at the targeted positions were assessed on final filtered sequences: ˜400K reads for plasmid library and ˜700K reads for AAV library were assessed. The results are shown in FIGS. 17A-D.

Single sequence occurrence analysis showed that roughly roughly 90% of sequences were unique in both the plasmid and the AAV library indicating a good library diversity and minimal bias during AAV production.

NGS analysis showed the expected amino acid distribution at the randomized positions in the plasmid library. As expected, the AAV library exhibited a slightly stronger bias as compared to the plasmid library, which is however considered marginal for an AAV library.

### Example 7. In-Vivo Selection of Lib8 Sso7d-VP2 AAV1Sil1

The Lib8 Sso7d-VP2 AAV1Sil1 library was injected in humanized (hCD34) mice and biodistribution/NGS analysis was conducted on extracted DNA/RNA from different tissues with the aim to identify enriched AAV cargo sequences in the different organs.

### Example 7.1: Mobilized Humanized Mice Injection and Tissue Processing

For in vivo screening, NSG mice were utilized which were engrafted by human cord blood derived CD34+ cells. The humanization of the mice was confirmed by observing at least 30% human CD45+ cells over total CD45+ cells (mouse and human) using flow cytometry in the peripheral blood of the NSG mice 12 weeks post engraftment. Prior to administration of the Lib8 Sso7d-VP2 AAV1Sil1 library or undecorated AAV1Sil1 (ssCAG-EGFP-HPRE_NOX as cargo: DL144) viruses the HSCs of the mice were mobilized using a 5-day dose regimen. Briefly, G-CSF was administered through subcutaneous injection twice a day for four consecutive days at a dose of 125 μg/kg. On the morning of day 5, AMD3100 was administered through subcutaneous injection once at a dose of 5 mg/kg. Within one hour post AMD3100 injection, either the control (undecorated AAV1Sil1_ssCAG EGFP_NOX; 3 mice) or the library (Lib8 Sso7d-VP2 AAV1Sil1; 12 mice) viruses were administered through tail vein injection at a dose of 1.2E+13 vg/kg.

Five days post viral injection, mice were sacrificed and the following tissues were collected: whole blood, brain, liver, spleen, kidney, skeletal muscle, heart, lungs, and bone marrow. For whole blood, total blood from all mice in the same group was pooled (control n=3, library n=12). Pooled blood was spun at 500 g for 10 min at RT, plasma was removed, and the pellets were stored in −80° C. in aliquots. For all other tissues except for bone marrow, small sections (3 mm) of each tissue were cut off and pooled per group. Pooled tissues were cut into small pieces using sharp razors and stored in −80° C. in aliquots. One aliquot of pooled frozen tissue each (whole blood, brain, liver, spleen, kidney, skeletal muscle, heart, lungs) was homogenized in PBS using 5 mm stainless steel beads in a Qiagen TissueLyser II. DNA was purified from the homogenized lysates using a QIAmp DNA mini-Kit (Qiagen, 51306). Similarly, one aliquot of each tissue was homogenized in RLT buffer using the TissueLyser II, and the RNA was extracted using the RNeasy Plus Mini Kit (Qiagen, 74134). For bone marrow collection, clean bone ends were cut using sharp razors and were spun at 8000 g for 1 min at 4° C. The bone marrows from all the mice from the same group were pooled and were passed through a 100 μm strainer in FACS buffer (1×PBS, 0.5% BSA, 2 mM EDTA). An aliquot (1 million cells) of the pooled bone marrow cells was pelleted and stored at −80° C. as input and the rest of the cells (200 million for control, 900 million for Sso7d library) were used for purification of human CD34+ cells using magnetic beads conjugated anti-human CD34 antibody (Miltenyi, 130-097-047) with LS columns according to manufacturer's instructions. An aliquot of the purified CD34+ cells (0.3 million cells) was pelleted and stored at −80° C. as input and the rest of the cells (3.6 million for control, 64 million for Sso7d library) were stained with antibody cocktail of the following antibodies per million cells: 20 μl of anti-human CD34-FITC (BD Pharmagin, 555821), 2.5 μl of ani-mouse CD45p-eFluor610 (Themo, 61045182), 5 μl of ant-human CD45-V450 (BD Phamagin, 560367), 5 μl of ani-human CD90-APC (BD Phamagin, 559869), 5 3l of anti-human CD38-PE (BD Pharmagin, 555460), 5 μl of anti-human CD45RA-BV605 (BD Pharmagin, 562886). Cells were sorted for the following populations using an automated sorter (BD FACS ARIA II): two populations for the control group 1) mCD45-hCD45+hCD34+hCD45RA+2) mCD45-hCD45+hCD34+hCD45RA-; four populations for the library group 1) mCD45-hCD45+hCD34+hCD45RA+2) mCD45-hCD45+hCD34+hCD45RA-3) mCD45-hCD45+hCD34+hCD45RA-hCD38+hCD90-4) mCD45-hCD45+hCD34+hCD45RA-hCD38-hCD90+(LT-HSC). Sorted populations were centrifuged at 350 g for 5 m at 4C and were stored as dry pellets in −80° C. The frozen pellets were used for both DNA and RNA purification using an Allprep DNA/RNA Mini Kit (Qiagen, 80204). Concentration of the eluted DNA/RNA was determined using an UV spectrophotometer. The results are summarized in Table 23.

TABLE 23
Concentration of the DNA and RNA extracted from humanized mice tissues

| | | | RNA | RNA | DNA | DNA |
|---|---|---|---|---|---|---|
| Tissue | | Number of | elution | conc. | elution | conc. |
| source | Tissue description | Cells | (μl) | (ng/μl) | (μl) | (ng/μl) |
| Library | mCD45− hCD45+ hCD34+ | 2.00E+06 | 30 | 37 | 50 | 53 |
| sorted cells | hCD45RA+ (MPP1) | | | | | |
| Library | mCD45− hCD45+ hCD34+ | 1.26E+05 | 30 | 4 | 50 | 21 |
| sorted cells | hCD45RA− (MPP2) | | | | | |
| Library | mCD45− hCD45+ hCD34+ | 3.40E+05 | 30 | 5 | 50 | 21 |
| sorted cells | hCD45RA− hCD38+ hCD90− | | | | | |
| | (MPP3) | | | | | |
| Library | mCD45− hCD45+ hCD34+ | 8.00E+03 | 30 | 0.4 | 50 | 1.7 |
| sorted cells | hCD45RA− hCD38− hCD90+ | | | | | |
| | (LT-HSC) | | | | | |
| Control | mCD45− hCD45+ hCD34+ | 1.50E+06 | 30 | 40 | 50 | 21 |
| sorted cells | hCD45RA+ (MPP1) | | | | | |
| Control | mCD45− hCD45+ hCD34+ | 1.16E+05 | 30 | 1.7 | 50 | 30 |
| sorted cells | hCD45RA− (MPP2) | | | | | |
| Library | hCD34+ purified from whole | 3.00E+05 | 30 | 6.7 | 50 | 32 |
| | bone marrow (BM_hCD34+) | | | | | |
| Control | hCD34+ purified from whole | 3.00E+05 | 30 | 7.8 | 50 | 20 |
| | bone marrow (BM_hCD34+) | | | | | |
| Library | Whole bone marrow | 1.00E+06 | 30 | 50 | 50 | 27 |
| Control | Whole bone marrow | 1.00E+06 | 30 | 67 | 50 | 28 |
| Library | Pooled heart tissue of 12 mice | representative | 30 | 135 | 200 | 43 |
| | | pooled tissue | | | | |
| Control | Pooled heart tissue of 3 mice | representative | 30 | 35 | 200 | 31 |
| | | pooled tissue | | | | |
| Library | Pooled muscle tissue of 12 mice | representative | 30 | 16 | 200 | 20 |
| | | pooled tissue | | | | |
| Control | Pooled muscle tissue of 3 mice | representative | 30 | 4 | 200 | 12 |
| | | pooled tissue | | | | |
| Library | Pooled lungs tissue of 12 mice | representative | 30 | 442 | 200 | 44 |
| | | pooled tissue | | | | |
| Control | Pooled lungs tissue of 3 mice | representative | 30 | 470 | 200 | 18 |
| | | pooled tissue | | | | |
| Library | Pooled whole blood of 12 mice | representative | 30 | 47 | 200 | 82 |
| | | pooled tissue | | | | |
| Control | Pooled whole blood of 3 mice | representative | 30 | 358 | 200 | 17 |
| | | pooled tissue | | | | |
| Library | Pooled kidney tissue of 12 mice | representative | 30 | 431 | 200 | 70 |
| | | pooled tissue | | | | |
| Control | Pooled kidney tissue of 3 mice | representative | 30 | 157 | 200 | 49 |
| | | pooled tissue | | | | |
| Library | Pooled spleen tissue of 12 mice | representative | 30 | 1122 | 200 | 132 |
| | | pooled tissue | | | | |
| Control | Pooled spleen tissue of 3 mice | representative | 30 | 1251 | 200 | 557 |
| | | pooled tissue | | | | |
| Library | Pooled brain tissue of 12 mice | representative | 30 | 96 | 200 | 43 |
| | | pooled tissue | | | | |
| Control | Pooled brain tissue of 3 mice | representative | 30 | 443 | 200 | 21 |
| | | pooled tissue | | | | |
| Library | Pooled liver tissue of 12 mice | representative | 30 | 753 | 200 | 233 |
| | | pooled tissue | | | | |
| Control | Pooled liver tissue of 3 mice | representative | 30 | 1290 | 200 | 100 |
| | | pooled tissue | | | | |

### Example 7.2: Bio-Distribution Study on Lib8 Sso7d-VP2 AAV1Sil1 Injected Mice ddPCR Multiplex from Extracted DNA Samples

The QX200 droplet digital PCR (ddPCR) system (Bio-Rad) was used to quantify the number of AAV genome copies per cell on extracted DNA samples, normalized to the RPP30 gene. Due to the presence of both human and mouse derived genomic DNA, two different experiments were performed using either a mouse or a human specific RPP30 oligo set. For AAV genome detection the SV40 pA specific oligo set described in Table 20 was used. Commercially available hRPP30 and mRPP30 HEX assays were used for normalization (Bio-Rad #10031243 and #10042962 respectively).

PCR was performed in a 20 μl volume containing 25 ng of genomic DNA, 900 nM of the forward and reverse SV40 pA primers, 125 nM of the SV40 pA probe, 1 μl of 20× mouse or human RPP30 HEX assay, 10 μl of 2×ddPCR supermix for probes (Bio-Rad) and 0.5 units of SphI-HF (NEB #R3182). Technical duplicates were performed for each sample. After 15 min at RT incubation, each ddPCR assay mixture was loaded into a disposable droplet generator cartridge (Bio-Rad). Then, 70 μL of droplet generation oil for probes (Bio-Rad) was loaded into each of the eight oil wells. The cartridge was then placed inside the QX200 droplet generator (Bio-Rad). When droplet generation was completed, the droplets were transferred to a 96-well PCR plate using a multichannel pipet.

The plate was heat-sealed with foil and placed in a C1000 Touch Thermal Cycler (Bio-Rad). Thermal cycling conditions were as follows: 95° C. for 10 min, then 40 cycles of 95° C. for 30 s and 60° C. for 1 min followed by a 10 min incubation at 98° C., and 4° C. indefinite hold. FAM fluorescent signal, labeling the AAV genome DNA sequence, and HEX fluorescent signal, labeling the RPP30 gene sequence in each droplet were counted using a QX200 digital droplet reader, and analyzed by QuantaSoft analysis software (Bio-Rad).

### Data Transformation:

Technical replicate averages (two for human and mouse RPP30, four for SV40 pA) and standard deviations were calculated for each reaction. Total number of diploid cells in each reaction was calculated by summing mouse and human RPP30 averaged droplets and dividing by 2. Representation of human cells in the different tissues was calculated by dividing the average number of human cells (hRPP30) by the average of the total number of cells (hRPP30+mRPP30). High variability was observed in the different samples indicating that initial DNA quantification by UV spectrophotometer was not accurate.

The number of AAV genomes per cell was calculated by dividing the average number of droplets of SV40 pA by the total number of cells (hRPP30+mRPP30) in the reaction.

In order to take in account error propagation, standard deviations for each transformed value were calculated using the on-line GraphPad tool (www.graphpad.com/quickcalcs/ErrorProp1.cfm).

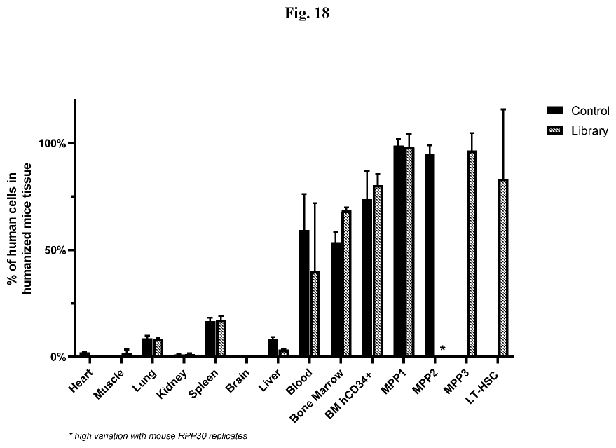

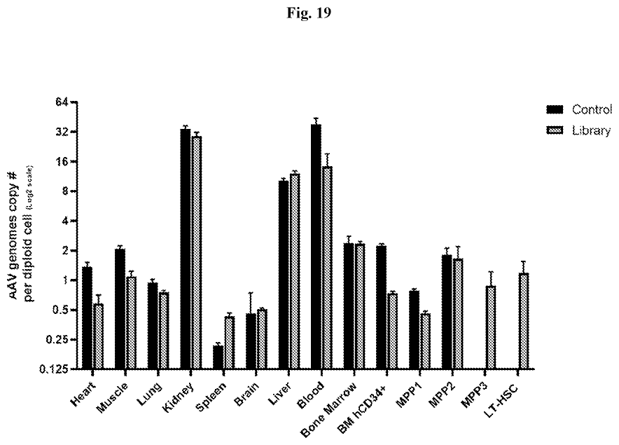

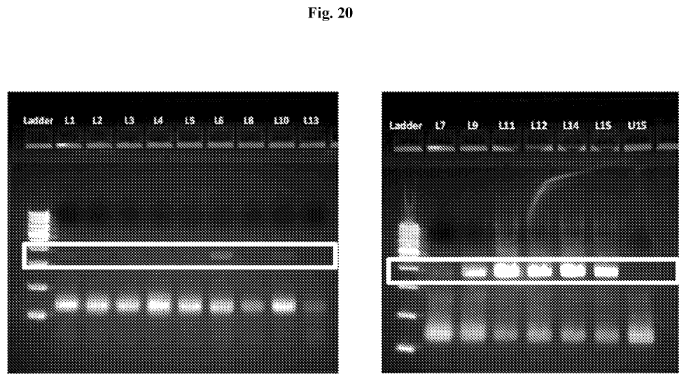

The results are shown in FIGS. 18 and 19.

ddPCR confirmed good humanization levels of the mice that were used for the Sso7d library selection (amounting to 50-60% based on the data from blood and bone marrow).

The human-derived cell there were observed in the spleen (>10%) could be a result of migration into this organ after mobilization.

The biodistribution analysis confirmed the presence of AAV genomes in all tested organs. AAV genome levels were low in spleen and brain (<1 copy/cell) and higher in kidney, liver and blood (10-30 copies per cell). Liver appears not to be the main sink of AAV infection as expected for sialic acid KO AAV1 (Sil1).

### Example 7.3: NGS Analysis on cDNA Prepared from Lib8 Sso7d-VP2 AAV1Sil1 Injected Mice

cDNA Preparation from Extracted RNA Samples:

cDNA was prepared using OneTaq® One-Step RT-PCR Kit (NEB #E5315), 10 pmol of LC0052/LC0053 primers described in example 6.1 and 10 μl of total RNA or 1 μg for the samples with an RNA concentration >100 μg/μl as template (see Table 24), in 50 μl total reaction volume. RT-PCR was carried out with the following cycling protocol: 30 min at 48° C., 2 min at 94° C.; 40 cycles of 94° C. for 15 s, 55° C. for 30 s, 68° C. for 30 s; followed by final amplification at 68° C. for 2 min and then holding at 4° C. The size of the products was assessed on 1 μl of RT-PCR reaction using Flash-Gel system (Flash-Gel DNA cassette, 2.2% Lonza #57031). The results are shown in FIG. 20. Reactions were purified using Qiaquick PCR purification Kit (Qiagen #28104) and eluted in 50 μl of water.

TABLE 24
Sample description and RNA amount used for RT-PCR reactions

| Sample | | | |
|---|---|---|---|
| ID | Description | Amount used | |
| L1 | mCD45− hCD45+ hCD34+ hCD45RA+ (MPP1) | 370 | ng |
| L2 | mCD45− hCD45+ hCD34+ hCD45RA− (MPP2) | 40 | ng |
| L3 | mCD45− hCD45+ hCD34+ hCD45RA− hCD38+ | 50 | ng |
| | hCD90− (MPP3) | | |
| L4 | mCD45− hCD45+ hCD34+ hCD45RA− hCD38− | 4 | ng |
| | hCD90+ (LT-HSC) | | |
| L5 | BM hCD34+ | 67 | ng |
| L6 | Bone marrow | 500 | ng |
| L7 | Heart | 1 | μg |
| L8 | Muscle | 160 | ng |
| L9 | Lung | 1 | μg |
| L10 | Blood | 1 | μg |
| L11 | Kidney | 1 | μg |
| L12 | Spleen | 1 | μg |
| L13 | Brain | 1 | μg |
| L14 | Liver | 1 | μg |

### Library Preparation and NGS on Illumina's MiSeq:

Amplicon libraries were generated from the cDNAs prepared from extracted RNA as described above in one single PCR reaction using a set of primers containing Illumina's adapter. The forward primer was used in combination with a reverse primer containing the Illumina TruSeq index to allow for identification of individual samples. The list of primers used in this example is shown in Table 25.

TABLE 25
List of primers used in this example
(bold: index, underlined: annealing region)

| Name | Primer seq 5′-3′ |
|---|---|
| LC0034 | AATGATACGGCGACCACCGAGATCTACACTCTTTCCCTACA |
| (for primer) | CGACGCTCTTCCGATCTCAGGTGGACATCAGCAAGATCAAG |
| LC0076 | CAAGCAGAAGACGGCATACGAGATGTAGCCGTGACTGGAGTTCA |
| (rev primer/L14) | GACGTGTGCTCTTCCGATCTCTTTAGGGGCGTCCTTTTCGGACAC |
| LC0077 | CAAGCAGAAGACGGCATACGAGATTACAAGGTGACTGGAGTTCA |
| (rev primer/L12) | GACGTGTGCTCTTCCGATCTCTTTAGGGGCGTCCTTTTCGGACAC |
| LC0078 | CAAGCAGAAGACGGCATACGAGATTTGACTGTGACTGGAGTTCA |
| (rev primer/L11) | GACGTGTGCTCTTCCGATCTCTTTAGGGGCGTCCTTTTCGGACAC |
| LC0079 | CAAGCAGAAGACGGCATACGAGATGGAACTGTGACTGGAGTTCA |
| (rev primer/L10) | GACGTGTGCTCTTCCGATCTCTTTAGGGGCGTCCTTTTCGGACAC |
| LC0080 | CAAGCAGAAGACGGCATACGAGATTGACATGTGACTGGAGTTCA |
| (rev primer/L9) | GACGTGTGCTCTTCCGATCTCTTTAGGGGCGTCCTTTTCGGACAC |
| LC0081 | CAAGCAGAAGACGGCATACGAGATGGACGGGTGACTGGAGTTCA |
| (rev primer/L8) | GACGTGTGCTCTTCCGATCTCTTTAGGGGCGTCCTTTTCGGACAC |
| LC0082 | CAAGCAGAAGACGGCATACGAGATGCGGACGTGACTGGAGTTCA |
| (rev primer/L7) | GACGTGTGCTCTTCCGATCTCTTTAGGGGCGTCCTTTTCGGACAC |
| LC0083 | CAAGCAGAAGACGGCATACGAGATTTTCACGTGACTGGAGTTCA |
| (rev primer/L6) | GACGTGTGCTCTTCCGATCTCTTTAGGGGCGTCCTTTTCGGACAC |
| LC0084 | CAAGCAGAAGACGGCATACGAGATGGCCACGTGACTGGAGTTCA |
| (rev primer/L5) | GACGTGTGCTCTTCCGATCTCTTTAGGGGCGTCCTTTTCGGACAC |
| LC0085 | CAAGCAGAAGACGGCATACGAGATCGAAACGTGACTGGAGTTCA |
| (rev primer/L3) | GACGTGTGCTCTTCCGATCTCTTTAGGGGCGTCCTTTTCGGACAC |
| LC0086 | CAAGCAGAAGACGGCATACGAGATCGTACGGTGACTGGAGTTCA |
| (rev primer/L2) | GACGTGTGCTCTTCCGATCTCTTTAGGGGCGTCCTTTTCGGACAC |
| LC0087 | CAAGCAGAAGACGGCATACGAGATCCACTCGTGACTGGAGTTCA |
| (rev primer/L1) | GACGTGTGCTCTTCCGATCTCTTTAGGGGCGTCCTTTTCGGACAC |
| LC0088 | CAAGCAGAAGACGGCATACGAGATATCAGTGTGACTGGAGTTCA |
| (rev primer/L13) | GACGTGTGCTCTTCCGATCTCTTTAGGGGCGTCCTTTTCGGACAC |
| LC0089 | CAAGCAGAAGACGGCATACGAGATAGGAATGTGACTGGAGTTCA |
| (rev primer/L4) | GACGTGTGCTCTTCCGATCTCTTTAGGGGCGTCCTTTTCGGACAC |

PCR reactions were prepared using the Q5 Hot Start High-Fidelity Master Mix (NEB #M0494), 10 pmol of each primer and 10 μl of purified cDNA samples as template in 25 μl total reaction volume. PCR was carried out with following cycling protocol: 2 min at 95° C.; 20 cycles of 98° C. for 10 s; 55° C. for 15 s; 72° C. for 25 s; followed by 2 min incubation at 72° C. and then holding at 4° C. Additional 10 cycles were added for L8 and L13 samples due to the low yield obtained with 20 cycles. PCR products were separated by agarose gel electrophoresis on a SybrSafe pre-stained 1.5%0 agarose gel (30 min at 100 V run). Bands at the expected size (243 bp) were then cut and purified using the Wizard SV Gel and PCR Clean-Up System (Promega #9280) and eluted in 30 μl of water. After quantitation using a Qubit instrument (all samples between 7-11 ng/μl except for 1.6 ng/μl for the L13 sample), libraries were equimolarly pooled at a final concentration of 8 μM and sequenced on a MiSeq System (Illumina) using the Illumina MiSeq Reagent Kit v3 (150 cycles, #MS-102-3001). The resulting read numbers are summarized in Table 26.

### Data Analysis Using Custom Script:

Generated fastq files were initially filtered by quality (Q>30, where Q indicates Phred quality score) and length (110-130 bp). After quality trim, the sequences were processed aiming to map Sso7d specific boundaries (ISKI to DAPK). The numbers of final filtered reads are reported in Table 26.

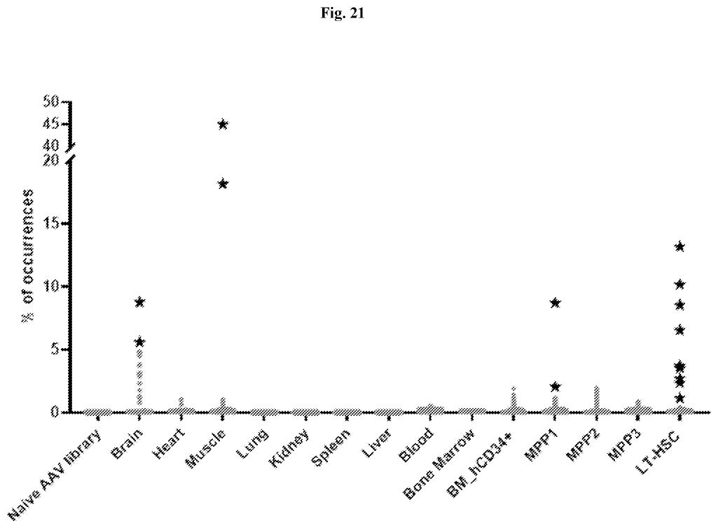

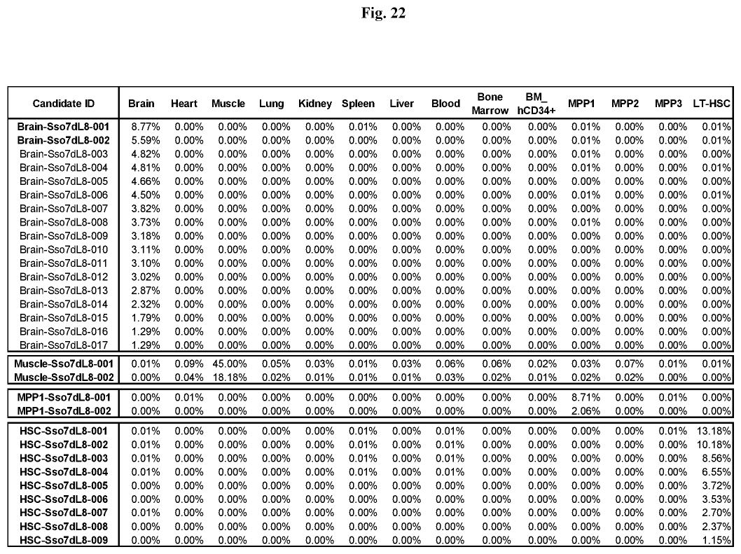

The distribution of single sequences in the different organs was assessed on final filtered sequences. The results are summarized in FIG. 21 (top 10′000 hits) and FIG. 22. Sequences selected for hit confirmation in Example 7 are shown in red.

TABLE 26
Initial and filtered NGS reads count

| Name | Initial reads | Filtered reads |
|---|---|---|
| L1-MPP1 | 875435 | 688058 |
| L2-MPP2 | 797296 | 633132 |
| L3-MPP3 | 790629 | 607226 |
| L4-LT_HSC | 897598 | 683457 |
| L5- BM_hCD34+ | 702645 | 562366 |
| L6-Bone marrow | 676977 | 522455 |
| L7-HEART | 720362 | 568901 |
| L8-MUSCLE | 1548187 | 1271261 |
| L9-LUNG | 1421675 | 1110392 |
| L10-BLOOD | 1167265 | 895851 |
| L11-KIDNEY | 1122478 | 868355 |
| L12-SPLEEN | 994747 | 633132 |
| L13-BRAIN | 1059484 | 862151 |
| L14-LIVER | 778890 | 606519 |

After the first round of in vivo selection, clear sequence enrichment could be observed in brain, muscle, LT-HSC and MPP1, but not in the other tissues. The sequences enriched in brain, muscle, LT-HSC and MPP1 appear to be specific and not enriched in the other tested samples.

Enrichment of specific sequences in the brain was probably promoted by the low permeability for AAV in this organ. In HSC the low complexity was expected due to the low amount of cells sorted (8K) and the low number of genomes per cell (˜1 genome/cell). In muscle, we observed difficulties to amplify cDNA despite the medium genome number of about 2 genome/cell, indicating possible non-optimal nuclear trafficking of AAV in these cells.

### Example 8. In-Vivo Confirmation of Selected Hits from 1st Selection Round Using Barcoded Library Approach

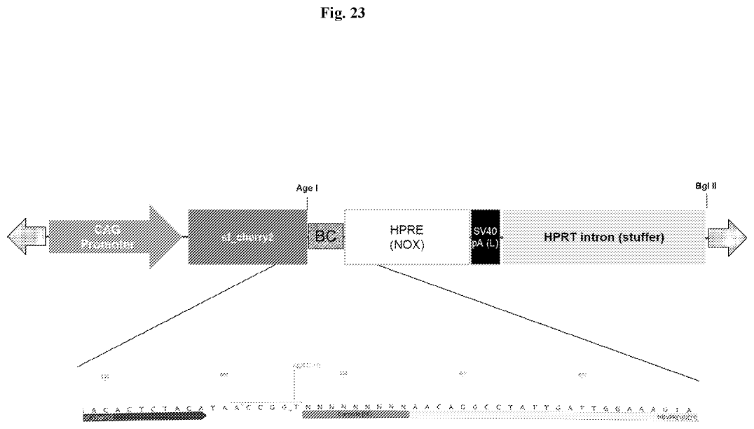

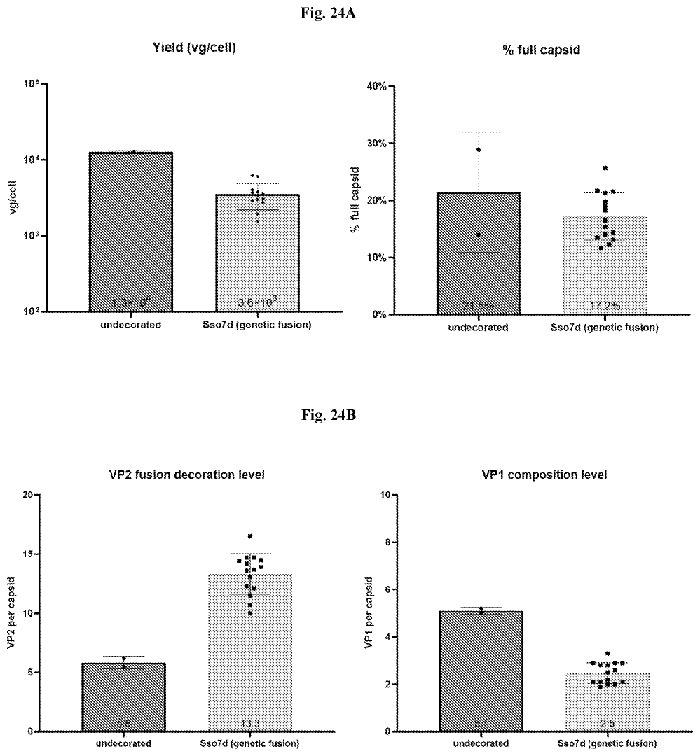

Confirmation of enriched Sso7d-AAV1Sil1 variants from the 1st selection round in humanized mice described in Example 7 was done by direct side-by-side comparison of modified AAV capsids containing cargo barcoded sequences in high-throughput and in the same animal, by combining RNA barcoding with multiplexed next-generation sequencing. In brief, distinct 9-mer barcodes were cloned into the 3′ untranslated region of a red fluorescent protein (sfCherry2) reporter driven by the ubiquitously active CAG promoter and followed by a post-transcriptional regulatory element HPRE (NOX). The single-stranded AAV genome was filled up with a stuffer sequence (HPRT intron) to have a size of 4476 bases closed to maximal AAV capacity. The barcoded cargo plasmid used in this example is schematically represented in FIG. 23. The top enriched Sso7d variants from LT-HSC (n=9), MPP1 (n=2), brain (n=2) and muscle (n=2) were cloned into separate plasmids at the N-terminus of AAV1Sil1 VP2 and used for AAV assembly in combination with the selected unique barcoded cargo plasmids listed in Table 27. After AAV assembly and purification, the different AAV preparations were equimolarly mixed thereby generating a barcoded AAV1Sil1 library. The barcoded library was then injected in humanized mice and Biodistribution/NGS analysis was conducted on extracted DNA/RNA from different tissues. In-vivo confirmation of the selected hits using this barcoded library approach is described in more detail below.

### Example 8.1 Barcoded Plasmid Design and AAV Production

### Barcoded Cargo Plasmids Generation:

AgC1295 plasmid comprising sfCherry2 under the control of a CAG promoter and an HPRE element (NOX) (pCargo_CBa_sfcherry2_HPRE_SV40 pA), was used as backbone for barcoded cargo generation exploiting the AgeI restriction site present right after the sfCherry2 CDS and the BglII restriction site present before the R-ITR. A 2634 bp fragment containing HPRE (NOX), SV40 pA(L) and HPRT1 stuffer was amplified from an internal plasmid called (AgC1294) pCargo_ss_173CMVp_EGFP_HPRE_SV40 pA_stuffer using JKU_1_fw (5′-GAG CCG AGG CCA GAC ACT CTA CAT AAC CGG TNN NNN NNN NAA CAG GCC TAT TGA TTG GAA AGT ATG-3′) and JKU_2_rev (5′-GAT TAA CCT GAT AGA TCT CTC GAC TTG GGC AAC AAA AGT GAA ACT CCA TC-3′) oligonucleotides. The forward oligonucleotide contains a 25 base stretch homologous to the AgeI digested AgC1295, an AgeI restriction site, a stretch of 9 randomized nucleotides (N, the barcode) followed by 26 bases acting as annealing region to the template. The reverse oligo contains a 25 base stretch homologous to the BglII digested AgC1295, a BglII restriction site followed by 32 bases acting as annealing region to the template. The PCR reaction was prepared using Q5 Hot Start High-Fidelity Master Mix (NEB #M0494), 10 pmol of each primer and 10 ng of the AgC1294 plasmid as template in 50 μl total reaction volume with the following cycling protocol: 30 s at 98° C.; 25 cycles of 98° C. for 10 s; 66° C. for 15 s; 72° C. for 1 min 30 s followed by a 2 min incubation at 72° C. To generate barcoded sfCherry cargo plasmids, 200 ng of the PCR product (2634 bp) and 100 ng of the AgeI-BglII digested AgC1295 acceptor plasmid were assembled using NEBuilder HiFi DNA Assembly Master Mix (NEB; E2621) in a final volume of 20 μl and incubated for 2 h at 50° C. One vial of One Shot TOP10 Chemically Competent E. coli cells (Thermo-Fisher #C404010) was transformed with 2 μl of assembly reaction and spreaded on LB+kanamycin plates. 96 colonies were then used to separately extract plasmid DNA using the NucleoSpin 96 Plasmid kit (Macherey-Nagel #740625.4) and analyzed by Sanger sequencing.

The barcode regions of all sequences were aligned using Geneious Prime software and barcode regions of interest were filtered using the following parameters yielding the 41 barcodes listed in Table 27:
Barcodes comprising homopolymers longer than 3 nt were excluded.
Barcodes with distinctions from all other barcodes in at least three positions were kept

The 41 selected barcoded cargo plasmids were upscaled and purified using the NucleoBond Xtra Midi plus kit (Macherey-Nagel #740410). Barcode identity of each plasmid preparation was confirmed by Sanger sequencing and the integrity of the AAV ITRs was confirmed by restriction digest with Srf I (NEB #R0629).

TABLE 27
List of selected barcodes

| ID | Barcode | Assigned to |
|---|---|---|
| A02 | ACAGGTCCGA | Undecorated CTR |
| A04 | CTTGCATGT | |
| A06 | GCGTGCGAG | HSC-Sso7dL8-001 |
| A07 | AGATCAACT | |
| A08 | GGCGCAGTT | HSC-Sso7dL8-002 |
| A09 | GGAGTGCGT | |
| B02 | GCTGAGATA | HSC-Sso7dL8-003 |
| B05 | TTAAGTTGG | |
| B06 | GCCTCTACG | HSC-Sso7dL8-004 |
| B07 | TGGAGAGGC | |
| B10 | TCAGTGGAT | HSC-Sso7dL8-005 |
| B12 | TGAACTAAG | |
| C01 | GGTCTTCGG | HSC-Sso7dL8-006 |
| C03 | ATTGCTTGG | |
| C05 | CTGCGCTGG | |
| C08 | ACATGCCTG | HSC-Sso7dL8-007 |
| C09 | AGGAGAACG | |
| D02 | TCGCCCACA | |
| D04 | ACCATGTGG | |
| D10 | CGGTGGTGT | |
| E07 | GGATTATAC | |
| E08 | TATAGATCT | |
| E09 | CTGCGGCTA | |
| E10 | TACTCGAGT | |
| E11 | GCTCGAAGG | |
| F04 | AATATTATA | |
| F06 | TAGAATTGA | |
| F10 | GGATGTGAG | |
| F11 | GATCTGCGT | |
| G03 | TTAGGCTTA | HSC-Sso7dL8-008 |
| G04 | TTCCTGAGG | MPP1-Sso7dL8-001 |
| G06 | CAGAATAAG | HSC-Sso7dL8-009 |
| G07 | TATGAGCAG | Muscle-Sso7dL8-001 |
| G08 | TAATAACGA | MPP1-Sso7dL8-002 |
| G10 | ACGTTAAGT | |
| G11 | CGTTGTATG | |
| G12 | AGGATGAAT | Muscle-Sso 7dL8-002 |
| H05 | TATTGCCAA | Brain-Sso7dL8-001 |
| H07 | AGTGCGCCG | Brain-Sso7dL8-002 |
| H09 | GTGACAATT | |
| H11 | TTCGCGCGA | |

Cloning of Selected Sso7d Sequences into AAV1Sil1 VP2 Expression Plasmids:

Mutations identified in positions 22, 24, 26, 29, 31, 33, 41, 43 and 45 (relative to Sso7d sequence including the first methionine) of the 15 selected hits from the first in-vivo selection round were in silico grafted into the rcSso7 scaffold described in Example 6.1 (containing a G4S linker at its C-terminus). The mutated sequences were ordered at GeneArt (Thermo-Fisher) as string DNA adding 25 bp homology to each end suitable for Gibson based cloning into the HindIII-AgeI digested AAV1 Sil1 VP2 expression plasmid (NC058).

To generate Sso7d-AAV1Sil1 VP2 expression plasmids, 10 ng of equimolarly mixed string DNA and 100 ng of AgeI-HindIII digested NC058 acceptor plasmid were assembled using the NEBuilder HiFi DNA Assembly Master Mix (NEB; E2621) in a final volume of 20 μl and incubated for 2 h at 50° C. One vial of One Shot TOP10 chemically competent E. coli cells (Thermo-Fisher #C404010) was transformed with 2 μl of assembly reaction and spreaded on LB+kanamycin plates. 96 colonies were then used separately to extract plasmid DNA using the NucleoSpin 96 Plasmid kit (Macherey-Nagel #740625.4) and plasmid identity was confirmed by Sanger sequencing.

Selected expression plasmids were upscaled and purified using the NucleoBond Xtra Midi plus kit (Macherey-Nagel #740410) and the identity of each plasmid preparation was confirmed by Sanger sequencing.

### Barcoded Sso7d-AAV1Sil1 Library Generation:

Separate DNA mixes for each of the selected hits from the first selection round containing an equimolar ratio of pHelper, (NC057) pRep2_Cap1_AAV1 Sil1 VP2 KO, the respective Sso7d AAV1Sil1 VP2 expression plasmid and the corresponding barcoded cargo (see Table 27) were prepared and transfected into 200 ml of HEK293 suspension cells at a concentration of 2E+06 vc/ml (1.1 μg total DNA/1E+06 cells) using the FectoVIR-AAV Transfection reagent (Polyplus #101000022); DNA:FectoVIR=1 μg:1 μl. Glucose at a final concentration of 2 g/l was added to the transfection mix after transfection and the mixture was incubated at 37° C., 110 rpm, 6% CO2. Two additional transfections with undecorated AAV1Sil1 in combination with its corresponding barcoded cargo plasmid (A02) were also performed.

Three days post transfection, benzonase to final concentration of 0.1 U/μl and 20 ml of lysis buffer (500 mM HEPES, 10% Tween 20, 20 mM MgCl₂) were added to the transfection reactions and the mixtures were incubated at 37° C. under agitation for 3 h. After this incubation step, 24 ml of sucrose salt solution (5 M NaCl, 7% sucrose) were added to the lysate and the samples were incubated for additional 20 min. The samples were then centrifuged for 15 min at 3500 g, the supernatants were filtered using a 0.22 μM filter, supplemented with EDTA at a final concentration of 5 mM and loaded at 1 ml/min on a AAVX Pre-packed Column, 0.5×5 cm, 1 ml (ThermoFisher #A36652) mounted on an AKTA Pure instrument. Bound AAV particles were washed with 10 CV of wash buffer, pH 7.3 (50 mM Tris-Cl, 0.5 M NaCl, 0.01% Pluronic F-68) and eluted with 5 CV of glycine elution buffer, pH 2.7 (0.1 M Glycine, 0. 2 M NaCl, 0.25 M L-arginine). Elution fractions (0.25 ml) were immediately neutralized with 25 μl of 1 M Tris-Cl, pH 10. Fractions containing AAV (as assessed by UV280 signal) were pooled and dialyzed, using the Float-A-Lyzer Dialysis Device 100 KD (Spectrum™ #G235071), O/N against PBS, pH 7.4+0.001% Pluronic F-68 and then passed through a 0.22 μm filter unit.

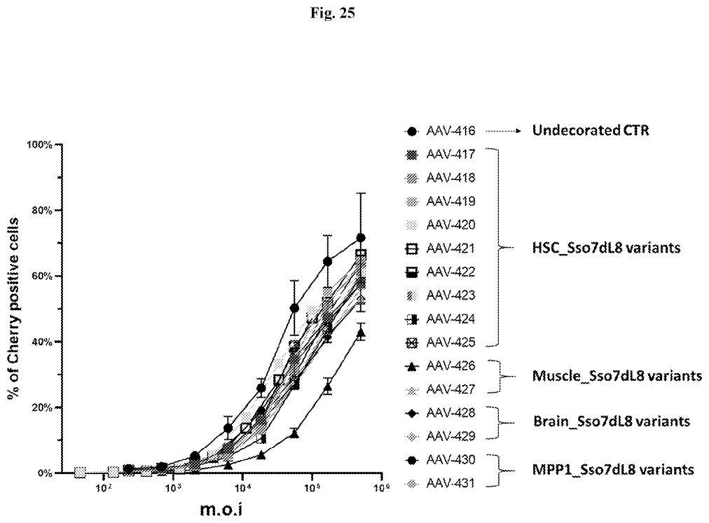

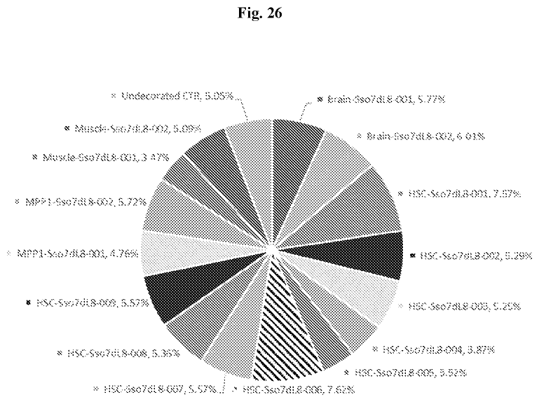

CE-SDS (Bioanalyzer) and ddPCR assays as described in Example 6.2 were used to assess decoration levels, as well as total and full particle concentration. The results are shown in FIG. 24A and FIG. 24B.

### In Vitro Infectivity of Sso7d Decorated AAV1Sil1 on HEK293 Suspension Cells:

Infectivity assessment was performed in order to ensure that the different barcodes have no impact on mRNA expression. At the day of infection, HEK293 suspension cells were resuspended in DMEM medium and seeded in a 96 well plate (1.5E+04 cells/well). AAV1Sil1 decorated preparations were diluted in DMEM medium and different concentrations were applied to the seeded cells. 3 h post transduction FBS at a final concentration of 5% was added to each well. AAV concentrations ranged from 5.0E+05 to 4.6E+01 moi based on viral genome. Three days post transduction, cells were detached and FACS analysis was performed to calculate the percentage of sfCherry2 positive cells. FIG. 25 shows the number of full particles (vg) versus the percentage of infected cells (sfCherry2+).

### Final Pooled Barcoded Library (AAV-471) Generation:

4E+11 vg of each single barcoded Sso7d-AAV1 Sil1 preparation (except for Sso7dL8-004 and -005, for which 1.5E+11 vg were used) and undecorated control were mixed, concentrated using an Amicon Ultra-15 MWCO 100,000 filter unit (Merck #UFC910008) and then passed through a 0.22 μm filter unit (final volume=1.1 ml).

CE-SDS and ddPCR assays as described in Example 6.2 were used to assess decoration levels, as well as total and full particle concentrations. The results are shown in Table 28.

TABLE 28
Barcoded library AAV-471 QC

| | | | | | VP3 | VP2- | VP1 |
|---|---|---|---|---|---|---|---|
| Library | Volume | vg/ml | vp/ml | % Full | per | fusion per | per |
| ID | (ml) | (ddPCR) | (Bioanalyzer) | particle | AAV | AAV | AAV |
| AAV- | 1.1 | 3.89E+12 | 3.69E+13 | 11% | 43.9 | 13.1 | 3.0 |
| 471 | | | | | | | |

### Example 8.2: Mobilized Humanized Mice Injection and Tissue Processing

For in vivo transduction assessment of the barcoded Sso7d library, NSG mice were utilized which were engrafted by human cord blood derived CD34+ cells. The humanization of the mice was confirmed by observing at least 30% human CD45+ cells over total CD45+ cells (mouse and human) using flow cytometry in the peripheral blood of the NSG mice 12 weeks post engraftment. Prior to administration of the barcoded Sso7d AAV1Sil1 library viruses the HSC of the mice were mobilized using a 5-day dose regimen. Briefly, G-CSF was administered through subcutaneous injection twice a day for four consecutive days at a dose of 125 μg/kg. On the morning of day 5, AMD3100 was administered via subcutaneous injection once at a dose of 5 mg/kg. Within one hour post AMD3100 injection the library virus was administered through tail vein injection at a dose of 1.2E+13 vg/kg. A total of 10 humanized NSG mice were injected with the virus library.

Five days post viral injection, mice were sacrificed and the following tissues were collected: whole blood, brain, liver, spleen, kidney, skeletal muscle, heart, lungs, and bone marrow. For whole blood, blood of all mice was pooled (n=10). Pooled blood was spun at 500 g for 10 min at RT, plasma was removed and the pellet was stored in −80° C. in 100 μl aliquots. For all other tissues except for bone marrow, small sections (3 mm) of each tissue were cut off and pooled. Pooled tissues were cut into small pieces using sharp razors and stored in −80° C. in aliquots. One aliquot of pooled frozen tissue each (whole blood, brain, liver, spleen, kidney, skeletal muscle, heart, lungs) was homogenized in RLT buffer (Qiagen) using 5 mm stainless steel beads in a Qiagen TissueLyser II. The homogenized tissue was spun at 1000 g for 2 min and the supernatant was transferred to fresh tubes and stored at −80° C. until further processing. For bone marrow collection, clean bone ends were cut using sharp razors and were spun at 8000 g for 1 min at 4° C. Bone marrow from all mice was pooled and passed through a 100 μm strainer in FACS buffer (1×PBS, 0.5% BSA, 2 mM EDTA). An aliquot (1 million cells) of the pooled bone marrow cells was pelleted and stored at −80° C. as input and the rest of the cells (273 million) were used for purification of human CD34+ cells using magnetic beads conjugated anti-human CD34 antibody (Miltenyi, 130-097-047) with LS columns according to manufacturer's instructions. An aliquot of the purified CD34+ cells (0.05 million cells) was pelleted and stored at −80° C. as input and the rest of the cells (12 million) were stained with an antibody cocktail of the following antibodies per 4 million cells: 10 μl of anti-human CD34-FITC (BD Pharmagin, 555821), 1 μl of anti-mouse CD45-BV650 (BioLegend, 563410), 1 μl of anti-human CD45-PE-Cy7 (BD Biosciences, 557748), 2 μl of anti-human CD90-APC (R&D Systems, FAB2067A-025), 1 μl of anti-human CD38-PE (BD Biosciences, 567146), 1 μl of anti-human CD45RA-BV605 (BD Biosciences, 562886), 1 μl of Lin-cocktail-V450 (custom), and Viability-APC-Cy7 (eBioscience, 65-0865-14). Cells were sorted for the following four populations using an automated sorter (BD FACS ARIA II): 1) Lin-mCD45-hCD45+hCD34+hCD45RA+(MPP1) 2) Lin-mCD45-hCD45+hCD34+hCD45RA-hCD38-hCD90−(MPP2) 3) Lin-mCD45-hCD45+hCD34+hCD45RA-hCD38+hCD90−(MPP3) 4) Lin-mCD45-hCD45+hCD34+hCD45RA-hCD38-hCD90+(LT-HSC). Sorted populations were centrifuged at 350 g for 5 min at 4° C. and were stored in RLT buffer in −80° C. until further processing. DNA and RNA were purified from the homogenized tissue lysates and stored at −80° C. using the Allprep DNA/RNA Mini Kit (Qiagen, 80204). The concentration of the eluted DNA/RNA was determined using an UV spectrophotometer. The results are summarized in Table 29.

TABLE 29
Concentration of the DNA and RNA extracted from humanized mice tissues

| | | | RNA | RNA | DNA | DNA |
|---|---|---|---|---|---|---|
| Tissue | | Number of | elution | conc. | elution | conc. |
| source | Tissue description | Cells | (μL) | (ng/μL) | (μL) | (ng/μL) |
| Library | mCD45− hCD45+ hCD34+ | 5.50E+06 | 30 | 41 | 50 | 76 |
| sorted cells | hCD45RA+ (MPP1) | | | | | |
| Library | mCD45− hCD45+ hCD34+ | 7.76E+03 | 11 | 0.5 | 20 | Undet |
| sorted cells | hCD45RA− hCD38− hCD90− | | | | | |
| | (MPP2) | | | | | |
| Library | mCD45− hCD45+ hCD34+ | 2.72E+04 | 11 | 1.34 | 20 | Undet |
| sorted cells | hCD45RA− hCD38+ hCD90− | | | | | |
| | (MPP3) | | | | | |
| Library | mCD45− hCD45+ hCD34+ | 1.13E+03 | 11 | 3.89 | 20 | Undet |
| sorted cells | hCD45RA− hCD38− hCD90+ | | | | | |
| | (LT-HSC) | | | | | |
| Library | hCD34+ purified from whole | 5.00E+04 | 30 | 16 | 50 | 10.2 |
| | bone marrow (BM_hCD34+) | | | | | |
| Library | Pooled whole bone marrow | 1.00E+06 | 30 | 375 | 50 | 250 |
| Library | Pooled heart tissue of 12 mice | representative | 30 | 24 | 50 | 32 |
| | | pooled tissue | | | | |
| Library | Pooled muscle tissue of 12 mice | representative | 30 | 3 | 50 | 29.4 |
| | | pooled tissue | | | | |
| Library | Pooled lungs tissue of 12 mice | representative | 30 | 276 | 50 | 58 |
| | | pooled tissue | | | | |
| Library | Pooled whole blood of 12 mice | representative | 10 | 5 | 40 | 11 |
| | | pooled tissue | | | | |
| Library | Pooled kidney tissue of 12 mice | representative | 30 | 18.3 | 50 | 126 |
| | | pooled tissue | | | | |
| Library | Pooled spleen tissue of 12 mice | representative | 30 | 188 | 50 | 488 |
| | | pooled tissue | | | | |
| Library | Pooled brain tissue of 12 mice | representative | 30 | 70 | 50 | 57.7 |
| | | pooled tissue | | | | |
| Library | Pooled liver tissue of 12 mice | representative | 30 | 418 | 50 | 71 |
| | | pooled tissue | | | | |

Undet: Undetermined

### Example 8.3: NGS Analysis on Total DNA from Barcoded Sso7d-VP2 AAV1Sil1 Library Injected Mice

### Library Preparation and NGS on Illumina's MiSeq:

Amplicon libraries were generated from extracted total DNA as described above in one single PCR reaction using a set of primers containing Illumina's adapter. The forward primer was used in combination with a reverse primer containing the Illumina TruSeq index to allow for identification of individual samples. The list of primers used in this example is shown in Table 23.

TABLE 23
List of primers used in this example (bold: index, underlined: annealing region)

| Name | Primer seq 5′-3′ |
|---|---|
| JKU_35_F | ATGATACGGCGACCACCGAGATCTACACTCTTTCCCTACCGACG |
| (for primer) | CTCTTCCGATCTGGATATCAAGCTGGACATCACC |
| JKU_11_R1 | CAAGCAGAAGACGGCATACGAGATCGTGATGTGACTGGAGTTC |
| (rev primer/Muscle) | AGACGTGTGCTCTTCCGATCTAGACCCACAATTCGTTGACATAC |
| JKU_12_R2 | CAAGCAGAAGACGGCATACGAGATACATCGGTGACTGGAGTTC |
| (rev primer/Heart) | AGACGTGTGCTCTTCCGATCTAGACCCACAATTCGTTGACATAC |
| JKU_13_R3 | CAAGCAGAAGACGGCATACGAGATGCCTAAGTGACTGGAGTTC |
| (rev primer/Kidney) | AGACGTGTGCTCTTCCGATCTAGACCCACAATTCGTTGACATAC |
| JKU_14_R4 | CAAGCAGAAGACGGCATACGAGATTGGTCAGTGACTGGAGTTC |
| (rev primer/Brain) | AGACGTGTGCTCTTCCGATCTAGACCCACAATTCGTTGACATAC |
| JKU_15_R5 | CAAGCAGAAGACGGCATACGAGATCACTGTGTGACTGGAGTTC |
| (rev primer/Spleen) | AGACGTGTGCTCTTCCGATCTAGACCCACAATTCGTTGACATAC |
| JKU_16_R6 | CAAGCAGAAGACGGCATACGAGATATTGGCGTGACTGGAGTTC |
| (rev primer/Lung) | AGACGTGTGCTCTTCCGATCTAGACCCACAATTCGTTGACATAC |
| JKU_17_R7 | CAAGCAGAAGACGGCATACGAGATGATCTGGTGACTGGAGTTC |
| (rev primer/Liver) | AGACGTGTGCTCTTCCGATCTAGACCCACAATTCGTTGACATAC |
| JKU_18_R8 | CAAGCAGAAGACGGCATACGAGATTCAAGTGTGACTGGAGTTC |
| (rev primer/LT-HSC) | AGACGTGTGCTCTTCCGATCTAGACCCACAATTCGTTGACATAC |
| JKU_19_R9 | CAAGCAGAAGACGGCATACGAGATCTGATCGTGACTGGAGTTC |
| (rev primer/CD38+ | AGACGTGTGCTCTTCCGATCTAGACCCACAATTCGTTGACATAC |
| CD90−) | |
| JKU_20_R10 | CAAGCAGAAGACGGCATACGAGATAAGCTAGTGACTGGAGTTC |
| (rev primer/CD38- | AGACGTGTGCTCTTCCGATCTAGACCCACAATTCGTTGACATAC |
| CD90−) | |
| JKU_21_R11 | CAAGCAGAAGACGGCATACGAGATGTAGCCGTGACTGGAGTTC |
| (rev primer/CDRA+) | AGACGTGTGCTCTTCCGATCTAGACCCACAATTCGTTGACATAC |
| JKU_22_R12 | CAAGCAGAAGACGGCATACGAGATTACAAGGTGACTGGAGTTC |
| (rev primer/Bone | AGACGTGTGCTCTTCCGATCTAGACCCACAATTCGTTGACATAC |
| Marrow) | |
| JKU_23_R13 | CAAGCAGAAGACGGCATACGAGATTTGACTGTGACTGGAGTTC |
| (rev primer/CD34+) | AGACGTGTGCTCTTCCGATCTAGACCCACAATTCGTTGACATAC |
| JKU_24_R14 | CAAGCAGAAGACGGCATACGAGATGGAACTGTGACTGGAGTTC |
| (rev primer/Blood) | AGACGTGTGCTCTTCCGATCTAGACCCACAATTCGTTGACATAC |
| JKU_25_R15 | CAAGCAGAAGACGGCATACGAGATTGACATGTGACTGGAGTTC |
| (rev primer/AAV-471) | AGACGTGTGCTCTTCCGATCTAGACCCACAATTCGTTGACATAC |

PCR reactions were prepared using the Q5 Hot Start High-Fidelity Master Mix (NEB TM0494), 10 pmol of each primer and extracted total DNA samples as template (the quantity used is shown in Table 31; in the case of AAV-471, 5 μl of purified AAV preparation was used as template) in 25 μl total reaction volume. PCR was carried out with following cycling protocol: 2 min at 95° C.; 40 cycles of 98° C. for 10 s; 55° C. for 15 s; 72° C. for 25 s; followed by 2 min incubation at 72° C. and then holding at 4° C. Reactions were loaded on agarose gel to confirm the presence of amplified products. Except for the lung sample, the expected band of about 267 bp could be observed in all other samples with no sign of unspecific amplification. For the lung sample it was later discovered that the lack of amplification was due to the low performance of the reverse oligo used. The PCR products were purified using the Qiaquick PCR purification kit (#28104) and eluted in 50 μl of water. After quantitation using a Qubit instrument, libraries were equimolarly pooled at a final concentration of 8 μM and sequenced on a MiSeq System (Illumina) using the Illumina MiSeq Reagent Kit v3 (150 cycles, #MS-102-3001).

TABLE 31
List of samples and total DNA used for PCR amplification

| | DNA | pre- | μl for | ng for |
|---|---|---|---|---|
| Tissue - DNA samples | (ng/μl) | dilution | PCR | PCR |
| CD34+ CD45+ CDRA+ | 76 | no | 1 | 76 |
| CD34+ CD45+ CDRA− | 0.22 | no | 5 | 1.1 |
| CD38− CD90− | | | | |
| CD34+ CD45+ CDRA− | 1.07 | no | 5 | 5.4 |
| CD38+ CD90− | | | | |
| LT-HSC | 0.06 | no | 5 | 0.3 |
| Bone Marrow | 250 | 10x | 1 | 25 |
| hCD34+ | 10.2 | no | 5 | 51 |
| Blood | 11 | no | 5 | 55 |
| Muscle | 29.4 | no | 1 | 29.4 |
| Heart | 32 | no | 1 | 32 |
| Kidney | 126 | 10x | 2 | 25.2 |
| Brain | 57.7 | no | 1 | 57.7 |
| Spleen | 488 | 10x | 1 | 48.8 |
| Lung | 58 | no | 1 | 58 |
| Liver | 71 | no | 1 | 71 |

### Data Analysis Using Custom Script:

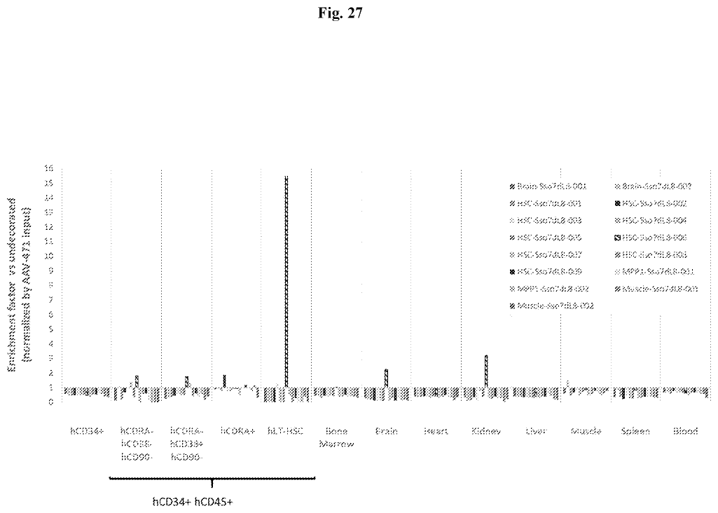

Generated fastq files were initially filtered by quality (Q>30, where Q indicates Phred quality score) and then processed aiming to map barcode specific boundaries on 100.000 hits for each sample. The percentages of unique barcodes in the different tissues were normalized first by dividing by the percentage found in the input AAV library AAV-417 (see FIG. 26) and then by the percentage of the barcode corresponding to the AAV1Sil1 undecorated control for each given tissue. The results are shown in FIG. 27.

NGS analysis confirmed a similar distribution of the 16 samples used to generate the barcoded Sso7d-VP2 AAV1Sil1 library (AAV-471).

HSC-Sso7dL8-006 showed a 15-fold enrichment in LT-HSC compared to the undecorated control. The same clone was found to be partially enriched in two other tissues (brain and kidney). Additional experiments will be needed to confirm these data. No specific enrichment was observed for the other tested variants.

### Example 9. In-Vivo Selection of Lib8 Sso7d-VP2 AAV1Sil1 (Second and Third Round)

### Introduction

After the first selection round, the majority of the identified hits could not be confirmed using the barcoded library approach, which is likely due to PCR bias during the RT-PCR step caused by either the low amount of initial RNA input from LT-HSC cells and/or by a suboptimal performance of the RT-PCR kit used. Two additional selection rounds were performed.

### Example 9.1: Enriched Library Generation from Bone Marrow CD34+ Cells Total RNA after First Selection Round

Due to the low RNA yield obtained from the LT-HSC population, bulk CD34+ derived RNA from the first selection round was used as template for the enriched library generation.

RT-PCR on Extracted RNA from Bone Marrow CD34+ Cells:

RT-PCR was performed using the LunaScript Multiplex One-Step RT-PCR Kit (NEB #E1555), 12.5 pmol of LC0052 (5′ CCCGGGGAACTCCTCCCGAGTCGAAATTCGCCACC 3′)/LC0053 (5′ GTTGCTGGAGGTT CTCCGAGAGGTTGTGGGTCGGG 3′) primers and ˜35 ng of total RNA from the BM_hCD34+ sample in 50 μl total reaction volume. RT-PCR was carried out with the following cycling protocol: 10 min at 55° C., 1 min at 98° C.; 40 cycles of 98° C. for 10 s, 55° C. for 20 s, 72° C. for 30 s; followed by final amplification at 72° C. for 5 min and then holding at 4° C. The reactions were run on an agarose gel and the expected band at 403 bp was excised. DNA was then extracted using the Wizard SV Gel and PCR Clean-UP System (Promega #A9281) and eluted in 50 μl of water with a final concentration of 1.63 ng/μl based on Qubit measurement. To increase the insert amount, it was amplified by PCR using the Q5 Hot Start High-Fidelity Master Mix (NEB #M0494), 10 pmol of each the LC052 and the LC053 primer and 5 ng of the gel extracted RT-PCR fragment as template in 50 μl total reaction volume with the following cycling protocol: 30 s at 98° C.; 15 cycles of 98° C. for 10 s; 55° C. for 20 s; 72° C. for 20 s followed by 72° C. for 2 min and then holding at 4° C. The PCR product was purified using the Qiaquick PCR purification Kit (Qiagen #28104) and eluted in 50 μl of water. The concentration was found to be 47 ng/μl based on Nanodrop spectrophotometer measurement.

### Plasmid Library Cloning:

To generate the first round enriched Sso7d-AAV1Sil1 plasmid library (NC109), 129 ng of PCR product (403 bp) and 1 μg of BsaI digested SK0036 acceptor plasmid were annealed using NEBuilder HiFi DNA Assembly Master Mix (NEB; E2621) in a final volume of 100 μl and incubated for 3 hours at 50° C. The reaction product was then treated for 1 h at 37° C. with Plasmid Safe (PS) DNase (Epicentre; E3105K) to digest any unassembled fragments and purified using the Wizard SV Gel and PCR Clean-up System (Promega #A9280). This reaction yielded ˜250 ng of assembled plasmid (as defined by the amount of DNA remaining after the PS DNase digestion step). One Shot TOP10 Electrocomp E. coli (ThermoFisher; C4040-52) were transformed with the assembled plasmid library in 5×50 μl vials (˜20 ng DNA/vial). Reactions were pooled and transferred into 11 Shake flask containing 200 ml of TB+kanamycin media. 100 μl were then removed from the flask, different dilutions were plated on LB+kanamycin plates and incubated over night at 37° C. Library practical size as determined by the number of CFU on agar plates was found to be 2.72E+08. Plasmid DNA was extracted from 200 ml culture after over night growth at 37° C. using the Maxiprep Kit (QIAGEN #12163) yielding the (NC109) pCargo_173CMV_SSo7dL8-CD34_AAV1Sil1-VP2_173CMV_sfCherry plasmid library.

### First Selection Round AAV Library Generation and QC:

2.8 liters of HEK293 suspension cells at a concentration of 2E+06 vc/ml were transfected with pHelper (˜40K plasmid/cell), (NC080) pAAV_Rep2Cap1_Sil1[V473D_N500E] VP2 KO [T138A]_KanR (˜40K plasmid/cell) and (NC109) pCargo_173CMV_SSo7dL8-CD34_AAV1Sil1-VP2_173CMV_sfCherry plasmid library (650 plasmids/cell) at 1.1 μg of total DNA/1E+06 cells using the FectoVIR-AAV Transfection reagent (Polyplus #101000022). The ratio of DNA:FectoVIR was 1 μg: 1 μl. Glucose to a final concentration of 2 g/l was added to the transfection mix after transfection and the mixture was incubated at 37° C., 110 rpm, 6% CO₂.

Two days post transfection, Benzonase was added to the transfection reactions to a final concentration of 0.1 U/μl and a volume equal to 10% of the total volume in lysis buffer (500 mM HEPES, 10% Tween 20, 20 mM MgCl₂). Following a 3 h incubation period at 37° C., a volume of sucrose salt solution (5 M NaCl, 7% sucrose) equal to 10% of the total volume was added to the lysate and the mixtures were incubated at 37° C. in agitation for 3 h. After this incubation step, a volume equal to 10% of the total volume in salt sucrose solution (5 M NaCl, 7% sucrose) was added and incubated for an additional 20 min. Samples were centrifuged for 15 min at 3500 g, and supernatants were filtered using a 0.22 μM filter, supplemented with EDTA at a final concentration of 5 mM. Next, the clarified lysates were loaded at 1 ml/min on an AAVX Pre-packed Column, 0.5×5 cm, 1 ml (ThermoFisher #A36652) mounted on an AKTA Pure instrument. Bound AAV particles were washed with 10 CV wash buffer, pH 7.3 (50 mM Tris-Cl, 0.5 M NaCl, 0.01% Pluronic F-68) and eluted with 5 CV glycine elution buffer, pH 2.7 (0.1 M Glycine, 0.2 M NaCl, 0.25 M L-arginine). Elution fractions (0.25 ml) were immediately neutralized with 25 μl 1 M Tris-Cl, pH 10. Fractions containing AAV (as assessed by UV280 signal) were pooled and dialyzed, using the Float-A-Lyzer Dialysis Device 100 KD (Spectrum™ #G235071), over night against PBS, pH 7.4+0.001% Pluronic F-68 and then passed through a 0.22 μm filter unit. Full and empty particles were separated on an iodixanol ultracentrifugation gradient and concentrated/buffer exchanged in PBS, pH 7.4+0.001% Pluronic F-68 using an Amicon Ultra-15 MWCO 100,000 Filter unit (Merck #UFC910008) and then passed through a 0.22 μm filter unit (final volume=2.2 ml).

### AAV Library Titer and Decoration Level Determination:

The QX200 droplet digital PCR (ddPCR) system (Bio-Rad) was used to determine the titer of AAV vectors using the SV40 pA specific oligo set described in Table 3. Purified preparations were pretreated as follows: 5 μl of AAV were incubated with 1 unit of DNase and 1× DNase buffer (Merck #04716728001) in 20 μl total volume for 30 min at 37° C. 1 μl of Proteinase K at 20 mg/ml (Life technologies #E00491) was added to the mixture and incubated for 1 h at 55° C. followed by an inactivation step at 95° C. for 15 min. Different 1/10 serial dilutions of treated preparations were prepared in 1×PCR Buffer II (Life technologies #N8080010), 2.5 mM MgCl2, 0.05% Pluronic F-68 (Sigma Aldrich #K4894), 2 ng/μl Sheared Salmon Sperm DNA (Invitrogen #15632011) and used as template for PCR amplification.

The ddPCR reaction mix was prepared by combining 5.5 μl pre-treated AAV dilution with 900 nM forward and reverse SV40 pA primers each, 125 nM SV40 pA probe and 11 μl 2× ddPCR supermix for probes (BioRad #1863024), in a final volume of 22 μl. Technical duplicates were performed for each sample. 20 μl of each ddPCR reaction mix was loaded into a disposable droplet generator cartridge (Bio-Rad). Then, 70 μL of droplet generation oil for probes (BioRad #1863005) was loaded into each of the eight oil wells. Next, the cartridge was placed inside the QX200 droplet generator (Bio-Rad). Following droplet generation, 40 μl were transferred to a 96-well PCR plate using a multichannel pipet.

The plate was heat-sealed with pierceable foil and placed into the C1000 Touch Thermal Cycler (Bio-Rad). Thermal cycling conditions were as follows: 95° C. for 10 min, then 40 cycles of 95° C. for 30 s and 60° C. for 1 min followed by a 10 min incubation at 98° C. and an indefinite hold at 4° C. FAM fluorescent signal, labeling the AAV genome DNA sequence in each droplet, was counted using a QX200 digital droplet reader, and analyzed by QuantaSoft analysis software (Bio-Rad). For calculation of AAV titers, the number of droplets were transformed by multiplying with the respective total dilution factor and additionally by a factor of 2 to take into account possible reannealing of single stranded genomes.

CE-SDS analysis (Agilent 2100 Bioanalyzer system, Protein 230 kit using denaturing conditions) was performed to evaluate the VP distribution in the purified samples and the total VP yield.

Calculation of total AAV particles/ml (vp/ml) and VP ratio:
ng/μl (=μg/ml) values are divided by the observed MW and then multiplied by 1E+06 (μg to g conversion) to calculate moles/ml values
moles/ml values are multiplied by 6.022E+23 (Avogadro nr) to calculate the number of molecules/ml
VP distribution: calculated based on molecules/ml values assuming one AAV is made of 60 VPs in total
AAV particles/ml (vp/ml): Sum of VP1+VP2+VP3 molecules ml are divided by 60

ddPCR and CE-SDS results are reported in Table 32.

TABLE 32
QC on 1^{st} round (BM_CD34+) enriched library prep

| | VP2 | | | |
|---|---|---|---|---|
| | fusion | | | % Full |
| Sample ID | per AAV | vg/ml | vp/ml | particle |
| (AAV-583) | 3.1 | 7.95E+12 | 3.65E+13 | 21.79% |
| ssAAV1Sil1_library_{—} | | | | |
| 173CMV_VP2-Sso7dL8- | | | | |
| CD34_173CMV_sfCherry | | | | |

### Example 9.2: Second Round of Mobilized Humanized Mice Injection and Tissue Processing

A second in-vivo selection was conducted on NSG mice (n=15) using (AAV-583) ssAAV1Sil1_library_173CMV_VP2-Sso7dL8-CD34_173CMV_sfCherry at a dose of 2.4E+13 vg/kg. The same mobilization, injection and tissue processing procedure as described in Example 7.1 was used.

Concentration of the eluted RNA was determined using a Qubit fluorometer. The results are summarized in Table 33.

TABLE 33
Concentration of RNA extracted from humanized mice tissues

| | | Qubit Conc |
|---|---|---|
| Tissue | Description | [ng/μl] |
| Bulk CD34+ | (AAV-583) Sso7d library sorted populations (pool of 15 | 23.5 |
| | mice) | |
| | mCD45− hCD45+ hCD34+ | |
| CD38+ Progenitor | (AAV-583) Sso7d library sorted populations (pool of 15 | 35.3 |
| | mice) | |
| | mCD45− hCD45+ hCD34+ hCD38+ | |
| CD38− CD45RA+ | (AAV-583) Sso7d library sorted populations (pool of 15 | 43.4 |
| CD90− Progenitor | mice) | |
| | mCD45− hCD45+ hCD34+ hCD38− hCD45RA+ hCD90− | |
| Multi-potent | (AAV-583) Sso7d library sorted populations (pool of 15 | <0.1 |
| progenitor | mice) | |
| | mCD45− hCD45+ hCD34+ hCD38− hCD45RA− hCD90− | |
| LT-HSC | (AAV-583) Sso7d library sorted populations (pool of 15 | <0.1 |
| | mice) | |
| | mCD45− hCD45+ hCD34+ hCD38− hCD45RA− hCD90+ | |
| Whole Bone Marrow | (AAV-583) Sso7d library sorted populations (pool of 15 | 12.3 |
| | mice) | |
| Blood | (AAV-583) Sso7d library sorted populations (pool of 15 | 77.0 |
| | mice) | |
| Muscle | (AAV-583) Sso7d library sorted populations (pool of 15 | <0.1 |
| | mice) | |
| Lung | (AAV-583) Sso7d library sorted populations (pool of 15 | 0.8 |
| | mice) | |
| Liver | (AAV-583) Sso7d library sorted populations (pool of 15 | <0.1 |
| | mice) | |
| Kidney | (AAV-583) Sso7d library sorted populations (pool of 15 | 1.14 |
| | mice) | |
| Heart | (AAV-583) Sso7d library sorted populations (pool of 15 | <0.1 |
| | mice) | |
| Brain | (AAV-583) Sso7d library sorted populations (pool of 15 | 7.84 |
| | mice) | |
| Spleen | (AAV-583) Sso7d library sorted populations (pool of 15 | 10.7 |
| | mice) | |

### Example 9.3: Enriched Library Generation from Bone Marrow LT-HSC Cell Total RNA after the Second Selection Round

RT-PCR on Extracted RNA from Bone Marrow LT-HSC Cells:

RT-PCR was performed using the LunaScript Multiplex One-Step RT-PCR Kit (NEB 4E1555), 12.5 pmol of LC0052 (5′ CCCGGGGAACTCCTCCCGAGTCGAAATTCGCCACC 3′)/LC0053 (5′ GTTGCTGGAGGTT CTCCGAGAGGTTGTGGGTCGGG 3′) primers and 5 μl (<0.5 ng) of total RNA from the BM_LT-HSC sample in 50 μl total reaction volume. RT-PCR was carried out with the following cycling protocol: 10 min at 55° C., 1 min at 98° C.; 35 cycles of 98° C. for 10 s, 55° C. for 20 s, 72° C. for 30 s; followed by final amplification at 72° C. for 5 min and then holding at 4° C. The reactions were run on an agarose gel and the expected band at 403 bp was excised. The DNA was then extracted using the Wizard SV Gel and PCR Clean-UP System (Promega #A9281) and eluted in 50 μl of water with a final concentration of 0.41 ng/μl based on Qubit measurement. To increase the insert amount, it was amplified by PCR using the Q5 Hot Start High-Fidelity Master Mix (NEB #M0494), 10 pmol of each the LC052 and the LC053 primer and 8 ng of the gel extracted RT-PCR fragment as template in 50 μl total reaction volume with the following cycling protocol: 30 s at 98° C.; 15 cycles of 98° C. for 10 s; 55° C. for 20 s; 72° C. for 20 s followed by 72° C. for 2 min and then holding at 4° C. The PCR product was purified using the Qiaquick PCR purification Kit (Qiagen #28104) and eluted in 50 μl of water. The concentration was found to be 46 ng/μl based on Nanodrop spectrophotometer measurement.

### Plasmid Library Cloning:

To generate the second round enriched Sso7d-AAV1Sil1 plasmid library (NC110), 129 ng of PCR product (403 bp) and 1 μg of BsaI digested SK0036 acceptor plasmid were annealed using the NEBuilder HiFi DNA Assembly Master Mix (NEB; E2621) in a final volume of 100 μl and incubated for 3 hours at 50° C. The reaction product was then treated for 1 h at 37° C. with Plasmid Safe (PS) DNase (Epicentre; E3105K) to digest any unassembled fragments and then purified using the Wizard SV Gel and PCR Clean-up System (Promega #A9280). The reaction yielded −200 ng of assembled plasmid (as defined by the amount of DNA remaining after the PS DNase digestion step). One Shot TOP10 Electrocomp E. coli (ThermoFisher; C4040-52) were transformed with the assembled plasmid library in 3×50 μl vials (˜14 ng DNA/vial). The reactions were pooled and transferred into a 11 Shake flask containing 200 ml of TB+kanamycin media. 100 μl were then removed from the flask, different dilutions were plated on LB+kanamycin plates and incubated over night at 37° C. Library practical size as determined by the number of CFU on agar plates was found to be 4.34E+07. Plasmid DNA was extracted from 200 ml culture after over night growth at 37° C. using the Maxiprep Kit (QIAGEN #12163) yielding the (NC110) pCargo_173CMV_SSo7dL8-LT-HSC-2_AAV1Sil1-VP2_173CMV_sfCherry plasmid library.

### Second Selection Round AAV Library Generation and QC:

2.8 liters of HEK293 suspension cells at a concentration of 2E+06 vc/ml were transfected with pHelper (˜40K plasmid/cell), (NC080) pAAV_Rep2Cap1_Sil1[V473D_N500E] VP2 KO [T138A]_KanR (˜40K plasmid/cell) and (NC110) pCargo_173CMV_SSo7dL8-LT-HSC-2_AAV1Sil1-VP2_173CMV_sfCherry plasmid library (1300 plasmids/cell) at 1.1 μg of total DNA/1E+06 cells using the FectoVIR-AAV Transfection reagent (Polyplus #101000022). The ratio of DNA:FectoVIR was 1 μg: 1 μl. Glucose was added to the transfection mix to a final concentration of 2 g/l after the transfection and the mixture was incubated at 37° C., 110 rpm, 6% CO2.

Two days post transfection, Benzonase was added to the transfection reactions to final concentration of 0.1 U/μl and a volume equal to 10% of the total volume in lysis buffer (500 mM HEPES, 10% Tween 20, 20 mM MgCl₂). Following a 3h incubation period at 37° C., a volume of sucrose salt solution (5 M NaCl, 7% sucrose) equal to 10% of the total volume was added to the lysate and the mixtures were incubated at 37° C. in agitation for 3 h. After this incubation step, a volume of salt sucrose solution (5 M NaCl, 7% sucrose) equal to 10% of the total volume was added and the samples were incubated for an additional 20 min. Samples were then centrifuged for 15 min at 3500 g, supernatants were filtered using a 0.22 μM filter and supplemented with EDTA to a final concentration of 5 mM. Next, the clarified lysates were loaded at 1 ml/min on an AAVX Pre-packed Column, 0.5×5 cm, 1 ml (ThermoFisher #A36652) mounted on an AKTA Pure instrument. Bound AAV particles were washed with 10 CV wash buffer, pH 7.3 (50 mM Tris-Cl, 0.5 M NaCl, 0.01% Pluronic F-68) and eluted with 5 CV glycine elution buffer, pH 2.7 (0.1 M Glycine, 0.2 M NaCl, 0.25 M L-arginine). Elution fractions (0.25 ml) were immediately neutralized with 25 μl 1 M Tris-Cl, pH 10. Fractions containing AAV (as assessed by UV280 signal) were pooled and dialyzed, using the Float-A-Lyzer Dialysis Device 100 KD (Spectrum™ #G235071), over night against PBS, pH 7.4+0.001% Pluronic F-68 and then passed through a 0.22 μm filter unit. Full and empty particles were separated on an iodixanol ultracentrifugation gradient and concentrated/buffer exchanged in PBS, pH 7.4+0.001% Pluronic F-68 using an Amicon Ultra-15 MWCO 100,000 Filter unit (Merck #UFC910008) and then passed through a 0.22 μm filter unit (final volume=2.2 ml).

### AAV Library Titer and Decoration Level Determination:

The QX200 droplet digital PCR (ddPCR) system (Bio-Rad) was used to determine the titer of the AAV vectors using the SV40 pA specific oligo set described in Table 3. Purified preparations were pretreated as follow: 5 μl of AAV were incubated with 1 unit of DNase and 1× DNase buffer (Merck #04716728001) in 20 μl total volume for 30 min at 37° C. 1 μl of Proteinase K at 20 mg/ml (Life technologies #EO0491) was added to the mixture and incubated for 1 h at 55° C. followed by an inactivation step at 95° C. for 15 min. Different 1/10 serial dilutions of treated preparations were prepared in 1×PCR Buffer II (Life technologies #N8080010), 2.5 mM MgCl₂, 0.05% Pluronic F-68 (Sigma Aldrich #K4894), 2 ng/μl Sheared Salmon Sperm DNA (Invitrogen #15632011) and used as template for PCR amplification.

The ddPCR reaction mix was prepared by combining 5.5 μl pre-treated AAV dilution with 900 nM forward and reverse SV40 pA primers each, 125 nM SV40 pA probe and 11 μl 2× ddPCR supermix for probes (BioRad #1863024), in a final volume of 22 μl. Technical duplicates were performed for each sample. 20 μl of each ddPCR reaction mix was loaded into a disposable droplet generator cartridge (Bio-Rad). Then, 70 μL of droplet generation oil for probes (BioRad #1863005) were loaded into each of the eight oil wells. Next, the cartridge was placed inside the QX200 droplet generator (Bio-Rad). Following droplet generation, 40 μl were transferred to a 96-well PCR plate using a multichannel pipet.

The plate was heat-sealed with pierceable foil and placed into the C1000 Touch Thermal Cycler (Bio-Rad). Thermal cycling conditions were as follows: 95° C. for 10 min, then 40 cycles of 95° C. for 30 s and 60° C. for 1 min followed by a 10 min incubation at 98° C. and an indefinite hold at 4° C. FAM fluorescent signal, labeling the AAV genome DNA sequence in each droplet, was counted using a QX200 digital droplet reader, and analyzed by QuantaSoft analysis software (Bio-Rad). For the calculation of AAV titers, the number of droplets was transformed by multiplying it with the respective total dilution factor and additionally with a factor of 2 to take into account possible reannealing of single stranded genomes.

CE-SDS analysis (Agilent 2100 Bioanalyzer system, Protein 230 kit using denaturing conditions) was performed to evaluate the VP distribution in the purified samples and the total VP yield.

Calculation of the total AAV particles/ml (vp/ml) and the VP ratio:
ng/μl (=μg/ml) values are divided by the observed MW and then multiplied by 1E+06 (μg to g conversion) to calculate moles/ml values
moles/ml values are multiplied by 6.022E+23 (Avogadro nr) to calculate the number of molecules/ml
VP distribution: calculated based on molecules/ml values assuming one AAV is made of 60 VPs in total
AAV particles/ml (vp/ml): Sum of VP1+VP2+VP3 molecules ml are divided by 60

ddPCR and CE-SDS results are reported in Table 34.

TABLE 34
QC on 2^{nd} round (BM_LT-HSC) enriched library prep

| | VP2 | | | |
|---|---|---|---|---|
| | fusion | | | % Full |
| Sample ID | per AAV | vg/ml | vp/ml | particle |
| (AAV-682) | 3.3 | 2.05E+13 | 4.61E+13 | 44.55% |
| ssAAV1Sil1_library_{—} | | | | |
| 173CMV_VP2-Sso7dL8- | | | | |
| LT-HSC- | | | | |
| 2_173CMV_sfCherry | | | | |

### Example 9.4: Third Round of Mobilized Humanized Mice Injection and Tissue Processing

A third in-vivo selection was conducted on NSG mice (n=15) using (AAV-682) ssAAV1Sil1_library_173CMV_VP2-Sso7dL8-LT-HSC-2_173CMV_sfCherry at a dose of 2.4E+13 vg/kg. The same mobilization, injection and tissue processing procedure as described in Example 7.1 was used.

The concentration of the eluted RNA was determined using a Qubit fluorometer. The results are summarized in Table 35.

TABLE 35
Concentration of RNA extracted from humanized mice tissues

| | | Qubit Conc |
|---|---|---|
| Tissue | Description | ng/μl |
| Bulk CD34+ | (AAV-682) Sso7d library sorted populations (pool of 15 | 20 |
| | mice) mCD45− hCD45+ hCD34+ | |
| CD38+ Progenitor | (AAV-682) Sso7d library sorted populations (pool of 15 | 41.3 |
| | mice) mCD45− hCD45+ hCD34+ hCD38+ | |
| CD38− CD45RA+ | (AAV-682) Sso7d library sorted populations (pool of 15 | 7.1 |
| Progenitor | mice) mCD45− hCD45+ hCD34+ hCD38− hCD45RA+ | |
| Multi-potent | (AAV-682) Sso7d library sorted populations (pool of 15 | <0.1 |
| progenitor | mice) mCD45− hCD45+ hCD34+ hCD38− hCD45RA− | |
| | hCD90− | |
| LT-HSC | (AAV-682) Sso7d library sorted populations (pool of 15 | <0.1 |
| | mice) mCD45− hCD45+ hCD34+ hCD38− hCD45RA− | |
| | hCD90+ | |
| Whole Bone | (AAV-682) Sso7d library sorted populations (pool of 15 | 86 |
| Marrow | mice) | |
| Blood | (AAV-682) Sso7d library sorted populations (pool of 15 | 128 |
| | mice) | |
| Muscle | (AAV-682) Sso7d library sorted populations (pool of 15 | 419 |
| | mice) | |
| Lung | (AAV-682) Sso7d library sorted populations (pool of 15 | 250 |
| | mice) | |
| Liver | (AAV-682) Sso7d library sorted populations (pool of 15 | 1370 |
| | mice) | |
| Kidney | (AAV-682) Sso7d library sorted populations (pool of 15 | 4560 |
| | mice) | |
| Heart | (AAV-682) Sso7d library sorted populations (pool of 15 | 890 |
| | mice) | |
| Brain | (AAV-682) Sso7d library sorted populations (pool of 15 | 1930 |
| | mice) | |
| Spleen | (AAV-682) Sso7d library sorted populations (pool of 15 | 2960 |
| | mice) | |

### Example 9.5: NGS Analysis on RNA Extracted after Third Selection Round

RT-PCR on Extracted RNA from Bone Marrow LT-HSC Cells:

RT-PCR was performed using the LunaScript Multiplex One-Step RT-PCR Kit (NEB #E1555), 12.5 pmol of LC0052 (5′ CCCGGGGAACTCCTCCCGAGTCGAAATTCGCCACC 3′)/LC0053 (5′ GTTGCTGGAGGTT CTCCGAGAGGTTGTGGGTCGGG 3′) primers and total RNA extracted from different tissues after the third selection round (see Table 36 for the amounts used) in 25 μl total reaction volume. RT-PCR was carried out with the following cycling protocol: 10 min at 55° C., 1 min at 98° C.; 30 cycles of 98° C. for 10 s, 55° C. for 20 s, 72° C. for 30 s; followed by final amplification at 72° C. for 5 min and then holding at 4° C.

TABLE 36
Total RNA amounts used for RT-PCR reactions

| | Total ng of RNA used in |
|---|---|
| Sample ID | RT-PCR reactions |
| Bulk CD34+ | 20 |
| CD38+ Progenitor | 41.3 |
| CD38− CD45RA+ Progenitor | 7.1 |
| Multi-potent progenitor | <0.1 |
| LT-HSC | <0.1 |
| Whole Bone Marrow | 86 |
| Blood | 128 |
| Muscle | 419 |
| Lung | 250 |
| Liver | 1370 |
| Kidney | 4560 |
| Heart | 890 |
| Brain | 1930 |
| Spleen | 2960 |

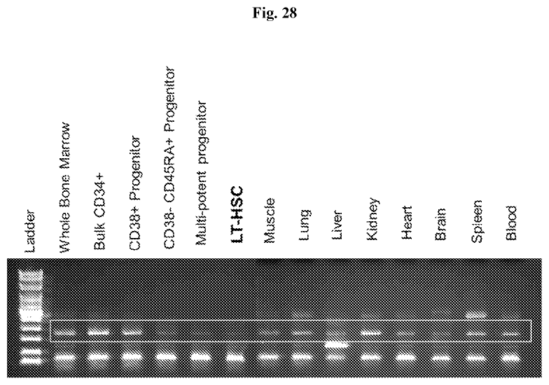

The product size was assessed on 2 μl of the RT-PCR reaction using the Flash-Gel system (Flash-Gel DNA cassette, 1.2% Lonza #57031 using Fast DNA Ladder NEB #N3238S) as shown in FIG. 28. The PCR product was purified using the Qiaquick PCR purification Kit (Qiagen #28104) and eluted in 50 μl of H₂O.

### Library Preparation and NGS on Illumina's MiSeq:

Amplicon libraries were generated from the cDNAs prepared from extracted RNA as described above in one single PCR reaction using a set of primers containing Illumina's adapter. The forward primer was used in combination with different reverse primers containing the Illumina TruSeq index to allow for the identification of individual samples. The list of primers used in this example is shown in Table 37. Additionally, an amplicon library was also prepared from the input AAV preparation used to perform the third selection round: (AAV-682) ssAAV1Sil1 library_173CMV_VP2-Sso7dL8-LT-HSC-2_173CMV_sfCherry.

TABLE 37
List of primers used in this example (bold: index, italic: annealing region)

| Name | Primer seq 5′-3′ |
|---|---|
| LC0034 | AATGATACGGCGACCACCGAGATCTACACTCTTTCCCTACACGACGC |
| (universal for | TCTTCCGATCTCAGGTGGACATCAGCAAGATCAAG |
| primer) | |
| LC0074 | CAAGCAGAAGACGGCATACGAGATCTGATCGTGACTGGAGTTCAGA |
| (rev primer/Whole | CGTGTGCTCTTCCGATCTCTTTAGGGGCGTCCTTTTCGGACAC |
| Bone Marrow) | |
| LC0075 | CAAGCAGAAGACGGCATACGAGATAAGCTAGTGACTGGAGTTCAGA |
| (rev primer/Bulk | CGTGTGCTCTTCCGATCTCTTTAGGGGCGTCCTTTTCGGACAC |
| CD34+) | |
| LC0076 | CAAGCAGAAGACGGCATACGAGATGTAGCCGTGACTGGAGTTCAGA |
| (rev primer/CD38+ | CGTGTGCTCTTCCGATCTCTTTAGGGGCGTCCTTTTCGGACAC |
| Progenitors) | |
| LC0077 | CAAGCAGAAGACGGCATACGAGATTACAAGGTGACTGGAGTTCAGA |
| (rev primer/CD38- | CGTGTGCTCTTCCGATCTCTTTAGGGGCGTCCTTTTCGGACAC |
| CD45RA+ | |
| Progenitor) | |
| LC0078 | CAAGCAGAAGACGGCATACGAGATTTGACTGTGACTGGAGTTCAGA |
| (rev primer/Multi- | CGTGTGCTCTTCCGATCTCTTTAGGGGCGTCCTTTTCGGACAC |
| potent progenitor) | |
| LC0079 | CAAGCAGAAGACGGCATACGAGATGGAACTGTGACTGGAGTTCA |
| (rev primer/LT- | GACGTGTGCTCTTCCGATCTCTTTAGGGGCGTCCTTTTCGGACAC |
| HSC) | |
| LC0080 | CAAGCAGAAGACGGCATACGAGATTGACATGTGACTGGAGTTCA |
| (rev primer/ | GACGTGTGCTCTTCCGATCTCTTTAGGGGCGTCCTTTTCGGACAC |
| Muscle) | |
| LC0081 | CAAGCAGAAGACGGCATACGAGATGGACGGGTGACTGGAGTTCA |
| (rev primer/Lung) | GACGTGTGCTCTTCCGATCTCTTTAGGGGCGTCCTTTTCGGACAC |
| LC0082 | CAAGCAGAAGACGGCATACGAGATGCGGACGTGACTGGAGTTCA |
| (rev primer/Liver) | GACGTGTGCTCTTCCGATCTCTTTAGGGGCGTCCTTTTCGGACAC |
| LC0083 | CAAGCAGAAGACGGCATACGAGATTTTCACGTGACTGGAGTTCA |
| (rev primer/ | GACGTGTGCTCTTCCGATCTCTTTAGGGGCGTCCTTTTCGGACAC |
| Kidney) | |
| LC0084 | CAAGCAGAAGACGGCATACGAGATGGCCACGTGACTGGAGTTCA |
| (rev primer/Heart) | GACGTGTGCTCTTCCGATCTCTTTAGGGGCGTCCTTTTCGGACAC |
| LC0085 | CAAGCAGAAGACGGCATACGAGATCGAAACGTGACTGGAGTTCA |
| (rev primer/Brain) | GACGTGTGCTCTTCCGATCTCTTTAGGGGCGTCCTTTTCGGACAC |
| LC0086 | CAAGCAGAAGACGGCATACGAGATCGTACGGTGACTGGAGTTCA |
| (rev primer/Spleen) | GACGTGTGCTCTTCCGATCTCTTTAGGGGCGTCCTTTTCGGACAC |
| LC0087 | CAAGCAGAAGACGGCATACGAGATCCACTCGTGACTGGAGTTCA |
| (rev primer/Blood) | GACGTGTGCTCTTCCGATCTCTTTAGGGGCGTCCTTTTCGGACAC |
| LC0088 | CAAGCAGAAGACGGCATACGAGATATCAGTGTGACTGGAGTTCA |
| (rev primer/AAV- | GACGTGTGCTCTTCCGATCTCTTTAGGGGCGTCCTTTTCGGACAC |
| 682 input) | |

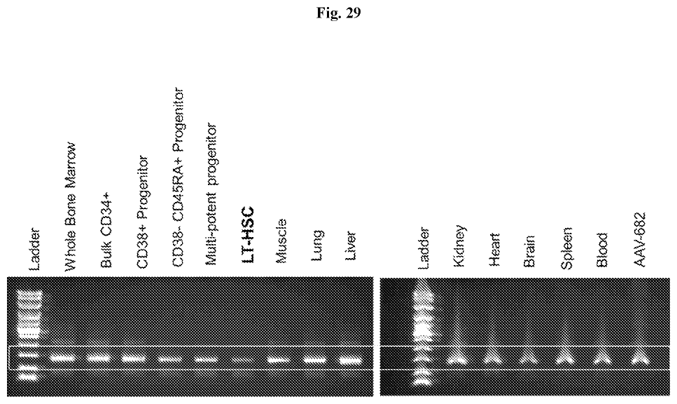

PCR reactions were prepared using the Q5 Hot Start High-Fidelity Master Mix (NEB #M0494), 10 pmol of each primer and 10 μl of purified cDNA samples as template in 25 μl total reaction volume. PCR was carried out with following cycling protocol: 2 min at 95° C.; 10 cycles of 98° C. for 10 s; 55° C. for 15 s; 72° C. for 25 s; followed by 2 min incubation at 72° C. and then holding at 4° C. Additional 10 cycles were added for L8 and L13 samples due to the low yield obtained with 20 cycles. PCR products were purified using the Qiaquick PCR purification Kit (Qiagen #28104) and eluted in 50 μl of water. The size of the products was assessed on 2 μl of RT-PCR reaction using the Flash-Gel system (Flash-Gel DNA cassette, 1.2% Lonza #57031 using Fast DNA Ladder NEB #N3238S) as shown in FIG. 29.

After quantitation using a Qubit fluorometer, libraries were equimolarly pooled at a final concentration of 8 μM and sequenced on a MiSeq System (Illumina) using the Illumina MiSeq Reagent Kit v3 (150 cycles, #MS-102-3001). The resulting read numbers are summarized in Table 38.

TABLE 38
NGS reads count

| Name | Initial reads |
|---|---|
| Whole Bone Marrow | 813307 |
| Bulk CD34+ | 812827 |
| CD38+ Progenitor | 597680 |
| CD38− CD45RA+ Progenitor | 716979 |
| Multi-potent progenitor | 725102 |
| LT-HSC | 548883 |
| Muscle | 799938 |
| Lung | 832640 |
| Liver | 706881 |
| Kidney | 831601 |
| Heart | 838244 |
| Brain | 922195 |
| Spleen | 866037 |
| Blood | 761597 |
| AAV-682 input | 700553 |

### Data Analysis Using Custom Script:

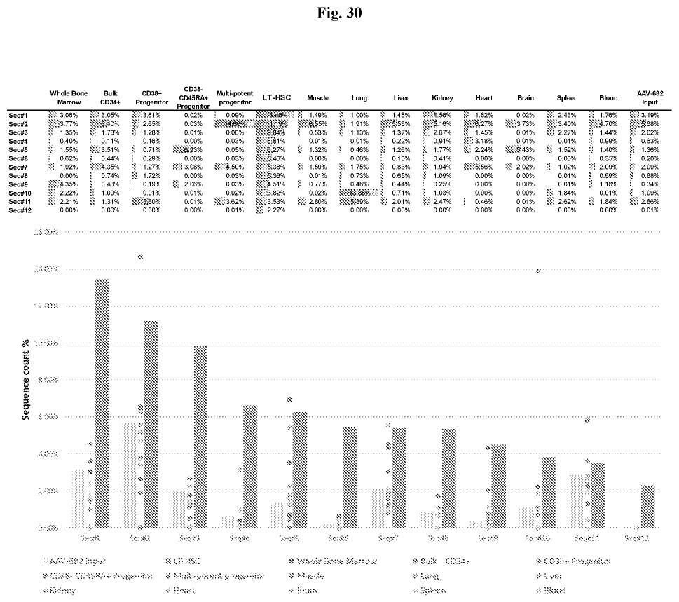

Generated fastq files were initially filtered by quality (Q>30, where Q indicates Phred quality score) and length (110-130 bp). After this quality trim, the sequences were processed aiming to map Sso7d specific boundaries (ISKI to DAPK) and 100′000 was retrieved for all samples. The distribution of single sequences in the different organs was assessed on final filtered sequences and the results are summarized in FIG. 30 focusing on the top twelve hits identified in the LT-HSC sample.

Most of the hits seem to have high specificity for LT-HSC, while some of them (e.g. Seq #5, Seq #7, Seq #9 and Seq #11) show unspecific enrichment in non-targeted organs/cells.

### Example 10: Identification of VP2 Variants with Increased N-Terminal Exposure

### Introduction

It has been suggested that both VP1 and VP2 are involved in forming globules inside the capsid of empty and full AAV particles as shown via electron cryo-microscopy. The presence of globules inside the capsid is linked to low accessibility of N-terminal domains on the outside of the capsid (Kronenberg et al., Journal of Virology, 2005). In light of these observations, it was hypothesized that the fact that the VP2 N-terminus naturally faces inward on the capsid shell might reduce the exposure of the Sso7d scaffold fused to it.

In this example, site saturation mutagenesis on the first 79 non-structured amino acids of AAV1Sil1 VP2 fused with different linker sequences to anti-EGFR Sso7d was employed. The AAV library was subjected to positive selection through infection of HKB11 EGFR overexpressing cells and negative selection through infection of HKB11 EGFR knockout cells to identify mutations that specifically improve receptor mediated uptake.

### Example 10.1 AAV1Sil1 VP2 N-Term Site Saturation Mutagenesis (SSM) Library Generation

Acceptor plasmids generation:

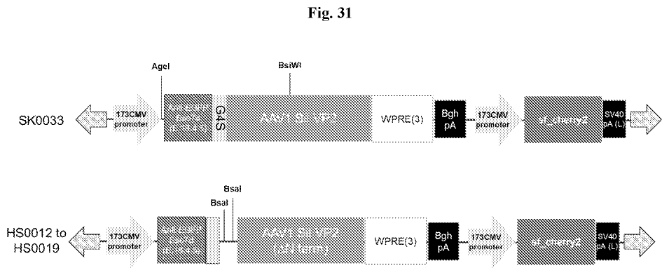

In a first step, acceptor plasmids that contain one of eight different linkers, either flexible—G₄S, (G₄S)₂, (G₄S)₃, (G₄S)₄—or rigid —(APS)₂, (APS)₃, (APS)₄, (APS)₅—were generated. These linker sequences were ordered as fragment DNA at GeneArt and were cloned into the backbone SK033_pCargo_173_CMV_Sso7d_EGFR_AAV1sil VP2_173_CMV_sfCherry as previously described using AgeI-BsiWI and removing the first amino acids of VP2 until D270 (AAV1 VP1 coordinates). Both acceptor plasmids and the library fragment described in the next section contain a BsaI restriction site to accommodate typeIIS cloning (see FIG. 31 and Table 39).

TABLE 39
Acceptor plasmid ID and added linkers

| | Linker |
|---|---|
| Plasmid ID | used |
| (HS0012)_pCargo_173_CMV_Sso7d_EGFR_(APS)x2_AAV1sil_VP2_173_CMV_sfCherry | APSx2 |
| (HS0013)_pCargo_173_CMV_Sso7d_EGFR_(APS)x3_AAV1sil_VP2_173_CMV_sfCherry | APSx3 |
| (HS0014)_pCargo_173_CMV_Sso7d_EGFR_(APS)x4_AAV1sil_VP2_173_CMV_sfCherry | APSx4 |
| (HS0015)_pCargo_173_CMV_Sso7d_EGFR_(APS)x5_AAVlsil_VP2_173_CMV_sfCherry | APSx5 |
| (HS0016)_pCargo_173_CMV_Sso7d_EGFR_(G4S)_AAV1sil_VP2_173_CMV_sfCherry | G4S |
| (HS0017)_pCargo_173_CMV_Sso7d_EGFR_(G4S)x2_AAV1sil_VP2_173_CMV_sfCherry | G4Sx2 |
| (HS0018)_pCargo_173_CMV_Sso7d_EGFR_(G4S)x3_AAV1sil_VP2_173_CMV_sfCherry | G4Sx3 |
| (HS0019)_pCargo_173_CMV_Sso7d_EGFR_(G4S)x4_AAV1sil_VP2_173_CMV_sfCherry | G4Sx4 |

### 79AA SSM Library Cloning (V-Exp Library):

The 79AA SSM library fragment was ordered at Twist Bioscience. It comprises a BsaI site at its 5′ for direct fusion to the different linkers present in HS0012-HS0019, a site saturation library from A139 to G217 (AAV1 VP1 coordinates), a stretch of bases coding from A218 to N271 of AAV1 and a second BsaI site for cloning into the previously mentioned acceptor plasmids. All amino acids except cysteine were considered for SSM (79 positions, 19 amino acids encoded by only 1 codon each, yielding a total complexity of 1501 variants).

To generate the V-Exp plasmid library, 160 ng of the 79AA SSM library fragment and 80 ng of equimolarly pooled HS0012 to HS0019 acceptor plasmids were mixed with 40 Units of BsaI-HF v2 (NEB; E3733), 2000 Units of T4 DNA Ligase (NEB; M0202T) and 1× assembly buffer (1×CutSmart Buffer+1 mM ATP) in a final volume of 50 μl. The reaction mixtures were then treated as follows: 30 min incubation at 37° C.; 30 cycles of 37° C. for 5 min followed by 16° C. for 5 min and a final step of 37° C. for 1 h. Before initiating the last step, 20 additional Units of BsaI-HF v2 were added to the mix. Next, the reaction product was treated with Plasmid Safe (PS) DNase (Epicentre; E3105K) to digest any unassembled fragments and purified using the Wizard SV Gel and PCR Clean-up System (Promega #A9280). The reaction yielded 1.5 ng of assembled plasmid (as defined by the amount of DNA remaining after the PS DNase digestion step). 2×50 μl One Shot TOP10 Electrocomp E. coli (ThermoFisher; C4040-52) were transformed with 0.1 ng each of the assembled plasmid library. The reactions were pooled and added to 100 ml TB+kanamycin medium after 1 h SOC incubation. 100 μl were then removed from the flask, different dilutions were plated on LB+kanamycin plates and incubated over night at 37° C. The number of CFU on agar plates was found to be 1.5E+05, approximately 10-fold higher than the theoretical library complexity of 1.2E+04 (8 linkers×1501 SSM library size). Plasmid DNA was extracted after over night growth at 37° C. from original 100 ml culture using the Maxiprep Kit (QIAGEN #12163) yielding the (HS0026) pCargo LTM V-Exp library_173_CMV_Sso7d_EGFR_AAV1sil\VP2_173_CMV_sfCherry.

ssAAV1Sil1 V-Exp Library Generation and QC:

One liter of HEK293 suspension cells at a concentration of 2E+06 vc/ml was transfected with pHelper (˜40K plasmid/cell), (NC080) pAAV_Rep2Cap1_Sil1[V473D_N500E] VP2 KO [T138A]_KanR (˜40K plasmid/cell) and (HS0026)_pCargo V-Exp lib_CMV_Sso7d_EGFR_AAV1sil\VP2_CMV_sfCherry plasmid library (1300 plasmids/cell) at 1.1 μg of total DNA/1E+06 cells using the FectoVIR-AAV Transfection reagent (Polyplus #101000022). The ratio of DNA:FectoVIR was 1 μg:1 μl. Glucose was added to the transfection mix to a final concentration of 2 g/l after the transfection and the mixture was incubated at 37° C., 110 rpm, 6% CO2.

Two days post transfection, Benzonase in lysis buffer (500 mM HEPES, 10% Tween 20, 20 mM MgCl₂) was added to the transfection reactions to a final concentration of 0.1 U/μl and a volume equal to 10% of the total volume. Following a 3 h incubation period at 37° C., a volume of sucrose salt solution (5 M NaCl, 7% sucrose) equal to 10% of the total volume was added to the lysate and the mixtures were incubated at 37° C. in agitation for 3 h. After this incubation step, a volume equal to 10% of the total volume in salt sucrose solution (5 M NaCl, 7% sucrose) was added and incubated for an additional 20 min. Samples were centrifuged for 30 min at 3500 g, supernatants were filtered using a 0.22 μM filter and supplemented with EDTA to a final concentration of 5 mM. Next, the clarified lysates were loaded at 1 ml/min on an AAVX Pre-packed Column, 0.5×5 cm, 1 ml (ThermoFisher #A36652) mounted on an AKTA Pure instrument. Bound AAV particles were washed with 10 CV wash buffer, pH 7.3 (50 mM Tris-Cl, 0.5 M NaCl, 0.01% Pluronic F-68) and eluted with 5 CV glycine elution buffer, pH 2.7 (0.1 M Glycine, 0.2 M NaCl, 0.25 M L-arginine). Elution fractions (0.25 ml) were immediately neutralized with 25 μl 1 M Tris-Cl, pH 10. Fractions containing AAV (as assessed by UV280 signal) were pooled and dialyzed, using the Float-A-Lyzer Dialysis Device 100 KD (Spectrum™ #G235071), over night against PBS, pH 7.4+0.001% Pluronic F-68 and then passed through a 0.22 μm filter unit. Full and empty particles were separated on an iodixanol ultracentrifugation gradient and concentrated/buffer exchanged in PBS, pH 7.4+0.001% Pluronic F-68 using an Amicon Ultra-15 MWCO 100,000 Filter unit (Merck #UFC910008) and then passed through a 0.22 μm filter unit (final volume=1 ml).

### Titer and Decoration Level Determination:

The QX200 droplet digital PCR (ddPCR) system (Bio-Rad) was used to determine the titer of the AAV vectors using the SV40 pA specific oligo set described in Table 3. Purified preparations were pretreated as follows: 5 μl of AAV were incubated with 1 unit of DNase and 1× DNase buffer (Merck #04716728001) in 20 μl total volume for 30 min at 37° C. 1 μl of Proteinase K at 20 mg/ml (Life technologies #E00491) was added to the mixture and incubated for 1 h at 55° C. followed by an inactivation step at 95° C. for 15 min. Different 1/10 serial dilutions of treated preparations were prepared in 1×PCR Buffer II (Life technologies #N8080010), 2.5 mM MgCl₂, 0.05% Pluronic F-68 (Sigma Aldrich #K4894), 2 ng/μl Sheared Salmon Sperm DNA (Invitrogen #15632011) and used as template for PCR amplification.

The ddPCR reaction mix was prepared by combining 5.5 μl pre-treated AAV dilution with 900 nM forward and reverse SV40 pA primers each, 125 nM SV40 pA probe and 11 μl 2× ddPCR supermix for probes (BioRad #1863024), in a final volume of 22 μl. Technical duplicates were performed for each sample. 20 μl of each ddPCR reaction mix was loaded into a disposable droplet generator cartridge (Bio-Rad). Then, 70 μL of droplet generation oil for probes (BioRad #1863005) was loaded into each of the eight oil wells. Next, the cartridge was placed inside the QX200 droplet generator (Bio-Rad). Following droplet generation, 40 μl were transferred to a 96-well PCR plate using a multichannel pipet.

The plate was heat-sealed with pierceable foil and placed into the C1000 Touch Thermal Cycler (Bio-Rad). Thermal cycling conditions were as follows: 95° C. for 10 min, then 40 cycles of 95° C. for 30 s and 60° C. for 1 min followed by a 10 min incubation at 98° C. and an indefinite hold at 4° C. FAM fluorescent signal, labeling the AAV genome DNA sequence in each droplet, was counted using a QX200 digital droplet reader and analyzed by QuantaSoft analysis software (Bio-Rad). For the calculation of AAV titers, the number of droplets were transformed by multiplying them with the respective total dilution factor and additionally with a factor of 2 to take into account possible reannealing of single stranded genomes.

CE-SDS analysis (Agilent 2100 Bioanalyzer system, Protein 230 kit using denaturing conditions) was performed to evaluate the VP distribution in the purified samples and the total VP yield.

Calculation of the total AAV particles/ml (vp/ml) and the VP ratio:
ng/μl (=μg/ml) values are divided by the observed MW and then multiplied by 1E+06 (μg to g conversion) to calculate moles/ml values
moles/ml values are multiplied by 6.022E+23 (Avogadro nr) to calculate the number of molecules/ml
VP distribution: calculated based on molecules/ml values assuming one AAV is made of 60 VPs in total
AAV particles/ml (vp/ml): Sum of VP1+VP2+VP3 molecules ml are divided by 60

The ddPCR and CE-SDS results are reported in Table 40.

TABLE 40
QC on ssAAV1Sil1 V-Exp library post UC

| | VP2 fusion | | | % Full |
|---|---|---|---|---|
| Sample ID | per AAV | vg/ml | vp/ml | particles |
| (AAV-540) | 3.7 | 6.85E+12 | 8.65E+12 | 79% |
| ssAAV1Sil1_V-Exp lib- | | | | |
| sfCherry | | | | |

### Library Preparation and NGS on Illumina's MiSeq:

Amplicon libraries were generated from the naïve plasmid and naïve libraries using a set of primers covering the region where the LTM library was inserted. For this purpose, either 50 ng plasmid DNA or 5 μl AAV sample was mixed with 13 μl 2× Q5 HiFi Hot Start Ready Mix (NEB #M0494), 10 pmol Sme0001f (5′ CAGTGAAGCTGACATACCAGGGC 3′)/Sme0002r (5′ ATTGTTGTTGATGAGTCGCTGC 3′) primers in a total volume of 50 μl. Next, a second PCR reaction was performed with the freshly generated amplicons and a primer set containing Illumina's adapters. In this set, the reverse primer also contains the Illumina TruSeq index to allow for identification of individual samples. The list of primers used in this example can be found in Table 41.

TABLE 41
List of primers used in this example for index PCR
(bold: index, italic: annealing region)

| | Primer | |
|---|---|---|
| Target | name | Sequence (5′-3′) |
| HS0026 | Sme0009_ | AATGATACGGCGACCACCGAGATCTACACTCTTTCCCTACACGAC |
| | NGS_f | GCTCTTCCGATCTTTGGAACAGGATGGGTGTCC; |
| | Sme0015_ | CAAGCAGAAGACGGCATACGAGATCACTGTGTGACTGGAGTTCA |
| | NGS_r | GACGTGTGCTCTTCCGATCTGGTGGTGATGACTCTGTCGC |
| | (R5) | |
| AAV- | Sme0009_ | AATGATACGGCGACCACCGAGATCTACACTCTTTCCCTACACGAC |
| 540 | NGS_f | GCTCTTCCGATCTTTGGAACAGGATGGGTGTCC; |
| | Sme0017_ | CAAGCAGAAGACGGCATACGAGATTGGTCAGTGACTGGAGTTCA |
| | NGS_r | GACGTGTGCTCTTCCGATCTGGTGGTGATGACTCTGTCGC |
| | (R4) | |

### Example 10.2 In-Vitro Selection for Improved N-Term Exposure (with NGS Results)

ssAAV1Sil1_V-Exp Lib-sfCherry Library Transduction and RNA Extraction:

HKB11 EGFR overexpressing cells (EGFR+) and HKB11 EGFR knockout cells (KO) were cultivated in suspension using V3 medium supplemented with 1% FBS and 200 μg/ml zeocine (EGFR+) or 2% FBS (EGFR KO) and incubated at 37° C., 110 rpm, 6% CO2. On the day of transduction, cells were seeded at 5E+05 cells/ml in 20 ml of V3 medium without additives in 125 ml shake flasks (4 flasks/cell line). ssAAV1Sil1_V-Exp lib-sfCherry (AAV-540) library was added at a multiplicity of infection of 10, 100, 1′000 and 10′000 to both cell lines and incubated at 37° C., 110 rpm, 6% CO2 (three hours post transduction supplements were added in each flask). Three days post-transduction, cells were counted and 1E+07 cells were pelleted, washed with PBS, and used for both DNA and RNA purification using the Allprep DNA/RNA Mini Kit (Qiagen, 80204). The RNA concentration was determined using a Qubit fluorometer, yielding approximately 100-200 ug per sample.

### RT-PCR on Extracted RNA Samples:

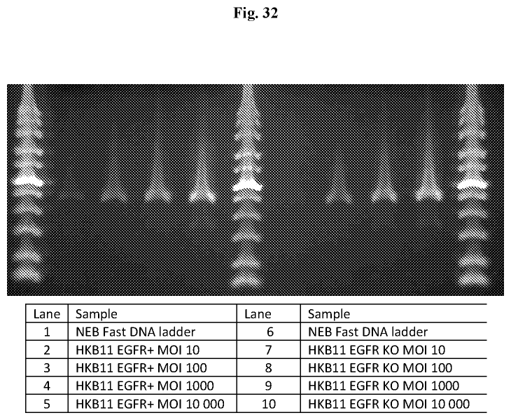

RT-PCR was performed using the LunaScript Multiplex One-Step RT-PCR Kit (NEB #E1555), 12.5 pmol of Sme0001f (5′ CAGTGAAGCTGACATACCAGGGC 3′)/Sme0002r (5′ ATTGTTGTTGATGAGTCGCTGC 3′) primers and 100 ng of total RNA in 50 μl total reaction volume. RT-PCR was carried out with the following cycling protocol: 10 min at 55° C., 1 min at 98° C.; 30 cycles of 98° C. for 10 s, 55° C. for 20 s, 72° C. for 30 s; followed by final amplification at 72° C. for 5 min and then holding at 4° C. Reactions were purified using the MinElute PCR purification Kit (Qiagen #28004) and eluted in 30 μl of water. The concentrations ranged from 10 ng/ul to 30 ng/ul based on Nanodrop spectrophotometer measurement. The size of the products was assessed on 1 μl of the RT-PCR reactions using the Flash-Gel system (Flash-Gel DNA cassette, 1.2% Lonza #57031) as shown in FIG. 32.

### Library Preparation and NGS on Illumina's MiSeq:

Amplicon libraries were generated from the different purified RT-PCR reactions using a set of primers containing Illumina's adapter. The forward primer was used in combination with a reverse primer containing the Illumina TruSeq index to allow for identification of individual samples. The list of primers used in this example is shown in Table 42.

TABLE 42
List of primers used for amplicon library
(bold: index, italic: annealing region)

| Target | Primer name | Sequence |
|---|---|---|
| EGFR+ MOI | Sme0009_NGS_f | AATGATACGGCGACCACCGAGATCTACACTCTTT |
| 10 | | CCCTACACGACGCTCTTCCGATCTTTGGAACAGGA |
| | | TGGGTGTCC; |
| | Sme0010_NGS_r | CAAGCAGAAGACGGCATACGAGATCGTGATGTG |
| | (R1) | ACTGGAGTTCAGACGTGTGCTCTTCCGATCTGGT |
| | | GGTGATGACTCTGTCGC |
| EGFR+ MOI | Sme0009_NGS_f | AATGATACGGCGACCACCGAGATCTACACTCTTT |
| 100 | | CCCTACACGACGCTCTTCCGATCTTTGGAACAGGA |
| | | TGGGTGTCC; |
| | Sme0011_NGS_r | CAAGCAGAAGACGGCATACGAGATGCCTAAGTG |
| | (R3) | ACTGGAGTTCAGACGTGTGCTCTTCCGATCTGGT |
| | | GGTGATGACTCTGTCGC |
| EGFR+ MOI | Sme0009_NGS_f | AATGATACGGCGACCACCGAGATCTACACTCTTT |
| 1000 | | CCCTACACGACGCTCTTCCGATCTTTGGAACAGGA |
| | | TGGGTGTCC; |
| | Sme0017_NGS_r | CAAGCAGAAGACGGCATACGAGATTGGTCAGTG |
| | (R4) | ACTGGAGTTCAGACGTGTGCTCTTCCGATCTGGT |
| | | GGTGATGACTCTGTCGC |
| EGFR+ MOI | Sme0009_NGS_f | AATGATACGGCGACCACCGAGATCTACACTCTTT |
| 10000 | | CCCTACACGACGCTCTTCCGATCTTTGGAACAGGA |
| | | TGGGTGTCC; |
| | Sme0015_NGS_r | CAAGCAGAAGACGGCATACGAGATCACTGTGTG |
| | (R5) | ACTGGAGTTCAGACGTGTGCTCTTCCGATCTGGT |
| | | GGTGATGACTCTGTCGC |
| EGFR KO | Sme0009_NGS_f | AATGATACGGCGACCACCGAGATCTACACTCTTT |
| MOI 10 | | CCCTACACGACGCTCTTCCGATCTTTGGAACAGGA |
| | | TGGGTGTCC; |
| | Sme0012_NGS_r | CAAGCAGAAGACGGCATACGAGATTCAAGTGTG |
| | (R8) | ACTGGAGTTCAGACGTGTGCTCTTCCGATCTGGT |
| | | GGTGATGACTCTGTCGC |
| EGFR KO | Sme0009_NGS_f | AATGATACGGCGACCACCGAGATCTACACTCTTT |
| MOI 100 | | CCCTACACGACGCTCTTCCGATCTTTGGAACAGGA |
| | | TGGGTGTCC; |
| | Sme0013_NGS_r | CAAGCAGAAGACGGCATACGAGATCTGATCGTG |
| | (R9) | ACTGGAGTTCAGACGTGTGCTCTTCCGATCTGGT |
| | | GGTGATGACTCTGTCGC |
| EGFR KO | Sme0009_NGS_f | AATGATACGGCGACCACCGAGATCTACACTCTTT |
| MOI 1000 | | CCCTACACGACGCTCTTCCGATCTTTGGAACAGGA |
| | | TGGGTGTCC; |
| | Sme0016_NGS_r | CAAGCAGAAGACGGCATACGAGATATTGGCGTG |
| | (R6) | ACTGGAGTTCAGACGTGTGCTCTTCCGATCTGGT |
| | | GGTGATGACTCTGTCGC |
| EGFR KO | Sme0009_NGS_f | AATGATACGGCGACCACCGAGATCTACACTCTTT |
| MOI 10000 | | CCCTACACGACGCTCTTCCGATCTTTGGAACAGGA |
| | | TGGGTGTCC; |
| | Sme0014_NGS_r | CAAGCAGAAGACGGCATACGAGATAAGCTAGTG |
| | (R10) | ACTGGAGTTCAGACGTGTGCTCTTCCGATCTGGT |
| | | GGTGATGACTCTGTCGC |

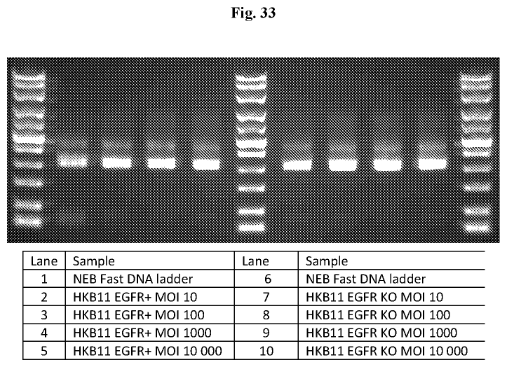

PCR reactions were prepared by combining the Q5 Hot Start High-Fidelity Master Mix (NEB 4M0494), 10 pmol of each primer and 5 ng of purified RT-PCR samples as template in 25 μl total reaction volume. PCR was carried out with following cycling protocol: 2 min at 95° C.; 15 cycles of 98° C. for 10 s; 55° C. for 15 s; 72° C. for 25 s; followed by 2 min incubation at 72° C. and then holding at 4° C. Reactions were purified with the MinElute PCR purification Kit (Qiagen #28004) and eluted in 30 μl of water. The size and quality of the products were assessed on 1 μl of purified amplicon PCR reactions using the Flash-Gel system (Flash-Gel DNA cassette, 1.200 Lonza 457031) as shown in FIG. 33.

After quantitation with the Qubit fluorometer (all between 15-30 ng/μL), the libraries were equimolarly pooled at a final concentration of 8 μM and sequenced on a MiSeq System (Illumina) using the Illumina MiSeq Reagent Kit v3 (150 cycles, #MS-102-3001).

### Data Analysis Using Custom Script:

Generated fastq files were initially filtered by quality (Q>30, where Q indicates the Phred quality score) and corresponding forward and reverse reads were merged. After this quality trim, the merged sequences were processed aiming to map Sso7d specific boundaries (MLEK to ADGV). The numbers of final filtered reads are reported in Table 43.

TABLE 43
Initial and filtered NGS reads count

| Name | Filtered reads |
|---|---|
| EGFR+ MOI 10 | 162628 |
| EGFR+ MOI 100 | 166839 |
| EGFR+ MOI 1000 | 126648 |
| EGFR+ MOI 10000 | 192245 |
| EGFR KO MOI 10 | 162596 |
| EGFR KO MOI 100 | 164315 |
| EGFR KO MOI 1000 | 149681 |
| EGFR KO MOI 10000 | 153137 |

### MOI Selection Using Occurrence Analysis:

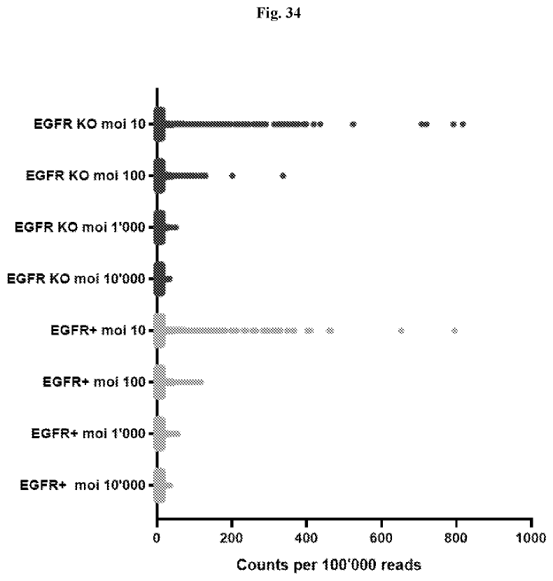

The distribution of occurrences in each sample were plotted to generate a graphical representation of selective pressure applied during ssAAV1Sil1_V-Exp lib-sfCherry library transduction in EGFR+ and EGFR KO cells. The broader distribution observed at moi 10 for both cell lines as depicted in FIG. 34, might be a potential indication of correct selective pressure applied and was therefore selected for the next steps of analysis.

### Identification and Prioritization of VP2 N-Term Variants Impacting Cell Transduction:

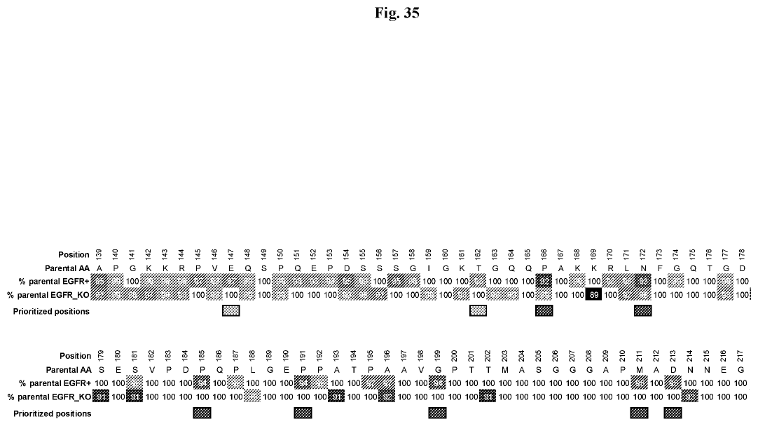

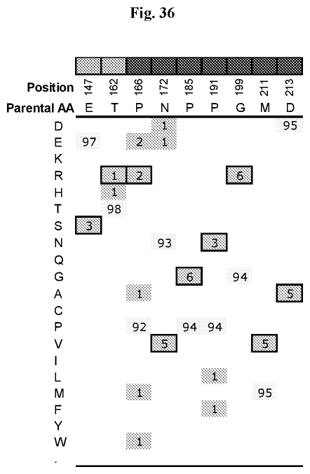

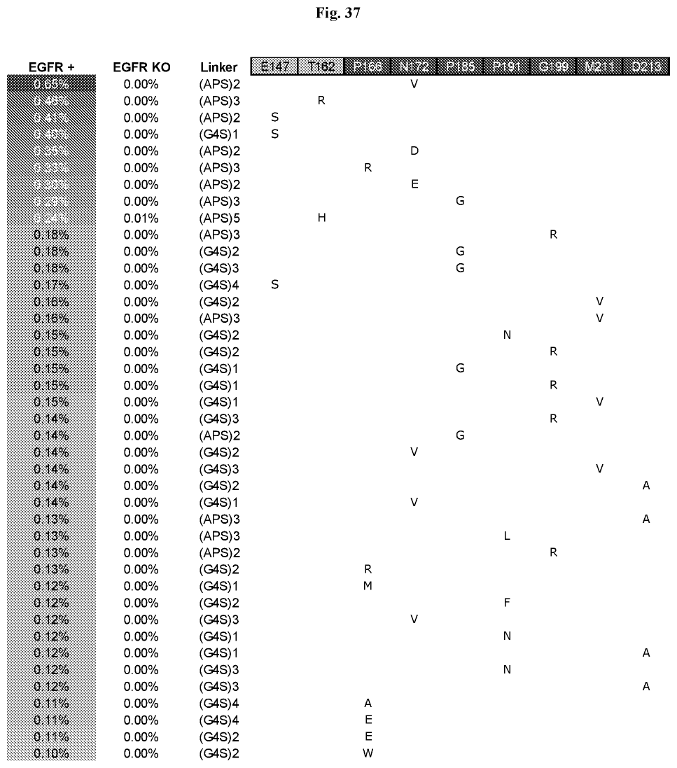

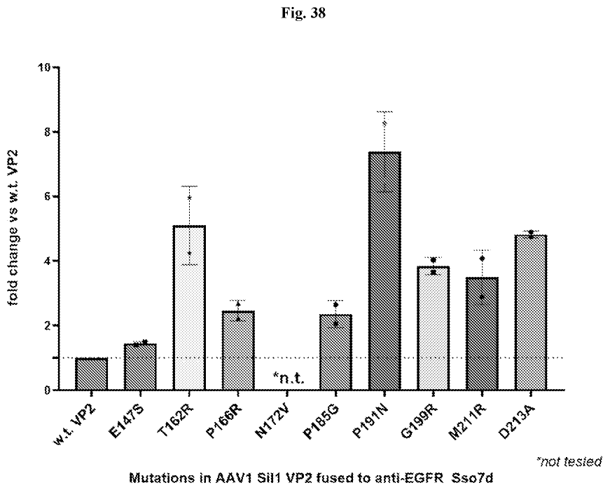

Variation analysis was conducted at each targeted positions on sequences that show at least 0.1% frequency in the V-exp library transduction in EGFR+ cells at moi 10 (n=87). At each given position the percentage of parental amino acid and the percentage of each single variant was calculated (see FIG. 35 and FIG. 36) for both positive (EGFR+) and negative (EGFR KO) selection. Nine positions were prioritized based on the high difference observed between EGFR+ and EGFR KO (dark box) or for the high absolute enrichment (light box, see also FIG. 37).

No clear enrichment was observed for the different linkers in the prioritized VP2 positions, but a general preference for small linkers (G4Sx1, APSx2 and APSx3) could be seen.

### Example 10.3: In-Vitro Validation of Identified Single VP2 N-Terminal Variants

### Introduction

In vitro selection of the LTM library aimed to increase N-terminal VP2-exposure resulted in the identification of nine single point mutations (as described in section 10.2). Each point mutation was cloned into the backbone NC070_pRS5a_AAV1 Sil1 VP2_(G4S)×3_anti-EGFR Sso7d, preserving the anti-EGFR Sso7d E18.4.5 sequence but replacing the (G4S)×3 linker with either (APS)×2, (APS)×3 or (G4S)×2 (see Table 44). Cloning and upscaling of the VP2 plasmids was outsourced to GeneArt. AAV samples were produced and their infectivity potential in EGFR OE and KO cells was evaluated. Binding to the Sso7d target (EGFR) was assessed as well to study the effect of the mutations on this aspect.

TABLE 44
List of VP2 expression plasmids with identified point mutations

| | Point |
|---|---|
| | muta- |
| Plasmid ID | tion |
| (HS0035)_pRS5a_anti- | N172V |
| EGFR_Sso7d_E18.4.5_(APS)x2_AAV1Sil1_N172V_VP2 | |
| (HS0036)_pRS5a_anti- | E147S |
| EGFR_Sso7d_E18.4.5_(APS)x2_AAV1Sil1_E147S_VP2 | |
| (HS0037)_pRS5a_anti- | P166R |
| EGFR_Sso7d_E18.4.5_(APS)x3_AAV1Sil1_P166R_VP2 | |
| (HS0038)_pRS5a_anti- | P185G |
| EGFR_Sso7d_E18.4.5_(APS)x3_AAV1Sil1_P185G_VP2 | |
| (HS0039)_pRS5a_anti- | G199R |
| EGFR_Sso7d_E18.4.5_(APS)x3_AAV1Sil1_G199R_VP2 | |
| (HS0040)_pRS5a_anti- | M211V |
| EGFR_Sso7d_E18.4.5_(APS)x3_AAV1Sil1_M211V_VP2 | |
| (HS0041)_pRS5a_anti- | D213A |
| EGFR_Sso7d_E18.4.5_(APS)x3_AAV1Sil1_D213A_VP2 | |
| (HS0042)_pRS5a_anti- | P191N |
| EGFR_Sso7d_E18.4.5_(G4S)x2_AAV1Sil1_P191N_VP2 | |
| (HS0043)_pRS5a_anti- | T162R |
| EGFR_Sso7d_E18.4.5_(APS)x3_AAV1Sil1_T162R_VP2 | |

### Decorated AAV1-Sil1 Production:

Plasmid DNA was mixed at equimolar ratio of pHelper, (AgC1294) 173CMV-EGFP-HPRE single stranded cargo, (NC080) pAAV Rep2Cap1 Sil1 VP2 KO, and the AAV1 VP2 expression plasmid from the above list. 200 ml Expi293F cells at a cell density of 2E+06 cells/ml were transfected (1.1 μg total DNA/1E+06 cells) using the FectoVIR-AAV Transfection reagent (Polyplus #101000022). The ratio of DNA:FectoVIR was 1 μg: 1 μl. An Sso7d decorated control with unmodified VP2 was also included at the transfection step. Glucose was added to the cell suspension to a final concentration of 2 g/l post transfection and the mixture was incubated at 37° C., 110 rpm, 6% CO2. Three days post transfection, benzonase in lysis buffer (500 mM HEPES, 10% Tween 20, 20 mM MgCl₂) was added to the transfected cell suspensions to a final concentration of 0.1 U/μl and a volume equal to 10% of the total volume. Following a 3 h incubation period at 37° C., a volume of sucrose salt solution (5 M NaCl, 7% sucrose) equal to 10% of the total volume was added to the lysate and the mixtures were incubated at 37° C. in agitation for 3 h. After this incubation step, a volume equal to 10% of the total volume in salt sucrose solution (5 M NaCl, 7% sucrose) was added and incubated for an additional 20 min. Samples were centrifuged for 15 min at 3500 g. The supernatant was filtered through a 0.22 μM filter and supplemented with EDTA to a final concentration of 5 mM. Next, the clarified lysates were loaded at 1 ml/min on an AAVX Pre-packed Column, 0.5×5 cm, 1 ml (ThermoFisher #A36652) mounted on an AKTA Pure instrument. Bound AAV particles were washed with 10 CV wash buffer, pH 7.3 (50 mM Tris-Cl, 0.5 M NaCl, 0.01% Pluronic F-68) and eluted with 5 CV glycine elution buffer, pH 2.7 (0.1 M Glycine, 0.2 M NaCl, 0.25 M L-arginine). Elution fractions (0.25 ml) were immediately neutralized with 25 μl 1 M Tris-Cl, pH 10. Fractions containing AAV (as assessed by UV280 signal) were pooled and dialyzed with the Float-A-Lyzer Dialysis Device 100 KD (Spectrum™ #G235071) over night against formulation buffer (20 mM Tris base, 1 mM MgCl₂, 200 mM NaCl, 0.005% Pluronic F-68, pH 8.1) and then passed through a 0.22 μm filter unit.

### Titer and Decoration Level Determination:

AAV vector titers were determined with the QX200 droplet digital PCR (ddPCR) system (Bio-Rad) using the SV40 pA specific oligo set described in Table 3. Pretreatment of purified AAV samples consisted of incubating 5 μl AAV with 1 unit of DNase and 1× DNase buffer (Merck #04716728001) in 20 μl total volume for 30 min at 37° C. Next, 1 μl Proteinase K at 20 mg/ml (Life technologies #E00491) was added to the reaction mix and incubated for 1 h at 55° C., followed by an inactivation step at 95° C. for 15 min. Different 1/10 serial dilutions of treated samples were prepared in 1×PCR Buffer II (Life technologies #N8080010), 2.5 mM MgCl2, 0.05% Pluronic F-68 (Sigma Aldrich #K4894), 2 ng/μl Sheared Salmon Sperm DNA (Invitrogen #15632011) and used as template for PCR amplification. The ddPCR reaction mix was prepared by combining 5.5 μl pre-treated AAV dilution with 900 nM forward and reverse SV40 pA primers each, 125 nM SV40 pA probe and 11 μl 2×ddPCR supermix for probes (BioRad #1863024), in a final volume of 22 μl. Technical duplicates were included for each sample. The ddPCR reaction mix was loaded in a semi-skirted 96-well plate (Bio-Rad #12001925) and placed in the automated droplet generator (Bio-Rad #1864101). Droplets were automatically generated using Bio-Rad's proprietary DG32 Automated Droplet Generator Cartridges (#1864108) and Automated Droplet Generation Oil for Probes (Bio-Rad #1864110). Following droplet generation, the device automatically deposited the generated droplets in a fresh semi-skirted 96-well plate.

The plate was heat-sealed with pierceable foil and placed into the C1000 Touch Thermal Cycler (Bio-Rad). Thermal cycling conditions were as follows: 95° C. for 10 min, then 40 cycles of 95° C. for 30 s and 60° C. for 1 min followed by a 10 min incubation at 98° C. and an infinite hold at 4° C. The FAM fluorescent signal, labeling the AAV genome DNA sequence in each droplet, was counted using a QX200 digital droplet reader and analyzed by QuantaSoft analysis software (Bio-Rad). For the calculation of AAV titers, the number of droplets was multiplied with the respective total dilution factor and then multiplied with a factor of 2 to consider possible reannealing of single stranded genomes.

CE-SDS analysis (Agilent 2100 Bioanalyzer system, Protein 230 kit using denaturing conditions) was performed to evaluate the VP distribution in the purified samples and the total VP yield.

Calculation of the total AAV particles/ml (vp/ml) and the VP ratio:
ng/μl (=μg/ml) values are divided by the observed MW and then multiplied by 1E+06 (μg to g conversion) to calculate moles/ml values
moles/ml values are multiplied by 6.022E+23 (Avogadro nr) to calculate the number of molecules/ml
VP distribution: calculated based on molecules/ml values assuming one AAV is made of 60 VPs in total
AAV particles/ml (vp/ml): Sum of VP1+VP2+VP3 molecules ml are divided by 60

The ddPCR and CE-SDS results are reported in Table 45.

TABLE 45
QC on decorated ssAAV1Sil1

| | VP2 fusion | | |
|---|---|---|---|
| Sample ID | per AAV | vg/cell | vp/cell |
| (AAV-637) ssAAV1Sil1_anti-EGFR Sso7d | 7.1 | 2.92E+04 | 5.41E+04 |
| E18.4.5_(G4S)x3- | | | |
| VP2_173CMVp_eGFP_HRPE | | | |
| (AAV-674) ssAAV1-Sil1_N172V_anti-EGFR | 8.3 | 1.31E+04 | 9.84E+04 |
| Sso7d E18.4.5_(APS)x2- | | | |
| VP2_173CMVp_eGFP_HRPE | | | |
| (AAV-639) ssAAV1-Sil1_E147S_anti-EGFR | 9.3 | 3.73E+04 | 6.34E+04 |
| Sso7d E18.4.5_(APS)x2- | | | |
| VP2_173CMVp_eGFP_HRPE | | | |
| (AAV-640) ssAAV1-Sil1_P166R_anti-EGFR | 9.0 | 3.14E+04 | 9.80E+04 |
| Sso7d E18.4.5_(APS)x3- | | | |
| VP2_173CMVp_eGFP_HRPE | | | |
| (AAV-641) ssAAV1-Sil1_P185G_anti-EGFR | 7.1 | 3.33E+04 | 9.40E+04 |
| Sso7d E18.4.5_(APS)x3- | | | |
| VP2_173CMVp_eGFP_HRPE | | | |
| (AAV-647) ssAAV1-Sil1_G199R_anti-EGFR | 13.8 | 2.36E+04 | 1.36E+05 |
| Sso7d E18.4.5_(APS)x3- | | | |
| VP2_173CMV_eGFP_HRPE | | | |
| (AAV-648) ssAAV1-Sil1_M211V_anti-EGFR | 11.0 | 1.58E+04 | 6.05E+04 |
| Sso7d E18.4.5_(APS)x3- | | | |
| VP2_173CMVp_eGFP_HRPE | | | |
| (AAV-649) ssAAV1-Sil1_D213A_anti-EGFR | 13.8 | 1.99E+04 | 7.43E+04 |
| Sso7d E18.4.5_(APS)x3- | | | |
| VP2_173CMVp_eGFP_HRPE | | | |
| (AAV-650) ssAAV1-Sil1_P191N_anti-EGFR | 12.7 | 1.90E+04 | 7.99E+04 |
| Sso7d E18.4.5_(G4S)x2- | | | |
| VP2_173CMVp_eGFP_HRPE | | | |
| (AAV-651) ssAAV1-Sil1_T162R_anti-EGFR | 12.2 | 1.83E+04 | 9.96E+04 |
| Sso7d E18.4.5_(APS)x3- | | | |
| VP2_173CMVp_eGFP_HRPE | | | |

### In Vitro Infectivity of Decorated AAV1Sil1 on Receptor Overexpressing and Knock-Out Cell Lines:

To assess whether the single mutations improved the decorated capsid's ability to infect cells, infectivity assays on EGFR overexpressing (OE) and knockout (KO) cells were performed. Cells at a concentration of 6.06E+05/mL in V3 medium were seeded in a flatbottom 96-well plate (33 μL cell suspension/well). Decorated AAVs were diluted in V3 medium and cells were infected at an MOI ranging from 1E+06 to 4.6E+01. With the final volume of 100 μl in the wells, the plate was incubated at 37° C. for 3 h, followed by addition of 100 μl V3 medium with 10% FBS. Three days post-transfection the cells were detached and eGFP fluorescence was measured by FACS. The EC50 value was calculated based on the percentage of eGFP positive cells for each AAV sample in both the overexpressing and the knockout cells. Next, the EC50 values from the EGFR KO conditions were divided by the corresponding EC50 values from the EGFR OE conditions. In FIG. 38 the fold change resulting from the aforementioned calculation is depicted.

### Assessing the Effect of VP2 Point Mutations on Target Binding by ELISA:

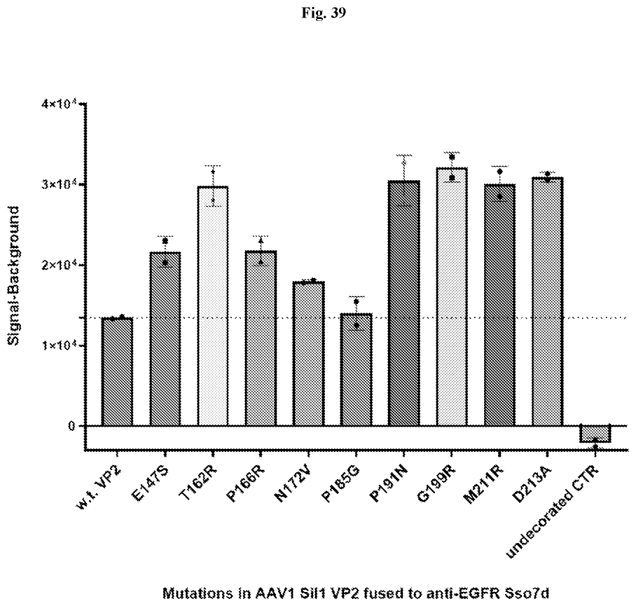

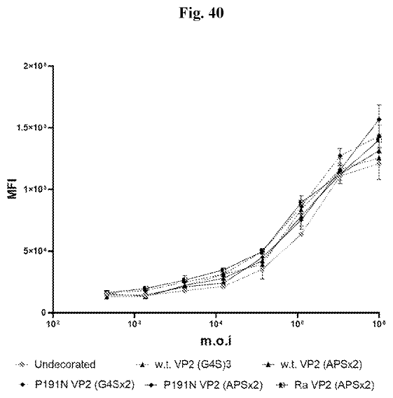

A black Maxisorp 384-well plate (Merck #P6366-1CS) was coated with hEGFR extracellular domain recombinant protein at 5 μg/mL or with BSA. Following overnight incubation at 4° C. the plate was washed with TBST buffer (TBS+0.05% Tween20) and blocked with 3% BSA/TBST buffer. In the next step, serial dilutions of decorated AAVs and an undecorated AAV1Sil1 control, produced in unrelated production rounds, were added onto the coated plate. After a 2 h incubation step at room temperature, the plate was washed three times with TBST and then incubated with an anti-AAVx biotinylated antibody (ThermoFisher Scientific #7103522100) for 1 h at room temperature. The plate was washed with TBST (4×) and incubated for 30 min at room temperature with a streptavidin-AP conjugate (Roche #11089161001). The plate was washed again four times and AttoPhos substrate (Roche #00000011681982001) was added to each well. The BSA plate signal was subtracted from the EGFR plate signal. The results are shown in FIG. 39 focusing on the 5E+11 vp AAV concentration.

Enhancement of transduction on EGFR over-expressing cells and protein binding can be observed with most of the prioritized single VP2 mutants. Without wishing to be bound by theory it is hypothesized that this improvement is linked to a better Sso7d exposure and/or potentially to the slightly higher amount of Sso7d fusion observed for these samples.

### Example 10.4 Combined VP2 N-Terminal Variants In Vitro Confirmation

### Introduction

In the previous section VP2 M-terminus mutations P191N, T162R and D213A were identified as top candidates. To examine whether these VP2 point mutations have an additive effect on the capsid's transduction potency, a new VP2 plasmid was designed containing all three mutations. String DNA encoding for the mutations described in Table 46 were cloned into the backbone NC070_pRS5a_AAV1 Sil1 VP2_(G4S)×3_anti-EGFR Sso7d, preserving the anti-EGFR Sso7d E18.4.5 sequence but replacing the (G4S)×3 linker with either (APS)×2, (APS)×3 or (G4S)×2 (see Table 46). Additionally, the effect of the linker on the infectivity and on target binding was also investigated, comparing the flexible (G4S)×2 and (G4S)×3 linkers with the more rigid (APS)×2. AAV samples were produced and their infectivity potential in EGFR OE and KO cells was evaluated. Binding to the Sso7d target (EGFR) was assessed as well to study the effect of the mutations on this aspect.

TABLE 46
List of VP2 expression plasmids with respective point mutations and linker

| | VP2 | |
|---|---|---|
| Plasmid ID | mutation | Linker |
| (NC079) pAAV_rep2cap1_AAV1-Sil1[V473D, N500E]_KanR | none | No Sso7d |
| (NC070) pRS5a_AAV1 Sil1 VP2_(G4S)x3_anti-EGFR Sso7d | none | (G4S)x3 |
| (SK0050)_pRS5a_anti- | none | (APS)x2 |
| EGFR_Sso7d_E18.4.5_(APS)x2_AAV1_Sil1_VP2 | | |
| (SK0058)_pRS5a_anti- | P191N | (APS)×2 |
| EGFR_Sso7d_E18.4.5_(APS)x2_AAV1Sil1_VP2_P191N | | |
| (SK0051)_pRS5a_anti- | P191N, | (APS)×2 |
| EGFR_Sso7d_E18.4.5_(APS)x2_AAV1_Sil1_Ra_VP2 | T162R, | |
| | D213A | |
| (HS0042)_pRS5a_anti- | P191N | (G4S)×2 |
| EGFR_Sso7d_E18.4.5_(G4S)x2_AAV1Sil1_P191N_VP2 | | |

### Decorated AAV1-Sil1 Production:

Plasmid DNA was mixed at equimolar ratio of pHelper, (AgC1294) 173CMV-EGFP-HPRE single stranded cargo, (NC080) pAAV Rep2Cap1 Sil1 VP2 KO, and the AAV1 VP2 expression plasmid from the above list. For the undecorated AAV control the following plasmids were used: pHelper, (AgC1294) 173CMV-EGFP-HPRE single stranded cargo and (NC079) pAAV_rep2cap1_AAV1-Sil1[V473D,N500E]_KanR. Expi293F cells were resuspended in fresh medium at a cell density of 2E+06 cells/ml and transfected (1.1 μg total DNA/1E+06 cells) using the FectoVIR-AAV Transfection reagent (Polyplus #101000022). The ratio of DNA:FectoVIR was 1 μg:1 μl. Shortly after transfection glucose was added to the cell suspension to a final concentration of 2 g/l and the mixture was incubated at 37° C., 110 rpm, 6% CO2. Three days post transfection, benzonase in lysis buffer (500 mM HEPES, 10% Tween 20, 20 mM MgCl₂) was added to the transfected cell suspensions to a final concentration of 0.1 U/μl and a volume equal to 10% of the total volume. Following a 3 h incubation period at 37° C., a volume of sucrose salt solution (5 M NaCl, 7% sucrose) equal to 10% of the total volume was added to the lysate and the mixtures were incubated at 37° C. in agitation for 3 h. After this incubation step, a volume equal to 10% of the total volume in salt sucrose solution (5 M NaCl, 7% sucrose) was added and incubated for an additional 20 min. Samples were centrifuged for 15 min at 3500 g. The supernatant was filtered through a 0.22 μM filter and supplemented with EDTA to a final concentration of 5 mM. Next, the clarified lysates were loaded at 1 ml/min on an AAVX Pre-packed Column, 0.5×5 cm, 1 ml (ThermoFisher #A36652) mounted on an AKTA Pure instrument. Bound AAV particles were washed with 10 CV wash buffer, pH 7.3 (50 mM Tris-Cl, 0.5 M NaCl, 0.01% Pluronic F-68) and eluted with 5 CV glycine elution buffer, pH 2.7 (0.1 M Glycine, 0.2 M NaCl, 0.25 M L-arginine). Elution fractions (0.25 ml) were immediately neutralized with 25 μl 1 M Tris-Cl, pH 10. Fractions containing AAV (as assessed by UV280 signal) were pooled and dialyzed with the Float-A-Lyzer Dialysis Device 100 KD (Spectrum™ #G235071), over night against formulation buffer (20 mM Tris base, 1 mM MgCl₂, 200 mM NaCl, 0.005% Pluronic F-68, pH 8.1) and then passed through a 0.22 μm filter unit.

### Titer and Decoration Level Determination:

AAV vector titers were determined with the QX200 droplet digital PCR (ddPCR) system (Bio-Rad) using the SV40 pA specific oligo set described in Table 3. Pretreatment of purified AAV samples consisted of incubating 5 μl of AAV with 1 unit of DNase and 1× DNase buffer (Merck #04716728001) in 20 μl total volume for 30 min at 37° C. Next, 1 μl Proteinase K at 20 mg/ml (Life technologies #E00491) was added to the reaction mix and the mix was incubated for 1 h at 55° C., followed by an inactivation step at 95° C. for 15 min. Different 1/10 serial dilutions of treated samples were prepared in 1×PCR Buffer II (Life technologies #N8080010), 2.5 mM MgCl2, 0.05% Pluronic F-68 (Sigma Aldrich #K4894), 2 ng/μl Sheared Salmon Sperm DNA (Invitrogen #15632011) and used as template for PCR amplification. The ddPCR reaction mix was prepared by combining 5.5 μl pre-treated AAV dilution with 900 nM forward and reverse SV40 pA primers each, 125 nM SV40 pA probe and 11 μl 2× ddPCR supermix for probes (BioRad #1863024), in a final volume of 22 μl. Technical duplicates were included for each sample. The ddPCR reaction mix was loaded in a semi-skirted 96-well plate (Bio-Rad #12001925) and placed in the automated droplet generator (Bio-Rad #1864101). Droplets were automatically generated using Bio-Rad's proprietary DG32 Automated Droplet Generator Cartridges (#1864108) and Automated Droplet Generation Oil for Probes (Bio-Rad #1864110). Following droplet generation, the device automatically deposited the generated droplets in a fresh semi-skirted 96-well plate.

The plate was heat-sealed with pierceable foil and placed into the C1000 Touch Thermal Cycler (Bio-Rad). Thermal cycling conditions were as follows: 95° C. for 10 min, then 40 cycles of 95° C. for 30 s and 60° C. for 1 min followed by a 10 min incubation at 98° C. and an indefinite hold at 4° C. The FAM fluorescent signal, labeling the AAV genome DNA sequence in each droplet, was counted using a QX200 digital droplet reader and analyzed by QuantaSoft analysis software (Bio-Rad). For calculation of the AAV titers, the number of droplets was multiplied with the respective total dilution factor and then multiplied with a factor of 2 to consider possible reannealing of single stranded genomes.

CE-SDS analysis (Agilent 2100 Bioanalyzer system, Protein 230 kit using denaturing conditions) was performed to evaluate the VP distribution in the purified samples and the total VP yield.

Calculation of the total AAV particles/ml (vp/ml) and the VP ratio:
ng/μl (=μg/ml) values are divided by the observed MW and then multiplied by 1E+06 (μg to g conversion) to calculate moles/ml values
moles/ml values are multiplied by 6.022E+23 (Avogadro nr) to calculate the number of molecules/ml
VP distribution: calculated based on molecules/ml values assuming one AAV is made of 60 VPs in total
AAV particles/ml (vp/ml): Sum of VP1+VP2+VP3 molecules ml are divided by 60

The ddPCR and CE-SDS results are reported in Table 47.

TABLE 47
QC on decorated ssAAV1Sil1

| | | VP2 | | |
|---|---|---|---|---|
| | | fusion | | |
| Sample ID | Short ID | per AAV | vg/cell | vp/cell |
| (PPB-26864) ssAAV1_Sil1-173CMV-eGFP | undecorated | 0 | 2.71E+04 | 4.93E+04 |
| (PPB-26865) ssAAV1_Sil1-173CMV-eGFP- | w.t. VP2 | 6.9 | 1.80E+04 | 4.48E+04 |
| anti-EGFR-Sso7d_E18.4.5_VP2_(G4S)3 | (G4Sx3) | | | |
| (PPB-26866)_ssAAV1_Sil1-173CMV- | w.t. VP2 | 8.2 | 1.77E+04 | 4.82E+04 |
| eGFP-anti-EGFR- | (APSx2) | | | |
| Sso7d_E18.4.5_VP2_(APS)2 | | | | |
| (PPB-26867)_ssAAV1_Sil1-173CMV- | P191N VP2 | 9.5 | 1.46E+04 | 4.23E+04 |
| eGFP-anti-EGFR- | (APSx2) | | | |
| Sso7d_E18.4.5_VP2_P191N_(APS)2 | | | | |
| (PPB-26868)_ssAAV1_Sil1-173CMV- | P191N VP2 | 9.7 | 1.59E+04 | 4.05E+04 |
| eGFP-anti-EGFR- | (G4Sx2) | | | |
| Sso7d_E18.4.5_VP2_P191N_(G4S)2 | | | | |
| (PPB-26869)_ssAAV1_Sil1-173CMV- | Ra VP2 | 10.8 | 2.04E+04 | 5.21E+04 |
| eGFP-NOX-anti-EGFR- | (APS×2) | | | |
| Sso7d_E18.4.5_VP2_RA_(APS)2 | | | | |

### In Vitro Infectivity of Decorated AAV1Sil1 on Receptor Overexpressing and Knock-Out Cell Lines:

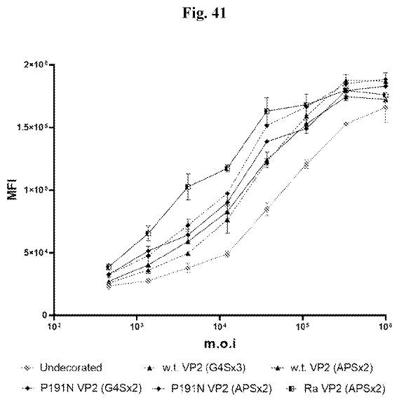

To assess whether the single mutations improved the decorated capsid's ability to infect cells, infectivity assays on EGFR overexpressing (OE) and knockout (KO) cells were performed. Cells at a concentration of 6.06E+05/mL in V3 medium were seeded in a flatbottom 96-well plate (33 μL cell suspension/well). Decorated AAVs were diluted in V3 medium and cells were infected at an MOI ranging from 1E+06 to 4.6E+01. With 100 μl final volume in the wells, the plate was incubated at 37° C. for 3 h, followed by addition of 100 μL V3 medium with 10% FBS. Three days post-transfection the cells were detached and the eGFP fluorescence was measured by FACS. Due to the high transduction level observed in this experiment, the analysis was conducted at the MFI level as shown in FIG. 40 and FIG. 41.

The combined mutations on the VP2 sequence (P191N, T162R, D213A=Ra) improve the transduction efficiency of anti-EGFR Sso7d targeted AAV1Sil1 compared to both w.t. VP2 and the single P191N mutant. No clear effect of the different linkers used on the transduction could be observed.

1. An adeno-associated virus (AAV) VP2 fusion polypeptide comprising an AAV VP2 capsid polypeptide and a polypeptide ligand, wherein the polypeptide ligand is fused to the N-terminus of the AAV VP2 capsid polypeptide and wherein the polypeptide ligand is selected from the group consisting of a GP2 polypeptide, an Sso7d polypeptide and an affibody.

2. The AAV VP2 fusion polypeptide of claim 1, wherein said polypeptide ligand is selected from the group consisting of an Sso7d polypeptide of SEQ ID NO: 1 and an Sso7d polypeptide having at least 80%, 85%, 90%, or 95% sequence identity therewith.

3. The AAV VP2 fusion polypeptide of claim 2, wherein said polypeptide ligand is selected from the group consisting of an Sso7d polypeptide of SEQ ID NO: 1, in which the amino acid residues X at positions 21, 23, 25, 28, 30, 32, 40, 42 and 44 are independently selected from D, R, H, N, A, I, Y and W, and an Sso7d polypeptide having at least 80%, 85%, 90%, or 95% sequence identity therewith.

4. The AAV VP2 fusion polypeptide of claim 1, further comprising a peptide linker between the polypeptide ligand and the AAV VP2 capsid polypeptide, wherein said peptide linker is selected from the group consisting of a glycine-serine (GS) linker and an alanine-proline-serine (APS) linker.

5. The AAV VP2 fusion polypeptide of claim 1, wherein said AAV VP2 capsid polypeptide is of an AAV serotype selected from the group consisting of AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV and avian AAV.5.

6. The AAV VP2 fusion polypeptide of claim 5, wherein the AAV VP2 capsid polypeptide comprises at least one mutation in at least one binding site for its natural receptor, wherein:
a) the AAV VP2 fusion polypeptide is of the AAV serotype AAV6 and comprises at least one amino acid substitution within its VP3 region selected from the group consisting of K531E, V473D, K459S, N500E, G266A, N269Q, and D590A relative to the VP1 amino acid sequence of SEQ ID NO: 3, or any combination thereof;
b) the AAV VP2 fusion polypeptide is of the AAV serotype AAV8 and comprises at least one amino acid substitution within its VP3 region selected from the group consisting of G268E, N271Q, S387A, A592Q and A592D relative to the VP1 amino acid sequence of SEQ ID NO: 4, or any combination thereof;
c) the AAV VP2 fusion polypeptide is of the AAV serotype AAV9 and comprises at least one amino acid substitution within its VP3 region selected from the group consisting of Q590A, W503A, N562A and E563A relative to the VP1 amino acid sequence of SEQ ID NO: 5, or any combination thereof;
d) the AAV VP2 fusion polypeptide is of the AAV serotype AAV2 and comprises the amino acid substitution R585A relative to the VP1 amino acid sequence of SEQ ID NO: 6 within its VP3 region; or
e) the AAV VP2 fusion polypeptide is of the AAV serotype AAV1 and comprises at least one amino acid substitution within its VP3 region selected from the group consisting of V473D, N500E and R514A relative to the VP1 amino acid sequence of SEQ ID NO: 7, or any combination thereof.

7. The AAV VP2 fusion polypeptide of claim 1, wherein a) the AAV VP2 fusion polypeptide is of the AAV serotype AAV1 and comprises at least one amino acid substitution selected from the group consisting of E147S, P185G, P166R, M211 V, G199R, D213A, T162R, and P191N relative to the VP1 amino acid sequence of SEQ ID NO: 7, or any combination thereof, or wherein b) the AAV VP2 fusion polypeptide is of an AAV serotype other than AAV1 and comprises at least one amino acid substitution corresponding to at least one of the amino acid substitution selected from the group consisting of E147S, P185G, P166R, M211V, G199R, D213A, T162R, and P191N relative to the VP1 amino acid sequence of SEQ ID NO: 7.

8. The AAV VP2 fusion polypeptide of claim 7, wherein the AAV VP2 fusion polypeptide further comprises the amino acid substitutions V473D and N500E relative to the VP1 amino acid sequence of SEQ ID NO: 7.

9. An isolated nucleic acid encoding the AAV VP2 fusion polypeptide of claim 1.

10. A cell comprising the AAV VP2 fusion polypeptide of claim 1, wherein the cell is selected from the group consisting of an insect cell and a HEK293 cell.

11. A recombinant AAV (rAAV) virion comprising the AAV VP2 fusion polypeptide of claim 1.

12. The rAAV virion of claim 11, further comprising a nucleic acid sequence encoding the AAV VP2 fusion polypeptide of claim 1, wherein the AAV VP2 fusion polypeptide comprised in the rAAV virion and the AAV VP2 fusion polypeptide encoded by said nucleic acid sequence are identical.

13. The rAAV virion of claim 11, further comprising a nucleic acid sequence encoding a therapeutic nucleic acid, a therapeutic protein, or a therapeutic antibody or antibody fragment.

14. The rAAV virion of claim 11, wherein the virion further comprises AAV VP1 and VP3 polypeptides, wherein the AAV VP2 fusion polypeptide and said AAV VP1 and VP3 polypeptides are of the same AAV serotype selected from the group consisting of AAV1, AAV2, AAV3, AAV4, AAV5, AAV6, AAV7, AAV8, AAV9, AAV10, AAV11, AAV12, AAV13, AAVrh.8, AAVrh.10, AAVrh.32.33, bovine AAV and avian AAV.5.

15. The rAAV virion of claim 14, wherein the AAV VP1, VP2 and VP3 polypeptides comprise at least one mutation in at least one binding site for the natural receptor of the AAV VP1, VP2 and VP3 capsid polypeptides, wherein the AAV VP1, VP2 and VP3 polypeptides comprise the same at least one mutation in the shared VP3 region of each AAV capsid polypeptide, wherein:
a) the AAV VP2 fusion polypeptide and said AAV VP1 and VP3 polypeptides are of the AAV serotype AAV6 and each comprise at least one amino acid substitution within the shared VP3 region selected from the group consisting of K531E, V473D, K459S, N500E, G266A, N269Q, and D590A relative to the VP1 amino acid sequence of SEQ ID NO: 3, or any combination thereof;
b) the AAV VP2 fusion polypeptide and said AAV VP1 and VP3 polypeptides are of the AAV serotype AAV8 and each comprise at least one amino acid substitution within the shared VP3 region selected from the group consisting of G268E, N271Q, S387A, A592Q and A592D relative to the VP1 amino acid sequence of SEQ ID NO: 4, or any combination thereof;
c) the AAV VP2 fusion polypeptide and said AAV VP1 and VP3 polypeptides are of the AAV serotype AAV9 and each comprise at least one amino acid substitution within the shared VP3 region selected from the group consisting of Q590A, W503A, N562A and E563A relative to the VP1 amino acid sequence of SEQ ID NO: 5, or any combination thereof;
d) the AAV VP2 fusion polypeptide and said AAV VP1 and VP3 polypeptides are of the AAV serotype AAV2 and each comprise the amino acid substitution R585A relative to the VP1 amino acid sequence of SEQ ID NO: 6 within the shared VP3 region; or
e) the AAV VP2 fusion polypeptide and said AAV VP1 and VP3 polypeptides are of the AAV serotype AAV1 and each comprise at least one amino acid substitution within the shared VP3 region selected from the group consisting of V473D, N500E and R514A relative to the VP1 amino acid sequence of SEQ ID NO: 7, or any combination thereof.

16. A pharmaceutical composition comprising the rAAV virion of claim 11 and a pharmaceutically acceptable excipient.

17. (canceled)

18. A library construct comprising a nucleic acid sequence encoding an AAV VP2 fusion polypeptide comprising an AAV VP2 capsid polypeptide and a polypeptide ligand, wherein the polypeptide ligand is fused to the N-terminus of the AAV VP2 capsid polypeptide and wherein said polypeptide ligand is selected from the group consisting of a GP2 polypeptide, an Sso7d polypeptide and an affibody.

19. The library construct of claim 18, wherein said polypeptide ligand is selected from the group consisting of an Sso7d polypeptide of SEQ ID NO: 1, in which the amino acid residues X at positions 21, 23, 25, 28, 30, 32, 40, 42 and 44 are independently selected from D, R, H, N, A, I, Y and W, and an Sso7d polypeptide having at least 80%, 85%, 90%, or 95% sequence identity therewith.

20. A library comprising a plurality of library constructs of claim 18, wherein the library comprises at least 10², 10³, 10⁴, 10⁵, 10⁶, 10⁷, 10⁸, or 10⁹ unique library constructs.

21. The library of claim 20, wherein each library construct is present within an rAAV virion, wherein said rAAV virion comprises an AAV VP2 fusion polypeptide of claim 2 or 3 and wherein the AAV VP2 fusion polypeptide comprised in a given rAAV virion and the AAV VP2 fusion polypeptide encoded by the library construct present in said given rAAV virion are identical.

22. A method of generating an AAV VP2 fusion polypeptide with at least one desired characteristic, the method comprising the steps: a) contacting the library of claim 20 with a plurality of cells, b) isolating nucleic acid molecules from at least a part of said cells, and c) determining at least a part of the sequence encoding the polypeptide ligand or a fragment thereof of at least one nucleic acid molecule isolated in step a), wherein the plurality of cells in step a is present within a non-human model animal.

23. (canceled)

24. An AAV VP2 fusion polypeptide comprising an AAV VP2 capsid polypeptide and a polypeptide ligand, wherein the polypeptide ligand is fused to the N-terminus of the AAV VP2 capsid polypeptide, wherein the polypeptide ligand has a molecular weight of up to 10 kDa, and wherein the AAV VP2 capsid polypeptide comprises one or more mutations that abolish or reduce binding to Heparan Sulphate Proteoglycan (HSPG) and/or Sialic Acid (SIA).

25. An AAV VP2 capsid polypeptide, wherein a) the AAV VP2 capsid polypeptide is of the AAV serotype AAV1 and comprises at least one amino acid substitution selected from the group consisting of E147S, P185G, P166R, M211V, G199R, D213A, T162R, and P191N relative to the VP1 amino acid sequence of SEQ ID NO: 7, or any combination thereof.

26. An rAAV virion comprising the AAV VP2 capsid polypeptide of claim 25.


SEQUENCE LISTING (84 sequences)

SEQ ID NO: 1 - protein, 61 aa, synthetic construct
ATVKFTYQGEEKQVDISKIKXVXRXGQXIXFXYDEGGGAXGXGXVSEKDAPKELLQMLEKQ

SEQ ID NO: 2 - DNA, 6592 bp, synthetic construct
  source 1..6592: mol_type = other DNA
taaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagttggaccggtcccggggaactcctcccgagtcgaaattcgccaccatggctacagtgaagttcacataccagggcgaagagaaacaggtggacatcagcaagatcaagnnngtgnnnagannnggccagnnnattnnnttcnnntacgacgaaggcggcggagccnnnggannnggannngtgtccgaaaaggacgcccctaaagaactgctgcagatgctggaaaagcaaggcggcggaggatccgctcctggaaagaaaagacccgtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtggatctccagctggcatgtctgaccagcccaaaaactggctacctggaccctgttatcggcagcagcgcgtttctaaaacaaaaacagacaacaacaacagcgaatttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtgatgagaattcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcttaaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgtacatgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaagttccccggggtacctcccgagtcgaaattcgccaccatggaagaggacaacatggccatcatcaaagagttcatgcggttcaaggtgcacatggaaggctccgtgaacggccacgagttcgagattgaaggcgaaggcgagggccatccttatgagggaacacagaccgccaagctgaaagtgaccaaaggcggccctctgccttttgcctgggacatcctgtctccacagtttatgtacggctccaaggcctacgtgaagcaccccgctgacatccccgattacctgaagctgtctttcccagagggcttcacctgggagagagtgatgaacttcgaggacggcggcgtggtcacagtgacccaagatagctctctgcaggacggccagttcatctacaaagtgaagctgctgggcatcaacttccccagcgacggccctgtgatgcagaaaaagaccatgggctgggaagcctccaccgagagaatgtaccctgaggatggcgctctgaagggcgagatcaaccagcggctgaagctgaaggatggcggccactacgacgccgaagtgaaaaccacctacaaggccaagaaacccgtgcagctgcctggcgcctacaacgtggatatcaagctggacatcaccagccacaacgaggactacaccatcgtggaacagtacgagagagccgaggccagacactctacatgataaaagcttctagctttatttgtgaaatttgtgatgctattgctttatttgtaaccattataagctgcaataaacaagttaacaacaacaattgcattcattttatgtttcaggttcagggggagatgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgttaataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatcacatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccgga

SEQ ID NO: 3 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 4 - protein, 738 aa, Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 5 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 735 aa, Adeno-associated virus 2 [sequence omitted]

SEQ ID NO: 7 - protein, 736 aa, Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 8 - DNA, 8318 bp, synthetic construct
  source 1..8318: mol_type = other DNA
catacgatttaggtgacactatagaataacatccactttgcctttctctccacaggtgtccactcccaggtccaactgcacggaagcttacgcgtgccgccaccatggctacagtgaagttcacataccagggcgaagagaaacaggtggacatcagcaagatcaagnnngtgnnnagannnggccagnnnattnnnttcnnntacgacgaaggcggcggagccnnnggannnggannngtgtccgaaaaggacgcccctaaagaactgctgcagatgctggaaaagcaaggcggcggaggatctggcggaggtggaagcggaggcggaggatctgctcctggaaagaaaagaccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgaccagcccaaaaactggctacctggaccctgttatcggcagcagcgcgtttctaaaacaaaaacagacaacaacaacagcgaatttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtgatgaggcgcgccgggcggccgctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaactagctcgaggcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaattttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgatcgacggatcgggagatccgggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgcggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcataca

SEQ ID NO: 9 - protein, 173 aa, synthetic construct
MRGSAWSHPQFEKGSDLGKKLLEAARAGQDDEVRILMANGADVNAHDFYGITPLHLAANFGHLEIVEVLLKHGADVNAFDYDNTPLHLAADAGHLEIVEVLLKYGADVNASDRDGHTPLHLAAREGHLEIVEVLLKNGADVNAQDKFGKTAFDISIDNGNEDLAEILQGGGGS

SEQ ID NO: 10 - protein, 173 aa, synthetic construct
MRGSAWSHPQFEKGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQGGGGS

SEQ ID NO: 11 - protein, 174 aa, synthetic construct
MRGSAWSHPQFEKGSDLGRKLLEAARAGQDDEVRILMANGADVNAADNTGTTPLHLAAYSGHLEIVEVLLKHGADVDASDVFGYTPLHLAAYWGHLEIVEVLLKNGADVNAMDSDGMTPLHLAAKWGYLEIVEVLLKHGADVNAQDKFGKTAFDISIDNGNEDLAEILQGGGGS

SEQ ID NO: 12 - protein, 60 aa, synthetic construct
MRGSAWSHPQFEKGSTSGVRATSKFAALIAAEIAREFGYTVDVQEKNGEWRVVFDGGGGS

SEQ ID NO: 13 - DNA, 25 bp, synthetic construct
  source 1..25: mol_type = other DNA
agcaatagcatcacaaatttcacaa

SEQ ID NO: 14 - DNA, 29 bp, synthetic construct
  source 1..29: mol_type = other DNA
ccagacatgataagatacattgatgagtt

SEQ ID NO: 15 - DNA, 36 bp, synthetic construct
  source 1..36: mol_type = other DNA
agcatttttttcactgcattctagttgtggtttgtc

SEQ ID NO: 16 - protein, 115 aa, synthetic construct
MVSDVPRDLEVVAATPTSLLISWFDYAVTYYRITYGETGGNSPVQEFTVPGWISTATISGLKPGVDYTITVYAVTDNSRWPFRSTPISTNYRTEIDKPPQGGGGSGGGGSGGGGS

SEQ ID NO: 17 - protein, 77 aa, synthetic construct
MATVKLTYQGEEKQVDISKITYVDRAGQFIWFEYDEGGGALGTGWVSEKDAPKELLQMLEKQGGGGSGGGGSGGGGS

SEQ ID NO: 18 - protein, 74 aa, synthetic construct
MVDNKFNKEMWAAWEEILHLPNLNGWQMTAFIASLVDDPSQSANLLAEAKKLNDAQAPKGGGGSGGGGSGGGGS

SEQ ID NO: 19 - protein, 63 aa, synthetic construct
MKFWATVSRGDSYWFEVPVYAETLDEALELAERQYPMYHIYYVTRVRPGGGGSGGGGSGGGGS

SEQ ID NO: 20 - protein, 71 aa, synthetic construct
MVDNKFNKEMRNAYWEIALLPNLNNQQKRAFIRSLYDDPSQSANLLAEAKKLNDAQAPKGGGSGGGSGGGS

SEQ ID NO: 21 - protein, 71 aa, synthetic construct
MVDNKFNKELVKAAAEIDALPNLNRRQWNAFIKSLVDDPSQSANLLAEAKKLNDAQAPKGGGSGGGSGGGS

SEQ ID NO: 22 - protein, 62 aa, synthetic construct
MKFWATVCSGHDGYCFEVPVYAETLDEALELAEWQYDSTYYDYAVTRVRPGGGSGGGSGGGS

SEQ ID NO: 23 - protein, 60 aa, synthetic construct
MKFWATVDCLYNDTAFEVPVYAETLDEALELAEWQYDPNYCIVTRVRPGGGSGGGSGGGS

SEQ ID NO: 24 - protein, 77 aa, synthetic construct
MATVKFTYQGEEKQVDISKIKRVDRYGQSIHFNYDEGGGAYGWGWVSEKDAPKELLQMLEKQGGGGSGGGGSGGGGS

SEQ ID NO: 25 - protein, 77 aa, synthetic construct
MATVKFTYQGEEKQVDISKIKNVLRYGQQIVFSYDEGGGAGGNGFVSEKDAPKELLQMLEKQGGGGSGGGGSGGGGS

SEQ ID NO: 26 - protein, 77 aa, synthetic construct
MATVKFTYQGEEKQVDISKIMYVIRGGQRIAFGYDEGDGAWGDGIVSEKDAPKELLQMLEKQGGGGSGGGGSGGGGS

SEQ ID NO: 27 - protein, 77 aa, synthetic construct
MATVKFTYQGEEKQVDISKIMYVIRAGQRIAFGYDEGDGAWGDGIVSEKDAPKELLQMLEKQGGGGSGGGGSGGGGS

SEQ ID NO: 28 - protein, 77 aa, synthetic construct
MATVKFTYQGEEKQVDISKIMYVIRGGQRIAFAYDEGDGAWGDGIVSEKDAPKELLQMLEKQGGGGSGGGGSGGGGS

SEQ ID NO: 29 - protein, 62 aa, synthetic construct
MATVKFTYQGEEKQVDISKIKXVXRXGQXIXFXYDEGGGAXGXGXVSEKDAPKELLQMLEKQ

SEQ ID NO: 30 - DNA, 82 bp, synthetic construct
  source 1..82: mol_type = other DNA
aatgatacggcgaccaccgagatctacactctttccctacacgacgctcttccgatctcaggtggacatcagcaagatcaag

SEQ ID NO: 31 - DNA, 35 bp, synthetic construct
  source 1..35: mol_type = other DNA
cccggggaactcctcccgagtcgaaattcgccacc

SEQ ID NO: 32 - DNA, 35 bp, synthetic construct
  source 1..35: mol_type = other DNA
gttgctggaggttctccgagaggttgtgggtcggg

SEQ ID NO: 33 - DNA, 27 bp, synthetic construct
  source 1..27: mol_type = other DNA
tgctttatttgtgaaatttgtgatgct

SEQ ID NO: 34 - DNA, 24 bp, synthetic construct
  source 1..24: mol_type = other DNA
ccctgaacctgaaacataaaatga

SEQ ID NO: 35 - DNA, 41 bp, synthetic construct
  source 1..41: mol_type = other DNA
tgtaaccattataagctgcaataaacaagttaacaacaaca

SEQ ID NO: 36 - DNA, 82 bp, synthetic construct; identical to SEQ ID NO: 30
  source 1..82: mol_type = other DNA

SEQ ID NO: 37 - DNA, 89 bp, synthetic construct
  source 1..89: mol_type = other DNA
caagcagaagacggcatacgagattggtcagtgactggagttcagacgtgtgctcttccgatctctttaggggcgtccttttcagacac

SEQ ID NO: 38 - DNA, 89 bp, synthetic construct
  source 1..89: mol_type = other DNA
caagcagaagacggcatacgagattcaagtgtgactggagttcagacgtgtgctcttccgatctctttaggggcgtccttttcagacac

SEQ ID NO: 39 - DNA, 89 bp, synthetic construct
  source 1..89: mol_type = other DNA
caagcagaagacggcatacgagatgtagccgtgactggagttcagacgtgtgctcttccgatctctttaggggcgtccttttcggacac

SEQ ID NO: 40 - DNA, 89 bp, synthetic construct
  source 1..89: mol_type = other DNA
caagcagaagacggcatacgagattacaaggtgactggagttcagacgtgtgctcttccgatctctttaggggcgtccttttcggacac

SEQ ID NO: 41 - DNA, 89 bp, synthetic construct
  source 1..89: mol_type = other DNA
caagcagaagacggcatacgagatttgactgtgactggagttcagacgtgtgctcttccgatctctttaggggcgtccttttcggacac

SEQ ID NO: 42 - DNA, 89 bp, synthetic construct
  source 1..89: mol_type = other DNA
caagcagaagacggcatacgagatggaactgtgactggagttcagacgtgtgctcttccgatctctttaggggcgtccttttcggacac

SEQ ID NO: 43 - DNA, 89 bp, synthetic construct
  source 1..89: mol_type = other DNA
caagcagaagacggcatacgagattgacatgtgactggagttcagacgtgtgctcttccgatctctttaggggcgtccttttcggacac

SEQ ID NO: 44 - DNA, 89 bp, synthetic construct
  source 1..89: mol_type = other DNA
caagcagaagacggcatacgagatggacgggtgactggagttcagacgtgtgctcttccgatctctttaggggcgtccttttcggacac

SEQ ID NO: 45 - DNA, 89 bp, synthetic construct
  source 1..89: mol_type = other DNA
caagcagaagacggcatacgagatgcggacgtgactggagttcagacgtgtgctcttccgatctctttaggggcgtccttttcggacac

SEQ ID NO: 46 - DNA, 89 bp, synthetic construct
  source 1..89: mol_type = other DNA
caagcagaagacggcatacgagattttcacgtgactggagttcagacgtgtgctcttccgatctctttaggggcgtccttttcggacac

SEQ ID NO: 47 - DNA, 89 bp, synthetic construct
  source 1..89: mol_type = other DNA
caagcagaagacggcatacgagatggccacgtgactggagttcagacgtgtgctcttccgatctctttaggggcgtccttttcggacac

SEQ ID NO: 48 - DNA, 89 bp, synthetic construct
  source 1..89: mol_type = other DNA
caagcagaagacggcatacgagatcgaaacgtgactggagttcagacgtgtgctcttccgatctctttaggggcgtccttttcggacac

SEQ ID NO: 49 - DNA, 89 bp, synthetic construct
  source 1..89: mol_type = other DNA
caagcagaagacggcatacgagatcgtacggtgactggagttcagacgtgtgctcttccgatctctttaggggcgtccttttcggacac

SEQ ID NO: 50 - DNA, 89 bp, synthetic construct
  source 1..89: mol_type = other DNA
caagcagaagacggcatacgagatccactcgtgactggagttcagacgtgtgctcttccgatctctttaggggcgtccttttcggacac

SEQ ID NO: 51 - DNA, 89 bp, synthetic construct
  source 1..89: mol_type = other DNA
caagcagaagacggcatacgagatatcagtgtgactggagttcagacgtgtgctcttccgatctctttaggggcgtccttttcggacac

SEQ ID NO: 52 - DNA, 89 bp, synthetic construct
  source 1..89: mol_type = other DNA
caagcagaagacggcatacgagataggaatgtgactggagttcagacgtgtgctcttccgatctctttaggggcgtccttttcggacac

SEQ ID NO: 53 - DNA, 66 bp, synthetic construct
  source 1..66: mol_type = other DNA
gagccgaggccagacactctacataaccggtnnnnnnnnnaacaggcctattgattggaaagtatg

SEQ ID NO: 54 - DNA, 50 bp, synthetic construct
  source 1..50: mol_type = other DNA
gattaacctgatagatctctcgacttgggcaacaaaagtgaaactccatc

SEQ ID NO: 55 - DNA, 10 bp, synthetic construct
  source 1..10: mol_type = other DNA
acaggtccga

SEQ ID NO: 56 - DNA, 78 bp, synthetic construct
  source 1..78: mol_type = other DNA
atgatacggcgaccaccgagatctacactctttccctaccgacgctcttccgatctggatatcaagctggacatcacc

SEQ ID NO: 57 - DNA, 87 bp, synthetic construct
  source 1..87: mol_type = other DNA
caagcagaagacggcatacgagatcgtgatgtgactggagttcagacgtgtgctcttccgatctagacccacaattcgttgacatac

SEQ ID NO: 58 - DNA, 87 bp, synthetic construct
  source 1..87: mol_type = other DNA
caagcagaagacggcatacgagatacatcggtgactggagttcagacgtgtgctcttccgatctagacccacaattcgttgacatac

SEQ ID NO: 59 - DNA, 87 bp, synthetic construct
  source 1..87: mol_type = other DNA
caagcagaagacggcatacgagatgcctaagtgactggagttcagacgtgtgctcttccgatctagacccacaattcgttgacatac

SEQ ID NO: 60 - DNA, 87 bp, synthetic construct
  source 1..87: mol_type = other DNA
caagcagaagacggcatacgagattggtcagtgactggagttcagacgtgtgctcttccgatctagacccacaattcgttgacatac

SEQ ID NO: 61 - DNA, 87 bp, synthetic construct
  source 1..87: mol_type = other DNA
caagcagaagacggcatacgagatcactgtgtgactggagttcagacgtgtgctcttccgatctagacccacaattcgttgacatac

SEQ ID NO: 62 - DNA, 87 bp, synthetic construct
  source 1..87: mol_type = other DNA
caagcagaagacggcatacgagatattggcgtgactggagttcagacgtgtgctcttccgatctagacccacaattcgttgacatac

SEQ ID NO: 63 - DNA, 87 bp, synthetic construct
  source 1..87: mol_type = other DNA
caagcagaagacggcatacgagatgatctggtgactggagttcagacgtgtgctcttccgatctagacccacaattcgttgacatac

SEQ ID NO: 64 - DNA, 87 bp, synthetic construct
  source 1..87: mol_type = other DNA
caagcagaagacggcatacgagattcaagtgtgactggagttcagacgtgtgctcttccgatctagacccacaattcgttgacatac

SEQ ID NO: 65 - DNA, 87 bp, synthetic construct
  source 1..87: mol_type = other DNA
caagcagaagacggcatacgagatctgatcgtgactggagttcagacgtgtgctcttccgatctagacccacaattcgttgacatac

SEQ ID NO: 66 - DNA, 87 bp, synthetic construct
  source 1..87: mol_type = other DNA
caagcagaagacggcatacgagataagctagtgactggagttcagacgtgtgctcttccgatctagacccacaattcgttgacatac

SEQ ID NO: 67 - DNA, 87 bp, synthetic construct
  source 1..87: mol_type = other DNA
caagcagaagacggcatacgagatgtagccgtgactggagttcagacgtgtgctcttccgatctagacccacaattcgttgacatac

SEQ ID NO: 68 - DNA, 87 bp, synthetic construct
  source 1..87: mol_type = other DNA
caagcagaagacggcatacgagattacaaggtgactggagttcagacgtgtgctcttccgatctagacccacaattcgttgacatac

SEQ ID NO: 69 - DNA, 87 bp, synthetic construct
  source 1..87: mol_type = other DNA
caagcagaagacggcatacgagatttgactgtgactggagttcagacgtgtgctcttccgatctagacccacaattcgttgacatac

SEQ ID NO: 70 - DNA, 87 bp, synthetic construct
  source 1..87: mol_type = other DNA
caagcagaagacggcatacgagatggaactgtgactggagttcagacgtgtgctcttccgatctagacccacaattcgttgacatac

SEQ ID NO: 71 - DNA, 87 bp, synthetic construct
  source 1..87: mol_type = other DNA
caagcagaagacggcatacgagattgacatgtgactggagttcagacgtgtgctcttccgatctagacccacaattcgttgacatac

SEQ ID NO: 72 - DNA, 89 bp, synthetic construct
  source 1..89: mol_type = other DNA
caagcagaagacggcatacgagatctgatcgtgactggagttcagacgtgtgctcttccgatctctttaggggcgtccttttcggacac

SEQ ID NO: 73 - DNA, 89 bp, synthetic construct
  source 1..89: mol_type = other DNA
caagcagaagacggcatacgagataagctagtgactggagttcagacgtgtgctcttccgatctctttaggggcgtccttttcggacac

SEQ ID NO: 74 - DNA, 23 bp, synthetic construct
  source 1..23: mol_type = other DNA
cagtgaagctgacataccagggc

SEQ ID NO: 75 - DNA, 22 bp, synthetic construct
  source 1..22: mol_type = other DNA
attgttgttgatgagtcgctgc

SEQ ID NO: 76 - DNA, 78 bp, synthetic construct
  source 1..78: mol_type = other DNA
aatgatacggcgaccaccgagatctacactctttccctacacgacgctcttccgatctttggaacaggatgggtgtcc

SEQ ID NO: 77 - DNA, 84 bp, synthetic construct
  source 1..84: mol_type = other DNA
caagcagaagacggcatacgagatcactgtgtgactggagttcagacgtgtgctcttccgatctggtggtgatgactctgtcgc

SEQ ID NO: 78 - DNA, 84 bp, synthetic construct
  source 1..84: mol_type = other DNA
caagcagaagacggcatacgagattggtcagtgactggagttcagacgtgtgctcttccgatctggtggtgatgactctgtcgc

SEQ ID NO: 79 - DNA, 84 bp, synthetic construct
  source 1..84: mol_type = other DNA
caagcagaagacggcatacgagatcgtgatgtgactggagttcagacgtgtgctcttccgatctggtggtgatgactctgtcgc

SEQ ID NO: 80 - DNA, 84 bp, synthetic construct
  source 1..84: mol_type = other DNA
caagcagaagacggcatacgagatgcctaagtgactggagttcagacgtgtgctcttccgatctggtggtgatgactctgtcgc

SEQ ID NO: 81 - DNA, 84 bp, synthetic construct
  source 1..84: mol_type = other DNA
caagcagaagacggcatacgagattcaagtgtgactggagttcagacgtgtgctcttccgatctggtggtgatgactctgtcgc

SEQ ID NO: 82 - DNA, 84 bp, synthetic construct
  source 1..84: mol_type = other DNA
caagcagaagacggcatacgagatctgatcgtgactggagttcagacgtgtgctcttccgatctggtggtgatgactctgtcgc

SEQ ID NO: 83 - DNA, 84 bp, synthetic construct
  source 1..84: mol_type = other DNA
caagcagaagacggcatacgagatattggcgtgactggagttcagacgtgtgctcttccgatctggtggtgatgactctgtcgc

SEQ ID NO: 84 - DNA, 84 bp, synthetic construct
  source 1..84: mol_type = other DNA
caagcagaagacggcatacgagataagctagtgactggagttcagacgtgtgctcttccgatctggtggtgatgactctgtcgc